(19) United States
(12) Patent Application Publication
(10) Pub. No.: US 2003/0207800 A1
(43) Pub. Date: Nov. 6, 2003

(54) Proteins, polynucleotides encoding them and methods of using the same

(76) Inventors: Uriel M. Malyankar, Branford, CT (US); Suresh G. Shenoy, Branford, CT (US); Kimberly A. Spytek, New Haven, CT (US); Bryan D. Zerhusen, Branford, CT (US); Meera Patturajan, Branford, CT (US); Xiaojia (Sasha) Guo, Branford, CT (US); Ramesh Kekuda, Stamford, CT (US); Esha A. Gangolli, Madison, CT (US); Richard A. Shimkets, West Haven, CT (US); Raymond J. Taupier JR., East Haven, CT (US); Li Li, Branford, CT (US); Muralidhara Padigaru, Branford, CT (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 10/015,115

(22) Filed: Nov. 13, 2001

Related U.S. Application Data
(60) Provisional application No. 60/248,153, filed on Nov. 13, 2000.
(60) Provisional application No. 60/249,598, filed on Nov. 17, 2000.
(60) Provisional application No. 60/164,240, filed on Nov. 8, 1999.
(60) Provisional application No. 60/266,127, filed on Feb. 2, 2001.
(60) Provisional application No. 60/269,562, filed on Feb. 16, 2001.
(60) Provisional application No. 60/304,348, filed on Jul. 10, 2001.
(60) Provisional application No. 60/309,261, filed on Jul. 31, 2001.
(60) Provisional application No. 60/313,283, filed on Aug. 17, 2001.

(51) Int. Cl. A61K 38/17; C12P 21/02; C12N 5/06; C07K 14/435; C07H 21/04

(52) U.S. Cl. 514/12; 435/69.1; 530/350; 536/23.5; 435/320.1; 435/325

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority to U.S. Ser. No. 60/248,153, filed Nov. 13, 2000; U.S. Ser. No. 60/249,598, filed Nov. 17, 2000; U.S. Ser. No. 60/264,240, filed Jan. 26, 2001; U.S. Ser. No. 60/266,127, filed Feb. 2, 2001; U.S. Ser. No. 60/269,562, filed Feb. 16, 2001; U.S. Ser. No. 60/304,348, filed Jul. 10, 2001; U.S. Ser. No. 60/309,261, filed Jul. 31, 2001, and U.S. Ser. No. 60/313,283, filed Aug. 17, 2001 each of which is incorporated by reference in its entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The present invention is based in part on nucleic acids encoding proteins that are new members of the following protein families: Membrane protein/neuropilin/metalloproteinase-like protein-like, Fibrillin-like, KIAA1589-like, WD 40 motif-like, Opioid Bing Cell Adhesion Molecule-like, Triacylglycerol lipase-like, IGE Receptor Beta Subunit-like, Munc 18-like, Immunoglobulin-like and Type II Cytokeratin-like. More particularly, the invention relates to nucleic acids encoding novel polypeptides, as well as vectors, host cells, antibodies, and recombinant methods for producing these nucleic acids and polypeptides.

Neuropilins play an active role in angiogenesis as they are receptors for VEGF and may regulate VEGF-induced angiogenesis. Neuropilins are also expressed by tumor cells and may play a role in tumor angiogenesis. Neuropilins also play a role in axon guidance as they bind to semaphorins and and in combination with plexins regulate the signal transduction events in neurons. Hence neuropilin-like molecules play an important role in embryonic development and the dedifferention events seen in cancer. These molecules probably play an important role in regulation of angiogenesis, cancer, development and neurological conditions. The MAM domain is characteristic of the extracellular region of membrane associated proteins such as meprin (a cell surface glycoprotein); A5 antigen (a developmentally-regulated cell surface protein); and receptor-like tyrosine protein phosphatase. These proteins although functionally diverse; resemble receptors comprising a signal peptide, an N-terminal extracellular domain, a single transmembrane domain and an intracellular domain. The MAM domain might play a role in cell adhesion.

Fibrillin is a very large molecule whose primary structure is now known from the cloning and sequencing of 10 kb of cDNA. Fibrillin is the major component of extracellular microfibrils and is widely distributed in connective tissue throughout the body. Mutations in the fibrillin-1 (FBN1) gene, on chromosome 15q21.1, have been found to cause Marfan syndrome, a dominantly inherited disorder characterised by clinically variable skeletal, ocular, and cardiovascular abnormalities. Fibrillin-1 mutations have also been found in several other related connective tissue disorders, such as severe neonatal Marfan syndrome, dominant ectopia lentis, familial ascending aortic aneurysm, isolated skeletal features of Marfan syndrome, and Shprintzen-Goldberg syndrome (PMID: 9401003, PMID: 8575254, PMID: 7584608).

Opioid binding cell adhesion molecules (OBCAMs) are members of the cell adhesion molecule family with homology to the immunoglobulin protein superfamily. OBCAMs are seen both in the developing nervous system as well as in the mature adult brain. They may play a role in neuronal outgrowth and development, probably by modulating cell-cell interactions. In addition, OBCAMs are known to affect the regulation and functioning of opioid receptors. Chronic morphine treatment downregulates the expression of at least one member of this family. Therefore, these proteins could mediate long-term effects on brain function by opioid usage and may be used as a therapeutic in that context.

The assimilation of dietary fats into the body requires that they be digested by lipases. One lipase, pancreatic triglyceride lipase, is essential for the efficient digestion of dietary fats. Pancreatic triglyceride lipase is the archetype of the lipase gene family that includes two homologues of pancreatic triglyceride lipase, pancreatic lipase-related proteins 1 and 2. The cDNA sequences encoding pancreatic triglyceride lipase and the related proteins have been described. Furthermore, the tertiary structure of human pancreatic triglyceride lipase has been determined alone and in a complex with colipase, a pancreatic protein required for lipase activity in the duodenum (Lowe, Molecular mechanisms of rat and human pancreatic triglyceride lipases. J Nutr 127(4):549-57, 1997).

The high-affinity receptor for immunoglobulin E, Fc epsilon RI, is found exclusively on mast cells and basophils. When multivalent allergens bind to the receptor-bound IgE, the consequent aggregation of the receptors leads to the release of mediators responsible for allergic symptoms. In rodents Fc epsilon RI is a tetrameric complex of non-covalently attached subunits: one IgE-binding alpha subunit, one beta subunit and a dimer of disulphide-linked gamma subunits (Blank et al., Complete structure and expression in transfected cells of high affinity IgE receptor. Nature 337(6203): 187-9, 1989). IgE receptors of mast cells, Fc epsilon RI, localize to coated pits and internalize after cross-linking. Studies have investigated whether any one of the receptor's four distinctive cytoplasmic domains regulates these phenomena. The conclusion of these studies was that no single cytoplasmic domain of the Fc epsilon RI uniquely controls its ligand-induced localization to coated pits and internalization (Mao et al., Effects of subunit mutation on the localization to coated pits and internalization of cross-linked IgE-receptor complexes. J Immunol 151(5):2760-74, 1993).

Sec1 family proteins are regulators of diverse exocytic processes, from yeast to man. Three mammalian homologues, Munc 18-1, -2, and -3 have been described. The Munc 18-2 gene comprises 19 exons whose sizes range from 50 to 158 bp, with a total gene size of approximately 11 kb. A single transcript of 2.1 kb is expressed in multiple non-neuronal murine tissues. Munc 18-2 has a striking resemblance to Munc 18-1 in structure despite only 60% sequence identity, suggesting a recent gene duplication event (Agrawal et al., Gene structure and promoter function of murine Munc 18-2, a non-neuronal exocytic Sec1 homolog. Biochem Biophys Res Commun 276(3):817-22, 2000).

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9 and NOV10 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1, 3, 5, 7, 9, 11, 13, 15, 17, 19, 21, 23, 25, 27, 29, 31, 33, 35, 37, 39, 41, 43, 45 and 47. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, 24, 26, 28, 30, 32, 34, 36, 38, 40, 42, 44, 46 and 48. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, 25, 27, 29, 31, 33, 35, 37, 39, 41, 43, 45and 47.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, 25, 27, 29, 31, 33, 35, 37, 39, 41, 43, 45 and 47) or a complement of said oligonucleotide. Also included in the invention are substantially purified NOVX polypeptides (SEQ ID NOS: 2, 4, 6, 8, 10, 12, 14, 16, 18, 20, 22, 24, 26, 28, 30, 32, 34, 36, 38, 40, 42, 44, 46 and 48). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., trauma, regeneration (in vitro and in vivo), viral/bacterial/parasitic infections, Von Hippel-Lindau (VHL) syndrome, Alzheimer's disease, stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, behavioral disorders, addiction, anxiety, pain, actinic keratosis, acne, hair growth diseases, allopecia, pigmentation disorders, endocrine disorders, connective tissue disorders, such as severe neonatal Marfan syndrome, dominant ectopia lentis, familial ascending aortic aneurysm, isolated skeletal features of Marfan syndrome, Shprintzen-Goldberg syndrome, genodermatoses, contractural arachnodactyly, inflammatory disorders such as osteo- and rheumatoid-arthritis, inflammatory bowel disease, Crohn's disease; immunological disorders, AIDS; cancers including but not limited to lung cancer, colon cancer, Neoplasm; adenocarcinoma; lymphoma; prostate cancer; uterus cancer, leukemia or pancreatic cancer; blood disorders; asthma; psoriasis; vascular disorders, hypertension, skin disorders, renal disorders including Alport syndrome, immunological disorders, tissue injury, fibrosis disorders, bone diseases, Ehlers-Danlos syndrome type VI, VII, type IV, S-linked cutis laxa and Ehlers-Danlos syndrome type V, osteogenesis imperfecta, Neurologic diseases, Brain and/or autoimmune disorders like encephalomyelitis, neurodegenerative disorders, immune disorders, hematopoietic disorders, muscle disorders, inflammation and wound repair, bacterial, fungal, protozoal and viral infections (particularly infections caused by HIV-1 or HIV-2), pain, acute heart failure, hypotension, hypertension, urinary retention, osteoporosis, Treatment of Albright Hereditary Ostoeodystrophy, angina pectoris, myocardial infarction, ulcers, benign prostatic hypertrophy, arthrogryposis multiplex congenita, osteogenesis imperfecta, keratoconus, scoliosis, duodenal atresia, esophageal atresia, intestinal malrotation, Pancreatitis, Obesity Systemic lupus erythematosus, Autoimmune disease, Emphysema, Scleroderma, allergy, ARDS, Neuroprotection, Fertility Myasthenia gravis, Diabetes, obesity, Growth and reproductive disorders Hemophilia, Hypercoagulation, Idiopathic thrombocytopenic purpura, Immunodeficiencies, Graft vesus host, Adrenoleukodystrophy, Congenital Adrenal Hyperplasia, Endometriosis, Xerostomia, Ulcers, Cirrhosis, Transplantation, Diverticular disease, Hirschsprung's disease, Appendicitis, Arthritis, Ankylosing spondylitis, Tendinitis, Renal artery stenosis, Interstitial nephritis, Glomerulonephritis, Polycystic kidney disease, erythematosus, Renal tubular acidosis, IgA nephropathy, anorexia, bulimia, psychotic disorders, including anxiety, schizophrenia, manic depression, delirium, dementia, severe mental retardation and dyskinesias, such as Huntington's disease and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | | | |
|---|---|---|---|---|
| | | SEQ ID | | |
| | | NO | | |
| NOVX | | (nucleic | SEQ ID NO | |
| Assignment | Internal Identification | acid) | (polypeptide) | Homology |

| | | | | |
|---|---|---|---|---|
| 1a | SC40376139 | 1 | 2 | Membrane Protein/ |
| | | | | Neuropilin/Metalloproteinase-like |
| 1b | CG55014-02 | 3 | 4 | Membrane Protein/ |
| | | | | Neuropilin/Metalloproteinase-like |
| 2a | GMAC022146_A | 5 | 6 | Fibrillin-like |
| 2b | 153568997 | 7 | 8 | Fibrillin-like |
| 2c | CG88987-01 | 9 | 10 | Fibrillin-like |
| 2d | CG88987-02 | 11 | 12 | Fibrillin-like |
| 2e | CG88987-03 | 13 | 14 | Fibrillin-like |
| 2f | CG88987-05 | 15 | 16 | Fibrillin-like |
| 3 | GSAL442663.1_A | 17 | 18 | K1AA1589-like |
| 4 | GSAL442663.1_B | 19 | 20 | WD 40 motif-like |
| 5a | 139785504 | 21 | 22 | Oploid Bing Cell Adhesion |
| | | | | molecule-like |
| 5b | 139785504_da1 | 23 | 24 | Opioid Bing Cell Adhesion |
| | | | | molecule-like |
| 5c | CG51027-03 | 25 | 26 | Opioid Bing Cell Adhesion |
| | | | | molecule-like |
| 5d | CG51027-05 | 27 | 28 | Opioid Bing Cell Adhesion |
| | | | molecule-like | |
| 6a | SC122982104_A | 29 | 30 | Triacylglycerol lipase-like |
| 6b | CG58608-02 | 31 | 32 | Triacylglycerol lipase-like |
| 7a | SC126624027_A | 33 | 34 | IGE Receptor Beta Subunit-like |
| 7b | CG55760-02 | 35 | 36 | IGE Receptor Beta Subunit-like |
| 8 | SC138745558_A | 37 | 38 | Munc 18-like |
| 9a | SC138673511_A | 39 | 40 | Immunoglobulin-like |
| 9b | CG106625-02 | 41 | 42 | Immunoglobulin-like |
| 10a | GSAC055715.12_D | 43 | 44 | Type II Cytokeratin-like |
| 10b | GSAC055715_C | 45 | 46 | Type II Cytokeratin-like |
| 10c | GSAC055715_B | 47 | 48 | Type II Cytokeratin-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to a Membrane Protein/Neuropilin/Metalloproteinase-like family of proteins. Thus, the NOVI nucleic acids, polypeptides, antibodies and related compounds according to the invention will be useful in therapeutic and diagnostic applications implicated in, for example; cancer, trauma, regeneration (in vitro and in vivo), viral/bacterial/parasitic infections, Von Hippel-Lindau (VHL) syndrome, Alzheimer's disease, stroke, Parkinson's disease, Huntington's disease, Cerebral palsy, Multiple sclerosis, hair growth diseases, endocrine disorders and/or other pathologies/disorders.

NOV2 is homologous to a Fibrillin-like family of proteins. Thus NOV2 nucleic acids, polypeptides, antibodies and related compounds according to the invention will be useful in therapeutic and diagnostic applications implicated in, for example; connective tissue disorders, such as severe neonatal Marfan syndrome, Marfan syndrome, inflammatory disorders such as osteo- and rheumatoid-arthritis, inflammatory bowel disease, Crohn's disease; immunological disorders, AIDS; cancers including but not limited to lung cancer, colon cancer, Neoplasm; adenocarcinoma; lymphoma; prostate cancer; uterus cancer, leukemia or pancreatic cancer; Neurologic diseases, neurodegenerative disorders, Alzheimer's Disease, Parkinson's Disorder, immune disorders, inflammation, Parkinson's disease, osteoporosis, multiple sclerosis; angina pectoris, myocardial infarction, benign prostatic and/or other pathologies/disorders.

NOV3 is homologous to a family of KIAA1589-like proteins. Thus, the NOV3 nucleic acids and polypeptides, antibodies and related compounds according to the invention will be useful in therapeutic and diagnostic applications implicated in, for example: inflammatory disorders such as osteo- and rheumatoid-arthritis, inflammatory bowel disease; immunological disorders, AIDS; cancers including but not limited to lung cancer, colon cancer, leukemia or pancreatic cancer.; skin disorders, Neurologic diseases, neurodegenerative disorders, Alzheimer's Disease, Parkinson's Disorder, immune disorders, and hematopoietic disorders, endocrine diseases, muscle disorders, inflammation and wound repair; multiple sclerosis; angina pectoris, myocardial infarction, ulcers, benign prostatic hypertrophy and/or other pathologies/disorders.

NOV4 is homologous to the WD 40 motif-like family of proteins. Thus, NOV4 nucleic acids, polypeptides, antibodies and related compounds according to the invention will be useful in therapeutic and diagnostic applications implicated, for example; cancer, Diabetes, Von Hippel-Lindau (VHL) syndrome, Obesity Systemic lupus erythematosus, Autoimmune disease, Asthma, Von Hippel-Lindau (VHL) syndrome, Alzheimer's disease, Stroke, Parkinson's disease, Huntington's disease, Cerebral palsy, Epilepsy, Lesch-Nyhan syndrome, Multiple sclerosis, Neuroprotection Fertility Myasthenia gravis, Neuroprotection Endocrine dysfunctions, obesity, Immunodeficiencies, Graft vesus host and/or other pathologies/disorders.

NOV5 is homologous to the Opioid Bing Cell Adhesion Molecule-like protein family. Thus NOV5 nucleic acids, polypeptides, antibodies and related compounds according to the invention will be useful in therapeutic and diagnostic applications implicated in, for example: Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration and/or other pathologies/disorders.

NOV6 is homologous to the Triacylglycerol lipase-like family of proteins. Thus NOV6 nucleic acids, polypeptides, antibodies and related compounds according to the invention will be useful in therapeutic and diagnostic applications implicated in, for example: Diabetes, Von Hippel-Lindau (VHL) syndrome, Obesity, Von Hippel-Lindau (VHL) syndrome, Transplantation, Inflammatory bowel disease, Arthritis, Alzheimer's disease, Stroke, Parkinson's disease, Huntington's disease, Endocrine dysfunctions, Diabetes, Growth and reproductive disorders, Multiple sclerosis, Autoimmune disease, Acne, Hair growth, allopecia, Hypercalceimia, Lesch-Nyhan syndrome and/or other pathologies/disorders.

NOV7 is homologous to members of the IGE Receptor Beta Subunit-like family of proteins. Thus, the NOV7 nucleic acids, polypeptides, antibodies and related compounds according to the invention will be useful in therapeutic and diagnostic applications implicated in, for example; inflammatory disorders such as osteo- and rheumatoid-arthritis, inflammatory bowel disease, AIDS; cancers including but not limited to lung cancer, colon cancer, leukemia or pancreatic cancer.; asthma; psoriasis; skin disorders, renal disorders immunological disorders, bone diseases, neurodegenerative disorders, Alzheimer's Disease, Parkinson's Disorder, endocrine diseases, muscle disorders, wound repair, bacterial, fungal, protozoal and viral infections (particularly infections caused by HIV-1 or HIV-2), anorexia, bulimia, asthma, osteoporosis, multiple sclerosis; benign prostatic hypertrophy, schizophrenia, manic depression, delirium, dementia, severe mental retardation and dyskinesias, such as Huntington's disease, Gilles de la Tourette syndrome and/or other pathologies/disorders.

NOV8 is homologous to the Munc 18-like family of proteins. Thus, NOV8 nucleic acids and polypeptides, antibodies and related compounds according to the invention will be useful in therapeutic and diagnostic applications implicated in, for example; inflammatory disorders such as osteo- and rheumatoid-arthritis, inflammatory bowel disease, Crohn's disease; immunological disorders, AIDS; cancers including but not limited to lung cancer, colon cancer, leukemia or pancreatic cancer.; psoriasis; neurodegenerative disorders, Alzheimer's Disease, Parkinson's Disorder, immune disorders, hematopoietic disorders, endocrine diseases, muscle disorders, wound repair, bacterial, fungal, protozoal and viral infections (particularly infections caused by HIV-1 or HIV-2), Parkinson's disease, acute heart failure, hypotension, hypertension, osteoporosis, multiple sclerosis; angina pectoris, myocardial infarction, psychotic and neurological disorders, including anxiety, schizophrenia, manic depression, delirium, dementia, severe mental retardation and dyskinesias, such as Huntington's disease, Gilles de la Tourette syndrome and/or other pathologies/disorders.

NOV9 is homologous to members of the Immunoglobulin-like family of proteins. Thus, the NOV9 nucleic acids, polypeptides, antibodies and related compounds according to the invention will be useful in therapeutic and diagnostic applications implicated in, for example; inflammatory disorders such as osteo- and rheumatoid-arthritis, inflammatory bowel disease, Crohn's disease; immunological disorders, AIDS; cancers including but not limited to lung cancer, colon cancer, leukemia or pancreatic cancer.; psoriasis; neurodegenerative disorders, Alzheimer's Disease, Parkinson's Disorder, immune disorders, hematopoietic disorders, endocrine diseases, muscle disorders, wound repair, bacterial, fungal, protozoal and viral infections (particularly infections caused by HIV-1 or HIV-2), Parkinson's disease, acute heart failure, hypotension, hypertension, osteoporosis, multiple sclerosis; angina pectoris, myocardial infarction, psychotic and neurological disorders, including anxiety, schizophrenia, manic depression, delirium, dementia, severe mental retardation and dyskinesias, such as Huntington's disease, Gilles de la Tourette syndrome and/or other pathologies/disorders.

NOV10 is homologous to members of the Type II Cytokeratin-like family of proteins. Thus, the NOV10 nucleic acids, polypeptides, antibodies and related compounds according to the invention will be useful in therapeutic and diagnostic applications implicated in, for example; inflammatory and infectious diseases such as AIDS; Neurologic diseases, neurodegenerative disorders, Alzheimer's Disease, Parkinson's Disorder, immune disorders, and hematopoietic disorders, endocrine diseases, muscle disorders, wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease and/or other pathologies/disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes two novel Membrane protein/neuropilin/metalloproteinase-like proteins disclosed below. The disclosed proteins have been named NOV1a and NOV1b.

NOV1a

A disclosed NOV1a nucleic acid of 1668 nucleotides (also referred to as SC40376139) encoding a novel Membrane protein/neuropilin/metalloproteinase-like protein is shown in Table 1A. An open reading frame was identified beginning with an ATG initiation codon at nucleotides 7-9 and ending with a TGA codon at nucleotides 1453-1455. Putative untranslated regions upstream from the initiation codon and downstream from the termination codon are underlined in Table 1A, and the start and stop codons are in bold letters.

TABLE 1A

NOV1a Nucleotide Sequence (SEQ ID NO:1).
GGATCAATGCAAATGGAGAGTTATGATGGAACACTGAGGATTGTGAATGTATCTAGGGAAATGTCAGGAATGTACAGATGT
CAGACCAGCCAATACAATGGATTTAACGTGAAACCAAGGGAAGCCTTGGTGCAGCTCATCGTTCAGTATCCCCCTGCAGTG
GAACCAGCATTCTTGGAAATCCGGCAAGGACAGGATCGAAGTGTCACTATGAGTTGCAGAGTACTGAGAGCCTATCCAATA
CGGGTGCTGACCTATGAGTGGCGCTTGGGCAATAAATTATTACGGACGGGTCAATTTGACTCTCAGGAATACACAGAGTAC
GCTGTGAAGAGTCTTTCCAATGAAAACTATGGGGTTTATAACTGTAGCATCATAAATGAAGCTGGAGCTGGGAGATGCAGC
TTTCTTGTTACAGGTGGAAAGGCCTATGCTCCAGAATTCTATTATGATACCTACAATCCAGTATGGCAGAACAGACACCGT
GTTTATTCTTACAGTCTACAGTGGACACAGATGAATCCTGATGCAGTGGATCGGATTGTTGCATACCGGTTGGGCATCAGG
CAGGCTGGACAGCAGCGCTGGTGGGAGCAGGAGATTAAAATAAATGGGAATATTCAAAAGGGAGAATTAATTACATATAAC
TTGACAGAGCTAATTAAACCAGAAGCTTATGAAGTCCGACTGACTCCTCTCACCAAATTTGGTGAAGGAGATTCAACAATT
CGTGTGATCAAATATAGTCCTGTAAATCCTCATTTGAGTGAATTTCATTGTGGATTTGAAGATGGTAATATTTGTTTGTTC
ACTCAAGATGATACAGATAATTTTGACTGGACAAAGCAAAGTACAGCAACAAGAAATACAAAATATACTCCTAATACAGGA
CCTAATGCTGACCGTAGTGGCTCCAAAGAAGGTTTTTATATGTACATTGAGACATCACGACCCAGATTGGAAGGCGAAAAG
GCTCGACTTCTCAGCCCTGTTTTCAGCATAGCTCCAGCCCTGTTTTCAGCAAGACTTCTCAGCCCTGTTTTCAGCATAGCT
CCCAAAAACCCTTATGGACCCACAAACACTGCATATTGTTTCAGCTTCTTTTATCACATGTATGGACAACATATAGGTGTC
TTAAATGTTTATCTACGTTTGAAAGGGCAAACAACAATAGAGAATCCACTGTGGTCTTCAAGTGGGAATAAAGGACAAAGA
TGGAATGAGGCTCATGTTAATATATACCCAATTACTTCATTTCAGCTCATTTTTGAAGGTATCCGAGGTCCTGGAATAGAA
GGTGACATTGCTATTGATGATGTATCAATTGCAGAAGGAGAATGTGCAAAACAAGACCTAGCAACTAAGAATTCCGTTGAT
GGTGCTGTTGGGATTTTGGTTCATATATGGCTTTTTCCCATTATCGTCCTCATCTCTATCTTAAGTCCTCGAAGGTGACCT
TATCCTGGCAGAGGCTATAAAAGATTCACCAGGCACTGGCATGAAGAAAGAGTCTTTGTAAATGGACATTGAACAAACAAA
CTACCAAAGATTCCTCCACTGACTACTGACTCAAAAATAAAATAATAAAAACAAATTTTTTTAAGCACTGGGGATAAAAAG
ACATCATGGAAGTATAACTTATTCAGACTAAACATAAAGATAATCTGA

The NOV1a nucleic acid sequence maps to chromosome 14 and has 1030 of 1128 bases (91%) identical to a Macaca fascicularis brain mRNA (gb:GENBANK-ID: AB047834|acc:AB047834.1) (E=3.2e⁻²⁰¹). Similiarity information was assessed using public nucleotide databases including all GenBank databases and the GeneSeq patent database. Chromosome information was assigned using OMIM and the electronic northern tool from Curatools to derive the the chromosomal mapping of the SeqCalling assemblies, Genomic clones, and/or EST sequences that were included in the invention.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1a BLAST analysis, e.g., Macaca fascicularis brain mRNA, matched the Query NOV1a sequence purely by chance is 3.2e⁻²⁰¹. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNN”) or the letter “X” in protein sequences (e.g., “XXX”). Low-complexity regions can result in high scores that reflect compositional bias rather than significant position-by-position alignment. Wootton and Federhen, Methods Enzymol 266:554-571, 1996.

The disclosed NOV1a polypeptide (SEQ ID NO: 2) encoded by SEQ ID NO: 1 has 482 amino acid residues and is presented in Table 1B using the one-letter amino acid code. Signal P, Psort and/or Hydropathy results predict that NOV1 a does not contain a signal peptide and is likely to be localized to the plasma membrane with a certainty of 0.7000.

TABLE 1B

Encoded NOV1a protein sequence (SEQ ID NO:2).
MQMESYDGTLRIVNVSREMSGMYRCQTSQYNGFNVKPREALVQLIVQYPPAVEPAFLEIRQGQDRSVTMSCRVLRAYPIRV
LTYEWRLGNKLLRTGQFDSQEYTEYAVKSLSNENYGVYNCSIINEAGAGRCSFLVTGGKAYAPEFYYDTYNPVWQNRHRVY
SYSLQWTQMNPDAVDRIVAYRLGIRQAGQQRWWEQEIKINGNIQKGELITYNLTELIKPEAYEVRLTPLTKFGEGDSTIRV
IKYSPVNPHLSEFHCGFEDGNICLFTQDDTDNFDWTKQSTATRNTKYTPNTGPNADRSGSKEGFYMYIETSRPRLEGEKAR
LLSPVFSIAPALFSARLLSPVFSIAPKNPYGPTNTAYCFSFFYHMYGQHIGVLNVYLRLKGQTTIENPLWSSSGNKGQRWN
EAHVNIYPITSFQLIFEGIRGPGIEGDIAIDDVSIAEGECAKQDLATKNSVDGAVGILVHIWLFPIIVLISILSPRR

The NOV1a amino acid sequence has 445 of 464 amino acid residues (95%) identical to, and 445 of 464 amino acid residues (95%) similar to, a Macaca fascicularis 448 amino acid residue hypothetical 51.2 kda protein (ptnr:TREMBLNEW-ACC:BAB12260) (E=1.0e⁻²⁴¹). The NOV1a amino acid sequence also has 71 of 197 amino acid residues (36%) identical to, and 108 of 197 amino acid residues (55%) similar to, a Xenopus laevis 928 amino acid residue neuropilin-1 precursor protein (ptnr:SWISSNEW-ACC:P28824) (E=1.0e⁻¹⁷).

NOV1 a is predicted to be expressed in brain tissues because of the expression pattern of a closely related Macaca fascicularis brain cDNA, clone:QccE-16296 homolog (GENBANK-ID: gb:GENBANK-ID:AB047834|acc:AB047834.1).

NOV1b

A disclosed NOV1b nucleic acid of 1608 nucleotides (also referred to as CG55014-02) encoding a novel Membrane protein/neuropilin/metalloproteinase-like protein is shown in Table 1C. An open reading frame was identified beginning with an ATG initiation codon at nucleotides 6-8 and ending with a TGA codon at nucleotides 1404-1406. Putative untranslated regions upstream from the initiation codon and downstream from the termination codon are underlined in Table 1C, and the start and stop codons are in bold letters.

TABLE 1C

NOV1b Nucleotide Sequence (SEQ ID NO:3).
GATCAATGCAAATGGAGAGTTATGATGGAACACTGAGGATTGTGAATGTATCTAGGGAAATGTCAGGAATGTACAGATGTC
AGACCAGCCAATACAATGGATTTAACGTGAAACCAAGGGAAGCCTTGGTGCAGCTCATCGTTCAGTATCCCCCTGCAGTGG
AACCAGCATTCTTGGAAATCCGGCAAGGACAGGATCGAAGTGTCACTATGAGTTGCAGAGTACTGAGAGCCTATCCAATAC
GGGTGCTGACCTATGAGTGGCGCTTGGGCAATAAATTATTACGGACGGGTCAATTTGACTCTCAGGAATACACAGAGTACG
CTGTGAAGAGTCTTTCCAATGAAAACTATGGGGTTTATAACTGTAGCATCATAAATGAAGCTGGAGCTGGGAGATGCAGCT
TTCTTGTTACAGGAAAGGCCTATGCTCCAGAATTCTATTATGATACCTACAATCCAGTATGGCAGAACAGACACCGTGTTT
ATTCTTACAGTCTACAGTGGACACAGATGAATCCTGATGCAGTGGATCGGATTGTTGCATACCGGTTGGGCATCAGGCAGG
CTGGACAGCAGCGCTGGTGGGAGCAGGAGATTAAAATAAATGGGAATATTCAAAAGGGAGAATTAATTACATATAACTTGA
CAGAGCTAATTAAACCAGAAGCTTATGAAGTCCGACTGACTCCTCTCACCAAATTTGGTGAAGGAGATTCAACAATTCGTG
TGATCAAATATAGTGCTCCTGTAAATCCTCATTTGAGAGAATTTCATTGTGGATTTGAAGATGGTAATATTTGTTTGTTCA
CTCAAGATGATACAGATAATTTTGACTGGACAAAGCAAAGTACAGCAACAAGAAATACAAAATATACTCCTAATACAGGAC
CTAATGCTGACCGTAGTGGCTCCAAAGAAGGTTTTTATATGTACATTGAGACATCACGACCCAGATTGGAAGGCGAAAAGG
CTCGACTTCTCAGCCCTGTTTTCAGCATAGCTCCCAAAAACCCTTATGGACCCACAAACACTGCATATTGTTTCAGCTTCT
TTTATCACATGTATGGACAACATATAGGTGTCTTAAATGTTTATCTACGTTTGAAAGGGCAAACAACAATAGAGAATCCAC
TGTGGTCTTCAAGTGGGAATAAAGGACAAAGATGGAATGAGGCTCATGTTAATATATACCCAATTACTTCATTTCAGCTCA
TTTTTGAAGGTATCCGAGGTCCTGGAATAGAAGGTGACATTGCTATTGATGATGTATCAATTGCAGAAGGAGAATGTGCAA
AACAAGACCTAGCAACTAAGAATTCCGTTGATGGTGCTGTTGGGATTTTGGTTCATATATGGCTTTTTCCCATTATCGTCC
TCACCTCTATCTTAAGTCCTCGAAGGTGACCTTATCCTGGCAGAGGCTATAAAAGATTCACCAGGCACTGGCATGAAGAAA
GAGTCTTTGTAAATGGACATTGAACAAACAAACTACCAAAGATTCCTCCACTGACTACTGACTCAAAAATAAAATAATAAA
AACAAATTTTTTTAAGCACTGGGGATAAAAAGACATCATGGAAGTATAACTTATTCAGACTAAACATAA

The NOV1b nucleic acid sequence has 1538 of 1557 bases (98%) identical to a Macaca fascicularis brain mRNA (gb:GENBANK-ID:AB047834|acc:AB047834.1) (E=0.0).

The disclosed NOV1b polypeptide (SEQ ID NO: 4) encoded by SEQ ID NO: 3 has 466 amino acid residues and is presented in Table 1D using the one-letter amino acid code. Signal P, Psort and/or Hydropathy results predict that NOV1b does not contain a signal peptide and is likely to be localized to the plasma membrane with a certainty of 0.7000.

TABLE 1D

Encoded NOV1b protein sequence (SEQ ID NO:4).
MQMESYDGTLRIVNVSREMSGMYRCQTSQYNGFNVKPREALVQLIVQYPPAVEPAFLEIRQGQDRSVTMSCRVLRAYPIRV
LTYEWRLGNKLLRTGQFDSQEYTEYAVKSLSNENYGVYNCSIINEAGAGRCSFLVTGKAYAPEFYYDTYNPVWQNRHRVYS
YSLQWTQMNPDAVDRIVAYRLGIRQAGQQRWWEQEIKINGNIQKGELITYNLTELIKPEAYEVRLTPLTKFGEGDSTIRVI
KYSAPVNPHLREFHCGFEDGNICLFTQDDTDNFDWTKQSTATRNTKYTPNTGPNADRSGSKEGFYMYIETSRPRLEGEKAR
LLSPVFSIAPKNPYGPTNTAYCFSFFYHMYGQHIGVLNVYLRLKGQTTIENPLWSSSGNKGQRWNEAHVNIYPITSFQLIF
EGIRGPGIEGDIAIDDVSIAEGECAKQDLATKNSVDGAVGILVHIWLFPIIVLTSILSPRR

The NOV1b amino acid sequence has 446 of 448 amino acid residues (99%) identical to, and 446 of 448 amino acid residues (99%) similar to, a Macaca fascicularis 448 amino acid residue hypothetical 51.2 kda protein (ptnr:TREMBLNEW-ACC:BAB12260) (E=3.1e^{31 248}).

NOV1b is expressed in at least the following tissues: brain. This information was derived by determining the tissue sources of the sequences that were included in the invention including but not limited to SeqCalling sources, Public EST sources, Literature sources, and/or RACE sources. In addition, NOV1b is predicted to be expressed in the following tissues because of the expression pattern of a closely related Macaca fascicularis brain cDNA, clone:QccE-16296 homolog (GENBANK-ID: gb:GENBANK-ID:AB047834|acc:AB047834.1):Brain.

Possible small nucleotide polymorphisms (SNPs) found for NOV1b are listed in Tables 1E and 1F. Depth represents the number of clones covering the region of the SNP. The putative allele frequence (PAF) is the fraction of these clones containing the SNP. A dash, when shown, means that a base is not present. The sign “>” means “is changed to.”

TABLE 1E
SNPs

| Consensus | | Base | |
|---|---|---|---|
| Position | Depth | Change | PAF |
| 53 | 16 | T > C | N/A |
| 229 | 27 | A > G | N/A |

TABLE 1F
SNPs

| | | | Amino | |
|---|---|---|---|---|
| | Neucleotide | Base | Acid | Base |
| Variant | Position | Change | Position | Change |
| 13375763 | 58 | A > G | 18 | Glu > Gly |
| 13375764 | 87 | A > G | 28 | Ser > Gly |
| 13375765 | 49 | A > G | 115 | Asn > Ser |
| 13375766 | 400 | G > A | 132 | Cys > Tyr |

NOV1a and NOV1b are vary closely homologous as is shown in the amino acid alignment in Table 1G.

[image]

[image]

Homologies to any of the above NOV1 proteins will be shared by the other NOV1 proteins insofar as they are homologous to each other as shown above. Any reference to NOV1 is assumed to refer to both of the NOV1 proteins in general, unless otherwise noted.

NOV1a also has homology to the amino acid sequences shown in the BLASTP data listed in Table 1H.

TABLE 1H
BLAST results for NOV1a

| Gene Index/ | | Length | Identity | Positives | |
|---|---|---|---|---|---|
| Identifier | Protein/Organism | (aa) | (%) | (%) | Expect |
| gi\|9967117\|dbj\| | hypothetical | 448 | 445/465 | 445/465 | 0.0 |
| BAB12260.1\|(AB047834) | protein [Macaca | | (95%) | (95%) | |
| | fascicularis]) | | | | |
| gi\|7529597\|emb\| | dJ402N21.2 (novel | 273 | 126/246 | 166/246 | 2e−65 |
| CAB86653.1\|(AL049553) | protein with MAM | | (51%) | (67%) | |
| | domain) [Homo | | | | |
| | sapiens] | | | | |
| gi\|12836077\|dbj\| | putative [Mus | 267 | 125/248 | 166/248 | 3e−65 |
| BAB23491.1\|(AK004706) | musculus] | | (50%) | (66%) | |
| gi\|4929103\|gb\| | metalloproteinase | 496 | 68/209 | 100/209 | 8e−21 |
| AAD33860.1\|AF140020_1 | 2 [Hydra vulgaris] | | (32%) | (47%) | |
| (AF140020) | | | | | |
| gi\|15929977\|gb\| | Unknown (protein | 232 | 69/198 | 95/198 | 3e−20 |
| AAH15417.1\|AAH15417 | for MGC:21981) | | (34%) | (47%) | |
| (BC015417) | [Homo sapiens] | | | | |

The homology between these and other sequences is shown graphically in the ClustalW analysis shown in Table 1I. In the ClustalW alignment of the NOV1 a protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

The presence of identifiable domains in NOV1 a, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV1a, as disclosed in Tables 1J and 1K, were collected from the Conserved Domain Database (CDD) with Reverse Position Specific BLAST analyses. This BLAST analysis software samples domains found in the Smart and Pfam collections. For Tables 1J, 1K and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Tables 1 J and 1K lists the domain description from DOMAIN analysis results against NOV1a. This indicates that the NOV1a sequence has properties similar to those of other proteins known to contain these domains.

TABLE 1

Domain Analysis of NOV1a

(SEQ ID NO:54)

gnl|Smart|smart00137, MAN, Domain in meprin, A5, receptor protein
tyrosine phosphatase mu (and others); Likely to have an adhesive
function. Mutations in the meprin MAM domain affect noncovalent
associations within meprin oligomers. In receptor tyrosine phosphatase
mu-like molecules the MAM domain is important for homophilic cell-cell
interactions.
Length = 163 residues, 99.4% aligned
Score =154 bits (390), Expect = 9e-39

| | | | |
|---|---|---|---|
| NOV1a | 254 | SEFHCGFEDGNICLFTQDDTDNFDWTKQSTATRNTKYTPNTGPNADRSGSKEGFYMYIET | 313 |
| \|+ +\| \|\|+\|\| \| + \|\| \|+ \| + \|+\|\| \| \|\|\| \| + \| \|\|+ \|\| | | | |
| 00137 | 2 | SDGNCDFEEGNTCGWHQDSNDDGPWERVSSAT------RNDGPNRDHTT-GNGHYMFFET | 54 |
| NOV1a | 314 | SRPRLEGEKARLLSPVFSIAPALFSARLLSPVFSIAPKNPYGPTNTAYCFSFFYHMYGQH | 373 |
| \| \|+ \|\|\|\|\|\| + +\| +\|+\|+\|\|\| | | | |
| 00137 | 55 | S-SGKPGQTARLLSPPLY------------------------ENRSTHCLTFWYYMYGSG | 89 |
| NOV1a | 374 | IGVLNVYLRLKGQTTIENPLWSSSGNKGQRWNEAHVNIYPITS-FQLIFEGIRGPGIEGD | 432 |
| +\| \|\|\|\|+\|+ + \|\|\| \|\| +\| +\| +\| \| + \|\|++\|\|\| \|\| \| \| | | | |
| 00137 | 90 | VGTLNVYVRVNN-GPQDTLLWSRSGTQGGQWLQAEVALSTSPQPFQVVFEGTRGGGPSGY | 148 |
| NOV1a | 433 | IAIDDVSIAEGECAK | 447 |
| \|\|+\|\|+ ++ \| \| \| | | | |
| 00137 | 149 | IALDDILLSNGPCGK | 163 |

TABLE 1K

Domain Analysis of NOV1a

(SEQ ID NO:55)

gn1|Smart|smart00230, CysPc, Calpain-like thiol protease family.;
Calpain-like thiol protease family (peptidase family C2). Calcium
activated neutral protease (large subunit).
Length = 323 residues, 99.1% aligned
Score = 342 bits (877), Expect = 4e-95

| | | | |
|---|---|---|---|
| NOV1a | 258 | CGFEDGNICLFTQDDTDNFDWTKQSTATRNTKYTPNTGPNADRSGSKEGFYMYIETSRPR | 317 |
| \| \|\|\|\|+ \| ++\|\| \|+ \|\|\|+ ++\|\| + \|\|\| \| + \| \|\|\|++\|\| | | | |
| 00230 | 1 | CDFEDGSHCGWSQDSGDDLDWTRVNSATGGS-----TGPRGDHTT-GNGHYMYVDTS-SG | 53 |
| NOV1a | 318 | KEGEKARLLSPVFSIAPALFSARLLSPVFDIAPKNPYGPTNTAYCFSFFYHMYGQHIGVL | 377 |
| \|\|+ \|\|\|\|\|\| \| + \| +\|+\|\|\|\|\| +\| \| | | | |
| 00230 | 54 | QEGQTARLLSPPLP------------------------PKRSPCCLTFWYHMYGSGVGTL | 89 |
| NOV1a | 378 | NVYLRLKGQTTIENPLWSSSGNKGQRWNEAHVNIYPITS-FQLIFEGIRGPGIEGDIAID | 436 |
| \|\|\|+\| + + \|\|\| \|\|++\| \| \| \| + \| \|\|++\|\|\| \|\| \| \| \|\|+\| | | | |
| 00230 | 90 | NVYVR-ENGGPSDRLLWSRSGHQGGSWLLAEVTLPTSTKPFQVVFEGTRGGGSRGGIALD | 148 |
| NOV1a | 437 | DVSIAEGECAK | 447 |
| \|+\|++\|\| \| + | | | |
| 00230 | 149 | DISLSEGPCNQ | 159 |

Recent immunohistochemistry studies have attemted to examine the expression of VEGF isoforms and their receptors in fibrovascular tissues obtained from proliferative diabetic retinopathy cases. RT-PCR analysis demonstrated the expression of VEGF receptors VEGF-R1, VEGF-R2, and neuropilin-1 in 12, 14, and 14 of 22 proliferative diabetic retinopathy cases, respectively. Notably, VEGF-R2 and neuropilin-1 were simultaneously expressed in the identical 14 tissues. The isoform VEGF121 was constitutively expressed in all the tissues examined, whereas the expression of VEGF165 was confined to the 7 tissues that also expressed VEGF-R2 and neuropilin-1. The vascular density of fibrovascular tissues evaluated by immunohistochemistry for CD34 was significantly higher in the cases with the expression of VEGF-R2 and neuropilin-1 than in those without their expression (P<0.01), whereas VEGF-R1 expression had no such relationship with the vascular density. The fibrovascular tissues that expressed VEGF165 together with VEGF-R2 and neuropilin-1 were found in significantly younger patients (P<0.01). In situ hybridization and immunohistochemical studies demonstrated that glial cells in the fibrovascular tissues express and produce VEGF. Coexpression of VEGF-R2 and neuropilin-1 is suggested to facilitate fibrovascular proliferation in diabetic retinopathy (Ishida et al., Coexpression of VEGF receptors VEGF-R2 and neuropilin-1 in proliferative diabetic retinopathy. Invest Ophthalmol Vis Sci 41(7):1649-56, 2000.)

In the developing nervous system axons navigate with great precision over large distances to reach their target areas. Chemorepulsive signals such as the semaphorins play an essential role in this process. The effects of one of these repulsive cues, semaphorin 3A (Sema3A), are mediated by the membrane protein neuropilin-1 (Npn-1). Recent work has shown that neuropilin-1 is essential but not sufficient to form functional Sema3A receptors and indicates that additional components are required to transduce signals from the cell surface to the cytoskeleton. It was shown that members of the plexin family interact with the neuropilins and act as co-receptors for Sema3A. Neuropilin/plexin interaction restricts the binding specificity of neuropilin-1 and allows the receptor complex to discriminate between two different semaphorins. Deletion of the highly conserved cytoplasmic domain of Plexin-A1 or -A2 creates a dominant negative Sema3A receptor that renders sensory axons resistant to the repulsive effects of Sema3A when expressed in sensory ganglia. These data suggest that functional semaphorin receptors contain plexins as signal-transducing and neuropilins as ligand-binding subunits (Rohm et al., Plexin/neuropilin complexes mediate repulsion by the axonal guidance signal semaphorin 3A. Mech Dev 93(1-2):95-104, 2000).

Mesangial cell proliferation and growth factor over-expression are characteristic features of several glomerular diseases. Vascular endothelial growth factor (VEGF), a potent mitogen, is expressed in podocytes in the glomerulus, and VEGF receptors (flt-1, KDR, and neuropilin-1) are present on endothelial cells and other cell types. In conclusion, flt-1, KDR, and neuropilin-1 are present on cultured HMC, and VEGF(165) induces HMC proliferation. In addition, the flt-1 and KDR receptors are expressed in the mesangium in mesangioproliferative disease (Thomas et al., Vascular endothelial growth factor receptors in human mesangium in vitro and in glomerular disease. J Am Soc Nephrol 2000 11(7): 1236-43, 2000).

Vascular endothelial growth factor (VEGF), a major regulator of angiogenesis, binds to two receptor tyrosine kinases, KDR/Flk-1 and Flt-1. A third VEGF receptor, one that binds VEGF165 but not VEGF121, was cloned and purified from tumor cells. This isoform-specific VEGF receptor (VEGF165R) is identical to human neuropilin-1, a receptor for the collapsin/semaphorin family that mediates neuronal cell guidance. When coexpressed in cells with KDR, neuropilin-1 enhances the binding of VEGF165 to KDR and VEGF165-mediated chemotaxis. Conversely, inhibition of VEGF 165 binding to neuropilin-1 inhibits its binding to KDR and its mitogenic activity for endothelial cells. It is proposed that neuropilin-1 is a novel VEGF receptor that modulates VEGF binding to KDR and subsequent bioactivity and therefore may regulate VEGF-induced angiogenesis (Soker et al., Neuropilin-1 is expressed by endothelial and tumor cells as an isoform-specific receptor for vascular endothelial growth factor. Cell 92(6):735-45, 1998).

The above defined information for NOV1 suggests that this Membrane protein/neuropilin/metalloproteinase-like protein may function as a member of the Membrane protein/neuropilin/metalloproteinase family. Therefore, the NOV1 nucleic acids and proteins of the invention are useful in potential therapeutic applications implicated in various diseases and disorders described below and/or other pathologies. For example, the NOV1 compositions of the present invention will have efficacy for treatment of patients suffering from cancer, trauma, regeneration (in vitro and in vivo), viral/bacterial/parasitic infections, Von Hippel-Lindau (VHL) syndrome, Alzheimer's disease, stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, behavioral disorders, addiction, anxiety, pain, psoriasis, actinic keratosis, acne, hair growth diseases, allopecia, pigmentation disorders and endocrine disorders. The NOV1 nucleic acid encoding Membrane protein/neuropilin/metalloproteinase-like protein, and the Membrane protein/neuropilin/metalloproteinase-like protein of the invention, or fragments thereof, may further be useful in diagnostic applications, wherein the presence or amount of the nucleic acid or the protein are to be assessed.

NOV2

NOV2 includes six novel Fibrillin-like proteins disclosed below. The disclosed proteins have been named NOV2a, NOV2b, NOV2c, NOV2d, NOV2e and NOV2f.

NOV2a

A disclosed NOV2a nucleic acid of 9993 nucleotides (also referred to as GMAC022146_A) encoding a novel Fibrillin-like protein is shown in Table 2A. An open reading frame was identified beginning with an ATG initiation codon at nucleotides 166-168 and ending with a TGA codon at nucleotides 9151-9153. Putative untranslated regions upstream from the intiation codon and downstream from the termination codon are underlined in Table 2A. The start and stop codons are in bold letters.

TABLE 2A

NOV2a nucleotide sequence (SEQ ID NO:5).
CCTCCCATTCCTCTCCTCTCTCTCCCATCTGGACAGCCCCCAGCCTTCTGACACCCTGTTTCCCCCCCGGGACCCTGCTGC
CCTTCCCCACACCACCTAACATGCATTTCTGACCTTGCTCCTCCCACTCAAAGCTTTTCAAGGGCTCTTCATTGTCCTTGA
CGAATGAGCAAAAGCTGCATGACACTGCTATGCCCACTCCTGCAACGCCTGGCCCCACCTGGCCCCACCTGGCCCTCTCTC
CCCTCCCTTCCACTTCCCACTCCCGTCTTGCCTTGGCTGTGCACCCTCAGCCCCAAGACCCTTCTCTAACATCACACCTCC
TCCAGGAAGCCTCCTCTGACTGTTCACCGCTTCCCGGTTGGGGCTGGATGCCTCCTTGGGCTCCCAGAGGCCCCGGGGCTG
CCCCATTTGGCAACCACCCTGCCCTCCTCGTTGTGTTGGGTCCCCCATGGCTAGGGTGGGGGGCTCATCTAGGGCTGACTC
TGCTTCCTCTCCGCAGCCTCCAGGGGACACGCCATGACTCTGGAGGGTCTGTATTTGGCAAGGGGCCCCCTGGCCCGGCTC
CTGCTGGCCTGGTCGGCCCTGTTGTGCATGGCAGGTGGCCAAGGCCGCTGGGACGGGGCCTTGGAGGCTGCAGGTCCTGGA
CGTGTGCGGAGGCGGGGCAGCCCAGGCATCTTGCAGGGGCCGAATGTGTGCGGCTCCCGGTTCCATGCCTACTGCTGTCCA
GGCTGGAGGACATTCCCTGGCAGGAGCCAGTGTGTCGTACCCATCTGTAGGCGCGCCTGCGGTGAAGGCTTCTGCTCCCAG
CCCAACCTGTGCACCTGTGCGGATGGGACGCTGGCTCCCAGCTGCGGGGTGAGCCGAGGGTCAGGGTGCAGTGTGAGCTGT
ATGAATGGGGGCACCTGCCGGGGGGCGTCCTGTCTGTGTCAGAAGGGCTACACAGGCACCGTGTGTGGGCAGCCCATCTGT
GACCGCGGCTGCCACAATGGGGGTCGCTGCATTGGGCCCAACCGCTGCGCCTGTGTGTATGGCTTCATGGGACCTCAATGT
GAGAGAGATTACCGGACGGGACCCTGCTTTGGCCAAGTAGGCCCCGAGGGGTGCCAGCATCAGCTGACGGGCCTCGTGTGC
ACCAAGGCACTTTGCTGTGCCACTGTGGGCCGTGCCTGGGGCCTTCCATGTGAACTTTGCCCTGCACAGCCACACCCCTGC
CGCCGCGGCTTCATCCCCAATATCCACACGGGGGCCTGCCAAGATGTGGATGAGTGCCAGGCTGTGCCAGGCCTGTGCCAG
GGAGGCAGCTGCGTCAACATGGTGGGCTCCTTCCATTGCCGCTGTCCAGTTGGACACCGGCTCAGTGACAGCAGCGCCGCA
TGTGAAGACTACGATGAATGCAGCACCATTCCTGGAATCTGTGAAGGGGGTGAATGTACAAACACAGTCAGCAGTTACTTT
TGCAAATGTCCTCCTGGTTTTTACACCTCTCCAGATGGTACTCTTCATGGACAGTCGCGGGCCGGCGCCTGCTTCTCAGTG
CTTTTCGGGGGCCGCTGTGCTGGAGACCTCGCCGGCCACTACACTCGCAGGCAGTGCTGCTGTGACAGGGGCAGGTGCTGG
GCAGCTGGCCCGGTCCCTGAGCTGTGTCCTCCTCGGGGCTCCAATGAATTCCAGCAACTGTGCGCCCAGCGGCTGCCGCTG
CTACCCGGCCACCCTGGCCTCTTCCCTGGCCTCCTGGGCTTCGGATCCAATGGCATGGGTCCCCCTCTTGGGCCAGCGCGA
CTCAACCCCCATGGCTCTGATGCGCGTGGGATCCCCAGCCTGGGCCCTGGCAACTCTAATATTGGCACTGCTACCCTGAAC
CAGACCATTGACATCTGCCGACACTTCACCAACCTGTGTCTGAATGGCCGCTGCCTGCCCACGCCTTCCAGCTACCGCTGC
GAGTGTAACGTGGGCTACACCCAGGACGTGCGCGGCGAGTGCATTGATGTAGACGAATGCACCAGCAGCCCCTGCCACCAC
GGTGACTGCGTCAACATCCCCGGCACCTACCACTGCCGGTGCTACCCGGGCTTCCAGGCCACGCCCACCAGGCAGGCATGC
GTGGATGTGGACGAGTGCATTGTCAGTGGTGGCCTTTGTCACCTGGGCCGCTGTGTCAACACAGAGGGCAGCTTCCAGTGT
GTCTGCAATGCAGGCTTCGAGCTCAGCCCTGACGGCAAGAACTGTGTGGACCACAACGAGTGTGCCACCAGCACCATGTGC
GTCAACGGCGTGTGTCTCAACGAGGATGGCAGCTTCTCCTGCCTCTGCAAACCCGGCTTCCTGCTGGCGCCTGGCGGCCAC
TACTGCATGGACATTGACGAGTGCCAGACGCCCGGCATCTGCGTGAACGGCCACTGTACCAACACCGAGGGCTCCTTCCGC
TGCCAGTGCCTGGGGGGGCTGGCGGTAGGCACGGATGGCCGCGTGTGCGTGGACACCCACGTGCGCAGCACCTGCTATGGG
GCCATCGAGAAGGGCTCCTGTGCCCGCCCCTTCCCTGGCACTGTCACCAAGTCCGAGTGCTGCTGTGCCAATCCGGACCAC
GGTTTTGGGGAGCCCTGCCAGCTTTGTCCTGCCAAAGACTCCGCTGAGTTCCAGGCACTGTGCAGCAGTGGGCTTGGCATT
ACCACGGATGGTCGAGACATCAACGAGTGTGCTCTGGATCCTGAGGTTTGTGCCAATGGCGTGTGCGAGAACCTTCGGGGC
AGCTACCGCTGTGTCTGCAACCTGGGTTATGAGGCAGGTGCCTCAGGCAAGGACTGCACAGACGTGGATGAGTGTGCCCTC
AACAGCCTCCTGTGTGACAACGGGTGGTGCCAGAATAGCCCTGGCAGCTACAGCTGCTCCTGCCCCCCCGGCTTCCACTTC
TGGCAGGACACGGAGATCTGCAAAGATGTCGACGAATGCCTGTCCAGCCCGTGTGTGAGTGGCGTCTGTCGGAACCTGGCC
GGCTCCTACACCTGCAAATGTGGCCCTGGCAGCCGGCTGGACCCCTCTGGTACCTTCTGTCTAGACAGCACCAAGGGCACC
TGCTGGCTGAAGATCCAGGAGAGCCGCTGTGAGGTGAACCTTCAGGGAGCCAGCCTGCGGTCTGAGTGCTGTGCCACCCTC
GGGGCAGCCTGGGGGAGCCCCTGCGAACGCTGCGAGATCGACCCTGCCTGTGCCCGGGGCTTTGCCCGGATGACGGGTGTC
ACCTGCGATGATGTGAACGAGTGTGAGTCCTTCCCGGGAGTCTGTCCCAACGGGCGTTGCGTCAACACTGCTGGGTCTTTC
CGCTGTGAGTGTCCAGAGGGCCTGATGCTGGACGCCTCAGGCCGGCTGTGCGTGGATGTGAGATTGGAACCATGTTTCCTG
CGATGGGATGAGGATGAGTGTGGGGTCACCCTGCCTGGCAAGTACCGGATGGACGTCTGCTGCTGCTCCATCGGGGCCGTG
TGGGGAGTCGAGTGCGAGGCCTGCCCGGATCCCGAGTCTCTGGAGTTCGCCAGCCTGTGCCCGCGGGGGCTGGGCTTCGCC
AGCCGGGACTTCCTGTCTGGCCGACCATTCTATAAAGATGTGAATGAATGCAAGGTGTTCCCTGGCCTCTGCACGCACGGT
ACCTGCAGAAACACGGTGGGCAGCTTCCACTGCGCCTGTGCGGGGGGCTTCGCCCTGGATGCCCAGGAACGGAACTGCACA
GATATCGACGAGTGTCGCATCTCTCCTGACCTCTGCGGCCAGGGCACCTGTGTCAACACGCCGGGCAGCTTTGAGTGCGAG
TGTTTTCCCGGCTACGAGAGTGGCTTCATGCTGATGAAGAACTGCATGGACGTGGACGAGTGTGCAAGGGACCCGCTGCTC
TGCCGGGGAGGCACTTGCACCAACACGGATGGGAGCTACAAGTGCCAGTGTCCCCCTGGGCATGAGCTGACGGCCAAGGGC
ACTGCCTGTGAGGACATCGATGAGTGCTCCCTGAGTGATGGCCTGTGTCCCCATGGCCAGTGTGTCAATGTCATCGGTGCC
TTCCAGTGCTCCTGCCATGCCGGCTTCCAGAGCACACCTGACCGCGGAGCTACAAGTGCCAGTTGCCCGACGGAAGGGCAT
GTGCAGGTCGTCCTGGGGCCTGGGGAGCAGATGTGTACTGGGTGGTCTATCAGGGCAAAGCTAAGCACAGTGGGGAACCCT
GCTCCCCCAGACGTGGACGAGTGTGAAGAGAACCCCCGCGTTTGTGACCAAGGCCACTGCACCAACATGCCAGGGGGTCAC
CGCTGCCTGTGCTATGATGGCTTCATGGCCACGCCAGACATGAGGACATGTGTTGATGTGGATGAGTGTGACCTGAACCCT
CACATCTGCCTCCATGGGGACTGCGAGAACACGAAGGGTTCCTTTGTCTGCCACTGTCAGCTGGGCTACATGGTCAGGAAG
GGGGCCACAGGCTGCTCTGATGTGGATGAATGCGAGGTTGGAGGACACAACTGTGACAGTCACGCCTCCTGTCTCAACATC
CCAGGGAGTTTCAGCTGTAGGTGCCTGCCAGGCTGGGTGGGGGATGGCTTCGAATGTCACGACCTGGATGAATGCGTCTCC
CAGGAGCACCGGTGCAGCCCAAGAGGTGACTGTCTCAATGTCCCTGGCTCCTACCGCTGCACCTGCCGCCAGGGCTTTGCC
GGGGATGGCTTCTTCTGCGAAGACAGGGATGAATGTGCCGAGAACGTGGACCTCTGTGACAACGGGCAGTGCCTCAATGCG
CCCGGCGGGTACCGCTGTGAATGTGAGATGGGCTTTGACCCCACCGAGGACCACCGGGCCTGCCAGGATGTGGACGAGTGT
GCGCAAGAGAACCTCTGTGCATTTGGGAGCTGTGAGAACCTGCCTGGAATGTTCCGCTGCATCTGCAATGGTGGCTACGAA
CTGGACCGAGGGGGTGGCAACTGCACAGACATCAACGAGTGTGCAGACCCAGTAAACTGCATCAACGGCGTGTGCATTAAC
ACCCCCGGCAGCTACCTCTGCAGCTGCCCCCAGGATTTTGAGCTGAACCCCAGCGGAGTGGGCTGCGTGGACACTCGGGCC
GGGAACTGTTTCCTGGAGACGCATGACCGAGGGGACAGTGGCATTTCCTGCAGTGCCGAGATCGGAGTTGGTGTCACCCGA
GCTTCCTGCTGTTGCTCCCTGGGCCGGGCTTGGGGCAATCCCTGTGAGCTGTGCCCTATGGCCAACACCACTGAGTACAGA
ACCCTGTGCCCGGGTGGTGAGGGCTTCCAGCCTAACCGCATCACTGTCATTCTGGAAGACATCGACGAGTGCCAAGAGCTG
CCAGGGCTGTGTCAGGGGGGTGACTGCGTCAACACGTTTGGCAGTTTCCAGTGTGAGTGCCCACCTGGCTACCACCTCAGT
GAGCACACCCGCATCTGTGAGGATATTGACGAATGCTCCACACACTCCGGCATCTGTGGCCCTGGCACCTGCTACAACACC
CTGGGGAACTACACCTGTGTCTGCCCTGCAGAGTACCTCCAAGTCAATGGTGGCAACAACTGCATGGATATGAGGAAGAGT
GTCTGCTTCCGGCACTATAACGGCACATGTCAAAATGAGCTGGCCTTCAACGTGACCCGGAAAATGTGTTGCTGCTCCTAC
AACATTGGCCAGGCCTGGAATAGACCCTGTGAGGCCTGCCCCACTCCCATCAGTCCTGACTACCAGATCCTGTGTGGAAAT
CAGGCCCCGGGATTCCTCACTGACATCCACACGGGGAAGCCCCTTGACATTGATGAGTGTGGGGAGATCCCCGCCATCTGT
GCCAATGGCATCTGCATAAACCAGATCGGGAGTTTCCGCTGCGAGTGCCCCGCAGGCTTCAACTACAACAGCATCCTGCTG
GCTTGTGAAGATGTCGATGAGTGTGGCAGCAGGGAGAGTCCCTGCCAGCAGAATGCTGACTGCATCAACATCCCCGGTAGC
TACCGCTGCAAGTGCACCCGAGGGTACAAACTGTCGCCAGGCGGGGCTTGTGTGGGACGGAATGAGTGTCGGGAGATCCCG
AATGTCTGTAGCCATGGTGACTGCATGGACACAGAAGGCAGCTACATGTGTCTGTGTCACCGTGGATTCCAGGCCTCTGCA
GACCAGACCCTGTGCATGGACATTGACGAGTGTGACCGGCAGCCTTGTGGAAATGGGACCTGCAAGAACATCATTGGCTCC
TACAACTGCCTCTGCTTCCCTGGCTTTGTGGTGACACACAATGGGGATTGTGTGGATTTTGATGAGTGTACTACCCTGGTG
GGGCAGGTGTGCCGATTTGGCCATTGCCTCAACACAGCTGGTTCCTTCCACTGCCTCTGCCAGGATGGCTTTGAGCTCACA
GCTGATGGGAAGAACTGTGTGGACACCAATGAGTGCCTCAGCCTTGCAGGAACCTGCCTACCCGGCACTTGCCAGAACCTC
GAGGGCTCCTTCCGCTGCATCTGTCCCCCTGGCTTCCAGGTGCACAGTGACCACTGCATTGATATCGACGAGTGCTCAGAG
GAGCCCAACCTCTGCCTCTTTGGCACCTGTACCAACAGCCCTGGGAGCTTCCAGTGCCTCTGCCCACCTGGCTTTGTCCTC
TCTGACAATGGGCACCGTTGCTTTGACACACGGCAGAGTTTCTGCTTCACCCGTTTTGAGGCTGGGAAGTGCTCGGTGCCC
AAAGCTTTCAACACCACCAAGACCCGCTGCTGCTGCAGTAAGAGGCCTGGGGAGGGCTGGGGAGACCCCTGCGAACTGTGT
CCCCAGGAGGGCAGCGCTGCCTTTCAGGAGCTCTGCCCCTTTGGCCACGGGGCAGTCCCAGGCCCGGATGACTCCCGAGAA
GACGTGAATGAGTGTGCAGAGAACCCTGGCGTCTGCACTAACGGCGTCTGTGTCAACACCGATGGATCCTTCCGCTGTGAG
TGTCCCTTTGGCTACAGCCTGGACTTCACTGGCATCAACTGTGTGGACACAGACGAGTGCTCTGTCGGCCACCCCTGTGGG
CAAGGGACATGCACCAATGTCATCGGAGGCTTCGAATGTGCCTGTGCTGACGGCTTTGAGCCTGGCCTCATGATGACCTGC
GAGGAGATCGACGAATGCTCCCTGAACCCGCTGCTCTGTGCCTTCCGCTGCCACAATACCGAGGGCTCCTACCTGTGCACC
TGTCCAGCCGGCTACACCCTGCGGGAGGATGGGGCCATGTGTGAAGATGTGGACGAGTGTGCAGATGGTCAGCAGGACTGC
CACGCCCGGGGCATGGAGTGCAAGAACCTCATCGGTACCTTCGCGTGCGTCTGTCCCCCAGGCATGCGGCCCCTGCCTGGC
TCTGGGGAGGGCTGCACAGATGACAATGAATGCCACGCTCAGCCTGACCTCTGTGTCAACGGCCGCTGTGTCAACACCGCG
GGCAGCTTCCGGTGCGACTGTGATGAGGGATTCCAGCCCAGCCCCACCCTTACCGAGTGCCACGACATCCGGCAGGGGCCC
TGCTTTGCCGAGGTGCTGCAGACCATGTGCCGGTCTCTGTCCAGCAGCAGTGAGGCTGTCACCAGGGCCGAGTGCTGCTGT
GGGGGTGGCCGGGGCTGGGGGCCCCGCTGCGAGCTCTGTCCCCTGCCCGGCACCTCTGCCTACAGGAAGCTGTGCCCCCAT
GGCTCAGGCTACACTGCTGAGGGCCGAGATGTAGATGAATGCCGTATGCTTGCTCACCTGTGTGCTCATGGGGAGTGCATC
AACAGCCTTGGCTCCTTCCGCTGCCACTGTCAGGCCGGGTACACACCGGATGCTACTGCTACTACCTGCCTGGATATGGAT
GAGTGCAGCCAGGTCCCCAAGCCATGTACCTTCCTCTGCAAAAACACGAAGGGCAGTTTCCTGTGCAGCTGTCCCCGAGGC
TACCTGCTGGAGGAGGATGGCAGGACCTGCAAAGACCTGGACGAATGCACCTCCCGGCAGCACAACTGTCAGTTCCTCTGT
GTCAACACTGTGGGCGCCTTCACCTGCCGCTGTCCGCCCGGCTTCACCCAGCACCACCAGGCCTGCTTCGACAATGATGAG
TGCTCAGCCCAGCCTGGCCCATGTGGTGCCCACGGGCACTGCCACAACACCCCGGGCAGCTTCCGCTGTGAATGCCACCAA
GGCTTCACCCTGGTCAGCTCAGGCCATGGCTGTGAAGATGTGAATGAATGTGATGGGCCCCACCGCTGCCAGCATGGCTGT
CAGAACCAGCTAGGGGGCTACCGCTGCAGCTGCCCCCAGGGTTTCACCCAGCACTCCCAGTGGGCCCAGTGTGTGGATGAG
AATGAGTGTGCCCTGTCGCCCCCCACCTGCGGGAGCGCCTCCTGTCGCAACACTCTTGGTGGCTTCCGCTGCGTCTGCCCC
TCTGGCTTTGACTTTGATCAGGCCCTCGGGGGCTGCCAGGAGGTGGATGAGTGCGCCGGACGGCGTGGCCCCTGTAGCTAC
AGCTGTGCCAACACGCCTGGTGGCTTCCTGTGCGGCTGTCCTCAAGGCTACTTCCGGGCTGGGCAAGGGCACTGTGTCTCC
GGCCTGGGCTTCAGCCCCGGACCCCAGGACACCCCGGACAAAGAGGAGCTGCTCTCGTCTGAAGCCTGCTACGAATGCAAG
ATCAATGGCCTCTCCCCTCGGGACCGGCCACGACGCAGTGCCCACAGGGACCACCAGGTGAACCTGGCCACCCTTGACTCC
GAGGCCCTGCTGACCTTGGGCCTGAACCTCTCACACCTGGGCCGGGCCGAGCGCATCCTGGAGCTCCGGCCGGCCCTGGAG
GGTCTAGAGGGCCGGATCCGCTACGTCATCGTCCGCGGAAACGAGCAAGGTTTCTTTCGCATGCATCACCTCCGTGGCGTC
AGCTCCCCGCAGCTGGGGCGGAGGCGGCCGGGGCCTGGAACCTACCGGCTGGAGGTGGTGAGCCACATGGCAGGACCCTGG
GGTGTCCAGCAAGAGGGGCAGCCAGGGCCATGGGGCCAGGCCTTGAGGCTGAAGGTGCAACTGTCAGTTGCTTTAGTTGGG
AGGAGCCTCAGTGGGCCCCAGCTGTCCAGAGAAGGGGGATTCTGGAACTGGGAAGGACTGATCCCCAGAAGCGATGGCTGA
CCAGATTGAACCCCGAAACTCAGGAAGAGTGAAATGCTACACGACAACCTCAGGCAAGCCCGGCCTCTGCCTGGGCCTCTG
TGCCAGCCCCGGGGGCCCCCCAGTTACTCAGTCTTTCCTGGAGACAGCAAGAAGCTGCAATGTGCAATCCCCCTGCCCCCA
CAGCCAAGGTCAGGAAGAGGCCCTGTGGTCACCGTGTCTGGCGAATCTCAGGCTTTCACTTCTGTACTGCACTGTGGCTTG
CCCTGGCGGGGGGCAGGGGGTTGGCAGGACATGGCAATGGGCAACTGGGGTGGGCACAGGGCTTATTCCTCGGAGTAGAAG
GGTGTACAGGGGGCCCAGACTCCACAGTGACTTGCCACATTTGCCCCCCATTTGGAGAATGCTTTTATATCAAAAGTGGAG
ACGATAATAAAGTTATTTTGGGTTAAGTCTGCCTGCCCTTTGGCAAGTTCTTGAAGTAAGTAGATGCTGCCCTCGGACTGG
GCGAGGCAGATCTTGTGCCTGGGGAAGCAGAAGGCCTTATGGGCTCCCCAGAATGGTAATAATGGCTCACGCTTCCTGACC
ACGTACTACATACCAGACACCATTCGATTTTTTTTTTATTTTTTCTGAGACAGGGTCTTGCTCTGTTGCCTAGGTTGGAGT
GCAGTGGCGCGATCGTGGCTCCCTGTGGTCGCCACTTTCCGGGCTCGAGCAATCCTCCCACCTCAGCCTCTCCCAAGTAGC
TGGGACCAAAGGTGCACGCCACCACACCCAGCTAATTTTTTTAAATTTCTTTTTCTTTCTCTTTCTTTTTGAGACCAGCCC
GGCCAACATGACGAAACCCCATCTCTAATA

The disclosed NOV2a nucleic acid sequence, localized to chromsome 19, has 3196 of 4382 bases (72%) identical to a Mus musculus fibrillin 2 (fbn2) mRNA (gb:GENBANK-ID:MUSFBN2|acc:L39790) (E=0.0).

A NOV2a polypeptide (SEQ ID NO: 6) encoded by SEQ ID NO: 5 has 2995 amino acid residues and is presented using the one-letter code in Table 2B. Signal P, Psort and/or Hydropathy results predict that NOV2a contains a signal peptide and is likely to be localized to the endoplasmic reticulum membrane with a certainty of 0.5500. The most likely cleavage site for a NOV2a peptide is between amino acids 54 and 55, at: TSS-RK.

TABLE 2B

Encoded NOV2a protein sequence (SEQ ID NO:6).
MSKSCMTLLCPLLQRLAPPGPTWPSLPSLPLPTPVLPWLCTLSPKTLLXHHTSSRKPPLTVHRFPVGAGCLLGLPEAPGLP
HLATTLPSSLCWVPHGXGGGLIXGXLCFLSAASRGHAMTLEGLYLARGPLARLLLAWSALLCMAGGQGRWDGALEAAGPGR
VRRRGSPGILQGPNVCGSRFHAYCCPGWRTFPGRSQCVVPICRRACGEGFCSQPNLCTCADGTLAPSCGVSRGSGCSVSCM
NGGTCRGASCLCQKGYTGTVCGQPICDRGCHNGGRCIGPNRCACVYGFMGPQCERDYRTGPCFGQVGPEGCQHQLTGLVCT
KALCCATVGRAWGLPCELCPAQPHPCRRGFIPNIHTGACQDVDECQAVPGLCQGGSCVNMVGSFHCRCPVGHRLSDSSAAC
EDYDECSTIPGICEGGECTNTVSSYFCKCPPGFYTSPDGTLHGQSRAGACFSVLFGGRCAGDLAGHYTRRQCCCDRCRCWA
AGPVPELCPPRGSNEFQQLCAQRLPLLPGHPGLFPGLLGFGSNGMGPPLGPARLNPHGSDARGIPSLGPGNSNIGTATLNQ
TIDICRHFTNLCLNGRCLPTPSSYRCECNVGYTQDVRGECIDVDECTSSPCHHGDCVNIPGTYHCRCYPGFQATPTRQACV
DVDECIVSGGLCHLGRCVNTEGSFQCVCNAGFELSPDGKNCVDHNECATSTMCVNGVCLNEDGSFSCLCKPGFLLAPGGHY
CMDIDECQTPGICVNGHCTNTEGSFRCQCLGGLAVGTDGRVCVDTHVRSTCYGAIEKGSCARPFPGTVTKSECCCANPDHG
FGEPCQLCPAKDSAEFQALCSSGLGITTDGRDINECALDPEVCANGVCENLRGSYRCVCNLGYEAGASGKDCTDVDECALN
SLLCDNGWCQNSPGSYSCSCPPGFHFWQDTEICKDVDECLSSPCVSGVCRNLAGSYTCKCGPGSRLDPSGTFCLDSTKGTC
WLKIQESRCEVNLQGASLRSECCATLGAAWGSPCERCEIDPACARGFARMTGVTCDDVNECESFPGVCPNGRCVNTAGSFR
CECPEGLMLDASGRLCVDVRLEPCFLRWDEDECGVTLPGKYRMDVCCCSIGAVWGVECEACPDPESLEFASLCPRGLGFAS
RDFLSGRPFYKDVNECKVFPGLCTHGTCRNTVGSFHCACAGGFALDAQERNCTDIDECRISPDLCGQGTCVNTPGSFECEC
FPGYESGFMLMKNCMDVDECARDPLLCRGGTCTNTDGSYKCQCPPGHELTAKGTACEDIDECSLSDGLCPHGQCVNVIGAF
QCSCHAGFQSTPDRGATSASCPTEGHVQVVLGPGEQMCTGWSIRAKLSTVPNPAPPDVDECEENPRVCDQGHCTNMPGGHR
CLCYDGFMATPDMRTCVDVDECDLNPHICLHGDCENTKGSFVCHCQLGYMVRKGATGCSDVDECEVGGHNCDSHASCLNIP
GSFSCRCLPGWVGDGFECHDLDECVSQEHRCSPRGDCLNVPGSYRCTCRQGFAGDGFFCEDRDECAENVDLCDNGQCLNAP
GGYRCECEMGFDPTEDHRACQDVDECAQENLCAFGSCENLPGMFRCICNGGYELDRGGGNCTDINECADPVNCINGVCINT
PGSYLCSCPQDFELNPSGVGCVDTRAGNCFLETHDRGDSGISCSAEIGVGVTRASCCCSLGRAWGNPCELCPMANTTEYRT
LCPGGEGFQPNRITVILEDIDECQELPGLCQGGDCVNTFGSFQCECPPGYHLSEHTRICEDIDECSTHSGICGPGTCYNTL
GNYTCVCPAEYLQVNGGNNCMDMRKSVCFRHYNGTCQNELAFNVTRKMCCCSYNIGQAWNRPCEACPTPISPDYQILCGNQ
APGFLTDIHTGKPLDIDECGEIPAICANGICINQIGSFRCECPAGFNYNSILLACEDVDECGSRESPCQQNADCINIPGSY
RCKCTRGYKLSPGGACVGRNECREIPNVCSHGDCMDTEGSYMCLCHRGFQASADQTLCMDIDECDRQPCGNGTCKNIIGSY
NCLCFPGFVVTHNGDCVDFDECTTLVGQVCRFGHCLNTAGSFHCLCQDGFELTADGKNCVDTNECLSLAGTCLPCTCQNLE
GSFRCICPPGFQVQSDHCIDIDECSEEPNLCLFGTCTNSPGSFQCLCPPGFVLSDNGHRCFDTRQSFCFTRFEAGKCSVPK
AFNTTKTRCCCSKRPGEGWGDPCELCPQEGSAAFQELCPFGHHAVPGPDDSREDVNECAENPGVCTNGVCVNTDGSFRCEC
PFGYSLDFTGINCVDTDECSVGHPCKQGTCTNVIGGFECACADGFEPGLMMTCEEIDECSLNPLLCAFRCHNTEGSYLCTC
PAGYTLREDGAMCEDVDECADGQQDCHARGMECKNLIGTFACVCPPGMRPLPGSGEGCTDDNECHAQPDLCVNGRCVNTAG
SFRCDCDEGFQPSPTLTECHDIRQGPCFAEVLQTMCRSLSSSSEAVTRAECCCGGGTGWGPRCELCPLPGTSAYRKLCPHG
SGYTAEGRDVDECRMLAHLCAHGECINSLGSFRCHCQAGYTPDATATTCLDMDECSQVPKPCTFLCKNTKGSFLCSCPRGY
LLEEDGRTCKDLDECTSRQHNCQFLCVNTVGAFTCRCPPGFTQHHQACFDNDECSAQPGPCGAHGHCHNTPGSFRCECHQG
FTLVSSGHGCEDVNECDGPHRCQHGCQNQLGGYRCSCPQGFTQHSQWAQCVDENECALSPPTCGSASCRNTLGGFRCVCPS
GFDFDQALGGCQEVDECAGRRGPCSYSCANTPGGFLCGCPQGYFRAGQGHCVSGLGFSPGPQDTPDKEELLSSEACYECKI
NGLSPRDRPRRSAHRDHQVNLATLDSEALLTLGLNLSHLGRAERILELRPALEGLEGRIRYVIVRGNEQGFFRMHHLRGVS
SPQLGRRRPGPGTYRLEVVSHMAGPWGVQQEGQPGPWGQALRLKVQLSVALVGRSLSGPQLSREGGFWNWEGLIPRSDG

The NOV2a amino acid sequence has 1895 of 2810 amino acid residues (67%) identical to, and 2257 of 2810 amino acid residues (80%) similar to, a Mus musculus 2907 amino acid residue fibrillin 2 precursor protein (ptnr:SWISSPROT-ACC:Q61555) (E=0.0).

The disclosed NOV2a is expressed in at least the following tissues: testes, ovary, lung, liver, B-cells, total-fetus, spleen, Nervous System, Brain, Prosencephalon/Forebrain, Diencephalon, Pituitary Gland, Hematopoietic Tissues, Lymphoid tissue, Lymph node and Whole Organism. This information was derived by determining the tissue sources of the sequences that were included in the invention. In addition, the NOV2a sequence is predicted to be expressed in the following tissues because of the expression pattern of a closely related Mus musculus fibrillin 2 (fbn2) gene (GENBANK-ID: gb:GENBANK-ID:MUSFBN2|acc:L39790): testes, ovary, lung, liver, total-fetus, brain, spleen, Nervous System, Brain, Pituitary Gland; Hematopoietic and Lymphatic System.

NOV2b

A disclosed NOV2b nucleic acid of 9894 nucleotides (also referred to as 153568997) encoding a novel Fibrillin-like protein is shown in Table 2C. An open reading frame was identified beginning with an ATG initiation codon at nucleotides 520-522 and ending with a TGA codon at nucleotides 9052-9054. Putative untranslated regions upstream from the intiation codon and downstream from the termination codon are underlined in Table 2C, and the start and stop codons are in bold letters.

TABLE 2C

NOV2b nucleotide sequence (SEQ ID NO:7).
CCTCCCATTCCTCTCCTCTCTCTCCCATCTGGACAGCCCCCAGCCTTCTGACACCCTGTTTCCCCCCCGGGACCCTGCTGC
CCTTCCCCACACCACCTAACATGCATTTCTGACCTTGCTCCTCCCACTCAAAGCTTTTCAAGGGCTCTTCATTGTCCTTGA
CGAATGAGCAAAAGCTGCATGACACTGCTATGCCCACTCCTGCAACGCCTGGCCCCACCTGGCCCCACCTGGCCCTCTCTC
CCCTCCCTTCCACTTCCCACTCCCGTCTTGCCTTGGCTGTGCACCCTCAGCCCCAAGACCCTTCTCTAACATCACACCTCC
TCCAGGAAGCCTCCTCTGACTGTTCACCGCTTCCCGGTTGGGGCTGGATGCCTCCTTGGGCTCCCAGAGGCCCCGGGGCTG
CCCCATTTGGCAACCACCCTGCCCTCCTCGTTGTGTTGGGTCCCCCATGGCTAGGGTGGGGGGCTCATCTAGGGCTGACTC
TGCTTCCTCTCCGCAGCCTCCAGGGGACACGCCATGACTCTGGAGGGTCTGTATTTGGCAAGGGGCCCCCTGGCCCGGCTC
CTGCTGGCCTGGTCGGCCCTGTTGTGCATGGCAGGTGGCCAAGGCCGCTGGGACGGGGCCTTGGAGGCTGCAGGTGGTGGA
CGTGTGCGGAGGCGGGGCAGCCCAGGCATCTTGCAGGGGCCGAATGTGTGCGGCTCCCGGTTCCATGCCTACTGCTGTCCA
GGCTGGAGGACATTCCCTGGCAGGAGCCAGTGTGTCGTACCCATCTGTAGGCGCGCCTGCGGTGAAGGCTTCTGCTCCCAG
CCCAACCTGTGCACCTGTGCGGATGGGACGCTGGCTCCCAGCTGCGGGGTGAGCCGAGGGTCAGGGTGCAGTGTGAGCTGT
ATGAATGGGGGCACCTGCCGGGGGGCGTCCTGTCTGTGTCAGAAGGGCTACACAGGCACCGTGTGTGGGCAGCCCATCTGT
GACCGCGGCTGCCACAATGGGGGTCGCTGCATTGGGCCCAACCGCTGCGCCTGTGTGTATGGCTTCATGGGACCTCAATGT
GAGAGAGATTACCGGACGGGATCCTGCTTTGGCCAAGTAGGCCCCGAGGGGTGCCAGCATCAGCTGACGGGCCTCGTGTGC
ACCAAGGCACTTTGCTGTGCCACTGTGGGCCGTGCCTGGGGCCTTCCATGTGAACTTTGCCCTGCACAGCCACACCCCTGC
CGCCGCGGCTTCATCCCCAATATCCACACGGGGGCCTGCCAAGATGTGGATGAGTGCCAGGCTGTGCCAGGCCTGTGCCAG
GGAGGCAGCTGCGTCAACATGGTGGGCTCCTTCCATTGCCGCTGTCCAGTTGGACACCGGCTCAGTGACAGCAGCGCCGCA
TGTGAAGACTACCGGGCCGGCGCCTGCTTCTCAGTGCTTTTCGGGGGCCGCTGTGCTGGAGACCTCGCCGGCCACTACACT
CGCAGGCAGTGCTGCTGTGACAGGGGCAGGTGCTGGGCAGCTGGCCCGGTCCCTGAGCTGTGTCCTCCTCGGGGCTCCAAT
GAATTCCAGCAACTGTGCGCCCAGCGGCTGCCGCTGCTACCCGGCCACCCTGGCCTCTTCCCTGGCCTCCTGGGCTTCGGA
TCCAATGGCATGGGTCCCCCTCTTGGGCCAGCGCGACTCAACCCCCATGGCTCTGATGCGCGTGGGATCCCCAGCCTGGGC
CCTGGCAACTCTAATATTGGCACTGCTACCCTGAACCAGACCATTGACATCTGCCGACACTTCACCAACCTGTGTCTGAAT
GGCCGCTGCCTGCCCACGCCTTCCAGCTACCGCTGCGAGTGTAACGTGGGCTACACCCAGGACGTGCGCGGCGAGTGCATT
GATGTAGACGAATGCACCAGCAGCCCCTGCCACCACGGTGACTGCGTCAACATCCCCGGCACCTACCACTGCCGGTGCTAC
CCGGGCTTCCAGGCCACGCCCACCAGGCAGGCATGCGTGGATGTGGACGAGTGCATTGTCAGTGGTGGCCTTTGTCACCTG
GGCCGCTGTGTCAACACAGAGGGCAGCTTCCAGTGTGTCTGCAATGCAGGCTTCGAGCTCAGCCCTGACGGCAAGAACTGT
GTGGACCACAACGAGTGTGCCACCAGCACCATGTGCGTCAACGGCGTGTGTCTCAACGAGGATGGCAGCTTCTCCTGCCTC
TGCAAACCCGGCTTCCTGCTGGCGCCTGGCGGCCACTACTGCATGGACATTGACGAGTGCCAGACGCCCGGCATGTGCGTG
AACGGCCACTGTACCAACACCGAGGGCTCCTTCCGCTGCCAGTGCCTGGGGGGGCTGGCGGTAGGCACGGATGGCCGCGTG
TGCGTGGACACCCACGTGCGCAGCACCTGCTATGGGGCCATCGAGAAGGGCTCCTGTGCCCGCCCCTTCCCTGGCACTGTC
ACCAAGTCGGAGTGCTGCTGTGCCAATCCGGACCACGGTTTTGGGGAGCCCTGCCAGCTTTGTCCTGCCAAAAACTCCGCT
GAGTTCCAGGCACTGTGCAGCAGTGGGCTTGGCATTACCACGGATGGTCGAGACATCAACGAGTGTGCTCTGGATCCTGAG
GTTTGTGCCAATGGCGTGTGCGAGAACCTTCGGGGCAGCTACCGCTGTGTCTGCAACCTGGGTTATGAGGCAGGTGCCTCA
GGCAAGGACTGCACAGACGTGGATGAGTGTGCCCTCAACAGCCTCCTGTGTGACAACGGGTGGTGCCAGAATAGCCCTGGC
AGCTACAGCTGCTCCTGCCCCCCCGGCTTCCACTTCTGGCAGGACACGGAGATCTGCAAAGATGTCGACGAATGCCTGTCC
AGCCCGTGTGTGAGTGGCGTTTGTCGGAACCTGGCCGGCTCCTACACCTGCAAATGTGGCCCTGGCAGCCGGCTGGACCCC
TCTGGTACCTTCTGTCTAGACAGCACCAAGGGCACCTGCTGGCTGAAGATCCAGGAGAGCCGCTGTGAGGTGAACCTTCAG
GGAGCCAGCCTGCGGTCTGAGTGCTGTGCCACCCTCGGGGCAGCCTGGGGGAGCCCCTGCGAACGCTGCGAGATCGACCCT
GCCTGTGCCCGGGGCTTTGCCCGGATGACGGGTGTCACCTGCGATGATGTGAACGAGTGTGAGTCCTTCCCGGGAGTCTGT
CCCAACGGGCGTTGCGTCAACACTGCTGGGTCTTTCCGCTGTGAGTGTCCAGAGGGCCTGATGCTGGACGCCTCAGGCCGG
CTGTGCGTGGATGTGAGATTGGAACCATGTTTCCTGCGATGGGATGAGGATGAGTGTGGGGTCACCCTGCCTGGCAAGTAC
CGGATGGACGTCTGCTGCTGCTCCATCGGGGCCGTGTGGGGAGTCGAGTGCGAGGCCTGCCCGGATCCCGAGTCTCTGGAG
TTCGCCAGCCTGTGCCCGCGGGGGCTGGGCTTCGCCAGCCGGGACTTCCTGTCTGGCCGACCATTCTATAAAGATGTGAAT
GAATGCAAGGTGTTCCCTGGCCTCTGCACGCACGGTACCTGCAGAAACACGGTGGGCAGCTTCCACTGCGCCTGTGCGGGG
GGCTTCGCCCTGGATGCCCAGGAACGGAACTGCACAGATATCGACGAGTGTCGCATCTCTCCTGACCTCTGCGGCCAGGGC
ACCTGTGTCAACACGCCGGGCAGCTTTGAGTGCGAGTGTTTTCCCGGCTACGAGAGTGGCTTCATGCTGATGAAGAACTGC
ATGGACGTGGACGAGTGTGCAAGGGACCCGCTGCTCTGCCGGGGAGGCACTTGCACCAACACGGATGGGAGCTACAAGTGC
CAGTGTCCCCCTGGGCATGAGCTGACGGCCAAGGGCACTGCCTGTGAGGACATCGATGAGTGCTCCCTGAGTGATGGCCTG
TGTCCCCATGGCCAGTGTGTCAATGTCATCGGTGCCTTCCAGTGCTCCTGCCATGCCGGCTTCCAGAGCACACCTGACCGC
CAGGGCTGCGTGGACATCAACGAATGCCGGGTCCAGAATGGTGGGTGTGACGTGCACCGTATTAACACTGAGGGCAGCTAC
CGGTGCAGCTGTGGGCAGGGCTACTCGCTGATGCCCGACGGAAGGGCATGTGCAGACGTGGACGAGTGTGAAGAGAACCCC
CGCGTTTGTGACCAAGGCCACTGCACCAACATGCCAGGGGGTCACCGCTGCCTGTGCTATGATGGCTTCATGGCCACGCCA
GACATGAGGACATGTGTTGATGTGGATGAGTGTGACCTGAACCCTCACATCTGCCTCCATGGGGACTGCGAGAACACGAAG
GGTTCCTTTGTCTGCCACTGTCAGCTGGGCTACATGGTCAGGAAGGGGGCCACAGGCTGCTCTGATGTGGATGAATGCGAG
GTTGGAGGACACAACTGTGACAGTCACGCCTCCTGTCTCAACATCCCGGGGAGTTTCAGCTGTAGGTGCCTGCCAGGCTGG
GTGGGGGATGGCTTCGAATGTCACGACCTGGATGAATGCGTCTCCCAGGAGCACCGGTGCAGCCCAAGAGGTGACTGTCTC
AATGTCCCTGGCTCCTACCGCTGCACCTGCCGCCAGGGCTTTGCCGGGGATGGCTTCTTCTGCGAAGACAGGGATGAATGT
GCCGAGAACGTGGACCTCTGTGACAACGGGCAGTGCCTCAATGCGCCCGGCGGGTACCGCTGTGAATGTGAGATGGGCTTT
GACCCCACCGAGGACCACCGGGCCTGCCAGGATGTGGACGAGTGTGCGCAAGAGAACCTCTGTGCATTTGGGAGCTGTGAG
AACCTGCCTGGAATGTTCCGCTGCATCTGCAATGGTGGCTACGAACTGGACCGAGGGGGTGGCAACTGCACAGACATCAAC
GAGTGTGCAGACCCAGTAAACTGCATCAACGGCGTGTGCATTAACACCCCCGGCAGCTACCTCTGCAGCTGCCCCCAGGAT
TTTGAGCTGAACCCCAGCGGAGTGGGCTGCGTGGACACTCGGGCCGGGAACTGTTTCCTGGAGACGCATGACCGAGGGGAC
AGTGGCATTTCCTGCAGTGCCGAGATCGGAGTTGGTGTCACCCGAGCTTCCTGCTGTTGCTCCCTGGGCCGGGCTTGGGGC
AATCCCTGTGAGCTGTGCCCTATGGCCAACACCACTGAGTACAGAACCCTGTGCCCGGGTGGTGAGGGCTTCCAGCCTAAC
CGCATCACTGTCATTCTGGAAGACATCGACGAGTGCCAAGAGCTGCCAGGGCTGTGTCAGGGGGGTGACTGCGTCAACACG
TTTGGCAGTTTCCAGTGTGAGTGCCCACCTGGCTACCACCTCAGTGAGCACACCCGCATCTGTGAGGATATTGACGAATGC
TCCACACACTCCGGCATCTGTGGCCCTGGCACCTGCTACAACACCCTGGGGAACTACACCTGTGTCTGCCCTGCAGAGTAC
CTCCAAGTCAATGGTGGCAACAACTGCATGGATATGAGGAAGAGTGTCTGCTTCCGGCACTATAACGGCACATGTCAAAAT
GAGCTGGCCTTCAACGTGACCCGGAAAATGTGTTGCTGCTCCTACAACATTGGCCAGGCCTGGAATAGACCCTGTGAGGCC
TGCCCCACTCCCATCAGTCCTGACTACCAGATCCTGTGTGGAAATCAGGCCCCGGGATTCCTCACTGACATCCACACGGGG
AAGCCCCTTGACATTGATGAGTGTGGGGAGATCCCCGCCATCTGTGCCAATGGCATCTGCATAAACCAGATCGGGAGTTTC
CGCTGCGAGTGCCCCGCAGGCTTCAACTACAACAGCATCCTGCTGGCTTGTGAAGATGTCGATGAGTGTGGCAGCAGGGAG
AGTCCCTGCCAGCAGAATGCTGACTGCATCAACATCCCCGGTAGCTACCGCTGCAAGTGCACCCGAGGGTACAAACTGTCG
CCAGGCGGGGCTTGTGTGGGACGGAATGAGTGTCGGGAGATCCCGAATGTCTGTAGCCATGGTGACTGCATGGACACAGAA
GGCAGCTACATGTGTCTGTGTCACCGTGGATTCCAGGCCTCTGCAGACCAGACCCTGTGCATGGACATTGACGAGTGTGAC
CGGCAGCCTTGTGGAAATGGGACCTGCAAGAACATCATTGGCTCCTACAACTGCCTCTGCTTCCCTGGCTTTGTGGTGACA
CACAATGGGGATTGTGTGGATTTTGATGAGTGTACTACCCTGGTGGGGCAGGTGTGCCGATTTGGCCATTGCCTCAACACA
GCTGGTTCCTTCCACTGCCTCTGCCAGGATGGCTTTGAGCTCACAGCTGATGGGAAGAACTGTGTGGACACCAATGAGTGC
CTCAGCCTTGCAGGAACCTGCCTACCCGGCACTTGCCAGAACCTCGAGGGCTCCTTCCGCTGCATCTGTCCCCCTGGCTTC
CAGGTGCAGAGTGACCACTGCATTGATATCGACGAGTGCTCAGAGGAGCCCAACCTCTGCCTCTTTGGCACCTGTACCAAC
AGCCCTGGGAGCTTCCAGTGCCTCTGCCCACCTGGCTTTGTCCTCTCTGACAATGGGCACCGTTGCTTTGACACACGGCAG
AGTTTCTGCTTCACCCGTTTTGAGGCTGGGAAGTGCTCGGTGCCCAAAGCTTTCAACACCACCAAGACCCGCTGCTGCTGC
AGTAAGAGGCCTGGGGAGGGCTGGGGAGACCCCTGCGAACTGTGTCCCCAGGAGGACTCCCCTCCCCCTCTCCGTCCAGCT
GCCTTTCAGGAGCTCTGCCCCTTTGGCCACGGGGCAGTCCCAGGCCCGGATGACTCCCGAGAAGACGTGAATGAGTGTGCA
GAGAACCCTGGCGTCTGCACTAACGGCGTCTGTGTCAACACCGATGGATCCTTCCGCTGTGAGTGTCCCTTTGGCTACAGC
CTGGACTTCACTGGCATCAACTGTGAGGACACAGACGAGTGCTCTGTCGGCCACCCCTGTGGGCAAGGGACATGCACCAAT
GTCATCGGAGGCTTCGAATGTGCCTGTGCTGACGGCTTTGAGCCTGGCCTCATGATGACCTGCGAGGACATCGACGAATGC
TCCCTGAACCCGCTGCTCTGTGCCTTCCGCTGCCACAATACCGAGGGCTCCTACCTGTGCACCTGTCCAGCCGGCTACACC
CTGCGGGAGGATGGGGCCATGTGTCGAGATGTGGACGAGTGTGCAGATGGTCAGCAGGACTGCCACGCCCGGGGCATGGAG
TGCAAGAACCTCATCGGTACCTTCGCGTGCGTCTGTCCCCCAGGCATGCGGCCCCTGCCTGGCTCTGGGGAGGGCTGCACA
GATGACAATGAATGCCACGCTCAGCCTGACCTCTGTGTCAACGGCCGCTGTGTCAACACCGCGGGCAGCTTCCGGTGCGAC
TGTGATGAGGGATTCCAGCCCAGCCCCACCCTTACCGAGTGCCACGACATCCGGCAGGGGCCCTGCTTTGCCGAGGTGCTG
CAGACCATGTGCCGGTCTCTGTCCAGCAGCAGTGAGGCTGTCACCAGGGCCGAGTGCTGCTGTGGGGGTGGCCGGGGCTGG
GGGCCCCGCTGCGAGCTCTGTCCCCTGCCCGGCACCTCTGCCTACAGGAAGCTGTGCCCCCATGGCTCAGGCTACACTGCT
GAGGGCCGAGATGTAGATGAATGCCGTATGCTTGCTCACCTGTGTGCTCATGGGGAGTGCATCAACAGCCTTGGCTCCTTC
GCTGCCACTGTCAGGCCGGGTACACACCGGATGCTACTGCTACTACCTGCCTGGATATGGATGAGTGCAGCCAGGTGCCCC
AAGCCATGTACCTTCCTCTGCAAAAACACGAAGGGCAGTTTCCTGTGCAGCTGTCCCCGAGGCTACCTGCTGGAGGAGGAT
GGCAGGACCTGCAAAGACCTGGACGAATGCACCTCCCGGCAGCACAACTGTCAGTTCCTCTGTGTCAACACTGTGGGCGCC
TTCACCTGCCGCTGTCCACCCGGCTTCACCCAGCACCACCAGGCCTGCTTCGACAATGATGAGTGCTCAGCCCAGCCTGGC
CCATGTGGTGCCCACGGGCACTGCCACAACACCCCGGGCAGCTTCCGCTGTGAATGCCACCAAGGCTTCACCCTGGTCAGC
TCAGGCCATGGCTGTGAAGATGTGAATGAATGTGATGGGCCCCACCGCTGCCAGCATGGCTGTCAGAACCAGCTAGGGGGC
TACCGCTGCAGCTGCCCCCAGGCTTTCACCCAGCACTCCCAGTGGGCCCAGTGTGTGGATGAGAATGAGTGTGCCCTGTCG
CCCCCCACCTGCGGGAGCGCCTCCTGTCGCAACACTCTTGGTGGCTTCCGCTGCGTCTGCCCCTCCGGCTTTGACTTTGAT
CAGGCCCTCGGGGGCTGCCAGGAGGTGGATGAGTGCGCCGGACGGCGTGGCCCCTGTAGCTACAGCTGTGCCAACACGCCT
GGTGGCTTCCTGTGCGGCTGTCCTCAAGGCTACTTCCGGGTTGGGCAAGGGCACTGTGTCTCCGGCCTGGGCTTCAGCCCC
GGACCCCAGGACACCCCGGACAAAGAGGAGCTGCTCTCGTCTGAAGCCTGCTACGAATGCAAGATCAATGGCCTCTCCCCT
CGGGACCGGCCACGACGCAGTGCCCACAGGGACCACCAGGTGAACCTGGCCACCCTTGACTCCGAGGCCCTGCTGACCTTG
GGCCTGAACCTCTCACACCTGGGCCGGGCCGAGCGCATCCTGGAGCTCCGGCCGGCCCTGGAGGGTCTAGAGGGCCGGATC
CGCTACGTCATCGTCCGCGGAAACGAGCAAGGTTTCTTTCGCATGCATCACCTCCGTGGCGTCAGCTCCCTGCAGCTGGGG
CGGAGGCGGCCGGGGCCTGGAACCTACCGGCTGGAGGTGGTGAGCCACATGGCAGGACCCTGGGGTGTCCAGCAAGAGGGG
CAGCCAGGGCCATGGGGCCAGGCCTTGAGGCTGAAGGTGCAACTGTCAGTTGCTTTAGTTGGGAGGAGCCTCAGTGGGCCC
CAGCTGTCCAGAGAAGGGGGATTCTGGAACTGGGAAGGACTGATGGGGAGAAGCGATGGCTGACCAGATTGAACCCCGAAA
CTCAGGAAGAGTGAAATGCTACACGACAACCTCAGGCAAGCCCGGCCTCTGCCTGGGCCTCTGTGCCAGCCCCGGGGGCCC
CCCAGTTACTCAGTCTTTCCTGGAGACAGCAAGAAGCTGCAATGTGCAATCCCCCTGCCCCCACAGCCAAGGTCAGGAAGA
GGCCCTGTGGTCACCGTGTCTGGCCAATCTCAGGCTTTCACTTCTGTACTGCACTGTGGCTTGCCCTGGCGGGGGGCAGGG
GGTTGGCAGGACATGGCAATGGGCAACTGGGGTGGGCACAGGGCTTATTCCTCGGAGTAGAAGGGTGTACAGGGGGCCCAG
ACTCCACAGTGACTTGCCACATTTGCCCCCCATTTGGAGAATGCTTTTATATCAAAAGTGGAGACGATAATAAAGTTATTT
TGGGTTAAGTCTGCCTGCCCTTTGGCAAGTTCTTGAAGTAAGTAGATGCTGCCCTCGGACTGGGCGAGGCAGATCTTGTGC
CTGGGGAAGCAGAAGGCCTTATGGGCTCCCCAGAATGGTAATAATGGCTCACGCTTCCTGACCACGTACTACATACCAGAC
ACCATTCGATTTTTTTTTTATTTTTTCTGAGACAGGGTCTTGCTCTGTTGCCTAGGTTGGAGTGCAGTGGCGCGATCGTGG
CTCCCTGTGGTCGCCACTTTCCGGGCTCGAGCAATCCTCCCACCTCAGCCTCTCCCAAGTAGCTGGGACCAAAGGTGCACG
CCACCACACCCAGCTAATTTTTTTAAATTTCTTTTTCTTTCTCTTTCTTTTTGAGACCAGCCCGGCCAACATGACGAAACC
CCATCTCTAATA

The disclosed NOV2b nucleic acid sequence, localized to chromsome 19, has 3617 of 5008 bases (72%) identical to a Rattuts norvegicus fibrillin 2 (fbn2) mRNA (gb:GENBANK-ID:AF135060|acc:AF135060.1) (E=0.0).

A NOV2b polypeptide (SEQ ID NO: 8) encoded by SEQ ID NO: 7 has 2844 amino acid residues and is presented using the one-letter code in Table 2D. Signal P, Psort and/or Hydropathy results predict that NOV2b contains a signal peptide and is likely to be localized to the nucleus with a certainty of 0.6000. Although PSORT suggests that the Fibrillin-like protein may be localized in the nucleus, the NOV2b protein is similar to the Fibrillin family, some members of which are released extracellularly. Therefore it is likely that NOV2b protein shows a similar localization. The most likely cleavage site for a NOV2b peptide is between amino acids 29 and 30, at: AGG-QG.

TABLE 2D

Encoded NOV2b protein sequence (SEQ ID NO:8).
MTLEGLYLARGPLARLLLAWSALLCMAGGQGRWDGALEAAGPGRVRRRGSPGILQGPNVCGSRFHAYCCPGWRTFPGRSQC
VVPICRRACGEGFCSQPNLCTCADGTLAPSCGVSRGSGCSVSCMNGGTCRGASCLCQKGYTGTVCCQPICDRGCHNGGRCI
GPNRCACVYGFMGPQCERDYRTGSCFGQVGPEGCQHQLTGLVCTKALCCATVGRAWGLPCELCPAQPHPCRRGFIPNTHTG
ACQDVDECQAVPGLCQGGSCVNMVGSFHCRCPVGHRLSDSSAACEDYRAGACFSVLFGGRCAGDLAGHYTRRQCCCDRGRC
WAAGPVPELCPPRGSNEFQQLCAQRLPLLPGHPGLFPGLLGFGSNGMGPPLGPARLNPHGSDARGIPSLGPGNSNIGTATL
NQTIDICRHFTNLCLNGRCLPTPSSYRCECNVGYTQDVRGECIDVDECTSSPCHHGDCVNIPGTYHCRCYPGFQATPTRQA
CVDVDECIVSGGLCHLGRCVNTEGSFQCVCNAGFELSPDGKNCVDHNECATSTMCVNGVCLNEDGSFSCLCKPGFLLAPGG
HYCMDIDECQTPGICVNGHCTNTEGSFRCQCLGGLAVGTDGRVCVDTHVRSTCYGAIEKGSCARPFPGTVTKSECCCANPD
HGFGEPCQLCPAKNSAEFQALCSSGLGITTDGRDINECALDPEVCANGVCENLRGSYRCVCNLGYEAGASGKDCTKVKECA
LNSLLCDNGWCQNSPGSYSCSCPPGFHFWQDTEICKDVDECLSSPCVSGVCRNLAGSYTCKCGPGSRLDPSGTFCLDSTKG
TCWLKIQESRCEVNLQGASLRSECCATLGAAWGSPCERCEIDPACARGFARMTGVTCDDVNECESFPGVCPNGRCVNTAGS
FRCECPEGLMLDASGRLCVDVRLEPCFLRWDEDECGVTLPGKYRMDVCCCSIGAVWGVECEACPDPESLEFASLCPRGLGF
ASRDFLSGRPFYKDVNECKVFPGLCTHGTCRNTVGSFHCACAGGFALDAQERNCTDIDECRISPDLCGQGTCVNTPGSFEC
ECFPGYESGFMLMKNCMDVDECARDPLLCRGGTCTNTDGSYKCQCPPGHELTAKGTACEDIDECSLSDGLCPHGQCVNVIG
AFQCSCHAGFQSTPDRQGCVDINECRVQNGGCDVHRINTEGSYRCSCGQGYSLMPDGRACADVDECEENPRVCDQGHCTNM
PGGHRCLCYDGFMATPDMRTCVDVDECDLNPHICLHGDCENTKGSFVCHCQLGYMVRKGATGCSDVDECEVGGHNCDSHAS
CLNIPGSFSCRCLPGWVGDGFECHDLDECVSQEHRCSPRGDCLNVPGSYRCTCRQGFAGDGFFCEDRDECAENVDLCDNGQ
CLNAPGGYRCECEMGFDPTEDHRACQDVDECAQENLCAFGSCENLPGMFRCICNGGYELDRGGGNCTDINECADPVNCING
VCINTPGSYLCSCPQDFELNPSGVGCVDTRAGNCFLETHDRGDSGISCSAEIGVGVTRASCCCSLGRAWGNPCELCPMANT
TEYRTLCPGGEGFQPNRITVILEDIDECQELPGLCQGGDCVNTFGSFQCECPPGYHLSEHTRICEDIDECSTHSGICGPGT
CYNTLGNYTCVCPAEYLQVNGGNNCMDMRKSVCFRHYNGTCQNELAFNVTRKMCCCSYNIGQAWNRPCEACPTPISPDYQI
LCGNQAPGFLTDIHTGKPLDIDECGEIPAICANGICINQIGSFRCECPAGFNYNSILLACEDVDECGSRESPCQQNADCIN
IPGSYRCKCTRGYKLSPGGACVGRNECREIPNVCSHGDCMDTEGSYMCLCHRGFQASADQTLCMDIDECDRQPCGNGTCKN
IIGSYNCLCFPGFVVTHNGDCVDFDECTTLVGQVCRFGHCLNTAGSFHCLCQDGFELTADGKNCVDTNECLSLAGTCLPGT
CQNLEGSFRCICPPGFQVQSDHCIDIDECSEEPNLCLFGTCTNSPGSFQCLCPPGFVLSDNGHRCFDTRQSFCFTRFEAGK
CSVPKAFNTTKTRCCCSKRPGEGWGDPCELCPQEDSPPPLRPAAFQELCPFGHGAVPGPDDSREDVNECAENPGVCTNGVC
VNTDGSFRCECPFGYSLDFTGINCEDTDECSVGHPCGQGTCTNVIGGFECACADGFEPGLMMTCEDIDECSLNPLLCAFRC
NTEDSYLCTCPAGYTLREDGAMCRDVDECADGQQDCHARGMECKNLIGTFACVCPPGMRPLPGSGEGCTDDNECHAQPLDL
CVNGRCVNTAGSFRCDCDEGFQPSPTLTECHDIRQGPCFAEVLQTMCRSLSSSSEAVTRAECCCGGGRGWGPRCELCPLPG
TSAYRKLCPHGSGYTAEGRDVDECRMLAHLCAHGECINSLGSFRCHCQAGYTPDATATTCLDMDECSQVPKPCTFLCKNTK
GSFLCSCPRGYLLEEDGRTCKDLDECTSRQHNCQFLCVNTVGAFTCRCPPGFTQHHQACFDNDECSAQPGPCGAHGHCHNT
PGSFRCECHQGFTLVSSGHGCEDVNECDGPHRCQHGCQNQLGGYRCSCPQAFTQHSQWAQCVDENECALSPPTCGSASCRN
TLGGFRCVCPSGFDFDQALGGCQEVDECAGRRGPCSYSCANTPGGFLCGCPQGYFRVGQGHCVSGLGFSPGPQDTPDKEEL
LSSEACYECKINGLSPRDRPRRSAHRDHQVNLATLDSEALLTLGLNLSHLGRAERILELRPALEGLEGRIRYVIVRGNEQG
FFRMHHLRGVSSLQLGRRRPGPGTYRLEVVSHMAGPWGVQQEGQPGPWGQALRLKVQLSVALVGRSLSGPQLSREGGFWNW
EGLIPRSDG

The NOV2b amino acid sequence has 1797 of 2750 amino acid residues (65%) identical to, and 2161 of 2750 amino acid residues (78%) similar to, a Homo sapiens 2911 amino acid residue fibrillin 2 precursor protein (ptnr:SWISSNEW-ACC:P35556) (E=0.0).

NOV2b is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the NOV2b sequence.

NOV2c

A disclosed NOV2c nucleic acid of 9993 nucleotides (also referred to as CG88987-01) encoding a novel Fibrillin-like protein is shown in Table 2E. An open reading frame was identified beginning with an ATG initiation codon at nucleotides 520-522 and ending with a TGA codon at nucleotides 9151-9153. Putative untranslated regions upstream from the intiation codon and downstream from the termination codon are underlined in Table 2E, and the start and stop codons are in bold letters.

TABLE 2E

NOV2c nucleotide sequence (SEQ ID NO:9).
CCTCCCATTCCTCTCCTCTCTCTCCCATCTGGACAGCCCCCAGCCTTCTGACACCCTGTTTCCCCCCCGGGACCCTGCTGC
CCTTCCCCACACCACCTAACATGCATTTCTGACCTTGCTCCTCCCACTCAAAGCTTTTCAAGGGCTCTTCATTGTCCTTGA
CGAATGAGCAAAAGCTGCATGACACTGCTATGCCCACTCCTGCAACGCCTGGCCCCACCTGGCCCCACCTGGCCCTCTCTC
CCCTCCCTTCCACTTCCCACTCCCGTCTTGCCTTGGCTGTGCACCCTCAGCCCCAAGACCCTTCTCTAACATCACACCTCC
TCCAGGAAGCCTCCTCTGACTGTTCACCGCTTCCCGGTTGGGGCTGGATGCCTCCTTGGGCTCCCAGAGGCCCCGGGGCTG
CCCCATTTGGCAACCACCCTGCCCTCCTCGTTGTGTTGGGTCCCCCATGGCTAGGGTGGGGGGCTCATCTAGGGCTGACTC
TGCTTCCTCTCCGCAGCCTCCAGGGGACACGCCATGACTCTGGAGGGTCTGTATTTGGCAAGGGGCCCCCTGGCCCGGCTC
CTGCTGGCCTGGTCGGCCCTGTTGTGCATGGCAGGTGGCCAAGGCCGCTGGGACGGGGCCTTGGAGGCTGCAGGTCCTGGA
CGTGTGCGGAGGCGGGGCAGCCCAGGCATCTTGCAGGGGCCGAATGTGTGCGGCTCCCGGTTCCATGCCTACTGCTGTCCA
GGCTGGAGGACATTCCCTGGCAGGAGCCAGTGTGTCGTACCCATCTGTAGGCGCGCCTGCGGTGAAGGCTTCTGCTCCCAG
CCCAACCTGTGCACCTGTGCGGATGGGACGCTGGCTCCCAGCTGCGGGGTGAGCCGAGGGTCAGGGTGCAGTGTGAGCTGT
ATGAATGGGGGCACCTGCCGGGGGGCGTCCTGTCTGTGTCAGAAGGGCTACACAGGCACCGTGTGTGGGCAGCCCATCTGT
GACCGCGGCTGCCACAATGGGGGTCGCTGCATTGGGCCCAACCGCTGCGCCTGTGTGTATGGCTTCATGGGACCTCAATGT
GAGAGAGATTACCGGACGGGACCCTGCTTTGGCCAAGTAGGCCCCGAGGGGTGCCAGCATCAGCTGACGGGCCTCGTGTGC
ACCAAGGCACTTTGCTGTGCCACTGTGGGCCGTGCCTGGGGCCTTCCATGTGAACTTTGCCCTGCACAGCCACACCCCTGC
CGCCGCGGCTTCATCCCCAATATCCACACGGGGGCCTGCCAAGATGTGGATGAGTGCCAGGCTGTGCCAGGCCTGTGCCAG
GGAGGCAGCTGCGTCAACATGGTGGGCTCCTTCCATTGCCGCTGTCCAGTTGGACACCGGCTCAGTGACAGCAGCGCCGCA
TGTGAAGACTACGATGAATGCAGCACCATTCCTGGAATCTGTGAAGGGGGTGAATGTACAAACACAGTCAGCAGTTACTTT
TGCAAATGTCCTCCTGGTTTTTACACCTCTCCAGATGGTACTCTTCATGGACAGTCGCGGGCCGGCGCCTGCTTCTCAGTG
CTTTTCGGGGGCCGCTGTGCTGGAGACCTCGCCGGCCACTACACTCGCAGGCAGTGCTGCTGTGACAGGGGCAGGTGCTGG
GCAGCTGGCCCGGTCCCTGAGCTGTGTCCTCCTCGGGGCTCCAATGAATTCCAGCAACTGTGCGCCCAGCGGCTGCCGCTG
CTACCCGGCCACCCTGGCCTCTTCCCTGGCCTCCTGGGCTTCGGATCCAATGGCATGGGTCCCCCTCTTGGGCCAGCGCGA
CTCAACCCCCATGGCTCTGATGCGCGTGGGATCCCCAGCCTGGGCCCTGGCAACTCTAATATTGGCACTGCTACCCTGAAC
CAGACCATTGACATCTGCCGACACTTCACCAACCTGTGTCTGAATGGCCGCTGCCTGCCCACGCCTTCCAGCTACCGCTGC
GAGTGTAACGTGGGCTACACCCAGGACGTGCGCGGCGAGTGCATTGATGTAGACGAATGCACCAGCAGCCCCTGCCACCAC
GGTGACTGCGTCAACATCCCCGGCACCTACCACTGCCGGTGCTACCCGGGCTTCCAGGCCACGCCCACCAGGCAGGCATGC
GTGGATGTGGACGAGTGCATTGTCAGTGGTGGCCTTTGTCACCTGGGCCGCTGTGTCAACACAGAGGGCAGCTTCCAGTGT
GTCTGCAATGCAGGCTTCGAGCTCAGCCCTGACGGCAAGAACTGTGTGGACCACAACGAGTGTGCCACCAGCACCATGTGC
GTCAACGGCGTGTGTCTCAACGAGGATGGCAGCTTCTCCTGCCTCTGCAAACCCGGCTTCCTGCTGGCGCCTGGCGGCCAC
TACTGCATGGACATTGACGAGTGCCAGACGCCCGGCATCTGCGTGAACGGCCACTGTACCAACACCGAGGGCTCCTTCCGC
TGCCAGTGCCTGGGGGGGCTGGCGGTAGGCACGGATGGCCGCGTGTGCGTGGACACCCACGTGCGCAGCACCTGCTATGGG
GCCATCGAGAAGGGCTCCTGTGCCCGCCCCTTCCCTGGCACTGTCACCAAGTCCGAGTGCTGCTGTGCCAATCCGGACCAC
GGTTTTGGGGAGCCCTGCCAGCTTTGTCCTGCCAAAGACTCCGCTGAGTTCCAGGCACTGTGCAGCAGTGGGCTTGGCATT
ACCACGGATGGTCGAGACATCAACGAGTGTGCTCTGGATCCTGAGGTTTGTGCCAATGGCGTGTGCGAGAACCTTCGGGGC
AGCTACCGCTGTGTCTGCAACCTGGGTTATGAGGCAGGTGCCTCAGGCAAGGACTGCACAGACGTGGATGAGTGTGCCCTC
AACAGCCTCCTGTGTGACAACGGGTGGTGCCAGAATAGCCCTGGCAGCTACAGCTGCTCCTGCCCCCCCGGCTTCCACTTC
TGGCAGGACACGGAGATCTGCAAAGATGTCGACGAATGCCTGTCCAGCCCGTGTGTGAGTGGCGTCTGTCGGAACCTGGCC
GGCTCCTACACCTGCAAATGTGGCCCTGGCAGCCGGCTGGACCCCTCTGGTACCTTCTGTCTAGACAGCACCAAGGGCACC
TGCTGGCTGAAGATCCAGGAGAGCCGCTGTGAGGTGAACCTTCAGGGAGCCAGCCTGCGGTCTGAGTGCTGTGCCACCCTC
GGGGCAGCCTGGGGGAGCCCCTGCGAACGCTGCGAGATCGACCCTGCCTGTGCCCGGGGCTTTGCCCGGATGACGGGTGTC
ACCTGCGATGATGTGAACGAGTGTGAGTCCTTCCCGGGAGTCTGTCCCAACGGGCGTTGCGTCAACACTGCTGGGTCTTTC
CGCTGTGAGTGTCCAGAGGGCCTGATGCTGGACGCCTCAGGCCGGCTGTGCGTGGATGTGAGATTGGAACCATGTTTCCTG
CGATGGGATGAGGATGAGTGTGGGGTCACCCTGCCTGGCAAGTACCGGATGGACGTCTGCTGCTGCTCCATCGGGGCCGTG
TGGGGAGTCGAGTGCGAGGCCTGCCCGGATCCCGAGTCTCTGGAGTTCGCCAGCCTGTGCCCGCGGGGGCTGGGCTTCGCC
AGCCGGGACTTCCTGTCTGGCCGACCATTCTATAAAGATGTGAATGAATGCAAGGTGTTCCCTGGCCTCTGCACGCACGGT
ACCTGCAGAAACACGGTGGGCAGCTTCCACTGCGCCTGTGCGGGGGGCTTCGCCCTGGATGCCCAGGAACGGAACTGCACA
GATATCGACGAGTGTCGCATCTCTCCTGACCTCTGCGGCCAGGGCACCTGTGTCAACACGCCGGGCAGCTTTGAGTGCGAG
TGTTTTCCCGGCTACGAGAGTGGCTTCATGCTGATGAAGAACTGCATGGACGTGGACGAGTGTGCAAGGGACCCGCTGCTC
TGCCGGGGAGGCACTTGCACCAACACGGATGGGAGCTACAAGTGCCAGTGTCCCCCTGGGCATGAGCTGACGGCCAAGGGC
ACTGCCTGTGAGGACATCGATGAGTGCTCCCTGAGTGATGGCCTGTGTCCCCATGGCCAGTGTGTCAATGTCATCGGTGCC
TTCCAGTGCTCCTGCCATGCCGGCTTCCAGAGCACACCTGACCGCGGAGCTACAAGTGCCAGTTGCCCGACGGAAGGGCAT
GTGCAGGTCGTCCTGGGGCCTGGGGAGCAGATGTGTACTGGGTGGTCTATCAGGGCAAAGCTAAGCACAGTCCCCAACCCT
GCTCCCCCAGACGTGGACGAGTGTGAAGAGAACCCCCGCGTTTGTGACCAAGGCCACTGCACCAACATGCCAGGGGGTCAC
CGCTGCCTGTGCTATGATGGCTTCATGGCCACGCCAGACATGAGGACATGTGTTGATGTGGATGAGTGTGACCTGAACCCT
CACATCTGCCTCCATGGGGACTGCGAGAACACGAAGGGTTCCTTTGTCTGCCACTGTCAGCTGGGCTACATGGTCAGGAAG
GGGGCCACAGGCTGCTCTGATGTGGATGAATGCGAGGTTGGAGGACACAACTGTGACAGTCACGCCTCCTGTCTCAACATC
CCAGGGAGTTTCAGCTGTAGGTGCCTGCCAGGCTGGGTGGGGGATGGCTTCGAATGTCACGACCTGGATGAATGCGTCTCC
CAGGAGCACCGGTGCAGCCCAAGAGGTGACTGTCTCAATGTCCCTGGCTCCTACCGCTGCACCTGCCGCCAGGGCTTTGCC
GGGGATGGCTTCTTCTGCGAAGACAGGGATGAATGTGCCGAGAACGTGGACCTCTGTGACAACGGGCAGTGCCTCAATGCG
CCCGGCGGGTACCGCTGTGAATGTGAGATGGGCTTTGACCCCACCGAGGACCACCGGGCCTGCCAGGATGTGGACGAGTGT
GCGCAAGAGAACCTCTGTGCATTTGGGAGCTGTGAGAACCTGCCTGGAATGTTCCGCTGCATCTGCAATGGTGGCTACGAA
CTGGACCGAGGGGGTGGCAACTGCACAGACATCAACGAGTGTGCAGACCCAGTAAACTGCATCAACGGCGTGTGCATTAAC
ACCCCCGGCAGCTACCTCTGCAGCTGCCCCCAGGATTTTGAGCTGAACCCCAGCGGAGTGGGCTGCGTGGACACTCGGGCC
GGGAACTGTTTCCTGGAGACGCATGACCGAGGGGACAGTGGCATTTCCTGCAGTGCCGAGATCGGAGTTGGTGTCACCCGA
GCTTCCTGCTGTTGCTCCCTGGGCCGGGCTTGGGGCAATCCCTGTGAGCTGTGCCCTATGGCCAACACCACTGAGTACAGA
ACCCTGTGCCCGGGTGGTGAGGGCTTCCAGCCTAACCGCATCACTGTCATTCTGGAAGACATCGACGAGTGCCAAGAGCTG
CCAGGGCTGTGTCAGGGGGGTGACTGCGTCAACACGTTTGGCAGTTTCCAGTGTGAGTGCCCACCTGGCTACCACCTCAGT
GAGCACACCCGCATCTGTGAGGATATTGACGAATGCTCCACACACTCCGGCATCTGTGGCCCTGGCACCTGCTACAACACC
CTGGGGAACTACACCTGTGTCTGCCCTGCAGAGTACCTCCAAGTCAATGGTGGCAACAACTGCATGGATATGAGGAAGAGT
GTCTGCTTCCGGCACTATAACGGCACATGTCAAAATGAGCTGGCCTTCAACGTGACCCGGAAAATGTGTTGCTGCTCCTAC
AACATTGGCCAGGCCTGGAATAGACCCTGTGAGGCCTGCCCCACTCCCATCAGTCCTGACTACCAGATCCTGTGTGGAAAT
CAGGCCCCGGGATTCCTCACTGACATCCACACGGGGAAGCCCCTTGACATTGATGAGTGTGGGGAGATCCCCGCCATCTGT
GCCAATGGCATCTGCATAAACCAGATCGGGAGTTTCCGCTGCGAGTGCCCCGCAGGCTTCAACTACAACAGCATCCTGCTG
GCTTGTGAAGATGTCGATGAGTGTGGCAGCAGGGAGAGTCCCTGCCAGCAGAATGCTGACTGCATCAACATCCCCGGTAGC
TACCGCTGCAAGTGCACCCGAGGGTACAAACTGTCGCCAGGCGGGGCTTGTGTGGGACGGAATGAGTGTCGGGAGATCCCG
AATGTCTGTAGCCATGGTGACTGCATGGACACAGAAGGCAGCTACATGTGTCTGTGTCACCGTGGATTCCAGGCCTCTGCA
GACCAGACCCTGTGCATGGACATTGACGAGTGTGACCGGCAGCCTTGTGGAAATGGGACCTGCAAGAACATCATTGGCTCC
TACAACTGCCTCTGCTTCCCTGGCTTTGTGGTGACACACAATGGGGATTGTGTGGATTTTGATGAGTGTACTACCCTGGTG
GGGCAGGTGTGCCGATTTGGCCATTGCCTCAACACAGCTGGTTCCTTCCACTGCCTCTGCCAGGATGGCTTTGAGCTCACA
GCTGATGGGAAGAACTGTGTGGACACCAATGAGTGCCTCAGCCTTGCAGGAACCTGCCTACCCGGCACTTGCCAGAACCTC
GAGGGCTCCTTCCGCTGCATCTGTCCCCCTGGCTTCCAGGTGCAGAGTGACCACTGCATTGATATCGACGAGTGCTCAGAG
GAGCCCAACCTCTGCCTCTTTGGCACCTGTACCAACAGCCCTGGGAGCTTCCAGTGCCTCTGCCCACCTGGCTTTGTCCTC
TCTGACAATGGGCACCGTTGCTTTGACACACGGCAGAGTTTCTGCTTCACCCGTTTTGAGGCTGGGAAGTGCTCGGTGCCC
AAAGCTTTCAACACCACCAAGACCCGCTGCTGCTGCAGTAAGAGGCCTGGGGAGGGCTGGGGAGACCCCTGCGAACTGTGT
CCCCAGGAAGGCAGCGCTGCCTTTCAGGAGCTCTGCCCCTTTGGCCACGGGGCAGTCCCAGGCCCGGATGACTCCCGAGAA
GACGTGAATGAGTGTGCAGAGAACCCTGGCGTCTGCACTAACGGCGTCTGTGTCAACACCGATGGATCCTTCCGCTGTGAG
TGTCCCTTTGGCTACAGCCTGGACTTCACTGGCATCAACTGTGTGGACACAGACGAGTGCTCTGTCGGCCACCCCTGTGGG
CAAGGGACATGCACCAATGTCATCGGAGGCTTCGAATGTGCCTGTGCTGACGGCTTTGAGCCTGGCCTCATGATGACCTGC
GAGGACATCGACGAATGCTCCCTGAACCCGCTGCTCTGTGCCTTCCGCTGCCACAATACCGAGGGCTCCTACCTGTGCACC
TGTCCAGCCGGCTACACCCTGCGGGAGGATGGGGCCATGTGTCGAGATGTGGACGAGTGTGCAGATGGTCAGCAGGACTGC
CACGCCCGGGGCATGGAGTGCAAGAACCTCATCGGTACCTTCGCGTGCGTCTGTCCCCCAGGCATGCGGCCCCTGCCTGGC
TCTGGGGAGGGCTGCACAGATGACAATGAATGCCACGCTCAGCCTGACCTCTGTGTCAACGGCCGCTGTGTCAACACCGCG
GGCAGCTTCCGGTGCGACTGTGATGAGGGATTCCAGCCCAGCCCCACCCTTACCGAGTGCCACGACATCCGGCAGGGGCCC
TGCTTTGCCGAGGTGCTGCAGACCATGTGCCGGTCTCTGTCCAGCAGCAGTGAGGCTGTCACCAGGGCCGAGTGCTGCTGT
GGGGGTGGCCGGGGCTGGGGGCCCCGCTGCGAGCTCTGTCCCCTGCCCGGCACCTCTGCCTACAGGAAGCTGTGCCCCCAT
GGCTCAGGCTACACTGCTGAGGGCCGAGATGTAGATGAATGCCGTATGCTTGCTCACCTGTGTGCTCATGGGGAGTGCATC
AACAGCCTTGGCTCCTTCCGCTGCCACTGTCAGGCCGGGTACACACCGGATGCTACTGCTACTACCTGCCTGGATATGGAT
GAGTGCAGCCAGGTCCCCAAGCCATGTACCTTCCTCTGCAAAAACACGAAGGGCAGTTTCCTGTGCAGCTGTCCCCGAGGC
TACCTGCTGGAGGAGGATGGCAGGACCTGCAAAGACCTGGACGAATGCACCTCCCGGCAGCACAACTGTCAGTTCCTCTGT
GTCAACACTGTGGGCGCCTTCACCTGCCGCTGTCCACCCGGCTTCACCCAGCACCACCAGGCCTGCTTCGACAATGATGAG
TGCTCAGCCCAGCCTGGCCCATGTGGTGCCCACGGGCACTGCCACAACACCCCGGGCAGCTTCCGCTGTGAATGCCACCAA
GGCTTCACCCTGGTCAGCTCAGGCCATGGCTGTGAAGATGTGAATGAATGTGATGGGCCCCACCGCTGCCAGCATGGCTGT
CAGAACCAGCTAGGGGGCTACCGCTGCAGCTGCCCCCAGGCTTTCACCCAGCACTCCCAGTGGGCCCAGTGTGTGGATGAG
AATGAGTGTGCCCTGTCGCCCCCCACCTGCGGGAGCGCCTCCTGTCGCAACACTCTTGGTGGCTTCCGCTGCGTCTGCCCC
TCCGGCTTTGACTTTGATCAGGCCCTCGGGGGCTGCCAGGAGGTGGATGAGTGCGCCGGACGGCGTGGCCCCTGTAGCTAC
AGCTGTGCCAACACGCCTGGTGGCTTCCTGTGCGGCTGTCCTCAAGGCTACTTCCGGGTTGGGCAAGGGCACTGTGTCTCC
GGCCTGGGCTTCAGCCCCGGACCCCAGGACACCCCGGACAAAGAGGAGCTGCTCTCGTCTGAAGCCTGCTACGAATGCAAG
ATCAATGGCCTCTCCCCTCGGGACCGGCCACGACGCAGTGCCCACAGGGACCACCAGGTGAACCTGGCCACCCTTGACTCC
GAGGCCCTGCTGACCTTGGGCCTGAACCTCTCACACCTGGGCCGGGCCGAGCGCATCCTGGAGCTCCGGCCGGCCCTGGAG
GGTCTAGAGGGCCGGATCCGCTACGTCATCGTCCGCGGAAACGAGCAAGGTTTCTTTCGCATGCATCACCTCCGTGGCGTC
AGCTCCCTGCAGCTGGGGCGGAGGCGGCCGGGGCCTGGAACCTACCGGCTGGAGGTGGTGAGCCACATGGCAGGACCCTGG
GGTGTCCAGCAAGAGGGGCAGCCAGGGCCATGGGGCCAGGCCTTGAGGCTGAAGGTGCAACTGTCAGTTGCTTTAGTTGGG
AGGAGCCTCAGTGGGCCCCAGCTGTCCAGAGAAGGGGGATTCTGGAACTGGGAAGGACTGATCCCCAGAAGCGATGGCTGA
CCAGATTGAACCCCGAAACTCAGGAAGAGTGAAATGCTACACGACAACCTCAGGCAAGCCCGGCCTCTGCCTGGGCCTCTG
TGCCAGCCCCGGGGGCCCCCCAGTTACTCAGTCTTTCCTGGAGACAGCAAGAAGCTGCAATGTGCAATCCCCCTGCCCCCA
CAGCCAAGGTCAGGAAGAGGCCCTGTGGTCACCGTGTCTGGCCAATCTCAGGCTTTCACTTCTGTACTGCACTGTGGCTTG
CCCTGGCGGGGGGCAGGGGGTTGGCAGGACATGGCAATGGGCAACTGGGGTGGGCACAGGGCTTATTCCTCGGAGTAGAAG
GGTGTACAGGGGGCCCAGACTCCACAGTGACTTGCCACATTTGCCCCCCATTTGGAGAATGCTTTTATATCAAAAGTGGAG
ACGATAATAAAGTTATTTTGGGTTAAGTCTGCCTGCCCTTTGGCAAGTTCTTGAAGTAAGTAGATGCTGCCCTCGGACTGG
GCGAGGCAGATCTTGTGCCTGGGGAAGCAGAAGGCCTTATGGGCTCCCCAGAATGGTAATAATGGCTCACGCTTCCTGACC
ACGTACTACATACCAGACACCATTCGATTTTTTTTTTATTTTTTCTGAGACAGGGTCTTGCTCTGTTGCCTAGGTTGGAGT
GCAGTGGCGCGATCGTGGCTCCCTGTGGTCGCCACTTTCCGGGCTCGAGCAATCCTCCCACCTCAGCCTCTCCCAAGTAGC
TGGGACCAAAGGTGCACGCCACCACACCCAGCTAATTTTTTTAAATTTCTTTTTCTTTCTCTTTCTTTTTGAGACCAGCCC
GGCAACATGACGAAACCCCATCTCTAATA

The disclosed NOV2c nucleic acid sequence, localized to chromsome 19, has 3194 of 4382 bases (72%) identical to a Mus musculus fibrillin 2 (fbn2) mRNA (gb:GENBANK-ID:MUSFBN2|acc:L39790.1) (E=0.0).

A NOV2c polypeptide (SEQ ID NO: 10) encoded by SEQ ID NO: 9 has 2877 amino acid residues and is presented using the one-letter code in Table 2F. Signal P, Psort and/or Hydropathy results predict that NOV2c contains a signal peptide and is likely to be localized to the nucleus with a certainty of 0.6000. Although PSORT suggests that the Fibrillin-like protein may be localized in the nucleus, the NOV2c protein is similar to the Fibrillin family, some members of which are released extracellularly. Therefore it is likely that NOV2c protein shows a similar localization. The most likely cleavage site for a NOV2c peptide is between amino acids 29 and 30, at: AGG-QG.

TABLE 2F

Encoded NOV2c protein sequence (SEQ ID NO:10).
MTLEGLYLARGPLARLLLAWSALLCMAGGQGRWDGALEAAGPGRVRRRGSPGILQGPNVCGSRFHAYCCPGWRTFPGRSQC
VVPICRRACGEGFCSQPNLCTCADGTLAPSCGVSRGSGCSVSCMNGGTCRGASCLCQKGYTGTVCGQPICDRGCHNGGRCI
GPNRCACVYGFMGPQCERDYRTGPCFGQVGPEGCQHQLTGLVCTKALCCATVGRAWGLPCELCPAQPHPCRRGFIPNIHTG
ACQDVDECQAVPGLCQGGSCVNMVGSFHCRCPVGHRLSDSSAACEDYDECSTIPGICEGGECTNTVSSYFCKCPPGFYTSP
DGTLHGQSRAGACFSVLFGGRCAGDLAGHYTRRQCCCDRGRCWAAGPVPELCPPRGSNEFQQLCAQRLPLLPGHPGLFPGL
LGFGSNGMGPPLGPARLNPHGSDARGIPSLGPGNSNIGTATLNQTIDICRHFTNLCLNGRCLPTPSSYRCECNVGYTQDVR
GECIDVDECTSSPCHHGDCVNIPGTYHCRCYPGFQATPTRQACVDVDECIVSGGLCHLGRCVNTEGSFQCVCNAGFELSPD
GKNCVDHNECATSTMCVNGVCLNEDGSFSCLCKPGFLLAPGGHYCMDIDECQTPGICVNGHCTNTEGSFRCQCLGGLAVGT
DGRVCVDTHVRSTCYGAIEKGSCARPFPGTVTKSECCCANPDHGFGEPCQLCPAKDSAEFQALCSSGLGITTDGRDINECA
LDPEVCANGVCENLRGSYRCVCNLGYEAGASGKDCTDVDECALNSLLCDNGWCQNSPGSYSCSCPPGFHFWQDTEICKDVD
ECLSSPCVSGVCRNLAGSYTCKCGPGSRLDPSGTFCLDSTKGTCWLKIQESRCEVNLQGASLRSECCATLGAAWGSPCERC
EIDPACARGFARMTGVTCDDVNECESFPGVCPNGRCVNTAGSFRCECPEGLMLDASGRLCVDVRLEPCFLRWDEDECGVTL
PGKYRMDVCCCSIGAVWGVECEACPDPESLEFASLCPRGLGFASRDFLSGRPFYKDVNECKVFPGLCTHGTCRNTVGSFHC
ACAGGFALDAQERNCTDIDECRISPDLCGQGTCVNTPGSFECECFPGYESGFMLMKNCMDVDECARDPLLCRGGTCTNTDG
SYKCQCPPGHELTAKGTACEDIDECSLSDGLCPHGQCVNVIGAFQCSCHAGFQSTPDRGATSASCPTEGHVQVVLGPGEQM
CTGWSIRAKLSTVPNPAPPDVDECEENPRVCDQGHCTNMPGGHRCLCYDGFMATPDMRTCVDVDECDLNPHICLHGDCENT
KGSFVCHCQLGYMVRKGATGCSDVDECEVGGHNCDSHASCLNIPGSFSCRCLPGWVGDGFECHDLDECVSQEHRCSPRGDC
LNVPGSYRCTCRQGFAGDGFFCEDRDECAENVDLCDNGQCLNAPGGYRCECEMGFDPTEDHRACQDVDECAQENLCAFGSC
ENLPGMFRCICNGGYELDRGGGNCTDINECADPVNCINGVCINTPGSYLCSCPQDFELNPSGVGCVDTRAGNCFLETHDRG
DSGISCSAEIGVGVTRASCCCSLGRAWGNPCELCPMANTTEYRTLCPGGEGFQPNRITVILEDIDECQELPGLCQGGDCVN
TFGSFQCECPPGYHLSEHTRICEDIDECSTHSGICGPGTCYNTLGNYTCVCPAEYLQVNGGNNCMDMRKSVCFRHYNGTCQ
NELAFNVTRKMCCCSYNIGQAWNRPCEACPTPISPDYQILCGNQAPGFLTDIHTGKPLDIDECGEIPAICANGICINQIGS
FRCECPAGFNYNSILLACEDVDECGSRESPCQQNADCINIPGSYRCKCTRGYKLSPGGACVGRNECREIPNVCSHGDCMDT
EGSYMCLCHRGFQASADQTLCMDIDECDRQPCGNGTCKNIIGSYNCLCFPGFVVTHNGDCVDFDECTTLVGQVCRFGHCLN
TAGSFHCLCQDGFELTADGKNCVDTNECLSLAGTCLPGTCQNLEGSFRCICPPGFQVQSDHCIDIDECSEEPNLCLFGTCT
NSPGSFQCLCPPGFVLSDNGHRCFDTRQSFCFTRFEAGKCSVPKAFNTTKTRCCCSKRPGEGWGDPCELCPQEGSAAFQEL
CPFGHGAVPGPDDSREDVNECAENPGVCTNGVCVNTDGSFRCECPFGYSLDFTGINCVDTDECSVGHPCGQGTCTNVIGGP
ECACADGFEPGLMMTCEDIDECSLNPLLCAFRCHNTEGSYLCTCPAGYTLREDGAMCRDVDECADGQQDCHARGMECKNLI
GTGACVCPPGMRPLPGSGEGCTDDNECHAQPDLCVNGRCVNTAGSFRCDCDEGFQPSPTLTECHDIRQGPCFAEVLQTMCR
SLSSSSEAVTRAECCCGGGRGWGPRCELCPLPGTSAYRKLCPHGSGYTAEGRDVDECRMLAHLCAHGECINSLGSFRCHCQ
AGYTPDATATTCLDMDECSQVPKPCTFLCKNTKGSFLCSCPRGYLLEEDGRTCKDLDECTSRQHNCQFLCVNTVGAFTCRC
PPGFTQHHQACFDNDECSAQPGPCGAHGHCHNTPGSFRCECHQGFTLVSSGHGCEDVNECDGPHRCQHGCQNQLGGYRCSC
PQAFTQHSQWAQCVDENECALSPPTCGSASCRNTLGGFRCVCPSGFDFDQALGGCQEVDECAGRRGPCSYSCANTPGGFLC
GCPQGYFRVGQGHCVSGLGFSPGPQDTPDKEELLSSEACYECKINGLSPRDRPRRSAHRDHQVNLATLDSEALLTLGLNLS
HLGRAERILELRPALEGLEGRIRYVIVRGNEQGFFRMHHLRGVSSLQLGRRRPGPGTYRLEVVSHMAGPWGVQQEGQPGPW
GQALRLKVQLSVALVGRSLSGPQLSREGGFWNWEGLIPRSDG

The NOV2c amino acid sequence has 1897 of 2810 amino acid residues (67%) identical to, and 2258 of 2810 amino acid residues (80%) similar to, a Mus musculus 2907 amino acid residue fibrillin 2 precursor protein (ptnr:SWISSPROT-ACC:Q61555) (E=0.0).

NOV2c is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the NOV2c sequence.

NOV2d

A disclosed NOV2d nucleic acid of 9418 nucleotides (also referred to as CG88987-02) encoding a novel Fibrillin-like protein is shown in Table 2G. An open reading frame was identified beginning with an ATG initiation codon at nucleotides 41-43 and ending with a TGA codon at nucleotides 8576-8578. Putative untranslated regions upstream from the intiation codon and downstream from the termination codon are underlined in Table 2G, and the start and stop codons are in bold letters.

TABLE 2G

NOV2d nucleotide sequence (SEQ ID NO:11).
CTGACTCTGCTTCCTCTCCGCAGCCTCCAGGGGACACGCCATGACTCTGGAGGGTCTGTATTTGGCAAGGGGCCCCCTGGC
CCGGCTCCTGCTGGCCTGGTCGGCCCTGTTGTGCATGGCAGGTGGCCAACCCCGCTGGGACGGGGCCTTGGAGGCTGCAGG
TCCTGGACGTGTGCGGAGGCGGGGCAGCCCAGGCATCTTGCAGGGGCCGAATGTGTGCGGCTCCCGGTTCCATGCCTACTG
CTGTCCAGGCTGGAGGACATTCCCTGGCAGGAGCCAGTGTGTCGTACCCATCTGTAGGCGCGCCTGCGGTGAAGGCTTCTG
CTCCCAGCCCAACCTGTGCACCTGTGCGGATGGGACGCTGGCTCCCAGCTGCGGGGTGAGCCGAGCCATCTGTGACCGCGG
CTGCCACAATGGGGGTCGCTGCATTGGGCCCAACCGCTGCGCCTGTGTGTATGGCTTCATGGGACCTCAATGTGAGAGAGA
TTACCGGACGGGACCCTGCTTTGGCCAAGTAGGCCCCGAGGGGTGCCAGCATCAGCTGACGGGCCTCGTGTGCACCAAGGC
ACTTTGCTGTGCCACTGTGGGCCGTGCCTGGGGCCTTCCATGTGAACTTTGCCCTGCACAGCCACACCCCTGCCGCCGCGG
CTTCATCCCCAATATCCACACGGGGGCCTGCCAAGATGTGGATGAGTGCCAGGCTGTGCCAGGCCTGTGCCAGGGAGGCAG
CTGCGTCAACATGGTGGGCTCCTTCCATTGCCGCTGTCCAGTTGGACACCGGCTCAGTGACAGCAGCGCCGCATGTGAAGA
CTACGATGAATGCAGCACCATTCCTGGAATCTGTGAAGGGGGTGAATGTACAAACACAGTCAGCAGTTACTTTTGCAAATG
TCCTCCTGGTTTTTACACCTCTCCAGATGGTACTCTTCATGGACAGTCGCGGGCCGGCGCCTGCTTCTCAGTGCTTTTCGG
GGGCCGCTGTGCTGGAGACCTCGCCGGCCACTACACTCGCAGGCAGTGCTGCTGTGACAGGGGCAGGTGCTGGGCAGCTGG
CCCGGTCCCTGAGCTGTGTCCTCCTCGGGGCTCCAATGAATTCCAGCAACTGTGCGCCCAGCGGCTGCCGCTGCTACCCGG
CCACCCTGGCCTCTTCCCTGGCCTCCTGGGCTTCGGATCCAATGGCATGGGTCCCCCTCTTGGGCCAGCGCGACTCAACCC
CCATGGCTCTGATGCGCGTGGGATCCCCAGCCTGGGCCCTGGCAACTCTAATATTGGCACTGCTACCCTGAACCAGACCAT
TGACATCTGCCGACACTTCACCAACCTGTGTCTGAATGGCCGCTGCCTGCCCACGCCTTCCAGCTACCGCTGCGAGTGTAA
CGTGGGCTACACCCAGGACGTGCGCGGCGAGTGCATTGATGTAGACGAATGCACCAGCAGCCCCTGCCACCACGGTGACTG
CGTCAACATCCCCGGCACCTACCACTGCCGGTGCTACCCGGGCTTCCAGGCCACGCCCACCAGGCAGGCATGCGTGGATGT
GGACGAGTGCATTGTCAGTGGTGGCCTTTGTCACCTGGGCCGCTGTGTCAACACAGAGGGCAGCTTCCAGTGTGTCTGCAA
TGCAGGCTTCGAGCTCAGCCCTGACGGCAAGAACTGTGTGGACCACAACGAGTGTGCCACCAGCACCATGTGCGTCAACGG
CGTGTGTCTCAACGAGGATGGCAGCTTCTCCTGCCTCTGCAAACCCGGCTTCCTGCTGGCGCCTGGCGGCCACTACTGCAT
GGACATTGACGAGTGCCAGACGCCCGGCATCTGCGTGAACGGCCACTGTACCAACACCGAGGGCTCCTTCCGCTGCCAGTG
CCTGGGGGGGCTGGCGGTAGGCACGGATGGCCGCGTGTGCGTGGACACCCACGTGCGCAGCACCTGCTATGGGGCCATCGA
GAAGGGCTCCTGTGCCCGCCCCTTCCCTGGCACTGTCACCAAGTCCGAGTGCTGCTGTGCCAATCCGGACCACGGTTTTGG
GGAGCCCTGCCAGCTTTGTCCTGCCAAAGACTCCGCTGAGTTCCAGGCACTGTGCAGCAGTGGGCTTGGCATTACCACGGA
TGGTCGAGACATCAACGAGTGTGCTCTGGATCCTGAGGTTTGTGCCAATGGCGTGTGCGAGAACCTTCGGGGCAGCTACCG
CTGTGTCTGCAACCTGGGTTATGAGGCAGGTGCCTCAGGCAAGGACTGCACAGACGTGGATGAGTGTGCCCTCAACAGCCT
CCTGTGTGACAACGGGTGGTGCCAGAATAGCCCTGGCAGCTACAGCTGCTCCTGCCCCCCCGGCTTCCACTTCTGGCAGGA
CACGGAGATCTGCAAAGATGTCGACGAATGCCTGTCCAGCCCGTGTGTGAGTGGCGTCTGTCGGAACCTGGCCGGCTCCTA
CACCTGCAAATGTGGCCCTGGCAGCCGGCTGGACCCCTCTGGTACCTTCTGTCTAGACAGCACCAAGGGCACCTGCTGGCT
GAAGATCCAGGAGAGCCGCTGTGAGGTGAACCTTCAGGGAGCCAGCCTGCGGTCTGAGTGCTGTGCCACCCTCGGGGCAGC
CTGGGGGAGCCCCTGCGAACGCTGCGAGATCGACCCTGCCTGTGCCCGGGGCTTTGCCCGGATGACGGGTGTCACCTGCGA
TGATGTGAACGAGTGTGAGTCCTTCCCGGGAGTCTGTCCCAACGGGCGTTGCGTCAACACTGCTGGGTCTTTCCGCTGTGA
GTGTCCAGAGGGCCTGATGCTGGACGCCTCAGGCCGGCTGTGCGTGGATGTGAGATTGGAACCATGTTTCCTGCGATGGGA
TGAGGATGAGTGTGGGGTCACCCTGCCTGGCAAGTACCGGATGGACGTCTGCTGCTGCTCCATCGGGGCCGTGTGGGGAGT
CGAGTGCGAGGCCTGCCCGGATCCCGAGTCTCTGGAGTTCGCCAGCCTGTGCCCGCGGGGGCTGGGCTTCGCCAGCCGGGA
CTTCCTGTCTGGCCGACCATTCTATAAAGATGTGAATGAATGCAAGGTGTTCCCTGGCCTCTGCACGCACGGTACCTGCAG
AAACACGGTGGGCAGCTTCCACTGCGCCTGTGCGGGGGGCTTCGCCCTGGATGCCCAGGAACGGAACTGCACAGATATCGA
CGAGTGTCGCATCTCTCCTGACCTCTGCGGCCAGGGCACCTGTGTCAACACGCCGGGCAGCTTTGAGTGCGAGTGTTTTCC
CGGCTACGAGAGTGGCTTCATGCTGATGAAGAACTGCATGGACGTGGACGAGTGTGCAAGGGACCCGCTGCTCTGCCGGGG
AGGCACTTGCACCAACACGGATGGGAGCTACAAGTGCCAGTGTCCCCCTGGGCATGAGCTGACGGCCAAGGGCACTGCCTG
TGAGGACATCGATGAGTGCTCCCTGAGTGATGGCCTGTGTCCCCATGGCCAGTGTGTCAATGTCATCGGTGCCTTCCAGTG
CTCCTGCCATGCCGGCTTCCAGAGCACACCTGACCGCGGAGCTACAAGTGCCAGTTGCCCGACGGAAGGGCATGTGCAGGT
CGTCCTGGGGCCTGGGGAGCAGATGTGTACTGGGTGGTCTATCAGGGCAAAGCTAAGCACAGTCCCCAACCCTGCTCCCCC
AGACGTGGACGAGTGTGAAGAGAACCCCCGCGTTTGTGACCAAGGCCACTGCACCAACATGCCAGGGGGTCACCGCTGCCT
GTGCTATGATGGCTTCATGGCCACGCCAGACATGAGGACATGTGTTGATGTGGATGAGTGTGACCTGAACCCTCACATCTG
CCTCCATGGGGACTGCGAGAACACGAAGGGTTCCTTTGTCTGCCACTGTCAGCTGGGCTACATGGTCAGGAAGGGGGCCAC
AGGCTGCTCTGATGTGGATGAATGCGAGGTTGGAGGACACAACTGTGACAGTCACGCCTCCTGTCTCAACATCCCAGGGAG
TTTCAGCTGTAGGTGCCTGCCAGGCTGGGTGGGGGATGGCTTCGAATGTCACGACCTGGATGAATGCGTCTCCCAGGAGCA
CCGGTGCAGCCCAAGAGGTGACTGTCTCAATGTCCCTGGCTCCTACCGCTGCACCTGCCGCCAGGGCTTTGCCGGGGATGG
CTTCTTCTGCGAAGACAGGGATGAATGTGCCGAGAACGTGGACCTCTGTGACAACGGGCAGTGCCTCAATGCGCCCGGCGG
GTACCGCTGTGAATGTGAGATGGGCTTTGACCCCACCGAGGACCACCGGGCCTGCCAGGATGTGGACGAGTGTGCGCAAGA
GAACCTCTGTGCATTTGGGAGCTGTGAGAACCTGCCTGGAATGTTCCGCTGCATCTGCAATGGTGGCTACGAACTGGACCG
AGGGGGTGGCAACTGCACAGACATCAACGAGTGTGCAGACCCAGTAAACTGCATCAACGGCGTGTGCATTAACACCCCCGG
CAGCTACCTCTGCAGCTGCCCCCAGGATTTTGAGCTGAACCCCAGCGGAGTGGGCTGCGTGGACACTCGGGCCGGGAACTG
TTTCCTGGAGACGCATGACCGAGGGGACAGTGGCATTTCCTGCAGTGCCGAGATGCCAGTTGGTGTCACCCGAGCTTCCTG
CTGTTGCTCCCTGGGCCGGGCTTGGGGCAATCCCTGTGAGCTGTGCCCTATGGCCAACACCACTGAGTACAGAACCCTGTG
CCCGGGTGGTGAGGGCTTCCAGCCTAACCGCATCACTGTCATTCTGGAAGACATCGACGAGTGCCAAGAGCTGCCAGGGCT
GTGTCAGGGGGGTGACTGCGTCAACACGTTTGGCAGTTTCCAGTGTGAGTGCCCACCTGGCTACCACCTCAGTGAGCACAC
CCGCATCTGTGAGGATATTGACGAATGCTCCACACACTCCGGCATCTGTGGCCCTGGCACCTGCTACAACACCCTGGGGAA
CTACACCTGTGTCTGCCCTGCAGAGTACCTCCAAGTCAATGGTGGCAACAACTGCATGGATATGAGGAAGAGTGTCTGCTT
CCGGCACTATAACGGCACATGTCAAAATGAGCTGGCCTTCAACGTGACCCGGAAAATGTGTTGCTGCTCCTACAACATTGG
CCAGGCCTGGAATAGACCCTGTGAGGCCTGCCCCACTCCCATCAGTCCTGACTACCAGATCCTGTGTGGAAATCAGGCCCC
GGGATTCCTCACTGACATCCACACGGGGAAGCCCCTTGACATTGATGAGTGTGGGGAGATCCCCGCCATCTGTGCCAATGG
CATCTGCATAAACCAGATCGGGAGTTTCCGCTGCGAGTGCCCCGCAGGCTTCAACTACAACAGCATCCTGCTGGCTTGTGA
AGATGTCGATGAGTGTGGCAGCAGGGAGAGTCCCTGCCAGCAGAATGCTGACTGCATCAACATCCCCGGTAGCTACCGCTG
CAAGTGCACCCGAGGGTACAAACTGTCGCCAGGCGGGGCTTGTGTGGGACGGAATGAGTGTCGGGAGATCCCGAATGTCTG
TAGCCATGGTGACTGCATGGACACAGAAGGCAGCTACATGTGTCTGTGTCACCGTGGATTCCAGGCCTCTGCAGACCAGAC
CCTGTGCATGGACATTGACGAGTGTGACCGGCAGCCTTGTGGAAATGGGACCTGCAAGAACATCATTGGCTCCTACAACTG
CCTCTGCTTCCCTGGCTTTGTGGTGACACACAATGGGGATTGTGTGGATTTTGATGAGTGTACTACCCTGGTGGGGCAGGT
GTGCCGATTTGGCCATTGCCTCAACACAGCTGGTTCCTTCCACTGCCTCTGCCAGGATGGCTTTGAGCTCACAGCTGATGG
GAAGAACTGTGTGGACACCAATGAGTGCCTCAGCCTTGCAGGAACCTGCCTACCCGGCACTTGCCAGAACCTCGAGGGCTC
CTTCCGCTGCATCTGTCCCCCTGGCTTCCAGGTGCAGAGTGACCACTGCATTGATATCGACGAGTGCTCAGAGGAGCCCAA
CCTCTGCCTCTTTGGCACCTGTACCAACAGCCCTGGGAGCTTCCAGTGCCTCTGCCCACCTGGCTTTGTCCTCTCTGACAA
TGGGCACCGTTGCTTTGACACACGGCAGAGTTTCTGCTTCACCCGTTTTGAGGCTGGGAAGTGCTCGGTGCCCAAAGCTTT
CAACACCACCAAGACCCGCTGCTGCTGCAGTAAGAGGCCTGGGGAGGGCTGGGGAGACCCCTGCGAACTGTGTCCCCAGGA
AGGCAGCGCTGCCTTTCAGGAGCTCTGCCCCTTTGGCCACGGGGCAGTCCCAGGCCCGGATGACTCCCGAGAAGACGTGAA
TGAGTGTGCAGAGAACCCTGGCGTCTGCACTAACGGCGTCTGTGTCAACACCGATGGATCCTTCCGCTGTGAGTGTCCCTT
TGGCTACAGCCTGGACTTCACTGGCATCAACTGTGTGGACACAGACGAGTGCTCTGTCGGCCACCCCTGTGGGCAAGGGAC
ATGCACCAATGTCATCGGAGGCTTCGAATGTGCCTGTGCTGACGGCTTTGAGCCTGGCCTCATGATGACCTGCGAGGACAT
CGACGAATGCTCCCTGAACCCGCTGCTCTGTGCCTTCCGCTGCCACAATACCGAGGGCTCCTACCTGTGCACCTGTCCAGC
CGGCTACACCCTGCGGGAGGATGGGGCCATGTGTCGAGATGTGGACGAGTGTGCAGATGGTCAGCAGGACTGCCACGCCCG
GGGCATGGAGTGCAAGAACCTCATCGGTACCTTCGCGTGCGTCTGTCCCCCAGGCATGCGGCCCCTGCCTGGCTCTGGGGA
GGGCTGCACAGATGACAATGAATGCCACGCTCAGCCTGACCTCTGTGTCAACGGCCGCTGTGTCAACACCGCGGGCAGCTT
CCGGTGCGACTGTGATGAGGGATTCCAGCCCAGCCCCACCCTTACCGAGTGCCACGACATCCGGCAGGGGCCCTGCTTTGC
CGAGGTGCTGCAGACCATGTGCCGGTCTCTGTCCAGCAGCAGTGAGGCTGTCACCAGGGCCGAGTGCTGCTGTGGGGGTGG
CCGGGGCTGGGGGCCCCGCTGCGAGCTCTGTCCCCTGCCCGGCACCTCTGCCTACAGGAAGCTGTGCCCCCATGGCTCAGG
CTACACTGCTGAGGGCCGAGATGTAGATGAATGCCGTATGCTTGCTCACCTGTGTGCTCATGGGGAGTGCATCAACAGCCT
TGGCTCCTTCCGCTGCCACTGTCAGGCCGGGTACACACCGGATGCTACTGCTACTACCTGCCTGGATATGGATGAGTGCAG
CCAGGTCCCCAAGCCATGTACCTTCCTCTGCAAAAACACGAAGGGCAGTTTCCTGTGCAGCTGTCCCCGAGGCTACCTGCT
GGAGGAGGATGGCAGGACCTGCAAAGACCTGGACGAATGCACCTCCCGGCAGCACAACTGTCAGTTCCTCTGTGTCAACAC
TGTGGGCGCCTTCACCTGCCGCTGTCCACCCGGCTTCACCCAGCACCACCAGGCCTGCTTCGACAATGATGAGTGCTCAGC
CCAGCCTGGCCCATGTGGTGCCCACGGGCACTGCCACAACACCCCGGGCAGCTTCCGCTGTGAATGCCACCAAGGCTTCAC
CCTGGTCAGCTCAGGCCATGGCTGTGAAGATGTGAATGAATGTGATGGGCCCCACCGCTGCCAGCATGGCTGTCAGAACCA
GCTAGGGGGCTACCGCTGCAGCTGCCCCCAGGCTTTCACCCAGCACTCCCAGTGGGCCCAGTGTGTGGATGAGAATGAGTG
TGCCCTGTCGCCCCCCACCTGCGGGAGCGCCTCCTGTCGCAACACTCTTGGTGGCTTCCGCTGCGTCTGCCCCTCCGGCTT
TGACTTTGATCAGGCCCTCGGGGGCTGCCAGGAGGTGGATGAGTGCGCCGGACGGCGTGGCCCCTGTAGCTACAGCTGTGC
CAACACGCCTGGTGGCTTCCTGTGCGGCTGTCCTCAAGGCTACTTCCGGGTTGGGCAAGGGCACTGTGTCTCCGGCCTGGG
CTTCAGCCCCGGACCCCAGGACACCCCGGACAAAGAGGAGCTGCTCTCGTCTGAAGCCTGCTACGAATGCAAGATCAATGG
CCTCTCCCCTCGGGACCGGCCACGACGCAGTGCCCACAGGGACCACCAGGTGAACCTGGCCACCCTTGACTCCGAGGCCCT
GCTGACCTTGGGCCTGAACCTCTCACACCTGGGCCGGGCCGAGCGCATCCTGGAGCTCCGGCCGGCCCTGGAGGGTGTAGA
GGGCCGGATCCGCTACGTCATCGTCCGCGGAAACGAGCAAGGTTTCTTTCGCATGCATCACCTCCGTGGCGTCAGCTCCCT
GCAGCTGGGGCGGAGGCGGCCGGGGCCTGGAACCTACCGGCTGGAGGTGGTGAGCCACATGGCAGGACCCTGGGGTGTCCA
GCAAGAGGGGCAGCCAGGGCCATGGGGCCAGGCCTTGAGGCTGAAGGTGCAACTGTCAGTTGCTTTAGTTGGGAGGAGCCT
CAGTGGGCCCCAGCTGTCCAGAGAAGGGGGATTCTGGAACTGGGAAGGACTGATCCCCAGAAGCGATGGCTGACCAGATTG
AACCCCGAAACTCAGGAAGAGTGAAATGCTACACGACAACCTCAGGCAAGCCCGGCCTCTGCCTGGGCCTCTGTGCCAGCC
CCGGGGGCCCCCCAGTTACTCAGTCTTTCCTGGAGACAGCAAGAAGCTGCAATGTGCAATCCCCCTGCCCCCACAGCCAAG
GTCAGGAAGAGGCCCTGTGGTCACCGTGTCTGGCCAATCTCAGGCTTTCACTTCTGTACTGCACTGTGGCTTGCCCTGGCG
GGGGGCAGGGGGTTGGCAGGACATGGCAATGGGCAACTGGGGTGGGCACAGGGCTTATTCCTCGGAGTAGAAGGGTGTACA
GGGGGCCCAGACTCCACAGTGACTTGCCACATTTGCCCCCCATTTGGAGAATGCTTTTATATCAAAAGTGGAGACGATAAT
AAAGTTATTTTGGGTTAAGTCTGCCTGCCCTTTGGCAAGTTCTTGAAGTAAGTAGATGCTGCCCTCGGACTGGGCGAGGCA
GATCTTGTGCCTGGGGAAGCAGAAGGCCTTATGGGCTCCCCAGAATGGTAATAATGGCTCACGCTTCCTGACCACGTACTA
CATACCAGAGACCATTCGATTTTTTTTTTATTTTTTCTGAGACAGGGTCTTGCTCTGTTGCCTAGGTTGGAGTGCAGTGGC
GCGATCGTGGCTCCCTGTGGTCGCCACTTTCCGGGCTCGAGCAATCCTCCCACCTCAGCCTCTCCCAAGTAGCTGGGACCA
AAGGTGCACGCCACCACACCCAGCTAATTTTTTTAAATTTCTTTTTCTTTCTCTTTCTTTTTGAGACCAGCCCGGCCAACA
TGACGAAACCCCATCTCTAATA

The disclosed NOV2d nucleic acid sequence, localized to chromsome 19, has 3194 of 4382 bases (72%) identical to a Mus musculus fibrillin 2 (fbn2) mRNA (gb:GENBANK-ID:MUSFBN2|acc:L39790.1) (E=0.0).

A NOV2d polypeptide (SEQ ID NO: 12) encoded by SEQ ID NO: 11 has 2845 amino acid residues and is presented using the one-letter code in Table 2H. Signal P, Psort and/or Hydropathy results predict that NOV2d contains a signal peptide and is likely to be localized to the nucleus with a certainty of 0.6000. Although PSORT suggests that the Fibrillin-like protein may be localized in the nucleus, the NOV2d protein is similar to the Fibrillin family, some members of which are released extracellularly. Therefore it is likely that NOV2d protein shows a similar localization. The most likely cleavage site for a NOV2d peptide is between amino acids 29 and 30, at: AGG-QG.

TABLE 2H

Encoded NOV2d protein sequence (SEQ ID NO:12).
MTLEGLYLARGPLARLLLAWSALLCMAGGQGRWDGALEAAGPGRVRRRGSPGILQGPNVCGSRFHAYCCPGWRTFPGRSQC
VVPICRRACGEGFCSQPNLCTCADGTLAPSCGVSRAICDRGCHNGGRCIGPNRCACVYGFMGPQCERDYRTGPCFGQVGPE
GCQHQLTGLVCTKALCCATVGRAWGLPCELCPAQPHPCRRGFIPNIHTGACQDVDECQAVPGLCQGGSCVNMVGSFHCRCP
VGHRLSDSSAACEDYDECSTIPGICEGGECTNTVSSYFCKCPPGFYTSPDGTLHGQSRAGACFSVLFGGRCAGDLAGEYTR
RQCCCDRGRCWAAGPVPELCPPRGSNEFQQLCAQRLPLLPGHPGLFPGLLGFGSNGMGPPLGPARLNPHGSDARGIPSLGP
GNSNIGTATLNQTIDICRHFTNLCLNGRCLPTPSSYRCECNVGYTQDVRGECIDVDECTSSPCHHGDCVNIPGTYHCRCYP
GFQATPTRQACVDVDECIVSGGLCHLGRCVNTEGSFQCVCNAGFELSPDGKNCVDHNECATSTMCVNGVCLNEDGSFSCLC
KPGFLLAPGGHYCMDIDECQTPGICVNGHCTNTEGSFRCQCLGGLAVGTDGRVCVDTHVRSTCYGAIEKGSCARPFPGTVT
KSECCCANPFHGFGEPCQLCPAKDSAEFQALCSSGLGITTDGRDINECALDPEVCANGVCENLRGSYRCVCNLGYEAGASG
KDCTDVDECALNSLLCDNGWCQNSPGSYSCSCPPGFHFWQDTEICKDVDECLSSPCVSGVCRNLAGSYTCKCGPGSRLDPS
GTFCLDSTKGTCWLKIQESRCEVNLQGASLRSECCATLGAAWGSPCERCEIDPACARGFARMTGVTCDDVNECESFPGVCP
NGRCVNTAGSFRCECPEGLMLDASGRLCVDVRLEPCFLRWDEDECGVTLPGKYRMDVCCCSIGAVWGVECEACPDPESLEF
ASLCPRGLGFASRDFLSGRPFYKDVNECKVFPGLCTHGTCRNTVGSFHCACAGGFALDAQERNCTDIDECRISPDLCGQGT
CVNTPGSFECECFPGYESGFMLMKNCMDVDECARDPLLCRGGTCTNTDGSYKCQCPPGHELTAKGTACEDIDECSLSDGLC
PHGQCVNVIGAFQCSCHAGFQSTPDRGATSASCPTEGHVQVVLGPGEQMCTGWSIRAKLSTVPNPAPPDVDECEENPRVCD
QGHCTNMPGGHRCLCYDGFMATPDMRTCVDVDECDLNPHICLHGDCENTKGSFVCHCQLGYMVRKGATGCSDVDECEVGGH
NCDSHASCLNIPGSFSCRCLPGWVGDGFECHDLDECVSQEHRCSPRGDCLNVPGSYRCTCRQGFAGDGFFCEDRDECAENV
DLCDNGQCLNAPGGYRCECEMGFDPTEDHRACQDVDECAQENLCAFGSCENLPGMFRCICNGGYELDRGGGNCTDINECAD
PVNCINGVCINTPGSYLCSCPQDFELNPSGVGCVDTRAGNCFLETHDRGDSGISCSAEIGVGVTRASCCCSLGRAWGNPCE
LCPMANTTEYRTLCPGGEGFQPNRITVILEDIDECQELPGLCQGGDCVNTFGSFQCECPPGYHLSEHTRICEDIDECSTHS
GICGPGTCYNTLGNYTCVCPAEYLQVNGGNNCMDMRKSVCFRHYNGTCQNELAFNVTRKMCCCSYNIGQAWNRPCEACPTP
ISPFYQILCGNQAPGFLTDIHTGKPLDIDECGEIPAICANGICINQIGSFRCECPAGFNYNSILLACEDVDECGSRESPCQ
QNADCINIPGSYRCKCTRGYKLSPGGACVGRNECREIPNVCSHGDCMDTEGSYMCLCHRGFQASADQTLCMDIDECDRQPC
GNGTCKNIIGSYNCLCFPGFVVTHNGDCVDFDECTTLVGQVCRFGHCLNTAGSFHCLCQDGFELTADGKNCVDTNECLSLA
GTCLPGTCQNLEGSDRCICPPGFQVQSDHCIDIDECSEEPNLCLFGTCTNSPGSFQCLCPPGFVLSDNGHRCFDTRQSFCF
TRFEAGKCSVPKAFNTTKTRCCCSKRPGEGWGDPCELCPQEGSAAFQELCPFGHGAVPGPDDSREDVNECAENPGVCTNGV
CVNTDGSFRCECPFGYSLDFTGINCVDTDECSVGHPCGQGTCTNVIGGFECACADGFEPGLMMTCEDIDECSLNPLLCAFR
CHNTEGSYLCTCPAGYTLREDGAMCRDVDECADGQQDCHARGMECKNLIGTFACVCPPGMRPLPGSGEGCTDDNECHAQPD
LCVNGRCVNTAGSFRCDCDEGFQPSPTLTECHDIRQGPCFAEVLQTMCRSLSSSSEAVTRAECCCGGGRGWGPRCELCPLP
GTSAYRKLCPHGSGYTAEGRDVDECRMLAHLCAHGECINSLGSFRCHCQAGYTPDATATTCLDMDECSQVPKPCTFLCKNT
KGSFLCSCPRGYLLEEDGRTCKDLDECTSRQHNCQFLCVNTVGAFTCRCPPGFTQHHQACFDNDECSAQPGPCGAHGHCHN
TPGSFRCECHQGFTLVSSGHGCEDVNECDGPHRCQHGCQNQLGGYRCSCPQAFTGHSQWAQCVDENECALSPPTCGSASVR
NTLGGFRCVCPSGFDFDQALGGCQEVDECAGRRGPCSYSCANTPGGFLCGCPQGYFRVGQGHCVSGLGFSPGPQDTPDKEE
LLSSEACYECKINGLSPRDRPRRSAHRDHQVNLATLDSEALLTLGLNLSHLGRAERILELRPALEGLEGRIRYVIVRGNEQ
GFFRMHHLRGVSSLQLGRRRPGPGTYRLEVVSHMAGPWGVQQEGQPGPWGQALRLKVQLSVALVGRSLSGPQLSREGGFWN
WEGLIPRSDG

The NOV2d amino acid sequence has 1836 of 2753 amino acid residues (66%) identical to, and 2187 of 2753 amino acid residues (79%) similar to, a Mus musculus 2907 amino acid residue fibrillin 2 precursor protein (ptnr:SWISSPROT-ACC:Q61 555) (E=0.0).

NOV2d is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the NOV2d sequence.

NOV2e

A disclosed NOV2e nucleic acid of 8219 nucleotides (also referred to as CG88987-03) encoding a novel Fibrillin-like protein is shown in Table 21. An open reading frame was identified beginning with an ATG initiation codon at nucleotides 49-51 and ending with a TGA codon at nucleotides 8134-8136. Putative untranslated regions upstream from the intiation codon and downstream from the termination codon are underlined in Table 21, and the start and stop codons are in bold letters.

TABLE 2I

NOV2e nucleotide sequence (SEQ ID NO:13).
ATCTAGGGCTGACTCTGCTTCCTCTCCGCAGCCTCCAGGGGACACGCCATGACTCTGGAGGGTCTGTATTTGGCAAGGGGC
CCCCTGGCCCGGCTCCTGCTGGCCTGGTCGGCCCTGTTGTGCATGGCAGGTGGCCAAGGCCGCTGGGACGGGGCCTTGGAG
GCTGCAGGTCCTGGACGTGTGCGGAGGCGGGGCAGCCCAGGCATCTTGCAGGGGCCGAATGTGTGCGGCTCCCGGTTCCAT
GCCTACTGCTGTCCAGGCTGGAGGACATTCCCTGGCAGGAGCCAGTGTGTCGTACCCATCTGTAGGCGCGCCTGCGGTGAA
GGCTTCTGCTCCCAGCCCAACCTGTGCACCTGTGCGGATGGGACGCTGGCTCCCAGCTGCGGGGTGAGCCGAGGGTCAGGG
TGCAGTGTGAGCTGTATGAATGGGGGCACCTGCCGGGGGGCGTCCTGTCTGTGTCAGAAGGGCTACACAGGCACCGTGTGT
GGGCAGCCCATCTGTGACCGCGGCTGCCACAATGGGGGTCGCTGCATTGGGCCCAACCGCTGCGCCTGTGTGTATGGCTTC
ATGGGACCTCAATGTGAGAGAGATTACCGGACGGGATCCTGCTTTGGCCAAGTAGGCCCCGAGGGGTGCCAGCATCAGCTG
ACGGGCCTCGTGTGCACCAAGGCACTTTGCTGTGCCACTGTGGGCCGTGCCTGGGGCCTTCCATGTGAACTTTGCCCTGCA
CAGCCACACCCCTGCCGCCGCGGCTTCATCCCCAATATCCACACGGGGGCCTGCCAAGATGTGGATGAGTGCCAGGCTGTG
CCAGGCCTGTGCCAGGGAGGCAGCTGCGTCAACATGGTGGGCTCCTTCCATTGCCGCTGTCCAGTTGGACACCGGCTCAGT
GACAGCAGCGCCGCATGTGAAGACTACCGGGCCGGCGCCTGCTTCTCAGTGCTTTTCGGGGGCCGCTGTGCTGGAGACCTC
GCCGGCCACTACACTCGCAGGCAGTGCTGCTGTGACAGGGGCAGGTGCTGGGCAGCTGGCCCGGTCCCTGAGCTGTGTCCT
CCTCGGGGCTCCAATGAATTCCAGCAACTGTGCGCCCAGCGGCTGCCGCTGCTACCCGGCCACCCTGGCCTCTTCCCTGGC
CTCCTGGGCTTCGGATCCAATGGCATGGGTCCCCCTCTTGGGCCAGCGCGACTCAACCCCCATGGCTCTGATGCGCGTGGG
ATCCCCAGCCTGGGCCCTGGCAACTCTAATATTGGCACTGCTACCCTGAACCAGACCATTGACATCTGCCGACACTTCACC
AACCTGTGTCTGAATGGCCGCTGCCTGCCCACGCCTTCCAGCTACCGCTGCGAGTGTAACGTGGGCTACACCCAGGACGTG
CGCGGCGAGTGCATTGATGTAGACGAATGCACCAGCAGCCCCTGCCACCACGGTGACTGCGTCAACATCCCCGGCACCTAC
CACTGCCGGTGCTACCCGGGCTTCCAGGCCACGCCCACCAGGCAGGCATGCGTGGATGTGGACGAGTGCATTGTCAGTGGT
GGCCTTTGTCACCTGGGCCGCTGTGTCAACACAGAGGGCAGCTTCCAGTGTGTCTGCAATGCAGGCTTCGAGCTCAGCCCT
GACGGCAAGAACTGTGTGGACCACAACGAGTGTGCCACCAGCACCATGTGCGTCAACGGCGTGTGTCTCAACGAGGATGGC
AGCTTCTCCTGCCTCTGCAAACCCGGCTTCCTGCTGGCGCCTGGCGGCCACTACTGCATGGACATTGACGAGTGCCAGACG
CCCGGCATCTGCGTGAACGGCCACTGTACCAACACCGAGGGCTCCTTCCGCTGCCAGTGCCTGGGGGGGCTGGCGGTAGGC
ACGGATGGCCGCGTGTGCGTGGACACCCACGTGCGCAGCACCTGCTATGGGGCCATCGAGAAGGGCTCCTGTGCCCGCCCC
TTCCCTGGCACTGTCACCAAGTCGGAGTGCTGCTGTGCCAATCCGGACCACGGTTTTGGGGAGCCCTGCCAGCTTTGTCCT
GCCAAAAACTCCGCTGAGTTCCAGGCACTGTGCAGCAGTGGGCTTGGCATTACCACGGATGGTCGAGACATCAACGAGTGT
GCTCTGGATCCTGAGGTTTGTGCCAATGGCGTGTGCGAGAACCTTCGGGGCAGCTACCGCTGTGTCTGCAACCTGGGTTAT
GAGGCAGGTGCCTCAGGCAAGGACTGCACAGACGTGGATGAGTGTGCCCTCAACAGCCTCCTGTGTGACAACGGGTGGTGC
CAGAATAGCCCTGGCAGCTACAGCTGCTCCTGCCCCCCCGGCTTCCACTTCTGGCAGGACACGGAGATCTGCAAAGATGTC
GACGAATGCCTGTCCAGCCCGTGTGTGAGTGGCGTTTGTCGGAACCTGGCCGGCTCCTACACCTGCAAATGTGGCCCTGGC
AGCCGGCTGGACCCCTCTGGTACCTTCTGTCTAGACAGCACCAAGGGCACCTGCTGGCTGAAGATCCAGGAGAGCCGCTGT
GAGGTGAACCTTCAGGGAGCCAGCCTGCGGTCTGAGTGCTGCGCCACCCTCGGGGCAGCCTGGGGGAGCCCCTGCGAACGC
TGCGAGATCGACCCTGCCTGTGCCCGGGGCTTTGCCCGGATGACGGGTGTCACCTGCGATGATGTGAACGAGTGTGAGTCC
TTCCCGGGAGTCTGTCCCAACGGGCGTTGCGTCAACACTGCTGGGTCTTTCCGCTGTGAGTGTCCAGAGGGCCTGATGCTG
GACGCCTCAGGCCGGCTGTGCGTGGATGTGAGATTGGAACCATGTTTCCTGCGATGGGATGAGGATGAGTGTGCAAGGGAC
CCGCTGCTCTGCTGGGGAGGCACTTGCACCAACACGGATGGGAGCTACAAGTGCCAGTGTCCCCCTGGGCATGAGCTGACG
GCCAAGGGCACTGCCTGTGAGGACATCGATGAGTGCTCCCTGAGTGATGGCCTGTGTCCCCATGGCCAGTGTGTCAATGTC
ATCGGTGCCTTCCAGTGCTCCTGCCATGCCGGCTTCCAGAGCACACCTGACCGCCAGGGCTGCGTGGACATCAACGAATGC
CGGGTCCAGAATGGTGGGTGTGACGTGCACTGTATTAACACTGAGGGCAGCTACCGGTGCAGCTGTGGGCAGGGCTACTCG
CTGATGCCCGACGGAAGGGCATGTGCAGACGTGGACGAGTGTGAAGAGAACCCCCGCGTTTGTGACCAAGGCCACTGCACC
AACATGCCAGGGGGTCACCGCTGCCTGTGCTATGATGGCTTCATGGCCACGCCAGACATGAGGACATGTGTTGATGTGGAT
GAGTGTGACCTGAACCCTCACATCTGCCTCCATGGGGACTGCGAGAACACGAAGGGTTCCTTTGTCTGCCACTGTCAGCTG
GGCTACATGGTCAGGAAGGGGGCCACAGGCTGCTCTGATGTGGATGAATGCGAGGTTGGAGGACACAACTGTGACAGTCAC
GCCTCCTGTCTCAACATCCCGGGGAGTTTCAGCTGTAGGTGCCTGCCAGGCTGGGTGGGGGATGGCTTCGAATGTCACGAC
CTGGATGAATGCGTCTCCCAGGAGCACCGGTGCAGCCCAAGAGGTGACTGTCTCAATGTCCCTGGCTCCTACCGCTGCACC
TGCCGCCAGGGCTTTGCCGGGGATGGCTTCTTCTGCTCAGACAGGGATGAATGTGCCGAGAACGTGGACCTCTGTGACAAC
GGGCAGTGCCTCAATGCGCCCGGCGGGTACCGCTGTGAATGTGAGATGGGCTTTGACCCCACCGAGGACCACCGGGCCTGC
CAGATGGACGAGTGTGCGCAAGGGAACCTCTGTGCATTTGGGAGCTGTGAGAACCTGCCTGGAATGTTCCGCTGCATCTGC
AATGGTGGCTACGAACTGGACCGAGGGGGTGGCAACTGCACAACAGACATCAACGAGTGTGCAGACCCAGTAAACTGCATC
AACGGCGTGTGCATTAACACCCCCGGCAGCTACCTCTGCAGCTGCCCCCAGGATTTTGAGCTGAACCCCAGCGGAGTGGGC
TGCGTGGACACTCGGGCCGGGAACTGTTTCCTGGAGACGCATGACCGAGGGGACAGTGGCATTTCCTGCAGTGCCGAGATC
GGAGTTGGTGTCACCCGAGCTTCCTGCTGTTGCTCCCTGGGCCGGGCTTGGGGCAATCCCTGTGAGCTGTGCCCTATGGCC
AACACCACTGAGTACAGAACCCTGTGCCCGGGTGGTGAGGGCTTCCAGCCTAACCGCATCACTGTCATTCTGGAAGACATC
GACGAGTGCCAAGAGCTGCCAGGGCTGTGTCAGGGGGGTGACTGCGTCAACACGTTTGGCAGTTTCCAGTGTGAGTGCCCA
CCTGGCTACCACCTCAGTGAGCACACCCGCATCTGTGAGGATATTGACGAATGCTCCACACACTCCGGCATCTGTGGCCCT
GGCACCTGCTACAACACCCTGGGGAACTACACCTGTGTCTGCCCTGCAGAGTACCTCCAAGTCAATGGTGGCAACAACTGC
ATGGATATGAGGAAGAGTGTCTGCTTCCGGCACTATAACGGCACATGTCAAAATGAGCTGGCCTTCAACGTGACCCGGAAA
ATGTGTTGCTGCTCCTACAACATTGGCCAGGCCTGGAATAGACCCTGTGAGGCCTGCCCCACTCCCATCAGTCCTGACTAC
CAGATCCTGTGTGGAAATCAGGCCCCGGGATTCCTCACTGACATCCACACGGGGAACGGGGTTGACATTGATGAGTGTGGG
GAGATCCCCGCCATCTGTGCCAATGGCATCTGCATAAACCAGATCGGGAGTTTCCGCTGCGAGTGCCCCGCAGGCTTCAAC
TACAACAGCATCCTGCTGGCTTGTGAAGATGTCGATGAGTGTGGCAGCAGGGAGAGTCCCTGCCAGCAGAATGCTGACTGC
ATCAACATCCCCGGTAGCTACCGCTGCAAGTGCACCCGAGGGTACAAACTGTCGCCAGGCGGGGCTTGTGTGGGACGGAAT
GAGTGTCGGGAGATCCCGAATGTCTGTAGCCATGGTGACTGCATGGACACAGAAGGCAGCTACATGTGTCTGTGTCACCGT
GGATTCCAGGCCTCTGCAGACCAGACCCTGTGCATGGACATTGACGAGTGTGACCGGCAGCCTTGTGGAAATGGGACCTGC
AAGAACATCATTGGCTCCTACAACTGCCTCTGCTTCCCTGGCTTTGTGGTGACACACAATGGGGATTGTGTGGATTTTGAT
GAGTGTACTACCCTGGTGGGGCAGGTGTGCCGATTTGGCCATTGCCTCAACACAGCTGGTTCCTTCCACTGCCTCTGCCAG
GATGGCTTTGAGCTCACAGCTGATGGGAAGAACTGTGTGGACACCAATGAGTGCCTCAGCCTTGCAGGAACCTGCCTACCC
GGCACTTGCCAGAACCTCGAGGGCTCCTTCCGCTGCATCTGTCCCCCTGGCTTCCAGGTGCAGAGTGACCACTGCATTGAT
ATCGACGAGTGCTCAGAGGAGCCCAACCTCTGCCTCTTTGGCACCTGTACCAACAGCCCTGGGAGCTTCCAGTGCCTCTGC
CCACCTGGCTTTGTCCTCTCTGACAATGGGCACCGTTGCTTTGACACACGGCAGAGTTTCTGCTTCACCCGTTTTGAGGCT
GGGAAGTGCTCGGTGCCCAAAGCTTTCAACACCACCAAGACCCGCTGCTGCTGCAGTAAGAGGCCTGGGGAGGGCTGGGGA
GACCCCTGCGAACTGTGTCCCCAGGAGGACTCCCCTCCCCCTCTCCGTCCAGCTGCCTTTCAGGAGCTCTGCCCCTTTGGC
CACGGGGCAGTCCCAGGCCCGGATGACTCCCGAGAAGACGTGAATGAGTGTGCAGAGAACCCTGGCGTCTGCACTAACGGC
GTCTGTGTCAACACCGATGGATCCTTCCGCTGTGAGTGTCCCTTTGGCTACAGCCTGGACTTCACTGGCATCAACTGTGAG
GACACAGACGAGTGCTCTGTCGGCCACCCCTGTGGGCAAGGGACATGCACCAATGTCATCGGAGGCTTCGAATGTGCCTGT
GCTGACGGCTTTGAGCCTGGCCTCATGATGACCTGCGAGGACATCGACGAATGCTCCCTGAACCCGCTGCTCTGTGCCTTC
CGCTGCCACAATACCGAGGGCTCCTACCTGTGCACCTGTCCAGCCGGCTACACCCTGCGGGAGGATGGGGCCATGTGTCGA
GATGTGGACGAGTGTGCAGATGGTCAGCAGGACTGCCACGCCCGGGGCATGGAGTGCAAGAACCTCATCGGTACCTTCGCG
TGCGTCTGTCCCCCAGGCATGCGGCCCCTGCCTGGCTCTGGGGAGGGCTGCACAGATGACAATGAATGCCACGCTCAGCCT
GACCTCTGTGTCAACGGCCGCTGTGTCAACACCGCGGGCAGCTTCCGGTGCGACTGTGATGAGGGATTCCAGCCCAGCCCC
ACCCTTACCGAGTGCCACGACATCCGGCAGGGGCCCTGCTTTGCCGAGGTGCTGCAGACCATGTGCCGGTCTCTGTCCAGC
AGCAGTGAGGCTGTCACCAGGGCCGAGTGCTGCTGTGGGGGTGGCCGGGGCTGGGGGCCCCGCTGCGAGCTCTGTCCCCTG
CCCGGCACCTCTGCCTACAGGAAGCTGTGCCCCCATGGCTCAGGCTACACTGCTGAGGGCCGAGATGTAGATGAATGCCGT
ATGCTTGCTCACCTGTGTGCTCATGGGGAGTGCATCAACAGCCTTGGCTCCTTCCGCTGCCACTGTCAGGCCGGGTACACA
CCGGATGCTACTGCTACTACCTGCCTGGATATGGATGAGTGCAGCCAGGTCCCCAAGCCATGTACCTTCCTCTGCAAAAAC
ACGAAGGGCAGTTTCCTGTGCAGCTGTCCCCGAGGCTACCTGCTGGAGGAGGATGGCAGGACCTGCAAAGACCTGGACGAA
TGCACCTCCCGGCAGCACAACTGTCAGTTCCTCTGTGTCAACACTGTGGGCGCCTTCACCTGCCGCTGTCCACCCGGCTTC
ACCCAGCACCACCAGGCCTGCTTCGACAATGATGAGTGCTCAGCCCAGCCTGGCCCATGTGGTGCCCACGGGCACTGCCAC
AACACCCCGGGCAGCTTCCGCTGTGAATGCCACCAAGGCTTCACCCTGGTCAGCTCAGGCCATGGCTGTGAAGATGTGAAT
GAATGTGATGGGCCCCACCGCTGCCAGCATGGCTGTCAGAACCAGCTAGGGGGCTACCGCTGCAGCTGCCCCCAGGCTTTC
ACCCAGCACTCCCAGTGGGCCCAGTGTGTGGATGAGAATGAGTGTGCCCTGTCGCCCCCCACCTGCGGGAGCGCCTCCTGT
CGCAACACTCTTGGTGGCTTCCGCTGCGTCTGCCCCTCCGGCTTTGACTTTGATCAGGCCCTCGGGGGCTGCCAGGAGGTG
GATGAGTGCGCCGGACGGCGTGGCCCCTGTAGCTACAGCTGTGCCAACACGCCTGGTGGCTTCCTGTGCGGCTGTCCTCAA
GGCTACTTCCGGGTTGGGCAAGGGCACTGTGTCTCCGGCCTGGGCTTCAGCCCCGGACCCCAGGACACCCCGGACAAAGAG
GAGCTGCTCTCGTCTGAAGCCTGCTACGAATGCAAGATCAATGGCCTCTCCCCTCGGGACCGGCCACGACGCAGTGCCCAC
AGGGACCACCAGGTGAACCTGGCCACCCTTGACTCCGAGGCCCTGCTGACCTTGGGCCTGAACCTCTCACACCTGGGCCGG
GCCGAGCGCATCCTGGAGCTCCGGCCGGCCCTGGAGGGTCTAGAGGGCCGGATCCGCTACGTCATCGTCCGCGGAAACGAG
CAAGGTTTCTTTCGCATGCATCACCTCCGTGGCGTCAGCTCCCTGCAGCTGGGGCGGAGGCGGCCGGGGCCTGGAACCTAC
CGGCTGGAGGTGGTGAGCCACATGGCAGGACCCTGGGGTGTCCAGCAAGAGGGGCAGCCAGGGCCATGGGGCCAGGCCTTG
AGGCTGAAGGTGCAACTGTCAGTTGCTTTAGTTGGGAGGAGCCTCAGTGGGCCCCAGCTGTCCAGAGAAGGGGGATTCTGG
AACTGGGAAGGACTGATCCCCAGAAGCGATGGCTGACCAGATTGAACCCCGAAACTCAGGAAGAGTGAAATGCTACACGAC
AACCTCAGGCAAGCCCGGCCTCTGCCTGGGCCTCTGTG

The disclosed NOV2e nucleic acid sequence, localized to chromsome 19, has 2166 of 2977 bases (72%) identical to a Mus musculus fibrillin 2 (fbn2) mRNA (gb:GENBANK-ID:MUSFBN2|acc:L39790.1) (E=0.0).

A NOV2e polypeptide (SEQ ID NO: 14) encoded by SEQ ID NO: 13 has 2695 amino acid residues and is presented using the one-letter code in Table 2J. Signal P. Psort and/or Hydropathy results predict that NOV2e contains a signal peptide and is likely to be localized to the nucleus with a certainty of 0.6000. Although PSORT suggests that the Fibrillin-like protein may be localized in the nucleus, the NOV2e protein is similar to the Fibrillin family, some members of which are released extracellularly. Therefore it is likely that NOV2e protein shows a similar localization. The most likely cleavage site for a NOV2e peptide is between amino acids 29 and 30, at: AGG-QG.

TABLE 2J

Encoded NOV2e protein sequence (SEQ ID NO:14).
MTLEGLYLARGPLARLLLAWSALLCMAGGQGRWDGALEAAGPGRVRRRGSPGILQGPNVCGSRFHAYCCPGWRTFPGRSQC
VVPICRRACGEGFCSQPNLCTCADGTLAPSCGVSRGSGCSVSCMNGGTCRGASCLCQKGYTGTVCGQPICDRGCHNGGRCI
GPNRCACVYGFMGPQCERDYRTGSCFGQVGPEGCQHQLTGLVCTKALCCATVGRAWGLPCELCPAQPHPCRRGFIPNIHTG
ACQDVDECQAVPGLCQGGSCVNMVGSFHCRCPVGHRLSDSSAACEDYRAGACFSVLFGGRCAGDLAGHYTRRQCCCDRGRC
WAAGPVPELCPPRGSNEFQQLCAQRLPLLPGHPGLFPGLLGFGSNGMGPPLGPARLNPHGSDARGIPSLGPGNSNIGTATL
NQTIDICRHFTNLCLNGRCLPTPSSYRCECNVGYTQDVRGECIDVDECTSSPCHHGDCVNIPGTYHCRCYPGFQATPTRQA
CVDVDECIVSGGLCHLGRCVNTEGSFQCVCNAGFELSPDGKNCVDHNECATSTMCVNGVCLNEDGSFSCLCKPGFLLAPGG
HYCMDIDECQTPGICVNGHCTNTEGSFRCQCLGGLAVGTDGRVCVDTHVRSTCYGAIEKGSCARPFPGTVTKSECCCANPD
HGFGEPCQLCPAKNSAEFQALCSSGLGITTDGRDINECALDPEVCANGVCENLRGSYRCVCNLGYEAGASGKDCTDVDECA
LNSLLCDNGWCQNSPGSYSCSCPPGFHFWQDTEICKDVDECLSSPCVSGVCRNLAGSYTCKCGPGSRLDPSGTFCLDSTKG
TCWLKIQESRCEVNLQGASLRSECCATLGAAWGSPCERCEIDPACARGFARMTGVTCDDVNECESFPGVCPNGRCVNTAGS
FRCECPEGLMLDASGRLCVDVRLEPCFLRWDEDECARDPLLCWGGTCTNTDGSYKCQCPPGHELTAKGTACEDIDECSLSD
GLCPHGQCVNVIGAFQCSCHAGFQSTPDRQGCVDINECRVQNGGCDVHCINTEGSYRCSCGQGYSLMPDGRACADVDECEE
NPRVCDQGHCTNMPGGHRCLCYDGFMATPDMRTCVDVDECDLNPHICLHGDCENTKGSFVCHCQLGYMVRKGATGCSDVDE
CEVGGHNCDSHASCLNIPGSFSCRCLPGWVGDGFECHDLDECVSQEHRCSPRGDCLNVPGSYRCTCRQGFAGDGFFCSDRD
ECAENVDLCDNGQCLNAPGGYRCECEMGFDPTEDHRACQMDECAQGNLCAFGSCENLPGMFRCICNGGYELDRGGGNCTTD
INECADPVNCINGVCINTPGSYLCSCPQDFELNPSGVGCVDTRAGNCFLETHDRGDSGISCSAEIGVGVTRASCCCSLGRA
WGNPCELCPMANTTEYRTLCPGGELFQPNRITVILEDIDECQELPGLCQGGDCVNTFGSFQCECPPGYHLSEHTRICEDID
ECSTHSGICGPGTCYNTLGNYTCVCPAEYLQVNGGNNCMDMRKSVCFRHYNGTCQNELAFNVTRKMCCCSYNIGQAWNRPC
EACPTPISPDYQILCGNQAPGFLTDIHTGKPLDIDECGEIPAICANGICINQIGSFRCECPAGFNYNSILLACEDVDECGS
RESPCQQNADCINIPGSYRCKCTRGYKLSPGGACVGRNECREIPNVCSHGDCMDTEGSYMCLCHRGFQASADQTLCMDIDE
CDRQPCGNGTCKNIIGSYNCLCFPGFVVTHNGDCVDFDECTTLVGQVCRFGHCLNTAGSFHCLCQDGFELTADGKNCVDTN
ECLSLAGTCLPGTCQNLEGSFRCICPPGFQVQSDHCIDIDECSEEPNLCLFGTCTNSPGSFQCLCPPGFVLSDNGHRCFDT
RQSFCFTRFEAGHCSVPKAFNTTKTRCCCSKRPGEGWGDPCELCPQEDSPPPLRPAAFQELCPFGHGAVPGPDDSREDVNE
CAENPGVCTNGVCVNTDGSFRCECPFGYSLDFTGINCEDTDECSVGHPCGQGTCTNVIGGFECACADGFEPGLMMTCEDID
ECSLNPLLCAFRCHNTEGSYLCTCPAGYTLREDGAMCRDVDECADGQQDCHARGMECKNLIGTFACVCPPGMRPLPGSGEG
CTDDNECHAQPDLCVNGRCVNTAGSFRCDCDEGFQPSPTLTECHDIRQGPCFAEVLQTMCRSLSSSSEAVTRAECCCGGGR
GWGPRCELCPLPGTSAYRKLCPHGSGYTAEGRDVDECRMLAHLCAHGECINSLGSFRCHCQAGYTPDATATTCLDMDECSQ
VPKPCTFLCKNTKGSFLCSCPRGYLLEEDGRTCKDLDECTSRQHNCQFLCVNTVGAFTCRCPPGFTQHHQACFDNDECSAQ
PGPCGAHGHCHNTPGSFRCECHQGFTLVSSGHGCEDVNECDGPHRCQHGCQNQLGGYRCSCPQAFTQHSQWAQCVDENECA
LSPPTCGSASCRNTLGGFRCVCPSGFDFDQALGGCQEVDECAGRRGPCSYSCANTPGGFLCGCPQGYFRVGQGHCVSGLGF
SPGPQDTPDKEELLSSEACYECKINGLSPRDRPRRSAHRDHQVNLATLDSEALLTLGLNLSHLGRAERILELRPALEGLEG
RIRYVIVRGNEQGFFRMHHLRGVSSLQLGRRRPGPGTYRLEVVSHMAGPWGVQQEGQPGPWGQALRLKVQLSVALVGRSLS
GPQLSREGGFWNWEGLIPRSDG

The NOV2e amino acid sequence has 1881 of 2256 amino acid residues (83%) identical to, and 1935 of 2256 amino acid residues (85%) similar to, a Homo sapiens 2809 amino acid residue fibrillin 3 protein (ptnr:TREMBLNEW-ACC:BAB47408) (E=0.0).

NOV2e is expressed in at least the following tissues: lung, colon, bone, trabecular bone cells, placenta, germ cell, melanocyte, heart, uterus, thyroid and brain. Expression information was derived from the tissue sources of the sequences that were included in the derivation of the NOV2e sequence.

NOV2f

A disclosed NOV2f nucleic acid of 9154 nucleotides (also referred to as CG88987-05) encoding a novel Fibrillin-like protein is shown in Table 2K. An open reading frame was identified beginning with an ATG initiation codon at nucleotides 41-43 and ending with a TGA codon at nucleotides 8312-8314. Putative untranslated regions upstream from the intiation codon and downstream from the termination codon are underlined in Table 2K, and the start and stop codons are in bold letters.

TABLE 2K

NOV2f nucleotide sequence (SEQ ID NO:15).
CTGACTCTGCTTCCTCTCCGCAGCCTCCAGGGGACACGCCATGACTCTGGAGGGTCTGTATTTGGCAAGGGGCCCCCTGGC
CCGGCTCCTGCTGGCCTGGTCGGCCCTGTTGTGCATGGCAGGTGGCCAAGGCCGCTGGGACGGGGCCTTGGAGGCTGCAGG
TCCTGGACGTGTGCGGAGGCGGGGCAGCCCAGGCATCTTGCAGGGGCCGAATGTGTGCGGCTCCCGGTTCCATGCCTACTG
CTGTCCAGGCTGGAGGACATTCCCTGGCAGGAGCCAGTGTGTCGTACCCATCTGTAGGCGCGCCTGCGGTGAAGGCTTCTG
CTCCCAGCCCAACCTGTGCACCTGTGCGGATGGGACGCTGGCTCCCAGCTGCGGGGTGAGCCGAGCCATCTGTGACCGCGG
CTGCCACAATGGGGGTCGCTGCATTGGGCCCAACCGCTGCGCCTGTGTGTATGGCTTCATGGGACCTCAATGTGAGAGAGA
TTACCGGACGGGACCCTGCTTTGGCCAAGTAGGCCCCGAGGGGTGCCAGCATCAGCTGACGGGCCTCGTGTGCACCAAGGC
ACTTTGCTGTGCCACTGTGGGCCGTGCCTGGGGCCTTCCATGTGAACTTTGCCCTGCACAGCCACACCCCTGCCGCCGCGG
CTTCATCCCCAATATCCACACGGGGGCCTGCCAAGATGTGGATGAGTGCCAGGCTGTGCCAGGCCTGTGCCAGGGAGGCAG
CTGCGTCAACATGGTGGGCTCCTTCCATTGCCGCTGTCCAGTTGGACACCGGCTCAGTGACAGCAGCGCCGCATGTGAAGA
CTACGATGAATGCAGCACCATTCCTGGAATCTGTGAAGGGGGTGAATGTACAAACACAGTCAGCAGTTACTTTTGCAAATG
TCCTCCTGGTTTTTACACCTCTCCAGATGGTACTCTTCATGGACAGTCGCGGGCCGGCGCCTGCTTCTCAGTGCTTTTCGG
GGGCCGCTGTGCTGGAGACCTCGCCGGCCACTACACTCGCAGGCAGTGCTGCTGTGACAGGGGCAGGTGCTGGGCAGCTGG
CCCGGTCCCTGAGCTGTGTCCTCCTCGGGGCTCCAATGAATTCCAGCAACTGTGCGCCCAGCGGCTGCCGCTGCTACCCGG
CCACCCTGGCCTCTTCCCTGGCCTCCTGGGCTTCGGATCCAATGGCATGGGTCCCCCTCTTGGGCCAGCGCGACTCAACCC
CCATGGCTCTGATGCGCGTGGGATCCCCAGCCTGGGCCCTGGCAACTCTAATATTGGCACTGCTACCCTGAACCAGACCAT
TGACATCTGCCGACACTTCACCAACCTGTGTCTGAATGGCCGCTGCCTGCCCACGCCTTCCAGCTACCGCTGCGAGTGTAA
CGTGGGCTACACCCAGGACGTGCGCGGCGAGTGCATTGATGTAGACGAATGCACCAGCAGCCCCTGCCACCACGGTGACTG
CGTCAACATCCCCGGCACCTACCACTGCCGGTGCTACCCGGGCTTCCAGGCCACGCCCACCAGGCAGGCATGCGTGGATGT
GGACGAGTGCATTGTCAGTGGTGGCCTTTGTCACCTGGGCCGCTGTGTCAACACAGAGGGCAGCTTCCAGTGTGTCTGCAA
TGCAGGCTTCGAGCTCAGCCCTGACGGCAAGAACTGTGTGGACCACAACGAGTGTGCCACCAGCACCATGTGCGTCAACGG
CGTGTGTCTCAACGAGGATGGCAGCTTCTCCTGCCTCTGCAAACCCGGCTTCCTGCTGGCGCCTGGCGGCCACTACTGCAT
GGACATTGACGAGTGCCAGACGCCCGGCATCTGCGTGAACGGCCACTGTACCAACACCGAGGGCTCCTTCCGCTGCCAGTG
CCTGGGGGGGCTGGCGGTAGGCACGGATGGCCGCGTGTGCGTGGACACCCACGTGCGCAGCACCTGCTATGGGGCCATCGA
GAAGGGCTCCTGTGCCCGCCCCTTCCCTGGCACTGTCACCAAGTCCGAGTGCTGCTGTGCCAATCCGGACCACGGTTTTGG
GGAGCCCTGCCAGCTTTGTCCTGCCAAAGACTCCGCTGAGTTCCAGGCACTGTGCAGCAGTGGGCTTGGCATTACCACGGA
TGGTCGAGACATCAACGAGTGTGCTCTGGATCCTGAGGTTTGTGCCAATGGCGTGTGCGAGAACCTTCGGGGCAGCTACCG
CTGTGTCTGCAACCTGGGTTATGAGGCAGGTGCCTCAGGCAAGGACTGCACAGACGTGGATGAGTGTGCCCTCAACAGCCT
CCTGTGTGACAACGGGTGGTGCCAGAATAGCCCTGGCAGCTACAGCTGCTCCTGCCCCCCCGGCTTCCACTTCTGGCAGGA
CACGGAGATCTGCAAAGATGTCGACGAATGCCTGTCCAGCCCGTGTGTGAGTGGCGTCTGTCGGAACCTGGCCGGCTCCTA
CACCTGCAAATGTGGCCCTGGCAGCCGGCTGGACCCCTCTGGTACCTTCTGTCTAGACAGCACCAAGGGCACCTGCTGGCT
GAAGATCCAGGAGAGCCGCTGTGAGGTGAACCTTCAGGGAGCCAGCCTGCGGTCTGAGTGCTGTGCCACCCTCGGGGCAGC
CTGGGGGAGCCCCTGCGAACGCTGCGAGATCGACCCTGCCTGTGCCCGGGGCTTTGCCCGGATGACGGGTGTCACCTGCGA
TGATGTGAACGAGTGTGAGTCCTTCCCGGGAGTCTGTCCCAACGGGCGTTGCGTCAACACTGCTGGGTCTTTCCGCTGTGA
GTGTCCAGAGGGCCTGATGCTGGACGCCTCAGGCCGGCTGTGCGTGGATGTGAGATTGGAACCATGTTTCCTGCGATGGGA
TGAGGATGAGTGTGGGGTCACCCTGCCTGGCAAGTACCGGATGGACGTCTGCTGCTGCTCCATCGGGGCCGTGTGGGGAGT
CGAGTGCGAGGCCTGCCCGGATCCCGAGTCTCTGGAGTTCGCCAGCCTGTGCCCGCGGGGGCTGGGCTTCGCCAGCCGGGA
CTTCCTGTCTGGCCGACCATTCTATAAAGATGTGAATGAATGCAAGGTGTTCCCTGGCCTCTGCACGCACGGTACCTGCAG
AAACACGGTGGGCAGCTTCCACTGCGCCTGTGCGGGGGGCTTCGCCCTGGATGCCCAGGAACGGAACTGCACAGATATCGA
CGAGTGTCGCATCTCTCCTGACCTCTGCGGCCAGGGCACCTGTGTCAACACGCCGGGCAGCTTTGAGTGCGAGTGTTTTCC
CGGCTACGAGAGTGGCTTCATGCTGATGAAGAACTGCATGGACGTGGACGAGTGTGCAAGGGACCCGCTGCTCTGCCGGGG
AGGCACTTGCACCAACACGGATGGGAGCTACAAGTGCCAGTGTCCCCCTGGGCATGAGCTGACGGCCAAGGGCACTGCCTG
TGAGGACATCGATGAGTGCTCCCTGAGTGATGGCCTGTGTCCCCATGGCCAGTGTGTCAATGTCATCGGTGCCTTCCAGTG
CTCCTGCCATGCCGGCTTCCAGAGCACACCTGACCGCGGAGCTACAAGTGCCAGTTGCCCGACGGAAGGGCATGTGCAGGT
CGTCCTGGGGCCTGGGGAGCAGATGTGTACTGGGTGGTCTATCAGGGCAAAGCTAAGCACAGTCCCCAACCCTGCTCCCCC
AGACGTGGACGAGTGTGAAGAGAACCCCCGCGTTTGTGACCAAGGCCACTGCACCAACATGCCAGGGGGTCACCGCTGCCT
GTGCTATGATGGCTTCATGGCCACGCCAGACATGAGGACATGTGTTGATGTGGATGAGTGTGACCTGAACCCTCACATCTG
CCTCCATGGGGACTGCGAGAACACGAAGGGTTCCTTTGTCTGCCACTGTCAGCTGGGCTACATGGTCAGGAAGGGGGCCAC
AGGCTGCTCTGATGTGGATGAATGCGAGGTTGGAGGACACAACTGTGACAGTCACGCCTCCTGTCTCAACATCCCAGGGAG
TTTCAGCTGTAGGTGCCTGCCAGGCTGGGTGGGGGATGGCTTCGAATGTCACGACCTGGATGAATGCGTCTCCCAGGAGCA
CCGGTGCAGCCCAAGAGGTGACTGTCTCAATGTCCCTGGCTCCTACCGCTGCACCTGCCGCCAGGGCTTTGCCGGGGATGG
CTTCTTCTGCGAAGACAGGGATGAATGTGCCGAGAACGTGGACCTCTGTGACAACGGGCAGTGCCTCAATGCGCCCGGCGG
GTACCGCTGTGAATGTGAGATGGGCTTTGACCCCACCGAGGACCACCGGGCCTGCCAGGATGTGGACGAGTGTGCGCAAGA
GAACCTCTGTGCATTTGGGAGCTGTGAGAACCTGCCTGGAATGTTCCGCTGCATCTGCAATGGTGGCTACGAACTGGACCG
AGGGGGTGGCAACTGCACAGACATCAACGAGTGTGCAGACCCAGTAAACTGCATCAACGGCGTGTGCATTAACACCCCCGG
CAGCTACCTCTGCAGCTGCCCCCAGGATTTTGAGCTGAACCCCAGCGGAGTGGGCTGCGTGGACACTCGGGCCGGGAACTG
TTTCCTGGAGACGCATGACCGAGGGGACAGTGGCATTTCCTGCAGTGCCGAGATCGGAGTTGGTGTCACCCGAGCTTCCTG
CTGTTGCTCCCTGGGCCGGGCTTGGGGCAATCCCTGTGAGCTGTGCCCTATGGCCAACACCACTGAGTACAGAACCCTGTG
CCCGGGTGGTGAGGGCTTCCAGCCTAACCGCATCACTGTCATTCTGGAAGACATCGACGAGTGCCAAGAGCTGCCAGGGCT
GTGTCAGGGGGGTGACTGCGTCAACACGTTTGGCAGTTTCCAGTGTGAGTGCCCACCTGGCTACCACCTCAGTGAGCACAC
CCGCATCTGTGAGGATATTGACGAATGCTCCACACACTCCGGCATCTGTGGCCCTGGCACCTGCTACAACACCCTGGGGAA
CTACACCTGTGTCTGCCCTGCAGAGTACCTCCAAGTCAATGGTGGCAACAACTGCATGGATATGAGGAAGAGTGTCTGCTT
CCGGCACTATAACGGCACATGTCAAAATGAGCTGGCCTTCAACGTGACCCGGAAAATGTGTTGCTGCTCCTACAACATTGG
CCAGGCCTGGAATAGACCCTGTGAGGCCTGCCCCACTCCCATCAGTCCTGACTACCAGATCCTGTGTGGAAATCAGGCCCC
CCCATTCCTCACTGACATCCACACGGGGAAGCCCCTTGACATTGATGAGTGTGGGGAGATCCCCGCCATCTGTGCCAATGG
CATCTGCATAAACCAGATCGGGAGTTTCCGCTGCGAGTGCCCCGCAGGCTTCAACTACAACAGCATCCTGCTGGCTTGTGA
AGATGTCGATGAGTGTGGCAGCAGGGAGAGTCCCTGCCAGCAGAATGCTGACTGCATCAACATCCCCGGTAGCTACCGCTG
CAAGTGCACCCGAGGGTACAAACTGTCGCCAGGCGGGGCTTGTGTGGGACGGAATGAGTGTCGGGAGATCCCGAATGTCTG
TAGCCATGGTGACTGCATGGACACAGAAGGCAGCTACATGTGTCTGTGTCACCGTGGATTCCAGGCCTCTGCAGACCAGAC
CCTGTGCATGGACATTGACGAGTGTGACCGGCAGCCTTGTGGAAATGGGACCTGCAAGAACATCATTGGCTCCTACAACTG
CCTCTGCTTCCCTGGCTTTGTGGTGACACACAATGGGGATTGTGTGGATTTTGATGAGTGTACTACCCTGGTGGGGCAGGT
GTGCCGATTTGGCCATTGCCTCAACACAGCTGGTTCCTTCCACTGCCTCTGCCAGGATGGCTTTGAGCTCACAGCTGATGG
GAAGAACTGTGTGGACACCAATGAGTGCCTCAGCCTTGCAGGAACCTGCCTACCCGGCACTTGCCAGAACCTCGAGGGCTC
CTTCCGCTGCATCTGTCCCCCTGGCTTCCAGGTGCAGAGTGACCACTGCATTGATATCGACGAGTGCTCAGAGGAGCCCAA
CCTCTGCCTCTTTGGCACCTGTACCAACAGCCCTGGGAGCTTCCAGTGCCTCTGCCCACCTGGCTTTGTCCTCTCTGACAA
TGGGCACCGTTGCTTTGACACACGGCAGAGTTTCTGCTTCACCCGTTTTGAGGCTGGGAAGTGCTCGGTGCCCAAAGCTTT
CAACACCACCAAGACCCGCTGCTGCTGCAGTAAGAGGCCTGGGGAGGGCTGGGGAGACCCCTGCGAACTGTGTCCCCAGGA
AGGCAGCGCTGCCTTTCAGGAGCTCTGCCCCTTTGGCCACGGGGCAGTCCCAGGCCCGGATGACTCCCGAGAAGACGTGAA
TGAGTGTGCAGAGAACCCTGGCGTCTGCACTAACGGCGTCTGTGTCAACACCGATGGATCCTTCCGCTGTGAGTGTCCCTT
TGGCTACAGCCTGGACTTCACTGGCATCAACTGTGAGGACACAGACGAGTGCTCTGTCGGCCACCCCTGTGGGCAAGGGAC
ATGCACCAATGTCATCGGAGGCTTCGAATGTGCCTGTGCTGACGGCTTTGAGCCTGGCCTCATGATGACCTGCGAGGACAT
CGACGAATGCTCCCTGAACCCGCTGCTCTGTGCCTTCCGCTGCCACAATACCGAGGGCTCCTACCTGTGCACCTGTCCAGC
CGGCTACACCCTGCGGGAGGACGGGGCCATGTGTCGAGATGACAATGAATGCCACGCTCAGCCTGACCTCTGTGTCAACGG
CCGCTGTGTCAACACCGCGGGCAGCTTCCGGTGCGACTGTGATGAGGGATTCCAGCCCAGCCCCACCCTTACCGAGTGCCG
CGACATCCGGCAGGGGCCCTGCTTTGCCGAGGTGCTGCAGACCATGTGCCGGTCTCTGTCCAGCAGCAGTGAGGCTGTCAC
CAGGGCCGAGTGCTGCTGTGGGGGTGGCCGGGGCTGGGGGCCCCGCTGCGAGCTCTGTCCCCTGCCCGGCACCTCTGCCTA
CAGGAAGCTGTGCCCCCATGGCTCAGGCTACACTGCTGAGGGCCGAGATGTAGATGAATGCCGTATGCTTGCTCACCTGTG
TGCTCATGGGGAGTGCATCAACAGCCTTGGCTCCTTCCGCTGCCACTGTCAGGCCGGGTACACACCGGATGCTACTGCTAC
TACCTGCCTGGATATGGATGAGTGCAGCCAGGTCCCCAAGCCATGTACCTTCCTCTGCAAAAACACGAAGGGCAGTTTCCT
GTGCAGCTGTCCCCGAGGCTACCTGCTGGAGGAGGATGGCAGGACCTGCAAAGACCTGGACGAATGCACCTCCCGGCAGCA
CAACTGTCAGTTCCTCTGTGTCAACACTGTGGGCGCCTTCACCTGCCGCTGTCCGCCCGGCTTCACCCAGCACCACCAGGC
CTGCTTCGATGTGAATGAATGTGATGGGCCCCACCGCTGCCAGCATGGCTGTCAGAACCAGCTAGGGGGCTACCGCTGCAG
CTGCCCCCAGGGTTTCACCCAGCACTCCCAGTGGGCCCAGTGTGTGGATGAGAATGAGTGTGCCCTGTCGCCCCCCACCTG
CGGGAGCGCCTCCTGTCGCAACACTCTTGGTGGCTTCCGCTGCGTCTGCCCCTCTGGCTTTGACTTTGATCAGGCCCTCGG
GGGCTGCCAGGAGGTGGATGAGTGCGCCGGACGGCGTGGCCCCTGTAGCTACAGCTGTGCCAACACGCCTGGTGGCTTCCT
GTGCGGCTGTCCTCAAGGCTACTTCCGGGCTGGGCAAGGGCACTGTGTCTCCGGCCTGGGCTTCAGCCCCGGACCCCAGGA
CACCCCGGACAAAGAGGAGCTGCTCTCGTCTGAAGCCTGCTACGAATGCAAGATCAATGGCCTCTCCCCTCGGGACCGGCC
ACGACGCAGTGCCCACAGGGACCACCAGGTGAACCTGGCCACCCTTGACTCCGAGGCCCTGCTGACCTTGGGCCTGAACCT
CTCACACCTGGGCCGGGCCGAGCGCATCCTGGAGCTCCGGCCGGCCCTGGAGGGTCTAGAGGGCCGGATCCGCTACGTCAT
CGTCCGCGGAAACGAGCAAGGTTTCTTTCGCATGCATCACCTCCGTGGCGTCAGCTCCCTGCAGCTGGGGCGGAGGCGGCC
GGGGCCTGGAACCTACCGGCTGGAGGTGGTGAGCCACATGGCAGGACCCTGGGGTGTCCAGCAAGAGGGGCAGCCAGGGCC
ATGGGGCCAGGCCTTGAGGCTGAAGGTGCAACTGTCAGTTGCTTTAGTTGGGAGGAGCCTCAGTGGGCCCCAGCTGTCCAG
AGAAGGGGGATTCTGGAACTGGGAAGGACTGATCCCCAGAAGCGATGGCTGACCAGATTGAACCCCGAAACTCAGGAAGAG
TGAAATGCTACACGACAACCTCAGGCAAGCCCGGCCTCTGCCTGGGCCTCTGTGCCAGCCCCGGGGGCCCCCCAGTTACTC
AGTCTTTCCTGGAGACAGCAAGAAGCTGCAATGTGCAATCCCCCTGCCCCCACAGCCAAGGTCAGGAAGAGGCCCTGTGGT
CACCGTGTCTGGCCAATCTCAGGCTTTCACTTCTGTACTGCACTGTGGCTTGCCCTGGCGGGGGGCAGGGGGTTGGCAGGA
CATGGCAATGGGCAACTGGGGTGGGCACAGGGCTTATTCCTCGGAGTAGAAGGGTGTACAGGGGGCCCAGACTCCACAGTG
ACTTGCCACATTTGCCCCCCATTTGGAGAATGCTTTTATATCAAAAGTGGAGACGATAATAAAGTTATTTTGGGTTAAGTC
TGCCTGCCCTTTGGCAAGTTCTTGAAGTAAGTAGATGCTGCCCTCGGACTGGGCGAGGCAGATCTTGTGCCTGGGGAAGCA
GAAGGCCTTATGGGCTCCCCAGAATGGTAATAATGGCTCACGCTTCCTGACCACGTACTACATACCAGACACCATTCGATT
TTTTTTTTATTTTTTCTGAGACAGGGTCTTGCTCTGTTGCCTAGGTTGGAGTGCAGTGGCGCGATCGTGGCTCCCTGTGGT
CGCCACTTTCCGGGCTCGAGCAATCCTCCCACCTCAGCCTCTCCCAAGTAGCTGGGACCAAAGGTGCACGCCACCACACCC
AGCTAATTTTTTTAAATTTCTTTTTCTTTCTCTTTCTTTTTGAGACCAGCCCGGCCAACATGACGAAACCCCATCTCTAAT
A

The disclosed NOV2f nucleic acid sequence, localized to chromsome 19, has 3401 of 3689 bases (92%) identical to a Homo sapiens KIAA1 776 protein (fibrillin3) mRNA (gb:GENBANK-ID:AB053450|acc:AB053450.2) (E=0.0).

A NOV2f polypeptide (SEQ ID NO: 16) encoded by SEQ ID NO: 15 has 2757 amino acid residues and is presented using the one-letter code in Table 2L. Signal P, Psort and/or Hydropathy results predict that NOV2f contains a signal peptide and is likely to be localized to the nucleus with a certainty of 0.6000. Although PSORT suggests that the Fibrillin-like protein may be localized in the nucleus, the NOV2f protein is similar to the Fibrillin family, some members of which are released extracellularly. Therefore it is likely that NOV2f protein shows a similar localization. The most likely cleavage site for a NOV2f peptide is between amino acids 29 and 30, at: AGG-QG.

TABLE 2L

Encoded NOV2f protein sequence (SEQ ID NO:16).
MTLEGLYLARGPLARLLLAWSALLCMAGGQGRWDGALEAAGPGRVRRRGSPGILQGPNVCGSRFHAYCCPGWRTFPGRSQC
VVPICRRACGEGFCSQPNLCTCADGTLAPSCGVSRAICDRGCHNGGRCIGPNRCACVYGFMGPQCERDYRTGPCFGQVGPE
GCQHQLTGLVCTKALCCATVGRAWGLPCELCPAQPHPCRRGFIPNIHTGACQDVDECQAVPGLCQGGSCVNMVGSFHCRCP
VGHRLSDSSAACEDYDECSTIPGICEGGECTNTVSSYFCKCPPGFYTSPDGTLHGQSRAGACFSVLFGGRCAGDLAGHYTR
RQCCCDRGRCWAAGPVPELCPPRGSNEFQQLCAQRLPLLPGHPGLFPGLLGFGSNGMGPPLGPARLNPHGSDARGIPSLGP
GNSNIGTATLNQTIDICRHFTNLCLNGRCLPTPSSYRCECNVGYTQDVRGECIDVDECTSSPCHHGDCVNIPGTYHCRCYP
GFQATPTRQACVDVDECIVSGGLCHLGRCVNTEGSFQCVCNAGFELSPDGKNCVDHNECATSTMCVNGVCLNEDGSFSCLC
KPGFLLAPGGHYCMDIDECQTPGICVNGHCTNTEGSFRCQCLGGLAVGTDGRVCVDTHVRSTCYGAIEKGSCARPFPGYVT
KSECCCANPDHGFGEPCQLCPAKDSAEFQALCSSGLGITTDGRDINECALDPEVCANGVCENLRGSYRCVCNLGYEAGASG
KDCTDVDECALNSLLCDNGWCQNSPGSYSCSCPPGFHFWQDTEICKDVDECLSSPCVSGVCRNLAGSYTCKCGPGSRLDPS
GTFCLDSTKGTCWLKIQESRCEVNLQGASLRSECCATLGAAWGSPCERCEIDPACARGFARMTGVTCDDVNECESFPGVCP
NGRCVNTAGSFRCECPEGLMLDASGRLCVDVRLEPCFLRWDEDECGVTLPGKYRMDVCCCSIGAVWGVECEACPDPESLEF
ASLCPRGLGFASRDFLSGRPFYKCVNECKVFPGLCTHGTCRNTVGSFHCACAGGFALDAQERNCTDIDECRISPDLCGQGT
CVNTPGSFECECFPGYESGFMLMKNCMDVDECARDPLLCRGGTCTNTDGSYKCQCPPGHELTAKGTACEDIDECSLSDGLC
PHGQCVNVIGAFQCSCHAGFQSTPDRGATSASCPTEGHVQVVLGPGEQMCTGWSIRAKLSTVPNPAPPDVDECEENPRVCD
NCDSHASCLNIPGSFSCRCLPGWVGDGFECHDLDECVSQEHRCSPRGDCLNVPGSYRCTCRQGFAGDGFFCEDRDECAENV
DLCDNGQCLNAPGGYRCECEMGFDPTEDHRACQDVDECAQENLCAFGSCENLPGMFRCICNGGYELDRGGGNCTDINECAD
PVNCINGVCINTPGSYLCSCPQDFELNPSGVGCVDTRAGNCFLETHDRGDSGISCSAEIGVGVTRASCCCSLGRAWGNPCE
LCPMANTTEYRTLCPGGEGFQPNRITVILEDIDECQELPGLCQGGDCVNTFGSFQCECPPGYHLSEHTRICEDIDECSTHS
GICGPGTCYNTLGNYTCVCPAEYLQVNGGNNCMDMRKSVCFRHYNGTCQNELAFNVTRKMCCCSYNIGQAWNRPCEACPTP
ISPDYQILCGNQAPGFLTDIHTGKPLDIDECGEIPAICANGICINQIGSFRCECPAGFNYNSILLACEDVDECGSRESPCQ
QNADCINIPGSYRCKCTRGYKLSPGGACVGRNECREIPNVCSHGDCMDTEGSYMCLCHRGFQASADQTLCMDIDECDRQPC
GNGTCKNIIGSYNCLCFPGFVVTHNGDCVDFDECTTLVGQVCRFGHCLNTAGSFHCLCQDGFELTADGKNCVDTNECLSLA
GTCLPGTCQNLEGSFRCICPPGFQVQSDHCIDIDECSEEPNLCLFGTCTNSPGSFQCLCPPGFVLSDNGHRCFDTRQSFCF
TRFEAGHCSVPKAFNTTKTRCCCSKRPGEGWGDPCELCPQEGSAAFQELCPFGHGAVPGPDDSREDVNECAENPGVCTNGV
CVNTDGSFRCECPFGYSLDFTGINCEDTDECSVGHPCGQGTCTNVIGGFECACADGFEPGLMMTCEDIDECSLNPLLCAFR
CHNTEGSYLCTCPAGYTLREDGAMCRDDNECHAQPDLCVNGRCVNTAGSFRCDCDEGFQPSPTLTECRDIRQGPCFAEVLQ
TMCRSLSSSSEAVTRAECCCGGGRGWGPRCELCPLPGTSAYRKLCPHGSGYTAEGRDVDECRMLAHLCAHGECINSLGSFR
CHCQAGYTPDATATTCLDMDECSQVPKPCTFLCKNTKGSFLCSCPRGYLLEEDGRTCKDLDECTSRQHNCQFLCVNTVGAF
TCRCPPGFTQHHQACFDVNECDGPHRCQHGCQNQLGGYRCSCPQGFTQHSQWAQCVDENECALSPPTCGSASCRNTLGGFR
CVCPSGFDFDQALGGCQEVDECAGRRGPCSYSCANTRGGFLCGCPQGYFRAGQGHCVSGLGFSPGPQDTPDKEELLSSEAC
YECKINGLSPRDRPRRSAHRDHQVNLATLDSEALLTLGLNLSHLGRAERILELRPALEGLEGRIRYVIVRGNEQGFFRMHH
LRGVSSLQLGRRRPGPGTYRLEVVSHMAGPWGVQQEGQPGPWGQALRLKVQLSVALVGRSLSGPQLSREGGFWNWEGLIPR
SDG

The NOV2f amino acid sequence has 2176 of 2568 amino acid residues (84%) identical to, and 2245 of 2568 amino acid residues (87%) similar to, a Homo sapiens 2809 amino acid residue fibrillin 3 protein (ptnr:TREMBLNEW-ACC:BAB47408) (E=0.0).

NOV2f is expressed in at least the following tissues: Mammalian Tissue, Brain and Lung. Expression information was derived from the tissue sources of the sequences that were included in the derivation of the NOV2f sequence.

In addition, NOV2f is predicted to be expressed in the following tissues because of the expression pattern of a closely related Homo sapiens mRNA homolog for KIAA1776 protein (fibrillin3) (gb:GENBANK-ID:AB053450|acc:AB053450.2): adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

Possible small nucleotide polymorphisms (SNPs) found for NOV2a are listed in Table 2M.

Table 2M
SNPs

Amino

| | | | | |
|---|---|---|---|---|
| | Neucleotide | Base | Acid Base | |
| Variant | Position | Change | Position | Change |

| | | | | |
|---|---|---|---|---|
| 13375333 | 4160 | A > G | 1214 | Gln > Arg |
| 13375332 | 4244 | A > G | 1242 | Asn > Ser |

NOV2a-NOV2f are very closely homologous as is shown in the amino acid alignment in Table 2N.

[image]

[image]

[image]

[image]

[image]

[image]

[image]

[image]

Homologies to any of the above NOV2 proteins will be shared by the other NOV2 proteins insofar as they are homologous to each other as shown above. Any reference to NOV2 is assumed to refer to both of the NOV2 proteins in general, unless otherwise noted.

NOV2a also has homology to the amino acid sequences shown in the BLASTP data listed in Table 2O.

TABLE 2O
BLAST results for NOV2a

| Gene Index/ | Protein/ | Length | Identity | Positives | |
|---|---|---|---|---|---|
| Identifier | Organism | (aa) | (%) | (%) | Expect |
| gi\|1335064\|emb\| | fibrillin | 3002 | 1723/2862 | 2149/2862 | 0.0 |
| CAA45118.1\|(X63556) | [Homo sapiens] | | (60%) | (74%) | |
| gi\|4557591\|ref\| | fibrillin 1; | 2871 | 1723/2848 | 2147/2848 | 0.0 |
| NP_000129.1\| | Fibrillin-1 | | (60%) | (74%) | |
| (NM_000138) | [Homo sapiens] | | | | |
| gi\|663111\|gb\| | fibrillin-2 | 1062 | 720/1067 | 865/1067 | 0.0 |
| AAA62177.1\|(U20217) | [Mus musculus] | | (67%) | (80%) | |
| gi\|1706768\|sp\|P98133 | FIBRILLIN 1 | 2871 | 1725/2851 | 2139/2851 | 0.0 |
| \|FBN1_BOVIN | PRECURSOR | | (60%) | (74%) | |
| | (MP340) (Bos | | | | |
| | taurus) | | | | |
| gi\|13929180\|ref\| | fibrillin-2 | 2906 | 1884/2821 | 2243/2821 | 0.0 |
| NP_114014.1\| | [Rattus | | (66%) | (78%) | |
| (NM_031826) | norvegicus] | | | | |

The homology of these sequences is shown graphically in the ClustalW analysis shown in Table 2P.

[image]

[image]

[image]

[image]

[image]

[image]

[image]

[image]

Tables 2Q -2X list the domain description from DOMAIN analysis results against NOV2a. This indicates that the NOV2a sequence has properties similar to those of other proteins known to contain these domains.

TABLE 2Q

Domain Analysis of NOV2a

gnl|Pfam|pfam00683, TB, TB domain.
This domain is also known as the 8 cysteine domain.
This family includes the hybrid domains. This
cysteine rich repeat is found in TGF binding protein
and fibrillin.
Length = 42 residues, 95.2% aligned
Score = 50.1 bits (118), Expect = 2e − 06

| | | | | |
|---|---|---|---|---|
| NOV2a | 1663 | CSAEIGVGVTRASCCCSLGRAWGNPCELCPMANTTEYRTL | 1702 | (SEQ ID NO: 61) |
| | | \|\| + \|\|++ \|\|\|\|\|\|\|\|\|\| \|\|\| \|\|+ \| \|\|+\|\| | | |
| 00683 | 3 | CSNPLPGRVTKSECCCSLGRAWGTPCEPCPVPGTAEYKTL | 42 | |

TABLE 2R

Domain Analysis of NOV2a
gnl|Pfam|pfam00683, TB, TB domain.
Length = 42 residues, 100.0% aligned
Score = 46.2 bits (108), Expect = 3e − 05

| | | | | |
|---|---|---|---|---|
| NOV2a | 2181 | GKCSVPKAFNTTKTRCCCSKRPGEGWGDPCELCPQEGSAAFQEL | 2224 | (SEQ ID NO:62) |
| | | \|+\|\| \| \|\|+ \|\|\|\| \| \|\| \|\|\| \|\| \|+\| ++ \| | | |
| 00683 | 1 | GRCSNPLPGRVTKSECCCS--LGRAWGTPCEPCPVPGTAEYKTL | 42 | |

TABLE 2S

Domain Analysis OF NOV2a
gnl|Pfam|pfam00683, TB TB domain.
Length = 42 residues, 100.0% aligned
Score = 43.9 bits (102), Expect = 1e − 04

| | | | | |
|---|---|---|---|---|
| NOV2a | 1816 | GTCQNELAFNVTRKMCCCSYNIGQAWNRPCEACPTPISPDYQIL | 1859 | (SEQ ID NO:63) |
| | | \| \| \| \| \|\|+ \|\|\|\| \|+\|\| \|\|\| \|\| \| + +\|+ \| | | |
| 00683 | 1 | GRCSNPLPGRVTKSECCCSL--GRAWGTPCEPCPVPGTAEYKTL | 42 | |

TABLE 2T

Domain Analysis of NOV2a
gnl|Pfam|pfam00683, TB, TB domain.
Length = 42 residues, 95.2% aligned
Score = 43.5 bits (101), Expect = 2e − 04

| | | | | |
|---|---|---|---|---|
| NOV2a | 314 | CQHQLTGLVCTKALCCATVGRAWGLPCELCPAQPHPCRRGF | 354 | (SEQ ID NO:64) |
| | | \| + \| \| \| \|\|+ \|\| ++\|\|\|\|\| \|\|\| \|\| + | | |
| 00683 | 3 | CSNPLPGRV-TKSECCCSLGRAWGTPCEPCPVPGTAEYKTL | 42 | |

TABLE 2U

Domain Analysis of NOV2a
gnl|Pfam|pfam00683, TB, TB domain.
Length = 42 residues, 100.0% aligned
Score = 43.1 bits (100), Expect = 2e − 04

| | | | | |
|---|---|---|---|---|
| NOV2a | 787 | GSCARPFPGTVTKSECCCANPDHGFGEPCQLCPAKDSAEFQAL | 829 | (SEQ ID NO:65) |
| | | \| \|+ \| \|\| \|\|\|\|\|\|\|\|+ +\| \|\|+ \|\| +\|\|++ \| | | |
| 00683 | 1 | GRCSNPLPGRVTKSECCCSLGR-AWGTPCEPCPVPGTAEYKTL | 42 | |

TABLE 2V

Domain Analysis of NOV2a
gnl|Pfam|pfam00683, TB, TB domain
Length = 42 residues, 95.2% aligned
Score = 42.7 bits (99), Expect = 3e − 04

| | | | | |
|---|---|---|---|---|
| NOV2a | 979 | SRCEVNLQGASLRSECCATLGAAWGSPCERCEIDPACARG | 1018 | (SEQ ID NO:66) |
| | | \|\| \| \| +\|\|\|\| +\|\| \|\|\|+\|\|\| \| + | | |
| 00683 | 1 | GRCSNPLPGRVTKSECCCSLGRAWGTPCEPCPVPGTAEYK | 40 | |

TABLE 2W

Domain Analysis of NOV2a
gnl|Pfam|pfam00683, TB, TB domain.
Length = 42 residues, 100.0% aligned
Score = 39.3 bits (90), Expect = 0.003

| | | | | |
|---|---|---|---|---|
| NOV2a | 462 | GRCAGDLAGHYTRRQCCCDRGRCWAAGPVPELCPPRGSNEFQQL | 505 | (SEQ ID NO:67) |
| | | \|\|\|+ \| \| \|+ +\|\|\| \|\| \| \| \| \|\| \|+ \|++ \| | | |
| 00683 | 1 | GRCSNPLPGRVTKSECCCSLGRAW--GTPCEPCPVPGTAEYKTL | 42 | |

TABLE 2X

Domain Analysis of NOV2a
gnl|Pfam|pfam00683, TB, TB domain.
Length = 42 residues, 97.6% aligned
Score = 38.5 bits (88), Expect = 0.006

| | | | | |
|---|---|---|---|---|
| NOV2a | 1085 | ECGVTLPGKYRMDVCCCSIGAVWGVECEACPDPESLEFASL | 1125 | (SEQ ID NO:68) |
| | | \| \|\|\|+ \|\|\|\|+\| \|\| \|\| \|\| \| + \|+ +\| | | |
| 00683 | 2 | RCSNPLPGRVTKSECCCSLGRAWGTPCEPCPVPGTAEYKTL | 42 | |

Fibrillins 1 and 2 are the main constituents of the extracellular microfibrils responsible for the biomechanical properties of most tissues and organs. They are cysteine-rich glycoproteins predominantly made of multiple repeats homologous to the calcium-binding epidermal growth factor module, and are translated as precursor proteins cleaved by furine/PACE-like activities. Fibrillins polymerize extracellularly as parallel bundles of head-to-tail monomers. Binding to calcium rigidifies the structure of the monomers and the supramolecular organization of the macroaggregates. Elastic fibers form a network that contributes to the elasticity and resilience of tissues such as the skin. Histopathologic and ultrastructural abnormalities in the elastic fibers have been observed in several diseases of the skin and other tissues. Recent cloning of several genes involved in elastic fiber architecture has lead to the approach of the study of elastic fiber genodermatoses through molecular analysis. In recent years, mutations in several of the genes encoding elastic fiber proteins have been demonstrated in other diseases. Fibrillin-1 mutations result in the pleiotropic manifestations of Marfan syndrome, and fibrillin-2 alterations cause the overlapping phenotype of congenital contractural arachnodactyly, and, most recently, demonstration of abnormalities in the Menkes syndrome gene in X4inked cutis laxa. The first disorders to involve mutations in the elastin gene itself are, surprisingly, cardiovascular and neurobehavioral disorders, such as supravalvular aortic stenosis and Williams syndrome. It is hypothesized that fibrillin-2 guides elastogenesis, whereas fibrillin-1 provides force-bearing structural support (PMID: 10216958, PMID: 7963685).

The above defined information for NOV2 suggests that the NOV2 protein may function as a member of a family of novel Fibrillin-like proteins. Therefore, the NOV2 nucleic acids and proteins of the invention are useful in potential therapeutic applications implicated in various diseases and disorders described below and/or other pathologies. For example, the NOV2 compositions of the present invention will have efficacy for treatment of patients suffering from connective tissue disorders, such as severe neonatal Marfan syndrome, dominant ectopia lentis, familial ascending aortic aneurysm, isolated skeletal features of Marfan syndrome, and Shprintzen-Goldberg syndrome, genodermatoses, contractural arachnodactyly, inflammatory disorders such as osteo- and rheumatoid-arthritis, inflammatory bowel disease, Crohn's disease; immunological disorders, AIDS; cancers including but not limited to lung cancer, colon cancer, leukemia or pancreatic cancer.; blood disorders; asthma; psoriasis; vascular disorders, hypertension, skin disorders, renal disorders including Alport syndrome, immunological disorders, tissue injury, fibrosis disorders, bone diseases, Ehlers-Danlos syndrome type VI, VII, type IV, S-linked cutis laxa and Ehlers-Danlos syndrome type V, osteogenesis imperfecta, Neurologic diseases, Brain and/or autoimmune disorders like encephalomyelitis, neurodegenerative disorders, Alzheimer's Disease, Parkinson's Disorder, immune disorders, and hematopoietic disorders, endocrine diseases, muscle disorders, inflammation and wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, benign prostatic hypertrophy, contractural arachnodactyly/CCA, arthrogryposis multiplex congenita, osteogenesis imperfecta, keratoconus, scoliosis, duodenal atresia, esophageal atresia and intestinal malrotation. The NOV2 nucleic acid encoding Fibrillin-like proteins, and the Fibrillin-like proteins of the invention, or fragments thereof, may further be useful in diagnostic applications, wherein the presence or amount of the nucleic acid or the protein are to be assessed.

NOV3

A disclosed NOV3 nucleic acid of 2713 nucleotides (also referred to as GSAL442663.1_A) encoding a novel KIAA1589-like protein is shown in Table 3A. An open reading frame was identified beginning with a ATG initiation codon at nucleotides 1-3 and ending with a TAG codon at nucleotides 2711-2713. The start and stop codons are in bold letters in Table 3A.

TABLE 3A
NOV3 Nucleotide Sequence

| | |
|---|---|
| ATGGGCCGGGCACGGTGGGTCACATCTGTAATCCCAGCACTTTGGGAGGCCAAGTGTGGGCCTCATTCTAAGTCTGACTT | (SEQ ID NO:17) |
| CCCTAATGCTCAGAAGATGGCTATGTTGAGTCCACAAACTTTTGAATTCAGAAACAAGATTGAGTTGATTTCAGAAGCTC | |
| TCCCAGAAGATCAAGAAGAACTTTCCAGGATGTCCAGGAACCTGAATTGAGTCATACGCCAACAGTGTTCAGAACCCT | |
| GTGGAAGTTTCGTGCAGTCTTCAGACTCAAATCTTCGTCTTCACCCCCGGGGCAAGCTCAGTGACTATTATATGGTGGT | |
| GTGTTTCCTTACCAGCGTGAGTATGAGTGCCCAGACTTCCCCAGCAGAGAAGGGCCTGAATCCGGGGCTGATGTGCCAGG | |
| AAAGTTACGCTTGCAGCGGGACTGATGAAGCTATCTTTGAGTGTGATGAGTGCTGCAGTCTGCAGTGTCTCCGCTGCGAG | |
| GAGGAGCTCCATCGGCAGGAGCGCGTGAGAAACCATGAGCGGATAAGACTCAACCTGGCCATGTCCCTTACTGTGACCT | |
| CTGCAAGGGTCTCAGTGGGCATTTACCAGGTGTTAGGCAGAGGGCAATAGTGAGGTGCCAGACCTGCAAAATTAACTTGT | |
| GCCTGGAGTGCCAGAAGAGGACTCATTCTGGGGGTAACAAAAGGAGACACCCTGTTACTGTGTACAATGTCAGTAATCTC | |
| CAGGAGTCACTGGAGGCAGAAGAGATGGATGAGGAGACCAAGAGGAAGAAGATGACTGAGAAGGTGGTGAGTTTCCTCCT | |
| AGTAGACGAAAATGAAGAATTCAGGTAACAAATGAAGAAGACTTCATTAGAAAATTGGACTGCAAACCTGATCAGCATC | |
| TGAAAGTGGTTTCCATTTTTGGAAATACTGGTGATGGAAAGTCTCATACTCTCAACCACACTTTCTTTTATGGTCGTGAA | |
| CTCTTTAAAACCTCCCCGACCCAGGAGTCCTGCACTGTGGGAGTGTGGGCAGCCTATGACCCAGTTCACAAAGTAGCAGT | |
| GATCGATACGGAAGGGCTCCTGGGGGCCACCGTGAATCTAAGCCAGAGAACACGGCTGCTGCTTAAGGTCCTGGCCATCT | |
| CAGACCTCGTCATCTATCGAACTCATGCAGACCGGCTGCATAACGACCTCTTCAAATTCCTTGGGGATGCCTCAGAAGCT | |
| TATCTGAAGCACTTCACCAAGGAGCTCAAGGCCACCACTGCTCGCTGTGGCCTGGATGTCCCTTTATCCACACTGGGCCC | |
| TGCAGTTATCATCTTCCATGAGACCGTGCACACCCAGCTACTGGGCTCTGATCATCCCTCAGAGGTGCCAGAGAAGCTCA | |
| TCCAGGACCGGTTCCGGAAGCTGGGCCGTTTCCCTGAAGCCTTTAGTTCCATTCACTACAAGGGAACGAGGACTTACAAC | |
| CCTCCCACGGACTTTTCTGGGCTTCGGCGTGCTTTGGAACAGCTACTAGAGAATAACACCACCCGTTCTCCCCGGCACCC | |
| GGGAGTCATCTTCAAAGCCCTGAAGGCACTAAGTGACCGCTTCAGCGGTGAGATCCCCGATGACCAGATGGCGCACAGCT | |
| CCTTTTTTCCAGATGAGTATTTCACCTGCTCCTCCTTGTGCCTCAGCTGTGGGGTTGGATGTAAGAAAAGCATGAATCAT | |
| GGGAAGGAAGGAGTGCCTCATGAAGCCAAGAGCCGCTGCAGATACTCCCACCAGTATGACAACCGAGTGTATACCTGCAA | |
| GCCCTGCTATGAGAGAGGCGAGGAAGTCAGTGTAGTGCCCAAAACATCTGCTTCCACTGACTCCCCCTGGATGGGTCTCG | |
| CAAAATATGCCTGGTCTGGGTATGTGATCGAATGTCCTAACTGTGGCGTGGTCTATCGTAGTCGGCAGTACTGGTTTGGA | |
| AACCAAGATCCTGTGGATACGGTGGTGCGGACAGAGATTGTGCATGTGTGGCCTGGAACTGATGGGTTTCTGAAGGACAA | |
| CAACAATGCTGCCCAGCGCCTGTTGGACGGGATGAACTTCATGGCTCAGTCGGTGTCCGAGCTTAGCCTTGGACCCACCA | |
| AGGCTGTGACTTCCTGGCTGACAGACCAGATCGCCCCTGCCTACTGGAGGCCCAACTCCCAGATTCTGAGCTGCAACAAG | |
| TGTGCGACGTCCTTTAAAGATAACGACACTAAGCATCACTGCCGAGCCTGTGGGGAGGGCTTCTGTGACAGCTGTTCATC | |
| AAAGACTCGGCCAGTGCCTGAGCGGGGCTGGGGCCCTGCGCCAGTGCGGGTCTGTGACAACTGCTACGAAGCCAGGAACG | |
| TCCAGTTAGCTGTTACCGAGGCACAAGTGGACGATGAAGGTGGAACGCTCATTGCTCGGAAGGTGGGCGAGGCCGTGCAG | |
| AACACTCTGGGAGCCGTGGTGACAGCCATTGACATACCACTAGGTCTGGTAAAGGACGCGGCCAGGCCTGCGTACTGGGT | |
| GCCTGACCACGAAATCCTCCACTGCCACAACTGCCGGAAGGAGTTCAGCATCAAGCTCTCCAAGCACCACTGCCGGGCCT | |
| GCGGACAGGGCTTCTGTGATGAGTGCTCCCATGACCGCCGGGCTGTTCCTTCTCGTGGCTGGGACCATCCCGTCCGAGTC | |
| TGCTTCAACTGCAATAAAAGCCCGGTGACCTTTAACCCCAGCCCCCTCTCCGAGTCCTTCACAATTCCTTAG | |

The disclosed NOV3 nucleic acid sequence maps to chromosome 14 and has 2502 of 2518 bases (99%) identical to a Homo sapiens KIAA1589 protein mRNA (gb:GENBANK-ID:AB046809|acc:AB046809.1) (E=0.0).

A disclosed NOV3 protein (SEQ ID NO: 18) encoded by SEQ ID NO: 17 has 891 amino acid residues, and is presented using the one-letter code in Table 3B. Signal P, Psort and/or Hydropathy results predict that NOV3 contains a signal peptide, and is likely to be localized to the nucleus with a certainty of 0.6000 and to the mitochondrial matrix space with a certainty of 0.4811. The most likely cleavage site for a NOV3 peptide is between amino acids 19 and 20, at: AKC-GP.

TABLE 3B

Encoded NOV3 protein sequence.

| | |
|---|---|
| MGRARWVTSVIPALWEAKCGPHSKSDFPNAQKMAMLSPQTFEFRNKIELISEALPEDQERTFQDLQEPELSHTPNSVQNP | (SEQ ID NO:18) |
| VEVSCSLQTQIFVFTPGASSVTIIWWVCFLTSVSMSAQTSPAEKGLNPGLMCQESYACSGTDEAIFECDECCSLQCLRCE | |
| EELHRQERLRNHERIRLKPGHVPYCDLCKGLSGHLPGVRQRAIVRCQTCKINLCLECQKRTHSGGNKRRHPVTVYNVSNL | |
| QESLEAEEMDEETKRKKMTEKVVSFLLVDENEEIQVTNEEDFIRKLDCKPDQHLKVVSIFGNTGDGKSHTLNHTFFYGRE | |
| VFKTSPTQESCTVGVWAAYDPVHKVAVIDTEGLLGATVNLSQRTRLLLKVIAISDLVIYRTHADRLHNDLFKFLGDASEA | |
| YLKHFTKELKATTARCGLDVPLSTLGPAVIIFHETVHTQLLGSDHPSEVPEKLIQDRFRKLGRFPEAFSSIHYKGTRTYN | |
| PPTDFSGLRRALEQLLENNTTRSPRHPGVIFKALKALSDRFSGEIPDDQMAHSSFFPDEYFTCSSLCLSCGVGCKKSMNH | |
| GKEGVPHEAKSRCRYSHQYDNRVYTCKACYERGEEVSVVPKTSASTDSPWMGLAKYAWSGYVIECPNCGVVYRSRQYWFG | |
| NQDPVDTVVRTEIVHVWPGTDGFLKDNNNAAQRLLDGMNFMAQSVSELSLGPTKAVTSWLTDQIAPAYWRPNSQILSCNK | |
| CATSFKCNDTKEHCRACGEGFCDSCSSKTRPVPERGWGPAPVRVCDNCYEARNVQLAVTEAQVDDEGGTLIARKVGEAVQ | |
| NTLGAVVTAIDIPLGLVKDAARPAYWVPDHEILHCHNCRKEFSIKLSKHHCRACGQGFCDECSHDRRAVPSRGWDHPVRV | |
| CFNCNKKPGDL | |

The NOV3 amino acid sequence has 816 of 816 amino acid residues (100%) identical to, and 816 of 816 amino acid residues (100%) similar to, a Homo sapiens 816 amino acid residue KIAA1589 protein (ptnr:TREMBLNEW-ACC:BAB13415) (E=0.0).

NOV3 is expressed in at least the following tissues: Adrenal Gland/Suprarenal gland, Ascending Colon, Bone, Bone Marrow, Brain, Colon, Heart, Kidney, Liver, Lung, Lymphoid tissue, Mammary gland/Breast, Oesophagus, Ovary, Pituitary Gland, Prostate, Retina, Skeletal Muscle, Small Intestine, Testis, Thyroid, Tongue, Umbilical Vein, Uterus and Whole Organism. This information was derived by determining the tissue sources of the sequences that were included in the invention including but not limited to SeqCalling sources, Public EST sources, Genomic Clone sources, Literature sources, and/or RACE sources. In addition, NOV3 is predicted to be expressed in the following tissues because of the expression pattern of a closely related Homo sapiens KIAA 1589 protein mRNA homolog, (GENBANK-ID: gb:GENBANK-ID:AB046809|acc:AB046809.1):Adrenal Gland/Suprarenal gland, Ascending Colon, Bone, Bone Marrow, Brain, Colon, Heart, Kidney, Liver, Lung, Lymphoid tissue, Mammary gland/Breast, esophagus, Ovary, Pituitary Gland, Prostate, Retina, Skeletal Muscle, Small Intestine, Testis, Thyroid, Tongue, Umbilical Vein, Uterus and Whole Organism.

NOV3 also has homology to the amino acid sequences shown in the BLASTP data listed in Table 3C.

TABLE 3C
BLAST results for NOV3

| Gene Index/ | Protein/ | Length | Identity | Positives | |
|---|---|---|---|---|---|
| Identifier | Organism | (aa) | (%) | (%) | Expect |
| gi\|14749609\|ref\| | hypothetical | 777 | 777/777 | 777/777 | 0.0 |
| XP_027303.1\| | protein | | (100%) | (100%) | |
| (XM_027303) | XP_027303 | | | | |
| | [Homo | | | | |
| | sapiens] | | | | |
| gi\|14042048\|dbj\| | unnamed | 759 | 759/759 | 759/759 | 0.0 |
| BAB55085.1\|(AK027399) | protein | | (100%) | (100%) | |
| | product [Homo | | | | |
| | sapiens] | | | | |
| gi\|10047253\|dbj\| | KIAA1589 | 816 | 816/816 | 816/816 | 0.0 |
| BAB13415.1\|(AB046809) | protein [Homo | | (100%) | (100%) | |
| | sapiens] | | | | |
| gi\|14749607\|ref\| | zinc finger | 362 | 362/362 | 362/362 | 0.0 |
| XP_027304.1\| | protein, | | (100%) | (100%) | |
| (XM_027304) | subfamily 2A | | | | |
| | (FYVE domain | | | | |
| | containing), | | | | |
| | 1 [Homo | | | | |
| | sapiens] | | | | |
| gi\|13958036\|gb\| | FYVE-finger | 600 | 33/69 | 41/69 | 7e−12 |
| AAK50771.1\|AF361055_1 | protein EIP1 | | (47%) | (58%) | |
| (AF361055) | [Homo | | | | |
| | sapiens] | | | | |

The homology of these sequences is shown graphically in the ClustalW analysis shown in Table 3D.

[image]

[image]

[image]

Tables 3E -3H list the domain description from DOMAIN analysis results against NOV3. This indicates that the NOV3 sequence has properties similar to those of other proteins known to contain these domains.

TABLE 3E
Domain Analysis of NOV3

gn1|Smart|smart00064, FYVE, Protein present in Fab1, YOTB, Vac1, and
EEA1.; Zinc-binding domain, possibly involved in endosomal targetting.
Recent data indicates that these domains bind PtdIns(3)P. (SEQ ID NO:74)
Length = 69 residues, 92.8% aligned
Score = 70.9 bits (172), Expect = 3e−13

| | | | |
|---|---|---|---|
| NOV3 | 822 | RPAYWVPDHEILHCHNCRKEFSIKLSKHHCRACGQGFCDECSHDRRAVPSRGWDHPVRVC | 881 |
| | | +\|+\|\| \| +\| \| \|\|\|++ +\|\|\|\| \|\|+ \|\| +\|\| + +\| \| + \|\|\|\|\| | |
| 00064 | 2 | VRPHWIPDVEASNCMGCGKEFNLTKRRHHCRNCGRIFCSKCSSKKAPLPKLGNEDPVRVC | 61 |
| NOV3 | 882 | FNCN 885 | |
| | | +\| | |
| 00064 | 62 | DDCY 65 | |

TABLE 3F

Domain Analysis of NOV3

gn1|Smart|smart00064, FYVE. (SEQ ID NO:75)
Length = 69 residues, 89.9% aligned
Score = 62.8 bits (151), Expect = 18e−11

| | | | |
|---|---|---|---|
| NOV3 | 708 | YWRPNSQILSCNKCATSFKDNDTKHHCRACGEGFCDSCSSKTRPVPERGWGPAPVRVCDN | 767 |
| | | +\| \|+ + +\| \| \| +\|\|\|\| \|\| \|\| \|\|\|\| \|+\|+ \| \|\|\|\|\|\|+ | |
| 00064 | 5 | HWIPDVEASNCMGCGKEFNLTKRRHHCRNCGRIFCSKCSSKKAPLPKLGNED-PVRVCDD | 63 |
| NOV3 | 768 | CYE 770 | |
| | | \|\|\| | |
| 00064 | 64 | CYE 66 | |

TABLE 3G

Domain Analysis of NOV3

gn1|Pfam|pfam01363, FYVE, FYVE zinc finger. The FYVE zinc finger is
named after four proteins that it has been found in: Fab1,
YOTB/ZK632.12, Vac1, and EEA1. The FYVE finger has been shown to bind
two Zn++ ions. The FYVE finger has eight potential zinc coordinating
cysteine positions. Many members of this family also include two
histidines in a motif R+HHC+XCG, where + represents a charged residue
and X any residue. We have included members which do not conserve
these histidine residues but are clearly related. (SEQ ID NO:76)
Length = 66 residues, 92.4% aligned
Score = 67.4 bits (163), Expect = 3e−12

| | | | |
|---|---|---|---|
| NOV3 | 824 | AYWVPDHEILHCHNCRKEFSIKLSKHHCRACGQGFCDECSHDRRAVPSRGWDHPVRVCFN | 883 |
| | | +\|\|\|\| \|+ +\| \| \| \|++ +\|\|\|\|\|\|\|+ \|\| \|\| +\| \| + \|\|\|\|\| + | |
| 01363 | 1 | PHWVPDEEVSNCMRCGKPFTLTKRRHHCRACGRIFCSSCSSKTVPLPPMG-ERPVRVCDS | 59 |
| NOV3 | 884 | CN 885 | |
| | | \| | |
| 01363 | 60 | CY 61 | |

TABLE 3H

Domain Analysis of NOV3

gn1|pfam|pfam01363, FYVE, FYVE zinc finger. (SEQ ID NO:77)
Length = 66 residues, 98.5% aligned
Score = 64.7 bits (156), Expect = 2e−11

| | | | |
|---|---|---|---|
| NOV3 | 707 | AYWRPNSQILSCNKCATSFKDNDTKHHCRACGEGFCDSCSSKTRPVPERGWGPAPVRVCD | 766 |
| | | +\| \|+ ++ +\| +\| \| +\|\|\|\|\|\|\| \|\| \|\|\|\|\|\| \|+\| \| \|\|\|\|\|\| | |
| 01363 | 1 | PHWVPDEEVSNCMRCGKPFTLTKRRHHCRACGRIFCSSCSSKTVPLPPM--GERPVEVCD | 58 |
| NOV3 | 767 | NCYEARN 773 | |
| | | +\|\|+ \| | |
| 01363 | 59 | SCYDLLN 65 | |

The above defined information for NOV3 suggests that this NOV3 protein may function as a member of a KIAA1589 protein family. Therefore, the NOV3 nucleic acids and proteins of the invention are useful in potential therapeutic and diagnostic applications. For example, a cDNA encoding the NOV3 protein may be useful in gene therapy, and the NOV3 protein may be useful when administered to a subject in need thereof. By way of nonlimiting example, the compositions of the present invention will have efficacy for treatment of patients suffering from inflammatory disorders such as osteo- and rheumatoid-arthritis, inflammatory bowel disease, Crohn's disease; immunological disorders, AIDS; cancers including but not limited to lung cancer, colon cancer, leukemia or pancreatic cancer.; blood disorders; asthma; psoriasis; vascular disorders, hypertension, skin disorders, renal disorders including Alport syndrome, immunological disorders, tissue injury, fibrosis disorders, bone diseases, Ehlers-Danlos syndrome type VI, VII, type IV, S-linked cutis laxa and Ehlers-Danlos syndrome type V, osteogenesis imperfecta, Neurologic diseases, Brain and/or autoimmune disorders like encephalomyelitis, neurodegenerative disorders, Alzheimer's Disease, Parkinson's Disorder, immune disorders, and hematopoietic disorders, endocrine diseases, muscle disorders, inflammation and wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), Parkinson's disease, acute heart failure, hypotension, hypertension, urinary retention, osteoporosis; multiple sclerosis; and Treatment of Albright Hereditary Ostoeodystrophy, angina pectoris, myocardial infarction, ulcers and benign prostatic hypertrophy. The NOV3 nucleic acid encoding KIAA1589-like protein, and the KIAA1589-like protein of the invention, or fragments thereof, may further be useful in diagnostic applications, wherein the presence or amount of the nucleic acid or the protein are to be assessed.

NOV4

A disclosed NOV4 nucleic acid of 1761 nucleotides (designated CuraGen Acc. No.

GSAL442663.1_B) encoding a novel WD40-motif protein-like protein is shown in Table 4A. An open reading frame was identified beginning with an ATG initiation codon at nucleotides 48-50and ending with a TAA codon at nucleotides 1536-1538. Putative untranslated regions upstream from the initiation codon and downstream from the termination codon are underlined in Table 4A, and the start and stop codons are in bold letters.

TABLE 4A

NOV4 Nucleotide Sequence

(SEQ ID NO:19)

AGCTGAGCCGGAGGGAATCCGGAAGGACACGCTGAACAGGAACAGAAATGAATAAAAGTCGCTGGCAGAGTAGAAGACG
ACATGGGAGAAGAAGCCACCAGCAGAACCCTTGGTTCAGACTCCGTGATTCTGAAGACAGGTCTGACTCCCGGGCAGCA
CAGCCCGCTCACGATTCCGGCCACGGTGATGACGAGTCTCCGTCAACCTCGTCTGGCACAGCTGGGACCTCCTCTGTGC
CAGAGCTACCTGGGTTTTACTTTGACCCTGAAAAGAAACGCTACTTCCGCTTGCTCCCTGGACATAACAACTGCAACCC
CCTGACGAAAGAGAGCATCCGGCAGAAGGAGATGGAGAGCAAGAGACTGCGGCTGCTCCAGGAAGAAGACAGACGGAAA
AAGATTGCCAGGATGGGATTTAATGCATCTTCCATGCTACGAAAAAGCCAGCTGGGTTTTCTCAACGTCACCAATTACT
GCCATTTAGCCCACGAGCTGCGTCTCAGCTGCATGGAGAGGAAAAAGGTCCAGATTGCAAGCATGAGGGATCCCTCCGC
CTTGGCAAGCGACCGATTTAACCTCATACTGGCAGATACCAACAGTGACCGGCTCTTCACAGTGAACGATGTTAAAGTT
GGAGGCTCCAAGTATGGTATCATCAACCTGCAAAGTCTGAAGACCCCTACGCTCAAGGTGTTCATGCACGAAAACCTCT
ACTTCACCAACCGGAAGGTGAATTCGGTGTGCTGGGCCTCGCTGAATCACTTGGATTCCCACATTCTGCTATGCCTCAT
GGGACTCGCAGAGACTCCAGGCTGTGCCACCCTGCTCCCAGCATCACTGTTCGTCAATAGTCACCCAGGAATAGACCGG
CCTGGCATGCTCTGCAGTTTCCGGATCCCTGGTGCCTGGTCCTGTGCCTGGTCCCTGAATATCCAAGCAAATAACTGCT
TCAGTACAGGCTTGTCTCGGCGGGTCCTGTTGACCAACGTGGTGACGGGACACCGGCAGTCCTTTGGGACCAACAGTGA
TGTCTTGGCCCAGCAGTTTGCTCTCATGGCTCCTCTGCTGTTTAATGGCTGCCGCTCTGGGGAAATCTTTGCCATTGAT
CTGCGTTGTGGAAATCAAGGCAAGGGATGGAAGGCCACCCGCCTGTTTCATGATTCAGCAGTGACCTCTGTGCGGATCC
TCCAAGATGAGCAATACCTGATGGCTTCAGACATGGCTGGAAAGATCAAGCTGTGGGACCTGAGGACCACGAAGTGCGT
AAGGCAGTACGAAGGCCACGTGAATGAGTACGCCTACCTGCCCCTGCATGTGCACGAGGAAGAAGGAATCCTGGTGGCA
GTGGGCCAGGACTGCTACACGAGAATCTGGAGCCTCCACGATGCCCGCCTACTGAGAACCATACCCTCCCCGTACCCTG
CCTCCAAGGCCGACATTCCCAGTGTGGCCTTCTCGTCGCGGCTGGGGGGCTCCCGGGGCGCGCCGGGGCTGCTCATGGC
TGTCGGGCAGGACCTTTACTGTTACTCCTACAGCTAATTCTGCAGGGCACAGCCCAGAGCCATGTGGATTTGACTTACG
GGAGTAAAGCGTAACTTTTTACTGCATCTAATGAGGGTGTTTTAAGTGACACTCAGTGTACACAGATCCCATCCTCTGG
CTGCTAGGAGAGAAGTGCTGAATGTTCCGTGTGGAGATGCTCAGGAAAGTTATTTGAGTTAAATTGCTGGCTGAGAGAG
CTTGGAAGTCCTTTTCATAAAAG

The nucleic acid sequence of NOV4 maps to chromosome 14 and has 1282 of 1287 bases (99%) identical to a Homo sapiens DKFZp434K114 mRNA (gb:GENBANK-ID:HSM800674|acc:AL080157.1) (E=2.6e−²⁸²).

A NOV4 polypeptide (SEQ ID NO: 20) encoded by SEQ ID NO: 19 is 496 amino acid residues and is presented using the one letter code in Table 4B. Signal P, Psort and/or Hydropathy results predict that NOV4 does not contain a signal peptide and is likely to be localized to the nucleus with a certainty of 0.9600.

TABLE 4B
NOV4 protein sequence

(SEQ ID NO:20)

MNKSRWQSRRRHGRRSHQQNPWFRLRDSEDRSDSRAAQPAHDSGHGDDESPSTSSGTAGTSSVPELPGFYFDPEKKRYFRLLP
GHNNCNPLTKESIRQKEMESKRLRLLQEEDRRKKIARMGFNASSMLRKSQLGFLNVTNYCHLAHELRLSCMERKKVQIRSMRD
PSALASDRFNLILADTNSDRLFTVNDVKVGGSKYGIINLQSLKTPTLKVFMHENLYFTNRKVNSVCWASLNHLDSHILLCLMG
LAETPGCATLLPASLFVNSHPGIDRPGMLCSFRIPGAWSCAWSLNIQANNCFSTGLSRRVLLTNVVTGHRQSFGTNSDVLAQQ
FALMAPLLFNGCRSGEIFAIDLRCGNQGKGWKATRLFHDSAVTSVRILQDEQYLMASDMAGKIKLWDLRTTKCVRQYEGHVNE
YAYLPLHVHEEEGILVAVGQDCYTRIWSLHDARLLRTIPSPYPASKADIPSVAFSSRLGGSRGAPGLLMAVGQDLYCYSYS

The NOV4 amino acid sequence has 425 of 428 amino acid residues (99%) identical to, and 425 of 428 amino acid residues (99%) similar to, a Homo Sapiens 430 amino acid residue hypothetical 48.5 kDa protein (ptnr:SPTREMBL-ACC:Q9Y4P5) (E=1.0e⁻²³²).

NOV4 is expressed in at least the following tissues: cancer tissue, pancreas, fetal lung NbHL19W, testis NHT, B-cell and brain. This information was derived by determining the tissue sources of the sequences that were included in the invention including but not limited to SeqCalling sources, Public EST sources, Genomic Clone sources, Literature sources, and/or RACE sources. In addition, NOV4 is predicted to be expressed in the following tissues because of the expression pattern of a closely related Homo sapiens DKFZp434Kl 14 mRNA homolog (GENBANK-ID: gb:GENBANK-ID: HSM800674|acc:AL080157.1): Rhabdomyosarcoma, neuroepithelium, pancreas, fetal lung, NbHL19W, testis NHT, B-cell and brain.

NOV4 also has homology to the amino acid sequences shown in the BLASTP data listed in Table 4C.

TABLE 4C
BLAST results for NOV4

| Gene Index/ | | Length | Identity | Positives | |
|---|---|---|---|---|---|
| Identifier | Protein/Organism | (aa) | (%) | (%) | Expect |
| gi\|14749605\|ref\| | hypothetical | 489 | 487/497 | 487/497 | 0.0 |
| XP_027300.1\| | protein XP_027300 | | (97%) | (97%) | |
| (XM_027300) | [Homo sapiens] | | | | |
| gi\|14042651\|dbj\| | unnamed protein | 489 | 485/497 | 487/497 | 0.0 |
| BAB55337.1\| (AK027745) | product [Homo | | (97%) | (97%) | |
| | sapiens] | | | | |
| gi\|7512629\|pir\|\| | hypothetical | 430 | 425/428 | 425/428 | 0.0 |
| T12541 | protein | | (99%) | (99%) | |
| | DKFZp434K114.1 — | | | | |
| | (fragment) [Homo | | | | |
| | sapiens] | | | | |
| gi\|13096981\|gb\| | Unknown (protein | 519 | 400/498 | 446/498 | 0.0 |
| AAH03284.1\|AAH03284 | for MGC:7874) [Mus | | (80%) | (89%) | |
| (BC003284) | musculus] | | | | |
| gi\|14749602\|ref\| | DKFZP434K114 | 333 | 331/332 | 331/332 | 0.0 |
| XP_027301.1\| | protein [Homo | | (99%) | (99%) | |
| (XM_027301) | sapiens] | | | | |

The homology of these sequences is shown graphically in the ClustalW analysis shown in Table 4D.

[image]

[image]

Recent studies have demonstrated the importance of F-box/WD40 proteins in the regulation of developmental processes, by a mechanism of specific ubiquitinization and subsequent proteolysis of target proteins belonging to the Wnt, Hh and NF-kappaB signaling pathways. A new human gene, Dactylin, encoding a novel member of the F-box/WD40 protein family was recently cloned and characterized. The Dactylin gene comprises nine exons distributed in more than 85 kb of genomic DNA and encoding a protein with four WD40 repeats and an F-box motif. The chromosomal location of Dactylin and its putative function as an F-box/WD40 repeat protein, likely to be involved in key signaling pathways crucial for normal limb development, make it a promising candidate gene for SHFM3 (Ianakiev et al., A novel human gene encoding an F-box/WD40 containing protein maps in the SHFM3 critical region on 10q24. Biochem Biophys Res Commun 261(1):64-70, 1999).

Several other proteins have been identified as containing WD40 repeat domains including the COP1 (constitutively photomorphogenic 1) protein in Arabidopsis which is a repressor of light-regulated development and it mammalian homologue which acts within the nucleus to repress photomorphogenic development (Wang et al., Evidence for functional conservation of a mammalian homologue of the light-responsive plant protein COP1. Curr Biol 9(13):711-4, 1999). Another protein which contains the WD40 repeat domain is the Schizosaccharomyces pombe 72 kDa TFIID subunit. This protein contains several significant highly conserved regions including the WD40 repeats, that are indispensable for the viability (Yamamoto et al., Molecular genetic elucidation of the tripartite structure of the Schizosaccharomyces pombe 72 kDa TFIID subunit which contains a WD40 structural motif. Genes Cells 2(4):245-54, 1997).

The above defined information for NOV4 suggests that this NOV4 protein may function as a member of a WD40-motif protein-like protein family. Therefore, the NOV4 nucleic acids and proteins of the invention are useful in potential therapeutic and diagnostic applications. For example, a cDNA encoding the NOV4 protein may be useful in gene therapy, and the NOV4 protein may be useful when administered to a subject in need thereof. By way of nonlimiting example, the compositions of the present invention will have efficacy for treatment of patients suffering from cancer, trauma, regeneration (in vitro and in vivo), viral/bacterial/parasitic infections Diabetes, Von Hippel-Lindau (VHL) syndrome, Pancreatitis, Obesity Systemic lupus erythematosus, Autoimmune disease, Asthma, Emphysema, Scleroderma, allergy, ARDS, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection Fertility Myasthenia gravis, Leukodystrophies, Pain, Neuroprotection Endocrine dysfunctions, Diabetes, obesity, Growth and reproductive disorders Hemophilia, Hypercoagulation, Idiopathic thrombocytopenic purpura, Immunodeficiencies and Graft vesus host. The NOV4 nucleic acid encoding WD40-motif protein-like protein, and the WD40-motif protein-like protein of the invention, or fragments thereof, may further be useful in diagnostic applications, wherein the presence or amount of the nucleic acid or the protein are to be assessed.

NOV5

NOV5 includes four novel Opioid Binding Cell Adhesion Molecule-like proteins disclosed below. The disclosed proteins have been named NOV5a, NOV5b, NOV5c and NOV5d.

NOV5a

A disclosed NOV5a nucleic acid of 1018 nucleotides (also referred to as 139785504) encoding a novel Opioid Binding Cell Adhesion Molecule-like protein is shown in Table 5A. An open reading frame lacking a signal peptide was identified beginning with an CTG at nucleotides 1-3 and ending with a TAG codon at nucleotides 958-960. A putative untranslated region downstream from the termination codon are underlined in Table 5A, and the start and stop codons are in bold letters.

TABLE 5A

NOV5a Nucleotide Sequence

(SEQ ID NO:21)

CTGGCCGGCTTGGCCGTCATCAGCCGAGGGCTGCTCTCCCAGAGCCTGGAGTTCAACTCTCCTGCCGACAACTACACAGTGT
GTGAAGGTGACAACGCCACCCTCAGCTGCTTCATCGACGAGCACGTGACCCGCGTGGCTTGGCTGAACCGCTCCAACATCCT
GTATGCCGGCAATGACCGCTGGACCAGCGACCCGCGGGTGCGGCTGCTCATCAACACCCCCGAGGAGTTCTCCATCCTCATC
ACCGAGGTGGGGCTCGGCGACGAGGGCCTCTACACCTGCTCCTTCCAGACCCGCCACCAGCCGTACACCACTCAGGTCTACC
TCATTGTCCACGTCCCTGCCCGCATTGTGAACATCTCGTCGCCTGTGACGGTGAATGAGGGGGGCAATGTGAACCTGCTTTG
CCTGGCCGTGGGGCGGCCAGAGCCCACGGTCACCTGGAGACAGCTCCGAGACGGCTTCACCTCGGAGGGAGAGATCCTGGAG
ATCTCTGACATCCAGCGGGGCCAGGCCGGGGAGTATGAGTGCGTGACTCACAACGGGGTTAACTCGGCGCCCGACAGCCGCC
GCGTGCTGGTCACAGTCAACTATCCTCCGACCATCACGGACGTGACCAGCGCCCGCACCGCGCTGGGCCGGACCGCCCTCCT
GCGCTGCGAAGCCATGGCGGTTCCCCCCGCGGATTTCCAGTGGTACAAGGATGACAGACTGCTGAGCAGCGGCACGGCCGAA
GGCCTGAAGGTGCAGACGGAGCGCACCCGCTCGATGCTTCTCTTTGCCAACGTGAGCGCCCGGCATTACGGCAACTATACGT
GTCGCGCCGCCAATCGACTGGGAGCGTCCAGCGCCTCCATGCGGCTCCTGCGCCCAGGATCCCTGGAGAACTCAGCCCCGAG
GCCCCCAGGGCTCCTGGCCCTCCTCTCCGCCCTGGGCTGGCTGTGGTGGAGAATGTAGGCGCAACCCAGTCCAGCTCACCTC
CCCCTGCAGGGGGCCTCAAACCAAGAGTGAGAGA

The NOV5a nucleic acid was identified on chromosome 7 and has 260 of 389 bases (66%) identical to a Homo sapiens (clone pHOM) opioid-binding cell adhesion molecule mRNA (gb:GENBANK-ID:HUMOBCAM|acc:L34774.1) (E=3.4e⁻⁵⁰).

A disclosed NOV5a polypeptide (SEQ ID NO: 22) encoded by SEQ ID NO: 21 is 319 amino acid residues and is presented using the one-letter code in Table 5B. Signal P, Psort and/or Hydropathy results predict that NOV5a does not contain a signal peptide and is likely to be localized in the mitochondrial matrix space with a certainty of 0.4686 and the cytoplasm with a certainty of 0.4500.

Although SignalP, Psort and/or hydropathy suggest that NOV5a protein may be localized in the mitochondrial matrix space and cytoplasm, the protein predicted here is similar to the Opioid Binding Cell Adhesion Molecule family, some members of which are released extracellularly. The closest homolog SWISSPROT-ACC:Q14982 opioid binding protein/cell adhesion molecule is a type Ia membrane protein that is localized to the plasma membrane extracellularly. This indicates that the signal peptide of the mature protein is cleaved. Therefore it is likely that NOV5a protein is available at the same sub-cellular localization and hence accessible to a diagnostic probe and for various therapeutic applications. It also indicates that the use of a heterologous signal peptide to target the novel protein to the appropriate location, i.e. extracellularly, is appropriate.

TABLE 5B

Encoded NOV5a protein sequence

(SEQ ID NO:22)

LAGLAVISRGLLSQSLEFNSPADNYTVCEGDNATLSCFIDEHVTRVAWLNRSNILYAGNDRWTSDPRVRLLINTPEEFSIL
ITEVGLGDEGLYTCSFQTRHQPYTTQVYLIVHVPARIVNISSPVTVNEGGNVNLLCLAVGRPEPTVTWRQLRDGFTSEGEI
LEISDIQRGQAGEYECVTHNGVNSAPDSRRVLVTVNYPPTITDVTSARTALGRTALLRCEAMAVPPADFQWYKDDRLLSSG
TAEGLKVQTERTRSMLLFANVSARHYGNYTCRAANRLGASSASMRLLRPGSLENSAPRPPGLLALLSALGWLWWRM

The NOV5a amino acid sequence has 162 of 300 amino acid residues (54%) identical to, and 213 of 00 amino acid residues (71%) similar to, a Homo Sapiens 345 amino acid residue opioid binding protein/cell adhesion molecule precursor (OBCAM)/opioid-binding cell adhesion molecule (OPCML) protein (ptnr:SWISSPROT-ACC:Q14982) (E=3.1e⁻⁸⁰).

NOV5a is expressed in at least the following tissues: Brain and Fetal brain. This information was derived by determining the tissue sources of the sequences that were included in the invention including but not limited to SeqCalling sources, Public EST sources, genomic clone sources, literature sources, and/or RACE sources. In addition, NOV5a is predicted to be expressed in brain tissues because of the expression pattern of a closely related Human (clone pHOM) opioid-binding cell adhesion molecule mRNA homolog (gb:GENBANK-ID:HUMOBCAM|acc:L34774.1).

NOV5b

A disclosed NOV5b nucleic acid of 1017 nucleotides (also referred to as 139785504_da1) encoding a novel Opioid Binding Cell Adhesion Molecule-like protein is shown in Table 5C. An open reading frame lacking the signal peptide was identified beginning with an GCC at nucleotides 3-5 and ending with a TAG codon at nucleotides 958-960. Putative untranslated regions upstream from the initiation codon and downstream from the termination codon are underlined in Table 5C, and the start and stop codons are in bold letters.

TABLE 5C

NOV5b Nucleotide Sequence

(SEQ ID NO:23)

CTGGCCGGCTTGGCCGTCATCAGCCGAGGGCTGCTCTCCCAGAGCCTGGAGTTCAACTCTCCTGCCGACAACTACACAG
TGTGTGAAGGTGACAACGCCACCCTCAGCTGCTTCATCGACGAGCACGTGACCCGCGTGGCCTGGCTGAACCGCTCCAA
CATCCTGTATGCCGGCAATGACCGCTGGACCAGCGACCCGCGGGTGCGGCTGCTCATCAACACCCCCGAGGAGTTCTCC
ATCCTCATCACCGAGGTGGGGCTCGGCGACGAGGGCCTCTACACCTGCTCCTTCCAGACCCGCCACCAGCCGTACACCA
CTCAGGTCTACCTCATTGTCCACGTCCCTGCCCGCATTGTGAACATCTCGTCGCCTGTGACGGTGAATGAGGGGGGCAA
TGTGAACCTGCTTTGCCTGGCCGTGGGGCGGCCAGAGCCCACGGTCACCTGGAGACAGCTCCGAGACGGCTTCACCTCG
GAGGGAGAGATCCTGGAGATCTCTGACATCCAGCGGGGCCAGGCCGGGGAGTATGAGTGCGTGACTCACAACGGGGTTA
ACTCGGCGCCCGACAGCCGCCGCGTGCTGGTCACAGTCAACTATCCTCCGACCATCACGGACGTGACCAGCGCCCGCAC
CGCGCTGGGCCGGGCCGCCCTCCTGCGCTGCGAAGCCATGGCGGTTCCCCCCGCGGATTTCCAGTGGTACAAGGATGAC
AGACTGCTGAGCAGCGGCACGGCCGAAGGCCTGAAGGTGCAGACGGAGCGCACCCGCTCGATGCTTCTCTTTGCCAACG
TGAGCGCCCGGCATTACGGCAACTATACGTGTCGCGCCGCCAACCGACTGGGAGCGTCCAGCGCCTCCATGCGGCTCCT
GCGCCCAGGATCCCTGGAGAACTCAGCCCCGAGGCCCCCAGGGCTCCTGGCCCTCCTCTCCGCCCTGGGCTGGCTGTGG
TGGAGAATGTAGGCCAACCCAGTCCAGCTCACCTCCCCCTGCAGGGGGCCTCAAACCAAGAGTGAGAGA

The NOV5b nucleic acid was identified on chromosome 7 and has 261 of 389 bases (67%) identical to a Homo sapiens (clone pHOM) opioid-binding cell adhesion molecule mRNA (gb:GENBANK-ID:HUMOBCAM|acc:L34774. 1) (E=1.3e⁻⁵⁰).

A disclosed NOV5b polypeptide (SEQ ID NO: 24) encoded by SEQ ID NO: 23 is 319 amino acid residues and is presented using the one-letter code in Table 5D. Signal P, Psort and/or Hydropathy results predict that NOV5b does not contain a signal peptide and is likely to be localized in the mitochondrial matrix space with a certainty of 0.4686 and the cytoplasm with a certainty of 0.4500.

Although SignalP, Psort and/or hydropathy suggest that NOV5b protein may be localized in the mitochondrial matrix space and cytoplasm, the protein predicted here is similar to the Opioid Binding Cell Adhesion Molecule family, some members of which are released extracellularly. The closest homolog SWISSPROT-ACC:Q14982 opioid binding protein/cell adhesion molecule is a type Ia membrane protein that is localized to the plasma membrane extracellularly. This indicates that the signal peptide of the mature protein is cleaved. Therefore it is likely that NOV5b protein is available at the same sub-cellular localization and hence accessible to a diagnostic probe and for various therapeutic applications. It also indicates that the use of a heterologous signal peptide to target the novel protein to the appropriate location, i.e. extracellularly, is appropriate.

TABLE 5D

Encoded NOV5b protein sequence.

(SEQ ID NO:24)

LAGLAVISRGLLSQSLEFNSPADNYTVCEGDNATLSCFIDEHVTRVAWLNRSNILYAGNDRWTSDPRVRLLINTPEEFSILITEV
GLGDEGLYTCSFQTRHQPYTTQVYLIVHVPARIVNISSPVTVNEGGNVNLLCLAVGRPEPTVTWRQLRDGFTSEGEILEISDIQR
GQAGEYECVTHNGVNSAPDSRRVLVTVNYPPTITDVTSARTALGRAALLRCEAMAVPPADFQWYKDDRLLSSGTAEGLKVQTERT
RSMLLFANVSARHYGNYTCRAANRLGASSASMRLLRPGSLENSAPRPPGLLALLSALGWLWWRM

The NOV5b amino acid sequence has 164 of 300 amino acid residues (54%) identical to, and 213 of 300 amino acid residues (71%) similar to, a Bos taurus 345 amino acid residue opioid binding protein/cell adhesion molecule precursor (OBCAM)/opioid-binding cell adhesion molecule (OPCML) protein (ptnr:SWISSPROT-ACC:P11834) (E=1.7e⁻⁸⁰).

NOV5b is expressed in at least the following tissues: Brain and Fetal brain. This information was derived by determining the tissue sources of the sequences that were included in the invention including but not limited to SeqCalling sources, Public EST sources, genomic clone sources, literature sources, and/or RACE sources. In addition, NOV5b is predicted to be expressed in brain tissues because of the expression pattern of a closely related Homo sapiens (clone pHOM) opioid-binding cell adhesion molecule mRNA homolog (gb:GENBANK-ID:HUMOBCAM|acc:L34774.1).

NOV5c

A disclosed NOV5c nucleic acid of 1136 nucleotides (also referred to as CG51027-03) encoding a novel Opioid Binding Cell Adhesion Molecule-like protein is shown in Table 5E. An open reading frame lacking a signal peptide was identified beginning with an TCC at nucleotides 2-4and ending with a TAG codon at nucleotides 923-925. Putative untranslated regions upstream from the initiation codon and downstream from the termination codon are underlined in Table 5E, and the start and stop codons are in bold letters.

TABLE 5E
NOV5c Nucleotide Sequence

(SEQ ID NO:25)

CTCCCAGAGCCTGGAGTTCAACTCTCCTGCCGACAACTACACATGTGTGAAGGTGACAACGCCACCCTCAGCTGCTTCATC
GACGAGCACGTGACCCGCGTGGCCTGGCTGAACCGCTCCAACATCCTGTATGCCGGCAATGACCGCTGGACCAGCGACCCGC
GGGTGTGGCTGCTCATCAACACCCCCGAGGAGTTCTCCATCCTCATCACCGAGGTGGGGCTCGGCGACGAGGGCCTCTACAC
CTGCTCCTTCCAGACCCGCCACCAGCCGTACACCACTCAGGTCTACCTCTTGTCCACGTCCCTGCCCGCATTGTGAACATC
TCGTCGCCTGTGATGGTGAATCAGGGGCGCAATGTGACCTGCTTTGCCTGGCCGTGGGGCGGCCACAGCCCACGGTCACCT
GGAGACAGCTCCGAGACGGCTTCACCTCGGAGGGAGAGATCCTGGAGATCTCTGACATCCAGCGGGGCCAGGCCGGGGAGTA
TGAGTGCGTGACTCACAACGGGGTTAACTCGGCGCCCGACAGCCGCCGCGTGCTGGTCACAGTCAACTATCCTCCGACCATC
ACGGACGTGACCAGCGCCCGCACCGCGCTGGGCCGGGCCGCCCTCCTGCGCTGCGAAGCCATGGCGGTTCCCCCCGCGGATT
TCCAGTGGTATAAGGATGACAGACTGCTGAGCAGCGGCACGGCCGAAGGCCTGAAGGTGCAGATGGAGCGCACCCGCTCGAT
GCTTCTCTTTGCCAACGTGAGCGCCCGGCATTACGGCAACTATACGTGTCGCGCCGCCAACCGACTGGGAGCGTCCAGCGCC
TCCATGCGGCTCCTGCGCCCAGGATCCCTGGAGAACTCAGCCCCGAGGCCCCCAGGGCTCCTGGCCCTCCTCTCCGCCCTGG
GCTGGCTGTGGTGGAGAATGTAGGCGCAACCCAGTGGAGCTCACCTCCCCCTGCAGGGGGCCTCAGGCCAAGAGTGAGAGAA
ACGGGGGAGCAAGAGCCGTGGGTCTCGTGGGGGCAGAAGAGCTCTCGGCCACCAAGGAAGAAGAGAGAGGAGAAGAGGAGGA
GGCACAGGAAGAAAGATCTTCAGACAACCCATCACTGTGACGGATAACGCAAAATTATGCATCTTTCTAC

The NOV5c nucleic acid was identified on chromosome 7 and has 274 of 389 bases (70%) identical to a Bos taurus opioid binding protein/cell adhesion molecule (OBCAM) mRNA (gb:GENBANK-ID:BTOBCAM|acc:X12672.1) (E=1.3e⁻⁵⁶).

A disclosed NOV5c polypeptide (SEQ ID NO: 26) encoded by SEQ ID NO: 25 is 307 amino acid residues and is presented using the one-letter code in Table 5F. Signal P, Psort and/or Hydropathy results predict that NOV5c does not contain a signal peptide and is likely to be localized in the mitochondrial matrix space with a certainty of 0.4686 and the cytoplasm with a certainty of 0.4500.

Although SignalP, Psort and/or hydropathy suggest that NOV5c protein may be localized in the mitochondrial matrix space and cytoplasm, the protein predicted here is similar to the Opioid Binding Cell Adhesion Molecule family, some members of which are released extracellularly. The closest homolog SWISSPROT-ACC:Q14982 opioid binding protein/cell adhesion molecule is a type Ia membrane protein that is localized to the plasma membrane extracellularly. This indicates that the signal peptide of the mature protein is cleaved. Therefore it is likely that NOV5c protein is available at the same sub-cellular localization and hence accessible to a diagnostic probe and for various therapeutic applications. It also indicates that the use of a heterologous signal peptide to target the novel protein to the appropriate location, i.e. extracellularly, is appropriate.

TABLE 5F

Encoded NOV5d protein sequence
+TR, 1(SEQ ID NO:26)

SQSLEFNSPADNYTVCEGDNATLSCFIDEHVTRVAWLNRSNILYAGNDRWTSDPRVWLLINTPEEFSILITEVGLGDEGLY
TCSFQTRHQPYTTQVYLIVHVPARIVNISSPVMVNEGGNVNLLCLAVGRPEPTVTWRQLRDGFTSEGEILEISDIQRGQAG
EYECVTHNGVNSAPDSRRVLVTVNYPPTITDVTSARTALGRAALLRCEAMAVPPADFQWYDDRLLSSGTAEGLKVQMERT
RSMLLFAVSARHYGNYTCRAANRLGASSASMRLLRPGSLENSAPRPPGLIALLSALGWLWWRM

The NOV5c amino acid sequence has 163 of 300 amino acid residues (54%) identical to, and 212 of 300 amino acid residues (70%) similar to, a Homo Sapiens 345 amino acid residue opioid binding protein/cell adhesion molecule precursor (OBCAM)/opioid-binding cell adhesion molecule (OPCML) protein (ptnr:SWISSPROT-ACC:Q14982) (E=1.2e⁻⁷⁹).

NOV5c is expressed in at least the following tissues: Brain and Fetal brain. This information was derived by determining the tissue sources of the sequences that were included in the invention including but not limited to SeqCalling sources, Public EST sources, genomic clone sources, literature sources, and/or RACE sources.

NOV5d

A disclosed NOV5d nucleic acid of 1169 nucleotides (also referred to as CG51027-05) encoding a novel Opioid Binding Cell Adhesion Molecule-like protein is shown in Table 5G. An open reading frame was identified beginning with an ATG codon at nucleotides 71-73 and ending with a TAG codon at nucleotides 1079-1081. Putative untranslated regions upstream from the initiation codon and downstream from the termination codon are underlined in Table 5G, and the start and stop codons are in bold letters.

TABLE 5G

NOV5d Nucleotide Sequence

| | |
|---|---|
| CTCCAACAAGCATAGCGCGCCCCGGACCGGCCCCCCCTTTCCCCTCCCCCTCCGTGCCGCCTCTGCCGCGATG | (SEQ ID NO:27) |
| CCCCCCGCTGCGCCCGGGGCCCGGCTCCGGCTTCTCGCCGCCGCCGCCCTGGCCGGCTTGGCCGTCATCAGCC | |
| GGGGGCTGCTCTCCCAGAGGCTGGAGTTCAACTCTCCTGCCGACAACTACACAGTGTGTGAAGGTGACAACGC | |
| CACCCTCAGCTGCTTCATGGACGAGCATGTGACCCGCGTGGCCTGGCTGAACCGCTCCAACATCCTGTACGCC | |
| GGCAACGACCGCAGGACCAGGGACCCGCGGGTGCGGCTGCTCATCAACACCCCCGAGGAGTTCTCCATCCTCG | |
| TCACCGAGGTGGGGCTCGGCGACGAGGGCCTCTACACCTGCTCCTTCCAGACCCGCCACCAGCCGTACACCAC | |
| TCAGGTCTACCTCATTGTCCACGTCCCTGCCCGCGTTGTGAACATCTCGTCGCCTGTGATGGTGAATGAGGGA | |
| GGTAATGTGAACCTGCTTTGCCTGGCCGTGGGGCGGCCAGAGCCCACGGTCACCTGGAGACAGCTCCGAGACG | |
| GCTTCACCTCGGAGGGAGAGATCCTGGAGATCTCTGACATCCTGCGGGGCCAGGCCCGGGAGTATGAGTGCGT | |
| GACTCACAACGGGGTTAACTCGGCGCCCGACAGCCGCCGCGTGCTGGTCACAGTCAACTATCCTCCGACCATC | |
| ACGGACGTGACCAGCGCCCGCACCGCGCTGGGGCCGGGCCGCCTACTGCGCTGCGAAGCCATGGCGGTTTCCC | |
| CCGCGGATTTCCAGTGGTATAAGGATGACAGACTACTGAGCAGCGGCACGGCCGAGGGCCTGAAGGTGCAGAT | |
| GGAGCGCCACTCGCTCGATGCTTCTCTTTGCCAACATGAGCGCCCGCATTACGGCAACTATACGTGTTGCGCC | |
| GCCAACCGGCTGGGAGCGTCCAGCGCCTCCATGCGGCTCCTGTGCCCAGGATCCCTGGAGAACTCAGCCCCGA | |
| GGCCCCCAGGGCCCCTGGCCCTCCTCTCCGCCCTGGGCTGGCTGTGGTGGAGAATGTAGGCGCAACCCAGTGG | |
| AGCTCGCCTCCCCCTGCAGGGGGCCTCAGGCCAAGAGTGAGAGAAAAGGGGGAGCAAGAGCCCTGGGTCTCGT | |
| +E, UNS G | |

The NOV5d nucleic acid was identified on chromosome 7 and has 257 of 389 bases (66%) identical to a Homo sapiens (clone pHOM) opioid-binding cell adhesion molecule mRNA (gb:GENBANK-ID:HUMOBCAM|acc:L34774.1) (E=2.0e⁻⁴⁶).

A disclosed NOV5d polypeptide (SEQ ID NO: 28) encoded by SEQ ID NO: 27 is 336 amino acid residues and is presented using the one-letter code in Table 5H. Signal P, Psort and/or Hydropathy results predict that NOV5d contains a signal peptide and is likely to be localized extracellularly with a certainty of 0.6329. The most likely cleavage site for a NOV5d peptide is between amino acids 30 and 31, at: LLS-QR.

TABLE 5H

Encoded NOV5d protein sequence

| | |
|---|---|
| MPPAAPGARLRLLAAAALAGLAVISRGLLSQRLEFNSPADNYTVCEGDNATLSCFMDEHVTRVAWLNRSNIL | (SEQ ID NO:28) |
| TAGNDRRTRDPRVRLLINTPEEFSILVTEVGLGDEGLYTCSFQTRHQPYTTQVYLIVHVPARVVNISSPVMV | |
| NEGGNVNLLCLAVGRPEPTVTWRQLRDGFTSEGEILEISDILRGQAGEYECVTHNGVNSAPDSRRVLVTVNY | |
| PPTITDVTSARTALGPGRLLRCEAMAVSPADFQWYKDDRLLSSGTAEGLKVQMERTRSMLLFANMSARHYGN | |
| YTCCAANRLGASSASMRLLCPGSLENSAPRPPGPLALLSALGWLWWRM | |

The NOV5d amino acid sequence has 160 of 327 amino acid residues (48%) identical to, and 218 of 327 amino acid residues (66%) similar to, a Bos taurus 345 amino acid residue opioid biniding protein/cell adhesion molecule precursor (OBCAM)/opioid-binding cell adhesion molecule (OPCML) protein (ptnr:SWISSNEW-ACC:P11834) (E=1.5e⁻⁷⁵).

NOV5d is expressed in at least the following tissues: Brain and Fetal brain. This information was derived by determining the tissue sources of the sequences that were included in the invention including but not limited to SeqCalling sources, Public EST sources, genomic clone sources, literature sources, and/or RACE sources. In addition, NOV5d is predicted to be expressed in occipital cortex tissues because of the expression pattern of a closely related Homo sapiens (clone pHOM) opioid-binding cell adhesion molecule mRNA homolog (GENBANK_ID:gb:GENBANK-ID:HUMOBCAM|acc:L34774.1).

Possible small nucleotide polymorphisms (SNPs) found for NOV5c are listed in Table 5I.

TABLE 5I
SNPs

| Consensus | | Base | |
|---|---|---|---|
| Position | Depth | Change | PAF |
| 141 | 25 | C > T | 0.080 |
| 190 | 32 | A > G | 0.062 |
| 205 | 38 | C > T | 0.053 |
| 246 | 84 | G > A | 0.429 |
| 280 | 88 | T > C | 0.023 |
| 335 | 94 | C > G | 0.043 |
| 360 | 94 | A > G | 0.255 |
| 385 | 94 | C > T | 0.277 |
| 398 | 100 | G > A | 0.270 |
| 401 | 100 | C > T | 0.270 |
| 433 | 99 | G > A | 0.020 |
| 463 | 100 | T > C | 0.020 |
| 514 | 100 | A > T | 0.300 |
| 575 | 94 | C > T | 0.043 |
| 619 | 62 | G > T | 0.032 |
| 620 | 62 | A > C | 0.032 |
| 665 | 58 | G > A | 0.190 |
| 670 | 58 | A > C | 0.362 |
| 695 | 57 | C > T | 0.368 |
| 719 | 55 | T > C | 0.400 |
| 734 | 55 | G > A | 0.400 |
| 755 | 47 | A > G | 0.234 |
| 772 | 43 | C > T | 0.302 |
| 782 | 41 | C > T | 0.293 |
| 807 | 35 | G > A | 0.286 |
| 840 | 24 | C > T | 0.167 |
| 854 | 20 | A > G | 0.150 |
| 888 | 19 | C > T | 0.158 |

NOV5a-NOV5d are very closely homologous as is shown in the amino acid alignment in Table 5J.

[image]

Homologies to any of the above NOV5 proteins will be shared by the other NOV5 proteins insofar as they are homologous to each other as shown above. Any reference to NOV5 is assumed to refer to both of the NOV5 proteins in general, unless otherwise noted.

NOV5a also has homology to the amino acid sequences shown in the BLASTP data listed in Table 5K.

TABLE 5K
BLAST results for NOV5a

| Gene Index/ | | Length | Identity | Positives | |
|---|---|---|---|---|---|
| Identifier | Protein/Organism | (aa) | (%) | (%) | Expect |
| gi\|129173\|sp\|P11834 | opioid binding | 345 | 164/302 | 213/302 | 3e−86 |
| \|OPCM_BOVIN | protein/cell | | (54%) | (70%) | |
| | adhesion molecule | | | | |
| | precursor (OBCAM) | | | | |
| | (opioid-binding | | | | |
| | cell adhesion | | | | |
| | molecule) (OPCML) | | | | |
| | [Bos taurus] | | | | |
| gi\|4505505\|ref\| | opioid-binding | 345 | 162/302 | 213/302 | 2e−85 |
| NP_002536.1\| | cell adhesion | | (53%) | (69%) | |
| (NM_002545) | molecule | | | | |
| | precursor; opiate | | | | |
| | binding-cell | | | | |
| | adhesion molecule | | | | |
| | [Homo sapiens] | | | | |
| gi\|1352640\|sp\| | opioid binding | 345 | 163/306 | 213/306 | 8e−85 |
| P32736\|OPCM_RAT | protein/cell | | (53%) | (69%) | |
| | adhesion molecule | | | | |
| | precursor (OBCAM) | | | | |
| | (opioid-binding | | | | |
| | cell adhesion | | | | |
| | molecule) (OPCML) | | | | |
| | [Rattus | | | | |
| | norvegicus] | | | | |
| gi\|112102\|pir\|\| | opioid-binding | 338 | 163/306 | 213/306 | 1e−84 |
| JC1238 | protein (clone | | (53%) | (69%) | |
| | DUZ1) [Rattus | | | | |
| | norvegicus] | | | | |
| gi\|3298456\|dbj\| | CEPU-1 [Gallus | 344 | 155/283 | 199/283 | 7e−81 |
| BAA31514.1\|(AB011810) | gallus] | | (54%) | (69%) | |

The homology of these sequences is shown graphically in the ClustalW analysis shown in Table 5L.

[image]

[image]

Tables 5M -5R list the domain description from DOMAIN analysis results against NOV5a. This indicates that the NOV5a sequence has properties similar to those of other proteins known to contain these domains.

TABLE 5M
Domain Analysis of NOV5a

gnl|Smart|smart00409, IG. Immunoglobulin
Length = 86 residues. 100.0% aligned
Score = 56.1 bits (126), Expect 2e−08

| | | | | |
|---|---|---|---|---|
| NOV5a | 122 | SSPVTVNEGGNVNLLCLAVGRPEPTVTWRQ-------LRDGFTSEGE----ILEISDIQR | 170 | (SEQ ID NO:88) |
| | | \|\|\| \|\| +\| \| \| \| \| \| \|\|\|\|\| + \| \|\|++ | | |
| 00409 | 1 | PPSVTVKEGESVTLSCEASGNPPPTVTWYKQGGKLLAESGRFSVSRSGGNSTLTISNVTP | 60 | |
| NOV5a | 171 | GQAGEYECVTBNGVNSAPDSRRVLVT 196 | | |
| | | +\| \| \| \|\| + \| | | |
| 00409 | 61 | EDSGTYTCAATNSSGSASSGTTLTVL 86 | | |

TABLE 5N

Domain Analysis of NOV5a

gnl|Smart|smart00409, IG. Immunoglobulin
Length = 86 residues, 87.2% aligned
Score = 49.3 bits (116), Expect = 3e−07

| | | | | |
|---|---|---|---|---|
| NOV5a | 214 | GRTALLRCEAMAVPPADFQWYKDDRLLSSGTAEGLKQTERTRSMLLFANVSARHYGNYT | 273 | (SEQ ID NO:89) |
| | | \| + \| \|\|\| \|\| \|\|\| \| + + \| \| \| +\|\|+ \| \|\| | | |
| 00409 | 9 | GESVTLSCEASGNPPPTVTWYKQGGKLLAES-GRFSVSRSGGNSTLTTSNVTPEDSGTYT | 67 | |
| NOV5a | 274 | CRAANRLGASSASMRL 289 | | |
| | | \| \| \| \|++\|+ \| | | |
| 00409 | 68 | CAATNSSGSASSGTTL 8 | | |

TABLE 5O

Domain Analysis of NOV5a

gnl|Smart|smart00409, IG. Immimoglobulin
Length = 86 residues, 97.7% aligned
Score = 43.9 bits (102), Expect = 1e−05

| | | | | |
|---|---|---|---|---|
| NOV5a | 24 | NYTVCEGDNATLSCFIDEHVT-RVAWLNRSNILYAGNDRWTSDPRVRLLINTPEEFSILI | 82 | (SEQ ID NO:90) |
| | | + \|\| \|\|++ \|\|\|\| + \| \| \| + \|++ + ++ \| | | |
| 00409 | 3 | SVTVKEGESVTLSCEASGNPPPTVTWYKQGGKLLAESGRFSVS-------RSGGNSTLTI | 55 | |
| NOV5a | 83 | TEVGLGDEGLYTCSFQTRHQPYTTQVYLIVH 113 | | |
| | | + \| \| \| \|\|\|+ ++ \| \| | | |
| 00409 | 56 | SNVTPEDSGTYTCAATNSSGSASSGTTLTVTl 86 | | |

TABLE 5P

Domain Analysis of NOV5a

gnl|Smart|smart00408, IGc2, Immunoglobulin C-2 Type
Length = 63 residues, 100.0% aligned
Score = 31.6 bits (122), Expect = 7e−08

| | | | | |
|---|---|---|---|---|
| NOV5a | 128 | NEGGNVNLLCLAVGRPEPTVTWR----QLRDG-FTSEGEILEISDIQRGQAGEYECVTHN | 182 | (SEQ ID NO:91) |
| | | \|\| +\| \| \| \| \| \| \| +\|\| \| + \| + + \| \| \| ++ +\| \| \|\| \| | | |
| 00408 | 1 | LEGESVTLTCPASGDPVPNITWLKDGKPLPESRVVASGSTLTIKNVSLEDSGLYTCVARN | 60 | |
| NOV5a | 183 | GVN 185 | | |
| | | \| | | |
| 00408 | 61 | SVG 63 | | |

TABLE 5Q

Domain Analysis of NOV5a

gnl|Smart|smart00408, IGc2, Immunoglobulin C-2 Type
Length = 63 residues, 96.8% aligned
Score = 50.8 bits (120), Expect = 1e−07

| | | | | |
|---|---|---|---|---|
| NOV5a | 214 | GRTALLRCEAMAVPPADFQWYKDDRLLSSGTAEGLKVQTERTRSMLLFANVSARHYGNYT | 273 | (SEQ ID NO:92) |
| | | \| + \| \| \| \| + \| \|\| + \| + + \| \| \|\|\| \| \|\| | | |
| 00408 | 3 | QESVTLTCPASGDPVPNITWLKDGKPLPES-------RVVASGSTLTIKNVSLEDSGLYT | 55 | |
| NOV5a | 274 | CRAANRLG 281 | | |
| | | \| \| \| +\| | | |
| 00408 | 56 | CVARNSVG 63 | | |

TABLE 5R

Domain Analysis of NOV5a

gnl|Pfam|pfam00047, ig, Immunoglobulin domain. Members of the
immunoglobulin superfamily are found in hundreds of proteins of
different functions. Examples include antibodies, the giant muscle
kinase titin and receptor tyrosine kineses. Immunoglobulin-like
domains may be involved in protein-protein and protein-ligand
interactions. The Pfam alignments do not include the first and last
strand of the immunoglobulin-like domain.
Length = 68 residues, 100.0% aligned
Score = 35.0 bits (79), Expect = 0.007

| | | | | |
|---|---|---|---|---|
| NOV5a | 214 | +TL,8GRTALLRCEAMAVPP-ADFQWYKDDRLLSSGTAEGLKVQTERTRSM----LLFANVSARH | 268 | (SEQ ID NO:93) |
| | | \| + \| \| \|\| \| +\| + + + +\| + \|+ \| ++\|+ | | |
| 00407 | 1 | GESVTLTCSVSGYPPDPTVTWLRDGKEIELLGSSESRVSSGCFSISSLSLTISSVTPED | 60 | |
| NOV5a | 269 | YGNYTCRA 276 | | |
| | | \| \|\|\| | | |
| 00407 | 61 | SGTYTCVV 68 | | |

Opioid-binding cell adhesion molecule (OBCAM), a neuron-specific protein, consists of three immunoglobulin (Ig)-like domains anchored to the membrane through a glycosylphosphatidylinositol (GPI)-tail. OBCAM has been presumed to play a role as a cell adhesion/recognition molecule, but its function has not been fully elucidated and may also play a role in early neuronal development (Hachisuka et al., Developmental expression of opioid-binding cell adhesion molecule (OBCAM) in rat brain. J Brain Res Dev Brain Res 122(2):183-91, 2000).

It has been previously reported that transfection of antisense OBCAM cDNA into NG108-15 neuroblastoma x glioma hybrid cells, which contain delta-opioid receptors, results in greatly reduced opioid binding (Ann et al., J. Biol. Chem. 267, 7921-7926, 1992) and more recently it has been reported that these cells show altered coupling between opioid receptors and G-proteins (Govitrapong et al., Transfection of NG108-15 cells with antisense opioid-binding cell adhesion molecule cDNA alters opioid receptor-G-protein interaction. J Biol Chem 268(24):18280-5, 1993).

Despite the recent cloning of mu, delta and kappa opioid receptors, a role in opioid receptor function for OBCAM is supported by several lines of evidence, including inhibition of opioid binding by opioid binding cell adhesion molecule antibodies, down-regulation of opioid binding cell adhesion molecule by chronic opioid agonist treatment of cultured NG108-15 cells, and reduction of opioid binding in NG108-15 cells by transfection of opioid binding cell adhesion molecule antisense cDNA (Kalyuzhny et al., An opioid binding protein is specifically down-regulated by chronic morphine treatment in dorsal root and trigeminal ganglia. Neuroscience 66(4):943-9, 1995).

The tissue distribution and brain localization of OBCAM was investigated in the adult rats. OBCAM was preferentially expressed in the central nervous system (CNS) and at a very low level in the spleen. Within the brain, OBCAM was distributed in almost all the gray matter, but little or no immunoreactive OBCAM was found in the white matter. Morphologically, the distribution pattern of OBCAM immunoreactivity was very similar to that of synaptophysin, suggesting a role in the synaptic machinery (Hachisuka et al., Localization of opioid-binding cell adhesion molecule (OBCAM) in adult rat brain. 842(2):482-6, 1999). Another study investigated the expression of two immunoglobulin superfamily (IgSF) proteins, Kilon and OBCAM. This study demonstrated the specific expression of Kilon and OBCAM in the hypothalamic magnocellular neurons, particularly in dendrites, suggesting that they confer on magnocellular neurons the ability to rearrange dendritic connectivity (Miyata et al., Expression of the IgLON cell adhesion molecules Kilon and OBCAM in hypothalamic magnocellular neurons. 424(1):74-85, 2000).

The above defined information for NOV5 suggests that this NOV5 protein may function as a member of a Opioid Binding Cell Adhesion Molecule-like protein family. Therefore, the NOV5 nucleic acids and proteins of the invention are useful in potential therapeutic applications implicated in various diseases and disorders described below and/or other pathologies. For example, the NOV5 compositions of the present invention will have efficacy for treatment of patients suffering from Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neurodegeneration. The NOV5 nucleic acid encoding Opioid Binding Cell Adhesion Molecule-like protein, and the Opioid Binding Cell Adhesion Molecule-like protein of the invention, or fragments thereof, may further be useful in diagnostic applications, wherein the presence or amount of the nucleic acid or the protein are to be assessed.

NOV6

NOV6 includes two novel triacylglycerol lipase-like proteins disclosed below. The disclosed proteins have been named NOV6a and NOV6b.

NOV6a

A disclosed NOV6a nucleic acid of 1377 nucleotides (also referred to as SC122982104_A) encoding a novel triacylglycerol lipase-like protein is shown in Table 6A. An open reading frame was identified beginning with an ATG initiation codon at nucleotides 91-93 and ending with a TAA codon at nucleotides 1243-1245. Putative untranslated regions upstream from the start codon and downstream from the termination codon are underlined in Table 6A, and the start and stop codons are in bold letters.

TABLE 6A
NOV6a Nucleotide Sequence

(SEQ ID NO:29)

CAGTTTTATAATGGTATTATTTCCTGCAATTTTAACTGTATAAACATCTCCCTTTTATATATAGGTCCAATGTGGTATC
TTTTCACAATGATGTATTTTATCCGAATTCTTGGAATTACTCATGGTGTCTTTATTATAATCTGTGACCTGAGC
AGATATGAATATTAGCCAGATTATTTCCTACTGGGGCTACCCTGATGAAGAATATGATATTGTAACCGAAGATGGTTATATC
CTTGGCCTTTATAGAATTCCTTATTGGAGGACAGACAATAATAAAAATCTAGGTAATTCAGCTCAGAGGGTTGTTGTATACT
TGCAACATGGTTTGCTTACATCTGCCAGCAGCTGGATTTCCAATCTTCCCAACAATAGTCTGGGCTTCATTCTGGCAGATGC
TGGTTATGATGTGTGGATGGGAAATAGCAGAGGAAATACCTGGTCCAGGAAACACTTGTACCTAGAAACGAGTTCCAAAGAA
TTCTGGGCTTTCAGGTACGCTCAAGGAGGCCTGCCTGCCTCTGTAGACTGCATCTTGGTGAAGAAGAGAGGAGAGAAAAATA
TATATCATTATATCTTTCATTCCCAAGTACATAGCCAGGGAACCTTAGGTTTCATAACATTTTCTACTATATCAAAGATAGC
TGAAAGAATCAAAATATTTTTTGCTTTAGCACCCTCATCCTCAGTCAAATATACCAAGTCAATTATTCTTAAACTTACATAC
AAATGGAAGTCAATAGGCAACAAAGACTTCTTGCCTAAAACCTCATTTAAAAAATTCATTGGTTCAAAGCTGTGTCCACTAC
AGATTTTTGATAAGATTTGCCTCAATATCTTGTTTATGATGTTTGGATATGACCCAAAAAACTTAAATATGAGTCGTTTGGA
TGTGTATTTTTCACACAACCCAGCAGGAACATCTGTTCAAAATATGCTTCATTGGAGTCAGGCTTATGACTGGGGCAGTCCT
GATCTGAACTTGGTTCATTATAATCAGACAACG5CTCCATTATACAACATGACAAACATGAATGTGGCAACTGCAATTTGGA
ATGGTAAAAGTGACTTGTTGGCTGACCCTGAAGACGTTAACATTTTACATTCTGAAATCACAAACCACATTTATTATAAAAC
TATTTCTTACTACAATCATATAGACTCTTTGTTTGGATTAGATGTCTATGATCAAGTTTACCATGAAATCATTGATATTATC
CAAGACAATCTATAAAGAACCATGGCGCTGTGTGTTTAAAGATCTACATCATTCCTAATGAAATCCAATTCTTATTTTTTTT
TACCTGTGTATGTTCTTTCATTTTTAAAACTAAATATGTAGTTTTTTCCTCTATATTCTCATTGA

The NOV6a nucleic acid was identified on chromosome 10 and has 367 of 543 bases (67%) identical to a rabbit gastric lipase precursor coding sequence mRNA (gb:GENBANK-ID:A26689|acc:A26689.1) (E=9.1e⁻⁸¹).

A disclosed NOV6a polypeptide (SEQ ID NO: 30) encoded by SEQ ID NO: 29 is 390 amino acid residues and is presented using the one-letter code in Table 6B. Signal P, Psort and/or Hydropathy results predict that NOV6a contains a signal peptide and is likely to be localized in the microbody (peroxisome) with a certainty of 0.7480. The most likely cleavage site for a NOV6a peptide is between amino acids 19 and 20, at: THG-VF.

TABLE 6B
Encoded NOV6a protein sequence

(SEQ ID NO:30)

MWYLFTMMYFIRILGITHGVFQNYRSVKPEADMNISQIISYWGYPDEEYDIVTEDGYILGLYRIPYWRTDNNKNLGNSAQR
VVVYLQHGLLTSASSWISNLPNNSLGFILADAGYDVWMGNSRGNTWSRKHLYLETSSKEFWAFRYAQGGLPASVDCILVKK
RGEKNIYHYIFHSQVHSQGTLGFITFSTISKIAERIKIFFALAPSSSVKYTKSIILKLTYKWKSIGNKDFLPKTSFKKFIG
SKLCPLQIFDKICLNILFMMFGYDPKNLNMSRLDVYFSHNPAGTSVQNMLHWSQAYDWGSPDLNLVHYNQTTSPLYNMTNM
NVATAIWNGKSDLLADPEDVNILHSEITNHIYYKTISYYNHIDSLFGLDVYDQVYHEIIDIIQDNL

The NOV6a amino acid sequence has 218 of 396 amino acid residues (55%) identical to, and 288 of 396 amino acid residues (72%) similar to, a Homo sapiens 398 amino acid residue (triacylglycerol lipase, gastric precursor (ec 3.1.1.3) (gastric lipase) protein (ptnr:SWISSPROT-ACC:P07098) (E=8.0⁻¹¹⁴)

NOV6a is expressed in at least the following tissues: Whole Organism, fetal lung. This information was derived by determining the tissue sources of the sequences that were included in the invention. SeqCalling sources: Whole Organism, PublicEST sources: fetal lung NbHL19W, testis NHT, and B-cell, pooled germ cell. In addition, NOV6a is predicted to be expressed in the following tissues because of the expression pattern of a closely related precursor of rabbit gastric lipase coding sequence homolog in species synthetic construct (gb:GENBANK-ID:A26689|acc:A26689.1): lung, testis, tumors.

NOV6b

A disclosed NOV6b nucleic acid of 1260 nucleotides (also referred to as CG58608-02) encoding a novel triacylglycerol lipase-like protein is shown in Table 6C. An open reading frame was identified beginning with an ATG initiation codon at nucleotides 31-33 and ending with a TAA codon at nucleotides 1234-1236. Putative untranslated regions upstream from the start codon and downstream from the termination codon are underlined in Table 6C, and the start and stop codons are in bold letters.

TABLE 6C
NOV6b Nucleotide Sequence

(SEQ ID NO:31)

ATTAAATTTCCTTTCCTAGGCAGATCCCAAATGTGGCAGCTTTTAGCAGCAGCATGCTGGATGCTTCTTCTTGGATCTA
TGTATGGTTATGACAAGAAAGGAAACAATGCAAACCCTGAAGCTAATATGAATATTAGCCAGATTATTTCCTACTGGGG
CTACCCTGATGAAGAATATGAPATTGTAACCGAAGATCGTTATATCCTTGGCCTTTATAGAATTCCTTATTCGAGGACA
GACAATAATAAAAATCTAGGTAATTCAGCTCAGAGGGTTGTTGTATACTTGCAACATGGTTTGCTTACATCTGCCAGCA
GCTGGATTTCCAATCTTCCCAACAATAGTCTGGGCTTCATTCTGGCAGATGCTGGTTATGATGTGTGGATGGGAAATAG
CAGAGGAAATACCTGGTCCAGGAAACACTTGTACCTAGAAACGAGTTCCAAAGAATTCTGGGCTTTCAGTTTTGATGAG
ATGGCAAAATATGACCTTCCAGCCTCTATTGATTTCACTGTGAAGCAAACCAGACAAGAGGAAATATTTTATGTAGGCC
ATTCACAGGGTACTACTATTGGTTTCATAACATTTTCTACTATATCAAAGATAGCTGAAAGAATCAAAATATTTTTTGC
TTTAGCACCAGTTTTTTCCACAAAGTACTTAAAAAGTCCTTTAATTAGAATGACATACAAATGGAAGTCAATAGTCATG
GCTTTTTCAGGCAACAAAGACTTCTTGCCTAAAACCTCATTTAAAAAATTCATTGGTTCAAAGCTGTGTCCACTACAGA
TTTTTGATAAGATTTGCCTCAATATCTTGTTTATGATGTTTGGATATGACCCAAAAAACTTAAATATGAGTCGTTTGGA
TGTGTATTTTTCACACAACCCAGCAGGAACATCTGTTCAAAATATGCTTCATTGGAGTCAGCTTTTAAATTCTACTCAT
TTGAAAGCTTATGACTGGGGCAGTCCTGATCTGAACTTGGTTCATTATAATCAGACAACGTCTCCATTATACAACATGA
CAAACATGAATGTGGCAACTGCAATTTGGAATGGTAAAAGTGACTTGTTGGCTGACCCTGAAGACGTTAACATTTTACA
TTCTGAAATCACAAACCACATTTATTATAAAACTATTTCTTACTACAATCATATAGACTCTTTGTTTGGATTAGATGTC
TATGATCAAGTTTACCATGAAATCATTGATATTATCCAAGACAATCTATAAGAACCATGGCGCTGTGTGTTTAA

The NOV6b nucleic acid was identified on chromosome 1 and has 711 of 997 bases (71%) identical to a Homo sapiens gastric lipase mRNA (gb:GENBANK-ID:A01046|acc:A01046.1) (E=1.3e⁻¹¹⁷)

A disclosed NOV6b polypeptide (SEQ ID NO: 32) encoded by SEQ ID NO: 31 is 401 amino acid residues and is presented using the one-letter code in Table 6D. Signal P, Psort and/or Hydropathy results predict that NOV6b contains a signal peptide and is likely to be localized in the lysosome (lumen) with a certainty of 0.5078. The most likely cleavage site for a NOV6b peptide is between amino acids 19 and 20, at: MYG-YD.

TABLE 6D
Encoded NOV6b protein sequence.

(SEQ ID NO:32)

MWQLLAAACWMLLLGSMYGYDKKGNNANPEANMNISQIISYWGYPDEEYDIVTEDGYILGLYRIPYWRTDNNKNLGNSAQRVVVY
LQHGLLTSASSWISNLPNNSLGFILADAGYDVWMGNSRGNTWSRKHLYLETSSKEFWAFSFDSMAKYDLPASIDFTVKQTRQEEI
FYVGHSQGTTIGFITFSTISKIAERIKIFFALAPVFSTKYLKSPLIRMTYKWKSIVMAFSGNKDFLPKTSFKKFIGSKLCPLQIF
DKICLNILFMMFGYDPKNLNMSRLDVYFSHNPAGTSVQNMLHWSQLLNSTHLKAYDWGSPDLNLVHYNQTTSPLYNMTNMNVATA
IWNGKSDLLADPEDVNILHSEITNHIYYKTISYYNHIDSLFGLDVYDQVYHEIIDIIQDNL

The NOV6b amino acid sequence has 224 of 400 amino acid residues (56%) identical to, and 298 of 400 amino acid residues (74%) similar to, a Homo sapiens 398 amino acid residue (triacylglycerol lipase, gastric precursor (EC 3.1.1.3) (gastric lipase) protein (ptnr:SWISSPROT-ACC:P07098) (E=3.5e⁻¹²⁴).

NOV6b is expressed in at least the following tissues: Mammalian Tissue. This information was derived by determining the tissue sources of the sequences that were included in the invention including but not limited to SeqCalling sources, Public EST sources, Literature sources, and/or RACE sources.

Possible small nucleotide polymorphisms (SNPs) found for NOV6a are listed in Table 6E.

TABLE 6E
SNPs

| | | | Amino | |
|---|---|---|---|---|
| | Neucleotide | Base | Acid | Base |
| Variant | Position | Change | Position | Change |
| 13375771 | 629 | T > C | 186 | Ile > Tyr |

NOV6a and NOV6b are very closely homologous as is shown in the amino acid alignment in Table 6F.

[image]

Homologies to any of the above NOV6 proteins will be shared by the other NOV6 proteins insofar as they are homologous to each other as shown above. Any reference to NOV6 is assumed to refer to both of the NOV6 proteins in general, unless otherwise noted.

NOV6a also has homology to the amino acid sequences shown in the BLASTP data listed in Table 6G.

TABLE 6G
BLAST results for NOV6a

| Gene Index/ | | Length | Identity | Positives | |
|---|---|---|---|---|---|
| Identifier | Protein/Organism | (aa) | (%) | (%) | Expect |
| gi\|4758676\|ref\| | lipase, gastric | 398 | 218/405 | 288/405 | 1e−117 |
| NP_004181.1 | [Homo sapiens] | | (53%) | (70%) | |
| (NM_004190) | | | | | |
| gi\|758064\|emb\| | gastric lipase | 392 | 214/399 | 284/399 | 1e−114 |
| CAA29414.1\|(X05997) | precursor [Homo | | (53%) | (70%) | |
| | sapiens] | | | | |
| gi\|3041702\|sp\|P80035 | triacylglycerol | 398 | 216/402 | 285/402 | 1e−113 |
| \|LIPG_CANFA | lipase, gastric | | (53%) | (70%) | |
| | precursor | | | | |
| | (gastric lipase) | | | | |
| | (GL) [Canis | | | | |
| | familiaris] | | | | |
| gi\|7546565\|pdb\|1HLG\|A | Chain A, Crystal | 371 | /377 | 274/377 | 1e−109 |
| | Structure Of | | (54%) | (72%) | |
| | Human Gastric | | | | |
| | Lipase | | | | |
| gi\|8394193\|ref\| | lingual lipase | 395 | 203/405 | 290/405 | 1e−107 |
| NP_059037.1\| | [Rattus | | (50%) | (71%) | |
| (NM_017341) | norvegicus] | | | | |

The homology of these sequences is shown graphically in the ClustalW analysis shown in Table 6H.

[image]

[image]

The hydrolysis of triglycerides under influence of lipoprotein lipase is among the first recognised and well defined processes in postprandial lipid metabolism. More recently, also hepatic lipase has been implicated in the clearing of postprandial lipoproteins. Lipoprotein lipase as well as hepatic lipase are also involved in the metabolism of several other lipoproteins. However, their capacity is limited. This may lead to interaction of different metabolic processes and competition for the available lipase by different lipoproteins. Indeed, it is generally accepted that the exaggerated postprandial response in subjects with hypertriglyceridemia is at least partially due to competition between endogenous (VLDL) and exogenous (chylomicrons) lipoproteins. Similar mechanisms may also take place in the liver where hepatic lipase plays a role in the metabolism of several lipoproteins (Jansen et al., Role of lipoprotein lipases in postprandial lipid metabolism. Atherosclerosis 141 Suppl 1:S31-4, 1998).

Hepatic lipase (HL) is an enzyme that is made primarily by hepatocytes (and also found in adrenal gland and ovary) and hydrolyzes phospholipids and triglycerides of plasma lipoproteins. It is secreted and bound to the hepatocyte surface and readily released by heparin. It is a member of the lipase superfamily and is homologous to lipoprotein lipase and pancreatic lipase. The enzyme can be divided into an NH2-terminal domain containing the catalytic site joined by a short spanning region to a smaller COOH-terminal domain. The NH2-terminal portion contains an active site serine in a pentapeptide consensus sequence, Gly-Xaa-Ser-Xaa-Gly, as part of a classic Ser-Asp-His catalytic triad, and a putative hinged loop structure covering the active site. The COOH-terminal domain contains a putative lipoprotein-binding site. The heparin-binding sites may be distributed throughout the molecule, with the characteristic elution pattern from heparin-sepharose determined by the COOH-terminal domain. Of the three N4inked glycosylation sites, Asn-56 is required for efficient secretion and enzymatic activity. HL is hypothesized to directly couple HDL lipid metabolism to tissue/cellular lipid metabolism. The potential significance of the HL pathway is that it provides the hepatocyte with a mechanism for the uptake of a subset of phospholipids enriched in unsaturated fatty acids and may allow the uptake of cholesteryl ester, free cholesterol, and phospholipid without catabolism of HDL apolipoproteins. HL can hydrolyze triglyceride and phospholipid in all lipoproteins, but is predominant in the conversion of intermediate density lipoproteins to LDL and the conversion of post-prandial triglyceride-rich HDL into the postabsorptive triglyceride-poor HDL. HL plays a secondary role in the clearance of chylomicron remnants by the liver. A rare family with HL deficiency has been described. Affected patients are compound heterozygotes for a mutation of Ser267 to Phe that results in an inactive enzyme and a mutation of Thr383 to Met that results in impaired secretion and reduced specific activity. Human HL deficiency in the context of a second factor causing hyperlipidemia is strongly associated with premature coronary artery disease (Connelly and Hegele, Hepatic lipase deficiency. Crit Rev Clin Lab Sci 35(6):547-72, 1998).

The above defined information for NOV6 suggests that NOV6 may function as a member of a triacylglycerol lipase family. Therefore, the NOV6 nucleic acids and proteins of the invention are useful in potential therapeutic applications implicated in various diseases and disorders described below and/or other pathologies. For example, the NOV6 compositions of the present invention will have efficacy for treatment of patients suffering from Adrenoleukodystrophy, Congenital Adrenal Hyperplasia, Diabetes, Von Hippel-Lindau (VHL) syndrome, Pancreatitis, Obesity, Endometriosis, Xerostomia, Scleroderma, Hypercalceimia, Ulcers Von Hippel-Lindau (VHL) syndrome, Cirrhosis, Transplantation, Inflammatory bowel disease, Diverticular disease, Hirschsprung's disease, Crohn's Disease, Appendicitis, Hypercalceimia, Arthritis, Ankylosing spondylitis, Arthritis, Tendinitis on Hippel-Lindau (VHL) syndrome, Alzheimer's disease, Stroke, Tuberous sclerosis, hypercalceimia, Parkinson's disease, Huntington's disease, Endocrine dysfunctions, Diabetes, Growth and reproductive disorders, Multiple sclerosis, Leukodystrophies, Pain, Myasthenia gravis, Autoimmune disease, Asthma, Emphysema, Scleroderma, allergy, ARDS, Psoriasis, Actinic keratosis, Tuberous sclerosis, Acne, Hair growth, allopecia, pigmentation disorders, endocrine disorders Diabetes, Renal artery stenosis, Interstitial nephritis, Glomerulonephritis, Polycystic kidney disease, erythematosus, Renal tubular acidosis, IgA nephropathy, Hypercalceimia, Lesch-Nyhan syndrome. The NOV6 nucleic acid encoding triacylglycerol lipase-like protein, and the triacylglycerol lipase-like protein of the invention, or fragments thereof, may further be useful in diagnostic applications, wherein the presence or amount of the nucleic acid or the protein are to be assessed.

NOV7

NOV7 includes two novel IGE receptor beta subunit-like proteins disclosed below. The disclosed proteins have been named NOV7a and NOV7b.

NOV7a

A disclosed NOV7a nucleic acid of 691 nucleotides (also referred to SC126624027_A) encoding a novel IGE receptor beta subunit-like protein is shown in Table 7A. An open reading frame was identified beginning with an ATG initiation codon at nucleotides 52-54 and ending with a TGA codon at nucleotides 652-654. Putative untranslated regions are found upstream from the initiation codon and downstream from the termination codon in Table 7A, and the start and stop codons are in bold letters.

TABLE 7A
NOV7a Nucleotide Sequence

(SEQ ID NO:33)

GACTGAAGTACCAACTAAGTCATCTCCTTTCAAATTATCACCGACACCATCATGGATTCAAGCACCGCACACAGTCCGGT
GTTTCTGGTATTTCCTCCAGAAATCACTGCTTCAGAATATGAGTCCACAGAACTTTCAGCCACGACCTTTTCAACTCAAA
GCCCCTTGCAAAAATTATTTGCTAGAAAAATGAAAATCTTAGGGACTATCCAGATCCTGTTTGGAATTATGACCTTTTCT
TTTGGAGTTATCTTCCTTTTCACTTTGTTAAAACCATATCCAAGGTTTCCCTTTATATTTCTTTCPGGATATCCATTCTG
GGGCTCTGTTTTGTTCATTAATTCTGGAGCCTTCCTAATTGCAGTGAAAAGAAAAACCACAGAAACTCTGATAATATTGA
GCCGAATAATGAATTTTCTTAGTGCCCTGGGAGCAATAGCTGGAATCATTCTCCTCACATTTGGTTTCATCCTAGATCAA
AACTACATTTGTGGTTATTCTCACCAAAATAGTCAGTGTAAGGCTGTTACTGTCCTGTTCTTGGGAATTTTGATTACATT
GATGACTTTCAGCATTATTGAATTATTCATTTCTCTGCCTTTCTCAATTTTGGGGTGCCACTCAGAGGATTGTGATTGTG
AACAATGTTGTGTACTAGCACTGTGAGAATAAAGATGTGTTAAAATAAAAA

The disclosed NOV7a nucleic acid sequence, localized to chromosome 15, has 325 of 560 bases (58%) identical to a Mus musculus mast cell high affinity IgE receptor (Fc-epsilon-RI) beta subunit mRNA (gb:GENBANK-ID:MUSFCERB|acc:J05019.1) (E=2.2e⁻⁰⁵).

A disclosed NOV7a polypeptide (SEQ ID NO: 34) encoded by SEQ ID NO: 33 is 200 amino acid residues and is presented using the one-letter amino acid code in Table 7B. Signal P, Psort and/or Hydropathy results predict that NOV7a contains a signal peptide and is likely to be localized in the plasma membrane with a certainty of 0.6000. The most likely cleavage site for a NOV7a peptide is between amino acids 20 and 21, at: ITA-SE.

TABLE 7B
Encoded NOV7a protein sequence.

(SEQ ID NO:34)

MDSSTAHSPVFLVFPPEITASEYESTELSATTFSTQSPLQKLFARKMKIL
GTIQILFGIMTFSFGVIFLFTLLKPYPRFPFIFLSGYPFWGSVLFINSGA
FLIAVKRKTTETLIILSRIMNFLSALGAIAGIILLTFGFILDQNYICGYS
HQNSQCKAVTVLFLGILITLMTFSIIELFISLPFSILGCHSEDCDCEQCC

The NOV7a amino acid sequence has 52 of 184 amino acid residues (28%) identical to, and 88 of 184 amino acid residues (47%) similar to the Homo Sapiens 214 amino acid residue IGE receptor beta subunit protein (ptnr:REMTREMBL-ACC:AAA62319) (E=2.9e^{31 13})

NOV7a is expressed in at least the following tissues: Testis Whole Organism Male Reproductive System; SeqCalling_celltypes: testis liver spleen parathyroid_tumor. This information was derived by determining the tissue sources of the sequences that were included in the invention. SeqCalling sources: Testis Whole Organism Male Reproductive System, PublicEST sources: testis liver spleen parathyroid_tumor. In addition, NOV7a is predicted to be expressed in the following tissues because of the expression pattern of a closely related Mus musculus mast cell high affinity IgE receptor (Fc-epsilon-RI) beta subunit homolog mRNA (GENBANK-ID: gb:GENBANK-ID:MUSFCERB|acc:J05019.1):Whole Organism Male Reproductive System.

NOV7b

A disclosed NOV7b nucleic acid of 500 nucleotides (also referred to CG55760-02) encoding a novel IGE receptor beta subunit-like protein is shown in Table 7C. An open reading frame was identified beginning with an ATG initiation codon at nucleotides 26-28 and ending with a TGA codon at nucleotides 473-475. Putative untranslated regions are found upstream from the initiation codon and downstream from the termination codon in Table 7C, and the start and stop codons are in bold letters.

TABLE 7C
NOV7b Nucleotide Sequence

(SEQ ID NO:35)

CTTTCAAATTATCACCGACACCATCATGGATTCAAGCACCGCACACAGTCCGGTGTTTCTGGTATTTCCTCCCAGAAATCA
CTGCTTCAGAATATGAGTCCACAGAACTTTCAGCCACGACCTTTTCAACTCAAAGCCCCTTGCAAAAATTATTTGCTAGA
AAAATGAAAATCTTAGGCACTATCCAGATCCTGTTTGOAATTATGACCTTTTCTTTTGGAGTTATCTTCCTTTTCACCTT
GTTAAAACCATATCCAAGGTTTCCCTTTATATTTCTTTCAGGATATCCATTCTGGGGCTCTGTTTTGTTCATTAATTCTG
GACCCTTCCTAATTGCAGTGAAAAGAAAAACCACAGAAACTCTGGGAATTTTGATTACATTGATGACTTTCAGCATTATT
GAATTATTCATTTCTCTGTCTTTCTCAATTTTGGGGTGCCACTCAGAGGATTGTGATTGTGAACAATGTTGTTGACTAGC
ACTGTGAGAATAAAGATGTG

The disclosed NOV7b nucleic acid sequence, localized to chromosome 15, has 167 of 269 bases (62%) identical to a Mus musculus mast cell high affinity IgE receptor (Fc-epsilon-RI) beta subunit mRNA (gb:GENBANK-ID:MUSFCERB|acc:J05019.1) (E=0.0023).

A disclosed NOV7b polypeptide (SEQ ID NO: 36) encoded by SEQ ID NO: 35 is 149 amino acid residues and is presented using the one-letter amino acid code in Table 7D. Signal P, Psort and/or Hydropathy results predict that NOV7b contains a signal peptide and is likely to be localized in the plasma membrane with a certainty of 0.6000. The most likely cleavage site for a NOV7b peptide is between amino acids 20 and 21, at: ITA-SE.

TABLE 7D
Encoded NOV7b protein sequence.

(SEQ ID NO:36)

MDSSTAHSPVFLVFPPEITASEYESTELSATTFSTQSPLQKLFARKMKIL
GTIQILFGIMTFSFGVIFLFTLLKPYPRFPFIFLSGYPFWGSVLPINSGA
FLIAVKRKTTETLGILITLMTFSIIELFISLSFSILGCHSEDCDCEQCC

The NOV7b amino acid sequence has 117 of 122 amino acid residues (95%) identical to, and 118 of 122 amino acid residues (96%) similar to the Homo Sapiens 200 amino acid residue MS4A5 protein (ptnr:TREMBLNEW-ACC:BAB18739) (E=5.7e⁻⁵⁷).

NOV7b is expressed in at least the following tissues: testis. This information was derived by determining the tissue sources of the sequences that were included in the invention including but not limited to SeqCalling sources, Public EST sources, Literature sources, and/or RACE sources.

Possible small nucleotide polymorphisms (SNPs) found for NOV7a are listed in Table 7E.

TABLE 7E
SNPs

| | | | Amino | |
|---|---|---|---|---|
| | Neucleotide | Base | Acid | Base |
| Variant | Position | Change | Position | Change |
| 13374029 | 162 | C > T | N/A | Silent |
| 13374028 | 468 | C > T | N/A | Silent |

NOV7a and NOV7b are very closely homologous as is shown in the amino acid alignment in Table 7F.

[image]

[image]

Homologies to any of the above NOV7 proteins will be shared by the other NOV7 proteins insofar as they are homologous to each other as shown above. Any reference to NOV7 is assumed to refer to both of the NOV7 proteins in general, unless otherwise noted.

NOV7a also has homology to the amino acid sequence shown in the BLASTP data listed in Table 7G.

TABLE 7G
BLAST results for NOV7a

| Gene Index/ | | | Identity | Positives | |
|---|---|---|---|---|---|
| Identifier | Protein/Organism | Length(aa) | (%) | (%) | Expect |
| gi\|13647408\|ref\| | hypothetical | 200 | 200/200 | 200/200 | 3e−92 |
| XP_015541.1\| | protein XP_015541 | | (100%) | (100%) | |
| (XM_015541) | [Homo sapiens] | | | | |
| gi\|12965205 ref\| | testis-expressed | 200 | 199/200 | 199/200 | 4e−92 |
| NP_076434.1\| | transmembrane-4 | | (99%) | (99%) | |
| (NM_023945) | protein [Homo | | | | |
| | sapiens] | | | | |
| gi\|16184634\|ref\| | hypothetical | 239 | 47/152 | 77/152 | 9e−13 |
| XP_043510.2\| | protein XP_043510 | | (30%) | (49%) | |
| (XM_043510) | [Homo sapiens] | | | | |
| gi\|14517598\|dbj\| | similar to Fc | 239 | 47/152 | 77/152 | 1e−12 |
| BAB61018.1\| | epsilon receptor | | (30%) | (49%) | |
| (AB022821) | beta subunit | | | | |
| | [Homo sapiens] | | | | |
| gi\|16184624\|ref\| | hypothetical | 220 | 47/152 | 77/152 | 1e−12 |
| XP_015539.3\| | protein XP_015539 | | (30%) | (49%) | |
| (XM_015539) | [Homo sapiens] | | | | |

The homology of these sequences is shown graphically in the ClustalW analysis shown in Table 7H.

[image]

[image]

The IgE receptor plays a central role in allergic disease, coupling allergen and mast cell to initiate the inflammatory and immediate hypersensitivity responses that are characteristic of disorders such as hay fever and asthma. The allergic response occurs when 2 or more high-affinity IgE receptors are crosslinked via IgE molecules that in turn are bound to an allergen (antigen) molecule. A perturbation occurs that brings about the release of histamine and proteases from the granules in the cytoplasm of the mast cell and leads to the synthesis of prostaglandins and leukotrienes—potent effectors of the hypersensitivity response. The IgE receptor consists of 3 subunits: alpha, beta (147138), and gamma (147139); only the alpha subunit is glycosylated. Shimizu et al. (1988) cloned and sequenced cDNAs for the rat and human alpha subunits of high-affinity IgE receptor. Both encode an NH2-terminal signal peptide, 2 immunoglobulin-like extracellular domains (encoded by discrete exons), a hydrophobic transmembrane region, and a positively charged cytoplasmic tail. The human and rat alpha subunits share similarities with each other and with the immunoglobulin gene family, suggesting origin from a common ancestral gene, and share structural homology with their ligands. Liu et al. (1988) used a synthetic oligonucleotide homologous to the amino-terminal sequence of the alpha subunit to screen a cDNA library from a rat basophilic leukemia cell line. Nucleotide sequencing demonstrated 4 distinct varieties of cloned cDNAs, differing at the 5-prime ends and within the region encoding the second extracellular domain, suggesting the existence of at least 4 distinct protein products of the gene. By study of somatic cell hybrid DNA and in situ hybridization, Tepler et al. (1989) assigned the alpha chain of the Fc IgE receptor to human Iq21-q23. Seldin (1989) assigned the homologous mouse gene to chromosome 1. By in situ hybridization, Le Coniat et al. (1990) assigned genes for both the alpha and the gamma subunits to Iq23. Garman et al. (1998) determined the x-ray crystal structure of the antibody-binding domains of the human IgE receptor alpha subunit at 2.4-angstrom resolution. The structure revealed a highly bent arrangement of immunoglobulin domains that form an extended convex surface of interaction with IgE. A prominent loop that confers specificity for IgE molecules extends from the receptor surface near an unusual arrangement of 4 exposed tryptophans. The crystal structure of the IgE receptor provides a foundation for the development of new therapeutic approaches to allergy treatment. Garman et al. (2000) solved the crystal structure of the human IgE-Fc-FCER1A complex to 3.5-angstrom resolution. The crystal structure revealed that 1 receptor binds 1 dimeric IgE-Fc molecule asymmetrically to interactions at 2 sites, each involving 1 C-epsilon-3 domain of the IgE-Fc. The interaction of 1 receptor with the IgE-Fc blocks the binding of a second receptor, and features of this interaction are conserved in other members of the Fc receptor family. (Garman et al., Crystal structure of the human high-affinity IgE receptor. Cell 95: 951-961, 1998; Garman et al., Structure of the Fc fragment of human IgE bound to its high-affinity receptor Fc-epsilon-RI-alpha. Nature 406: 259-266, 2000; Le Coniat et al., The human genes for the alpha and gamma subunits of the mast cell receptor for immunoglobulin E are located on human chromosome band 1q23. Immunogenetics 32: 183-186, 1990; Liu et al., cDNA heterogeneity suggests structural variants related to the high-affinity IgE receptor. Proc. Nat. Acad. Sci. 85: 5639-5643, 1988; Shimizu et al., Human and rat mast cell high-affinity immunoglobulin E receptors: characterization of putative alpha-chain gene products. Proc. Nat. Acad. Sci. 85: 1907-1911, 1988; Tepleret al., The gene for the human mast cell high-affinity IgE receptor alpha chain: chromosomal localization to 1q21-q23 and RFLP analysis. Am. J. Hum. Genet. 45: 761-765, 1989).

The above defined information for NOV7 suggests that this NOV7 protein may function as a member of a IGE receptor beta subunit protein family. Therefore, the NOV7 nucleic acids and proteins of the invention are useful in potential therapeutic applications implicated in various diseases and disorders described below and/or other pathologies. For example, the NOV7 compositions of the present invention will have efficacy for treatment of patients suffering from inflammatory disorders such as osteo- and rheumatoid-arthritis, inflammatory bowel disease, Crohn's disease; immunological disorders, AIDS; cancers including but not limited to lung cancer, colon cancer, leukemia or pancreatic cancer.; blood disorders; asthma; psoriasis; inflammatory skin disordersvascular disorders, hypertension, skin disorders, renal disorders including Alport syndrome, immunological disorders, inflammation including irritable bowel disease, and tissue injury, cancers, fibrosis disorders, bone diseases, Ehlers-Danlos syndrome type VI, VII, type IV, S-linked cutis laxa and Ehlers-Danlos syndrome type V, osteogenesis imperfecta.Immuno therapy of inflammatory and infectious diseases such as AIDS, cancer therapy, treatment of Neurologic diseases, Brain and/or autoimmune disorders like encephalomyelitis, neurodegenerative disorders, Alzheimer's Disease, Parkinson's Disorder, immune disorders, and hematopoietic disorders, endocrine diseases, muscle disorders, wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome. The NOV7 nucleic acid encoding IGE receptor beta subunit-like protein, and the IGE receptor beta subunit-like protein of the invention, or fragments thereof, may further be useful in diagnostic applications, wherein the presence or amount of the nucleic acid or the protein are to be assessed.

NOV8

A disclosed NOV8 nucleic acid of 1386 nucleotides (also referred to SC138745558_A) encoding a novel Munc 18-like protein is shown in Table 8A. An open reading frame was identified beginning with an ATG initiation codon at nucleotides 36-38 and ending with a TGA codon at nucleotides 1350-1352. Putitive untranslated regions upstream from the initiation codon and downstream from the termination codon are underlined in Table 8A, and the start and stop codons are in bold letters.

TABLE 8A
NOV8 Nucleotide Sequence

(SEQ ID NO:37)

CACTCTGGCCGTGGCCGACGTTGCTCCTCCGAAGCATGGCGCCGGCGGGAGTTGTAACCCGGGCTGTCCGGAGCGGGGAG
CTGCCCCTCACAAGCATGGCGTCAGCTGAAAATGAAGCCTGTGCTGTGCGGAGCGTCGCCTGCCCCTCACAAGCATGGCG
TCTGCAGAAGGTGCTGTGCGGCCGCTGCGGGGCGGCCAGCTGCCCTTCACAAACATGGCGGCCGAGGGGTGCGGGGAGTG
GCGGGGTAAGGATGGGAAGCCGAGCAGACGGCCCCAGAACAAGCGGTCATGTGACTGGGAAGATGGCCGTCTTTCCTTGG
CACTCCAGGAATAGGAACTACAAAGCTGAATTTGCATCATGCCGACTGGAGGCTGTACCATTGGAGTTTGGGGACTATCA
CCCTCTGAAACCCATAACTGTCACAGAGTCAAAGACAAAGAAAGTGAACCGGAAAGGAAGCACTTCTTCCACGTCCTCCT
CCTCCTCCAGCTCCGTGGTGGACCCGCTGAGCAGCGTCCTCGATGGGACTGACCCCCTCTCCATGTTTGCAGCCACTGCT
GACCCCGCAGCCTTGGCAGCTGCCATGGACAGCTCCAGAAGGAAACGTGATAGAGATGATAACTCCGTTGTAGGATCGCA
TTTTGAGCCTTGGACCAACAAACGGGGAGAAATCCTTGCCCGGTACACCACTACCGAAAAGCTGTCTATTAATCTGTTTA
TGGGATCTGAAAAAGGCAAAGCTGCGACTGCCACATTCCCAATGTCAGAGAAGGTGCGGACCCGGCTGGAGGAGCTGGAT
GACTTTGAGGAGCGTTCCCAAAAGGAGCTGTTGAACTTGACTCAGCAGGATTACGTGAACCGCATAGAGGAGCTCAACCA
ATCGCTGAAGGATGCCTGGGCCTCAGACCAGAAAGTGAAGGCTCTAAAAATAGTCATCCAGTGTTCAAAGCTTCTTTCAG
ACACCAGTGTTATTCAGTTCTACCCAAGCAAATTTGTCCTTATCACCGACATACTTGATACATTTGGAAAGCTCGTGTAC
GAGCGCATCTTTTCCATGTGTGTGGATAGCCGCAGCGTCTTACCAGATCACTTTTCTCCAGAGAATGCAAATGACACGGC
CAAGGAAACATGCCTAAATTGGTTTTTCAAGATTGCCTCCATCAGGGAACTCATTCCAAGATTTTACGTGGAGGCATCCA
TCCTGAAATGTAACAAATTCCTCTCCAAAACGGGAATTTCAGAGTGCCTGCCCCGGTTGACATGCATGATCAGAGGGATC
GGAGACCCACTAGTGTCGGTGTATGCCCGTGCCTACCTGTGCCGGGTAGGCCATGCGAGTCACTGCCCTTGACGGCACGG
CTGCCACCCGTCTCCTTGAATGTTCC

The disclosed NOV8 nucleic acid sequence is localized to chromosome 16.

A disclosed NOV8 polypeptide (SEQ ID NO: 38) encoded by SEQ ID NO: 37 is 438 amino acid residues and is presented using the one-letter amino acid code in Table 8B. Signal P, Psort and/or Hydropathy results predict that NOV8 does not contain a signal peptide and is likely to be localized to the mitochondrial matrix space with a certainty of 0.4363.

TABLE 8B
Encoded NOV8 protein sequence.

(SEQ ID NO:38)

MAPAGVVTRAVRSGELPLTSMASAENEACAVRSVACPSQAWRLQKVLCGRCGAASCPSQTWRPRGAGSGGVRMGSRADGP
RTSGHVTGKMAVFPWHSRNRNYKAEFASCRLEAVPLEFGDYHPLKPITVTESKTKKVNRKGSTSSTSSSSSSSVVDPLSS
VLDGTDPLSMFAATADPAALAAAMDSSRRKRDRDDNSVVGSDFEPWTNKRGEILARYTTTEKLSINLFMGSEKGKAGTAT
LAMSEKVRTRLEELDDFEEGSQKELLNLTQQDYVNRIEELNQSLKDAWASDQKVKALKIVIQCSKLLSDTSVIQFYPSKF
VLITDILDTFGKLVYERIFSMCVDSRSVLPDHFSPENANDTAKETCLNWFFKIASIRELIPRFYVEASILKCNKFLSKTG
ISECLPRLTCMIRGIGDPLVSVYARAYLCRVGHASHCP

The NOV8 amino acid sequence has 204 of 204 amino acid residues (100%) identical to, and 204 of 204 amino acid residues (100%) similar to, a Homo sapiens 824 amino acid residue FLJ21040 FIS, CLONE CAE10642 protein (ptnr:TREMBLNEW-ACC:BAB14965)(E=1.9e⁻¹⁰⁵).

NOV8a is expressed in at least the following tissues: Adrenal Gland/Suprarenal gland, Amygdala, Cervix, Pituitary Gland, Thymus, Tonsils, Whole Organism, SeqCalling_celltypes: liver, spleen, testis, tumor. This information was derived by determining the tissue sources of the sequences that were included in the invention. SeqCalling sources: Adrenal Gland/Suprarenal gland, Amygdala, Cervix, Pituitary Gland, Thymus, Tonsils, Whole Organism; PublicEST sources: liver, spleen, testis, tumor.

The disclosed NOV8 polypeptide has homology to the amino acid sequences shown in the BLASTP data listed in Table 8C.

TABLE 8C
BLAST results for NOV8

| Gene Index/ | | Length | Identity | Positives | |
|---|---|---|---|---|---|
| Identifier | Protein/Organism | (aa) | (%) | (%) | Expect |
| gi\|12858347\|dbj\| | putative [Mus | 434 | 317/343 | 324/343 | 1e−178 |
| BAB31285.1\| (AK018573) | musculus] | | (92%) | (94%) | |
| gi\|14777378\|ref\| | hypothetical | 824 | 204/204 | 204/204 | 1e−120 |
| XP_040052.1\| | protein XP_040052 | | (100%) | (100%) | |
| (XM_040052) | [Homo sapiens] | | | | |
| gi\|12847855\|dbj\| | putative [Mus | 241 | 213/244 | 222/244 | 1e−108 |
| BAB27735.1\| (AK011615) | musculus] | | (87%) | (90%) | |
| gi\|15292597\|gb\| | SD10311p | 942 | 146/303 | 199/303 | 9e−69 |
| AAK93567.1 (AY052143) | [Drosophila | | (48%) | (65%) | |
| | melanogaster] | | | | |
| gi\|7299081\|gb\| | CG8202 gene | 979 | 134/303 | 182/303 | 1e−56 |
| AAF54281.1\| (AE003680) | product | | (44%) | (59%) | |
| | [Drosophila | | | | |
| | melanogaster] | | | | |

The homology between these and other sequences is shown graphically in the ClustalW analysis shown in Table 8D.

[image]

[image]

[image]

The Sec1-related proteins bind to syntaxin family t-SNAREs with high affinity, thus controlling the interaction of syntaxins with their cognate SNARE partners. Munc 18-2 is a Sec1 homologue enriched in epithelial cells and forms a complex with syntaxin 3, a t-SNARE localized to the apical plasma membrane. Mutational studies implied that Munc 18-2 function in apical membrane trafficking involves aspects independent of the syntaxin 3 interaction (Riento et al., Munc 18-2, a functional partner of syntaxin 3, controls apical membrane trafficking in epithelial cells. J Biol Chem 275(18):13476-83, 2000).

The Q-SNARE syntaxin 1 is a central component of the synaptic membrane fusion machinery. Syntaxin probably interacts with multiple proteins during synaptic vesicle exocytosis. In vitro, the tightest binding partners for syntaxin 1 are other SNAREs (synaptobrevin/VAMP and SNAP-25) and munc18-1 (also known as rbsec1/nsec1) (Matos et al., The relation of protein binding to function: what is the significance of munc 18 and synaptotagmin binding to syntaxin 1, and where are the corresponding binding sites? Eur J Cell Biol 79(6):377-82, 2000).

Mint1 (X11/human Lin-10) and Mint2 are neuronal adaptor proteins that bind to Munc18-1 (n/rb-sec1), a protein essential for synaptic vesicle exocytosis. Mint1 has previously been characterized in a complex with CASK, another adaptor protein that in turn interacts with neurexins. Neurexins are neuron-specific cell surface proteins that act as receptors for the excitatory neurotoxin-latrotoxin. Hence, one possible function for Mint1 is to mediate the recruitment of Munc18 to neurexins. In agreement with this hypothesis, it was shown that the cytoplasmic tail of neurexins captures Munc18 via a multiprotein complex that involves Mint1. Furthermore, it was demonstrated that both Mint1 and Mint2 can directly bind to neurexins in a PDZ-domain mediated interaction. Various Mint and/or CASK containing complexes can be assembled on neurexins, and we demonstrate that Mint1 can bind to Munc18 and CASK simultaneously. These data support a model whereby one of the functions of Mints is to localize the vesicle fusion protein Munc18 to those sites at the plasma membrane that are defined by neurexins, presumably in the vicinity of points of exocytosis (Biederer and Sudhof, Mints as adaptors. Direct binding to neurexins and recruitment of munc18. J Biol Chem 275(51):39803-6,2000).

The above defined information for NOV8 suggests that NOV8 may function as a member of a Munc18 protein family. Therefore, the NOV8 nucleic acids and proteins of the invention are useful in potential therapeutic applications implicated in various diseases and disorders described below and/or other pathologies. For example, the NOV8 compositions of the present invention will have efficacy for treatment of patients suffering from inflammatory disorders such as osteo- and rheumatoid-arthritis, inflammatory bowel disease, Crohn's disease; immunological disorders, AIDS; cancers including but not limited to lung cancer, colon cancer, leukemia or pancreatic cancer.; blood disorders; asthma; psoriasis; inflammatory skin disordersvascular disorders, hypertension, skin disorders, renal disorders including Alport syndrome, immunological disorders, tissue injury, cancers, fibrosis disorders, bone diseases, Ehlers-Danlos syndrome type VI, VII, type IV, S-linked cutis laxa and Ehlers-Danlos syndrome type V, osteogenesis imperfecta, Immuno therapy of inflammatory and infectious diseases such as AIDS, treatment of Neurologic diseases, Brain and/or autoimmune disorders like encephalomyelitis, neurodegenerative disorders, Alzheimer's Disease, Parkinson's Disorder, immune disorders, and hematopoietic disorders, endocrine diseases, muscle disorders, wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, multiple sclerosis; and Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome. The NOV8 nucleic acid encoding Munc18-like protein, and the Munc18-like protein of the invention, or fragments thereof, may further be useful in diagnostic applications, wherein the presence or amount of the nucleic acid or the protein are to be assessed.

NOV9

NOV9 includes two novel Immunoglobulin-like proteins disclosed below. The disclosed proteins have been named NOV9a and NOV9b.

NOV9a

A disclosed NOV9a nucleic acid of 1514 nucleotides (also referred to SC138673511_A) encoding a novel Immunoglobulin-like protein is shown in Table 9A. An open reading frame was identified beginning with an ATG initiation codon at nucleotides 29-31 and ending with a TGA codon at nucleotides 1319-1321. Putative untranslated regions upstream from the initiation codon and downstream from the termination codon is underlined in Table 9A, and the start and stop codons are in bold letters.

TABLE 9A

NOV9a Nucleotide Sequence

| | |
|---|---|
| CTCTGTAAGGGCCAGAAGTGAGGCAGGGATGGGGGTGTTGGATGCCTGGATGGGCCAGAGGTCTGGGTCCCCCAGGGGG | (SEQ ID NO:39) |
| CAGCGGCCAAAGGTCCAGACCCCCCAAGTCCAGTGAGGGCAGTAGGGATTGGGTTGGGGGAAGATAACTGGGGGAAGGCC | |
| AGGGCCCGGCGCTTCCAGTGGCCGCTGCTGCTGCTGTGGCGGCCGCGGCGGGGCCAGGGGAGACAGGAAGTAAGAC | |
| AGAGAACGTGACAGTGGCTGAGGGTGGGGTGGCTGAGATCACCTGCCGTCTGCACCAGTATGATGGGTCCATAGTTGTCA | |
| TCCAGAACCCAGCCCGGCAGACCCTCTTCTTGATGGACCCGTGCCTTGAGGATGAGCGTTTCCAGCTTGAAAGTTC | |
| TCCCCACGCCGGGTGCGGATCCGGCTCTCAGATGCCCGCCTGGAGGACGAAAGGCTATTTCTGCCAGCTCTAAAGA | |
| AGACACGACCACCGATTGCCACGCTCACGGTACTAGTGGCCCCAGAGAATCCTGTCGTGAGGTCCGGGAGAGGCGG | |
| TAGAGGGCCGCGAGGTGGAGCTCAGCTGCCTCGTTCCGCGGTCCCGTCCGGCTGCACCCTGCGCTATACCGGACCGC | |
| AGGAGCTGAAGGAGTGAGCAGCAGCCAGGAAATGGCAAGGTCTGCAGCGTGGACCAAGTACATTTCGTGTGGA | |
| CCGTAAGGACGACGGTGGTATCATCATCTGTGAGGCGCAGAACCAGGCGCTGCCCTCCGGACACAGCAAGCAGACGCAGT | |
| ACGTGCTGGATGTGCAGTACTCCCCCACGGCCCGGATTCATGCCTCCCAAGCTGTGGTGAGGGAGGGAGACACGCTGGTG | |
| TTGACGTGTGCTGTCACGGGGAACCCCAGGCCAACCAGATCCGCTGGACCGCGGGATGAGTCTTTGCCGGAGAAGC | |
| GGAGGCCGTGGAGGACGCTCACGCTGCCGGGTCTGGTATCCGCGGATACACACCTACACTTGCAGGCGTCCATA | |
| AGCACGGCCTGCGAGGGCGCTCTACGTACTTGTGGTCTACGACCCTGGTGCGGTATAGAGGCTCAACGTCATTCCC | |
| TATGCCATTGTGGGCGGCATCCTGGCGCTGCTGGTGTTTCTGATCATATGTGTGCTAGTGGGATGGTCTGGTGCTCGGT | |
| ACGGCAGAAGGGTTCCTATCTGACCCACGAAGCCAGTGGCTTGGATGAAAAGGGAGCAGAAAGCCTTCCTCAATG | |
| GCAGCGACGGACACAAGAGGAAAGAGGAATTCTTCATCTGACCCTATCCCCAGCCAGCCTAACCTGACCTGGGCTGGG | |
| GTCCCCCCCACTGCCAGCTGCAAGGAACCAGCAAAGACATTTACCAGAGTCTGGGATGGTGGGCTTCTCCCCCCACCACT | |
| AACACCTCAGACGCTTGGGCAGGGATGGGGGTGTTGGATGCCTGGATCTCTGTAGGGCCAGAGTGAGGGCCC | |

The disclosed NOV9a nucleic acid sequence, maps to chromosome 19, has 510 of 852 bases (59%) identical to a Mus musculus immunosuperfamily protein B12 mRNA from (gb:GENBANK-ID:AF061260|acc:AF061260) (E=7.6e⁻²¹).

A disclosed NOV9a polypeptide (SEQ ID NO: 40) encoded by SEQ ID NO: 39 is 430 amino acid residues and is presented using the one-letter amino acid code in Table 9B. Signal P, Psort and/or Hydropathy results predict that NOV9a contains a signal peptide and is likely to be localized in the plasma membrane with a certainty of 0.7300. The most likely cleavage site for a NOV9a peptide is between amino acids 66 and 67, at: GAG-QE.

TABLE 9B

Encoded NOV9a protein sequence.

| | |
|---|---|
| MGVLDAWMGPEVWVPQGAGAKGPDPPSPVRAVGIGLGEDNWGKARARRFQWPLLLLWAAAAGPGAGQEVQTENVTVAEGG | (SEQ ID NO:40) |
| VAEITCRLHQYDGSIVVIQNPARQTLFFNGTRALKDERFQLEEPSPRRVRIRLSDARLEDEGGYFCQLYTEDTHHQIATL | |
| TVLVAPENPVVEVREQAVEGGEVELSCLVPRSRPAATLRWYRDRKELKGVSSSQENGKWSVASTVRFRVDRKDDGGIII | |
| CEAQNQALPSGHSKQTQYVLDVQYSPTARIHASQAVVREGDTLVLTCAVTGNPRPNQIRWRGNESLPERAEAVGETLTL | |
| PGLVSADNGTYTCEASNKHGHARALYVLVVYDPGAVVEAQTSVPYAIVGGIALLVFLIICVLVGMVWCSVRQKGSYLTH | |
| EASGLDEQGEAREAFLNGSDGHKRKEEFFI | |

The NOV9a amino acid sequence has 366 of 381 amino acid residues (96%) identical to, and 366 of 381 amino acid residues (96%) similar to, the Homo sapiens 381 amino acid residue F22162_{—}1 protein (ptnr:SPTREMBL-ACC:Q9Y4A4) (E=2.8e⁻¹⁹¹).

NOV9a is expressed in at least the following tissues: Amygdala, Brain, Coronary Artery, Heart, Hippocampus, Hypothalamus, Kidney, Lung, Pituitary Gland, Spinal Chord, Substantia Nigra, Thalamus, Whole Organism, SeqCalling_celltypes: glioblastoma total_fetus schizo brain brain oligodendroglioma Fetal brain. This information was derived by determining the tissue sources of the sequences that were included in the invention. SeqCalling sources: Amygdala, Brain, Coronary Artery, Heart, Hippocampus, Hypothalamus, Kidney, Lung, Pituitary Gland, Spinal Chord, Substantia Nigra, Thalamus, Whole Organism, PublicEST sources: glioblastoma total_fetus schizo brain brain oligodendroglioma Fetal brain. In addition, NOV9a is predicted to be expressed in the following tissues because of the expression pattern of a closely related Mus musculus immunosuperfamily protein B12 mRNA (GENBANK-ID: gb:GENBANK-ID:AF061260|acc:AF061260): glioblastoma total_fetus schizo brain brain oligodendroglioma Fetal brain.

NOV9b

A disclosed NOV9b nucleic acid of 1161 nucleotides (also referred to CG106625-02) encoding a novel Immunoglobulin-like protein is shown in Table 9C. An open reading frame lacking the signal peptide was identified beginning with an GCC codon at nucleotides 2-4 and ending with a TGA codon at nucleotides 1157-1159. Putative untranslated regions upstream from the initiation codon and downstream from the termination codon is underlined in Table 9C, and the start and stop codons are in bold letters.

TABLE 9C

NOV9b Nucleotide Sequence

| | |
|---|---|
| GGCCCGGCGCTTCCAGTGGCCGCTGCTGCTGCTGTGGGCGGCCGCGGCGGGGCGGAAGGAAGGAGTAAGACAG | (SEQ ID NO:41) |
| AGAACGTGACAGTGGCTGAGGTGGGGTGGCTGAGATACCTGCCGTCTGCACAGTATGATGGGTCATAGTTGTATC | |
| CAGAACCCAGCCCGGCAGACCCTCTTCTTCAATGGCACCCCTGCCTTGAGGATGACCGTTTCCAGCTTCAGGAGTTCTC | |
| CCCACGCCGGGTGCGGATCCGGCTCTCAGATGCCCGCCTGGAGGACGAGAGGGCTATTTCTGCGAGCTCTACACAGAG | |
| ACACCCACCACCAGATTGCCACGCTCACGGTACTAGTGGCCCCAGAGAATCCTGTGGTGGAGGTCCGGGAGCAGGCGGTA | |
| GAGGGCGGCGAGGTGGAGCTCAGCTGCCTCGTTCCGCGTCCCGTCCGGCTGCCACCCTGCGCTGGTACCAGACCGCA | |
| GCAGCTGAAAGQAGTGACCAGCACCCAGGAAATGCCAAGGTCTGGAGCGTGGACCAAGTACGGTTTCGTGTGCACC | |
| GTAAGACGACGTGGTATCATCATCTGTGAGGCGCAGAACCAGGCGCTGCCCTCCGGAAAGCAGCAGACGAGTAC | |
| GTGCTGGATGTGCAGTACTCCCCCACGGCCCGGATTCATGCCTCCCAAGCTGTGGTGAGGGAGGGAGACACGCTGGTGTT | |
| GACGTGTGGTGTCACGGGGAACCCCAGGCCAAACCAGATCCGCTGGAACCGCGGGAATCAGCTTTGCCGAGAGGGCGG | |
| AGGCCGTGGGAGAGACGCTCACGCTGCCGGGTCTGGTATCCGCGGATAACGGCACCTACACTTGCGAGGCGTCCAATAAG | |
| CACGGCCATGCOAGGGCQCTCTACGTACTTCTGGTCTACGACCCTGGTACGGTCGTAGACGCTAGACGTCCGTTCCCTA | |
| TGCCATTGTGGGCGGCATCCTGGCGCTGCTGGTGTTTCTGATCATATGTGTGCTAGTGGGCATGGTCTGGTGCTCGGTAC | |
| GGCAGAAGGGTTCCTATCTGACCCACGAAGCCAGTGGCTTGGATGAACAGGGAGAAGCAAGAGAAGCCTTCCTCAATGGC | |
| AGCGACGGACACAAGAGGAAAGAGGAATTCTTCATCTGACC | |

The disclosed NOV9b nucleic acid sequence, maps to chromosome 19, has 557 of 931 bases (59%) identical to a Mus musculus sgigsf mRNA for spermatogenic immunoglobulin superfamily protein (gb:GENBANK-ID:AB052293|acc:AB052293.1) (E=2.1e⁻²⁶).

A disclosed NOV9b polypeptide (SEQ ID NO: 42) encoded by SEQ ID NO: 41 is 385 amino acid residues and is presented using the one-letter amino acid code in Table 9D. Signal P, Psort and/or Hydropathy results predict that NOV9b contains a signal peptide and is likely to be localized in the plasma membrane with a certainty of 0.4600. The most likely cleavage site for a NOV9b peptide is between amino acids 21 and 22, at: GAG-QE.

TABLE 9D

Encoded NOV9b protein sequence.

| | |
|---|---|
| ARRFQWPLLLLWAAAAGPGAGQEVQTENVTVAEGGVAEITCRLHQYDGSIVVIQNPARQTLFFNGTRALKDERFQLEEFS | (SEQ ID NO:42) |
| PRRVRIRLSDARLEDEGGYFCQLYTEDTHHQIATLTVLVAPENPVVEVREQAVEGGEVELSCLVPRSRPAATLRWYRDRK | |
| ELKGVSSSQENGKVWSVASTVRFRVDRKDDGGIIICEAQNQALPSGHSKQTQYVLDVQYSPTARIHASQAVVREGDTLVL | |
| TCAVTGNPRPNQIRWNRGNESLPERAEAVGETLTLPGLVSADNGTYTCEASNKHGHARALYVLVVYDPGAVVEAQTSVPY | |
| AIVGGILALLVFLIICVLVGMVWCSVRQKGSYLTHEASGLDEQGEAREAFLNGSDGHKRKEEFFI | |

The NOV9b amino acid sequence has 366 of 381 amino acid residues (96%) identical to, and 366 of 381 amino acid residues (96%) similar to, the Homo sapiens 381 amino acid residue F22162_{—}1 protien (ptnr:SPTREMBL-ACC:Q9Y4A4) (E=3.7e⁻¹⁹¹).

NOV9b is expressed in at least the following tissues: Amygdala, Brain, Coronary Artery, Heart, Hippocampus, Hypothalamus, Kidney, Lung, Pituitary Gland, Spinal Chord, Substantia Nigra, Thalamus, Whole Organism. This information was derived by determining the tissue sources of the sequences that were included in the invention. SeqCalling sources: Amygdala, Brain, Cornoary, Artery, Heart, Hippocampus, Hypothalamus, Kidney, Lung, Pituitary Gland, Spinal Chord, Substantia Nigra, Thalamus, Whole Organism, PublicEST sources: glioblastoma total_fetus schizo brain brain oligodendroglioma Fetal brain. In addition, NOV9b is predicted to be expressed in the following tissues because of the expression pattern of a closely related Mus musculus sgigsf mRNA for spermatogenic immunoglobulin superfamily protein (gb:GENBANK-ID:AB052293|acc:AB052293.1): Fetal brain.

NOV9a and NOV9b are very closely homologous as is shown in the amino acid alignment in Table 9E.

[image]

[image]

Homologies to any of the above NOV9 proteins will be shared by the other NOV9 proteins insofar as they are homologous to each other as shown above. Any reference to NOV9 is assumed to refer to both of the NOV9 proteins in general, unless otherwise noted.

NOV9a also has homology to the amino acid sequences shown in the BLASTP data listed in Table 9F.

TABLE 9F
BLAST results for NOV9a

| Gene Index/ | | Length | Identity | Positives | |
|---|---|---|---|---|---|
| Identifier | Protein/Organism | (aa) | (%) | (%) | Expect |
| gi\|3451335\|gb\| | F22162_1 [Homo | 381 | 366/381 | 366/381 | 0.0 |
| AAC32740.1\| (AC005525) | sapiens] | | (96%) | (96%) | |
| gi\|7767239\|gb\| | nectin-like | 442 | 159/416 | 242/416 | 1e−68 |
| AAF69029.1\|AF132811_1 | protein 2 [Homo | | (38%) | (57%) | |
| (AF132811) | sapiens] | | | | |
| gi\|7657226\|ref\| | immunoglobulin | 442 | 158/416 | 242/416 | 2e−68 |
| NP_055148.1\| | superfamily, | | (37%) | (57%) | |
| (NM_014333) | member 4 [Homo | | | | |
| | sapiens] | | | | |
| gi\|14328885\|dbj\|BAB | spermatogenic | 445 | 160/420 | 243/420 | 1e−66 |
| 60686.1\| (AB052293) | immunoglobulin | | (38%) | (57%) | |
| | superfamily | | | | |
| | protein [Mus | | | | |
| | musculus] | | | | |
| gi\|12851464\|dbj\|BAB | putative [Mus | 494 | 160/431 | 246/431 | 5e−63 |
| 29050.1\| (AK013911) | musculus] | | (37%) | (56%) | |

The homology of these sequences is shown graphically in the ClustalW analysis shown in Table 9G.

[image]

[image]

Tables 9H -9P list the domain description from DOMAIN analysis results against NOV9. This indicates that the NOV9 sequence has properties similar to those of other proteins known to contain these domains.

TABLE 9H

Domain Analysis of NOV9a
gnl|Smart|smart00408, IGc2, Immunoglobulin C-2 Type
Length = 63 residues, 100.0% aligned
Score = 63.2 bits (152), Expect = 3e−11

| | | | | |
|---|---|---|---|---|
| NOV9a | 278 | REGDTLVLTCAVTGNPRPNQIRWNRGNESLPE-RAEAVGETLTLPGLVSADNGTYTCEAS | 336 | (SEQ ID NO:114) |
| | | \|\|+++ \|\|\| +\|+\| \|\| \| \| + + \|\|\| \| \| \| \|\|\|+ + \|+\| \|\|\| \| | | |
| 00408 | 1 | LEGESVTLTCPASGDPVPN-ITWLKDCKPLPESRVVASGSTLTIKNVSLEDSGLYTCVAR | 59 | |
| NOV9a | 337 | NKHG 340 | | |
| | | \| \| | | |
| 00408 | 60 | NSVG 63 | | |

TABLE 9I

| | |
|---|---|
| Domain Analysis of NOV9a | +HL,57 |

gnl|Smart|smart00408, IGc2, Immunoglobulin C-2 Type
Length = 63 residues, 96.8% aligned
Score = 40.4 bits (93), Expect = 2e−04

| NOV9a | 179 | EGGEVELSCLVPRSRPAATLRWYRDRKELKGVSSSQENQKVWSVASTVRFRVDRKDDGGI | 238 | (SEQ ID NO:115) |
|---|---|---|---|---|
| | | \|\| \| \|+\| \| + \| +\| \| \| \|\|+ + +\| \|+ | | |
| 00408 | 2 | EQESVTLTCPA-SGDPVPNITWLKDGKPLPESRVVASG------STLTIKNXTSLEDSGL | 53 | |
| NOV9a | 239 | IICEAQNQA 247 | | |
| | | \| \|+\| | | |
| 00408 | 54 | YTCVARNSV 62 | | |

TABLE 9J

Domain Analysis of NOV9a
gnl|Smart|smart00409, IG. Immunoglobulin
Length = 86 residues, 98.8% aligned
Score = 60.5 bits (145), Expect = 2e−10

| | | | | |
|---|---|---|---|---|
| NOV9a | 273 | SQAVVREGDTLVLTCAVTGNPRPNQIRWNRGNESL---PERAEAVGE----TLTLPGLVS | 325 | (SEQ ID NO:116) |
| | | \|+\|\|+++ \|+\| +\|\|\| \| + \| + \| \| \|\|\|+ + | | |
| 00409 | 2 | PSVTVKEGESVTLSCEASGNPPPATVTWYKQGGKLLAESGRFSVSRSGGNSTLTISNVTP | 60 | |
| NOV9a | 326 | ADNGTYTCEASNKEGHARALYVLVVY 351 | | |
| | | \|+\|\|\|\|\| \|+\| \| \| + \| \| | | |
| 00409 | 61 | EDSGTYTCAATNSSGSASSGTTLTVL 86 | | |

TABLE 9K

Domain Analysis of NOV9a
gnl|Smart|smart00409, IG, Immunoglobulin
Length = 86 residues, 98.8% aligned
Score = 44.7 bits (104), Expect = 1e−05

| | | | | |
|---|---|---|---|---|
| NOV9a | 72 | ENVTVAEGGVAEITCRLHQYDGSIVVIQNPARQTLFFNGTRALKDERFQLEEFSPRRVRI | 131 | (SEQ ID NO:117) |
| | | +\|\|\| \|\| ++\| \| + \| +\| \|\| + \| + | | |
| 00409 | 2 | PSVTVKEGESVTLSCEASGNPPPTVTWYKQGGKLLAESG-------RFSVSR-SGGNSTL | 53 | |
| NOV9a | 132 | RLSDARLEDEGGYFCQLYTED-THHQIATLTVL | 163 | |
| | | +\|+ \|\| \| \| \| + \|\|\|\|\| | | |
| 00409 | 54 | TISNVTPEDSGTYTCAATNSSGSASSGTTLTVL | 86 | |

TABLE 9L

Domain Analysis of NOV9a

gnl|Smart|smart004C9, 1G. Immunoglobulin
Length = 86 residues, 77.9% aligned
Score = 36.6 bits (83), Expect = 0.003

| | | | | |
|---|---|---|---|---|
| NOV9a | 177 | AVEGGEVELSCLVPRSRPAATLRWYRDRKELKGVSSSQENGKVWSVASTVRFRVD--RKD | 234 | (SEQ ID NO:118) |
| | | \|\| \| \|\|\| \| \|+ \|\|+ +\| + \| + + | | |
| 00409 | 6 | VKEGESVTLSCEAS-GNPPPTVTWYKQGGKL---LAESGRFSVSRSGGNSTLTISNVTPE | 61 | |
| NOV9e | 235 | DGGIIICEAQN 245 | | |
| | | \| \| \| \| \| | | |
| 00409 | 62 | DSGTYTCAATN 72 | | |

TABLE 9M

Domain Analysis of NOV9a
gnl|Pfam|pfam00047, ig, Immunoglobulin domain. Members of the
immunoglobulin superfamily are found in hundreds of proteins of
different functions. Examples include antibodies, the giant muscle
kinase titin and receptor tyrosine kinases. Immunoglobulin-like
domains may be involved in protein-protein and protein-ligand
interactions. The Pf am alignments do not include the first and last
strand of the immunoglobulin-like domain.
Length = 68 residues, 100.0% aligned
Score = 38.9 bits (89), Expect = 6e−04

| | | | | |
|---|---|---|---|---|
| NOV9a | 180 | GGEVELSCLVPRSRPAATLRWYRDRKELKGVSSSQE----NGKVWSVASTVRFRVDRKDD | 235 | (SEQ ID NO:119) |
| | | \| \| \|+\| \| \| \|+ \| \|\| \|\|++ + \|\|+ \|+ + ++ +\| | | |
| 00047 | 1 | GESVTLTCSVSGYPPDPTVTWLRDGKEIELLGSSESRVSSGGRFSISSLSLTISSVTPED | 60 | |
| NOV9a | 236 | GGIIICEA 243 | | |
| | | \| \| | | |
| 00047 | 61 | SGTYTCVV 68 | | |

TABLE 9N
Domain Analysis of NOV9a

gnl|Pfam|pfam00047, ig, Immunoglobulin domain. (SEQ ID NO:120)
Length = 68 residues, 100.0% aligned
Score = 38.9 bits (89), Expect = 6e−04

| | | | |
|---|---|---|---|
| NOV9a | 280 | GDTLVLTCAVTGNPRPNQIRWNRGNE------------SLPERAEAVGETLTLPGLVSAD | 327 |
| | | \|+++ \|\|\|+\|+\| \| + \| \| \| + \| \| +\|\|+ + \| | |
| 00047 | 1 | GESVTLTCSVSGYPPDPTVTWLRDGKEIELLGSSESRVSSGGRFSISSLSLTISSVTPED | 60 |
| NOV9a | 328 | NGTYTCEA 335 | |
| | | +\|\|\|\|\| | |
| 00047 | 61 | SGTYTCVV 68 | |

TABLE 9O
Domain Analysis of NOV9a

gnl|Smart|smart00294, 4.1 m, putative band 4.1
homologues' binding (SEQ ID NO:121) motif
Length = 19 residues, 100.0% aligned
Score = 38.5 bits (88), Expect = 8e-04

| | | | |
|---|---|---|---|
| NOV9a | 386 | MVWCSVRQKGSYLTHEASG | 404 |
| | | \| \| +\|\|\| \|\|\| \| | |
| 00294 | 1 | MYRYKHPDEGSYHTHEPKG | 19 |

TABLE 9P
Domain Analysis of NOV9a

gnl|Smart|smart00406, IGv, Immunoglobulin V-Type (SEQ ID NO:122)
Length = 80 residues, 97.5% aligned
Score = 37.0 bits (84), Expect = 0.002

| | | | |
|---|---|---|---|
| NOV9a | 82 | AEITCRLHQYDGSIVVI----QNPARQTLFF-------NGTRALKDERFQL-EEFSPRRV | 129 |
| | | ++\|+ + \| + \| \| + + + + \| \| + ++ \| \| | |
| 00406 | 2 | VTLSCKASGFTFSSYYVSWVRQPPGKGLEWLGYIGSDVSYSEASYKGRVTISKDNSKNDV | 61 |
| NOV9a | 130 | RIRLSDARLEDECGYFCQ 147 | |
| | | + +\|+ \|+\|\| \| \|+\| | |
| 00406 | 62 | SLTISNLRVEDTGTYYCA 79 | |

The effect of rIL-4 on the expression of low affinity receptor for the Fc part of IgE (Fc epsilon R2/CD23) and class II MHC antigens on Burkitt's lymphoma (BL) cell lines was investigated. Some of the BL lines contained low percentages of CD23 and HLA-DQ-positive cells, but virtually all cells expressed HLA-DR. IL-4 induced CD23 and class II MHC Ag expression on 7 of 9 BL. Optimal CD23 and class II MHC expression was observed after 48-72 h of incubation. Induction of CD23 and class II MHC Ag in the BL cell line BL2 by IL-4 was confirmed at the specific mRNA level. Significant activation of HLA-DQ mRNA was obtained after 6 h of incubation with IL-4 and gradually increased during prolonged incubation. Maximal induction of mRNA transcription occurred after 48 to 72 h. Optimal induction of HLA-DR and CD23 transcription in BL2 was also observed after 48 to 72 h. The induction of CD23 and class II MHC Ag seems to be specific for IL-4, because rIL-l, rIL-2, rIFN-gamma, recombinant granulocyte-macrophage-CSF, and a commercial source of low m.w. B cell growth factor were ineffective. In addition, the expression of class I MHC Ag, the transferrin receptor, CD38, CD25, CD10, CD20, and CD21 were not affected by IL-4. Interestingly, IFN-gamma and PGE2 suppressed the IL-4-induced membrane expression of CD23 and class II MHC Ag in a dose-dependent way. IFN-gamma also blocked IL-4-induced CD23 mRNA transcription in BL2 completely, whereas PGE2 (10(−7) M) was partially inhibitory. The induction of CD23 and class II MHC Ag by IL-4 required intact protein synthesis as shown by its inhibition by cycloheximide. These results indicate that the induction of CD23 and class II MHC Ag by IL-4 is regulated in a coordinated way (Rousset et al., Regulation of Fc receptor for IgE (CD23) and class II MHC antigen expression on Burkitt's lymphoma cell lines by human IL-4 and IFN-gamma. J Immunol 140(8):2625-32, 1988).

The B cell surface trigger(s) and the molecular mechanism(s) of somatic hypermutation remain unknown, partly because of the lack of amendable in vitro models. Recently, however, it was reported that upon B cell receptor cross-linking and coculture with activated T cells, the Burkitt's lymphoma cell line BL2 introduces mutations in its IgVH gene in vitro. The relevance of a culture model is confirmed by establishing that the entire spectrum of somatic mutations observed in vivo, including insertions and deletions, could be found in the DNA of BL2 cells. Additionally, it was showed that among four human B cell lines, only two with a centroblast-like phenotype can be induced to mutate. Triggering of somatic mutations in BL2 cells requires intimate T-B cell contacts and is independent of CD40-CD40-ligand (CD40L) interactions as shown by 1) the lack of effect of anti-CD40 and/or anti-CD40L blocking Abs on somatic mutation and 2) the ability of a CD40L-deficient T cell clone (isolated from an X-linked hyper-IgM syndrome patient) to induce somatic mutation in B cell receptor-engaged BL2 cells. Thus, the in vitro model reveals that T-B cell membrane interactions through surface molecules different from CD40-CD40L can trigger somatic hypermutation (Denepoux et al., T cells can induce somatic mutation in B cell receptor-engaged BL2 Burkitt's lymphoma cells independently of CD40-CD40 ligand interactions. J Immunol 164(3):1306-13, 2000).

A hybridoma-derived monoclonal antibody, produced by immunization with the Burkitt's tumor-derived B-lymphoblastoid cell line, B35M, was previously shown to detect a 68,000 dalton surface membrane protein, BL2, on the surface of peripheral blood B cells, which is absent from thymocytes, T cells, and granulocytes. In a recent study, the expression and distribution of BL2 on benign and malignant human lymphoid cells was investigated. Indirect immunofluorescent assay with this monoclonal antibody demonstrated that BL2 is expressed by cells within the fetal liver and by a variable proportion of lymph node, tonsil, and spleen B cells, but not by T cells. The neoplastic cells isolated from 18 T-cell malignancies were BL2-. BL2 was was heterogeneously expressed by a variable proportion of the malignant cells in 29/32 cases of B-chronic lymphocytic leukemia and 33/38 cases of B-cell lymphomas, but appeared to be lost in the terminal stages of B-cell differentiation, as myeloma plasma cells were BL2-. BL2 expression was not limited to B cells of a particular surface immunoglobulin isotype. Immunofluorescent staining for BL2 in cryostat tissue sections demonstrated that the majority, but not all, germinal center and interfollicular Ia+(non-T) cells are BL2+. These findings suggested that BL2 is a B-cell lineage-specific differentiation marker that may be useful in the study of B-cell ontogeny and in defining subgroups of the B-cell malignancies (Knowles et al., A new human B-lymphocyte surface antigen (BL 2) detectable by a hybridoma monoclonal antibody: distribution on benign and malignant lymphoid cells. Blood July 1983; 62(1):191-9, 1983).

The protein similarity information, expression pattern, and map location for the NOV9 suggest that NOV9 may have important structural and/or physiological functions characteristic of the Immunoglobulin protein family. Therefore, the NOV9 nucleic acids and proteins of the invention are useful in potential therapeutic applications implicated in various diseases and disorders described below and/or other pathologies. For example, the NOV9 compositions of the present invention will have efficacy for treatment of patients suffering from inflammatory disorders such as osteo- and rheumatoid-arthritis, inflammatory bowel disease, Crohn's disease; immunological disorders, AIDS; cancers including but not limited to lung cancer, colon cancer, leukemia or pancreatic cancer.; blood disorders; asthma; psoriasis; inflammatory skin disordersvascular disorders, hypertension, skin disorders, renal disorders including Alport syndrome, immunological disorders, tissue injury, cancers, fibrosis disorders, bone diseases, Ehlers-Danlos syndrome type VI, VII, type IV, S-linked cutis laxa and Ehlers-Danlos syndrome type V, osteogenesis imperfecta, Immuno therapy of inflammatory and infectious diseases such as AIDS, treatment of Neurologic diseases, Brain and/or autoimmune disorders like encephalomyelitis, neurodegenerative disorders, Alzheimer's Disease, Parkinson's Disorder, immune disorders, and hematopoietic disorders, endocrine diseases, muscle disorders, wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, multiple sclerosis; and Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome. The NOV9 nucleic acid encoding Immunoglobulin-like protein, and the Immunoglobulin-like protein of the invention, or fragments thereof, may further be useful in diagnostic applications, wherein the presence or amount of the nucleic acid or the protein are to be assessed.

NOV10

NOV10 includes three novel Type II Cytokeratin-like proteins disclosed below. The disclosed proteins have been named NOV10a, NOV10b and NOV10c.

NOV10a

A disclosed NOV10a nucleic acid of 1782 nucleotides (also referred to GSAC055715.12_D) encoding a Type II Cytokeratin-like protein is shown in Table 10A. An open reading frame was identified beginning with an ATG initiation codon at nucleotides 63-65 and ending with a TAA codon at nucleotides 1710-1712. Putative untranslated regions upstream from the intiation codon and downstream from the termination codon are underlined in Table 10A, and the start and stop codons are in bold letters.

TABLE 10A
NOV10a Nucleotide Sequence

(SEQ ID NO:43)

CAGAACCTTTTGGGATTTTGCCTTCCTCCCTCCCGCATCTGAGCTTTGTCTCCACCAGCAACATGAGCCGCCAATTCACC
TGCAAGTCGGGAGCTGCCGCCAAGGGGGGCTTCAGTGGCTGCTCAGCTGTGCTCTCAGGGGGCAGCTCATCCTCCTTCCG
GGCAGGGAGCAAGGGCTCAGTGGGGGCTTTGGCAGCCGGAGCCTCGCAGGGAGCAAAGGGCTCAGTGGGGGGCTTTGGCA
GCCGGAGCCTCTACAGCCTGGGGGGTGTCCGGAGCCTCAATGTGGCCAGTGGCAGCGGGAAGAGTGGAGGCTATGGATTT
GGCCGGGGCCGGGCCAGTGGCTTTGCTGGAAGCATGTTTGGCAGTGTGGCCCTGGGGCCTGTGTGCCCAACTGTATGCCC
ACCTGGAGGCATCCACCAGGTTACCATCAATGAGAGCCTCCTGGCCCCCTCAACGTGGAGCTGGACCCCCAAGATCCAGA
AAGTGCGTGCCCAGGAGCGAGAGCAGATCAAGGCTCTGAACAACAAGTTCGCCTCCTTCATCGACAAGGTGCGGTTCCTG
GAGCAGCAGAACCAGGTACTGGAGACCAAGTGGGAGCTGCTGCAGCAGCTGGACCTGAACAACTGCAAGAACAACCTGGA
GCCCATCCTCGAGGGCTACATCAGCAACCTGCGGAAGCAGCTGGAGACGCTGTCTGGGGACAGGGTGAGGCTGGACTCGG
AGCTGAGGAATGTGCGGGACGTAGTGGAGGACTACAAGAAGAGGTATGAGGAGGAAATCAACAAGCGGACAGCAGCAGAG
AACGAGTTTGTGCTGCTCAAGAAGGATGTGGATGCTGCTTACGCCAATAAGGTGGAACTGCAGGCCAAGGTGGAATCCAT
GGACCAGGAGATCAAGTTCTTCAGGTGTCTCTTTGAAGCCGAGATCACTGAGATCCAGTCCCACATCAGTGACATGTCTG
TCATCCTGTCCATGGACAACAACCGGAACCTAGACCTGGACAGCATCATTGACGAAGTCCGCACCCAGTATGAGGAGATT
GCCTTGAAGAGTAAGGCCCGAGGCTGAGGCCCTGTACCAGACCAAGTTCCAAGAGCTTCAGCTGGCAGCTGCAGGCATGG
GGACGACCTCAAAAACACCAAGAATGAAATCTCGGAGCTCACTCGGCTCATCCAGAGAATCCGCTCAGAGATCGAGAACG
TGAAGAAGCAGGCTTCCAACCTGGAGACAGCCATCGCTGATGCTGAGCAGCGGGGAGACAACGCCCTGAAGGATGCCCGG
GCCAAGCTGGACGAGCTGGAGGGCGCCCTGCACCAGGCCAAGGAGGAGCTGGCACGGATGCTGCGCGAGTACCAGGAGCT
CATGAGCCTGAAGCTGGCCCEGGACATGGAGATCGCCACCTATCGCAAGCTACTGGAGAGCCAGGAGTGCACGATGTCAG
GAGAATTTCCCTCCCCTGTCAGCATCTCCATCATCAGCAGCACCAGTGGCGGCAGTGTCAGTGGCTATGGCGGTGCCAGT
GGTGTCGGCAGTGGCTTAGGCCTGGGTGGAGGAAGCAGCTACTCCTATGGCAGTGGTCTTGGCGTTGGAGGTGGCTTCAG
TTCCAGCAGTGGCAGAGCCATTGGGGGTGGCCTCAGCTCTGTTGGAGGCGGCAGTTCCACCATCAAGTACACCACCACCT
CCTCCTCCAGCAGGAAGAGCTATAAGCACTAAAGTGCGTCTGCTAGCTCTCGGTCCCACAGTCCTCAGGCCCCCTCTCTGG
CTGCAGAGCCCTCTCCTCAGGT

The disclosed NOV10a nucleic acid sequence, localized to chromosome 12, has 1255 of 1478 bases (84%) identical Mus musculus type II cytokeratin mRNA (gb:GENBANK-ID:AB033744|acc:AB033744.1) (E=5.2e⁻²³¹).

A disclosed NOV10a polypeptide (SEQ ID NO: 44) encoded by SEQ ID NO: 43 is 549 amino acid residues and is presented using the one-letter amino acid code in Table 10B. Signal P, Psort and/or Hydropathy results predict that NOV10a does not contain a signal peptide and is likely to be localized in the cytoplasm with a certainty of 0.4500.

TABLE 10B
Encoded NOV10a protein sequence.

(SEQ ID NO:44)

MSRQFTCKSGAAAKGGFSGCSAVLSGGSSSSFRAGSKGLSGGFGSRSLAGSKGLSGGFGSRSLYSLGGVRSLNVASGSGK
SGGYGFGRGRASGFAGSMFGSVALGPVCPTVCPPGGIHQVTINESLLAPLNVELDPKIQKVRAQEREQIKALNNKFASFI
DKVRFLEQQNQVLETKWELLQQLDLNNCKNNLEPILEGYISNLRKQLETLSGDRVRLDSELRNVRDVVEDYKKRYEEEIN
KRTAAENEFVLLKKDVDAAYANKVELQAKVESMDQEIKFFRCLFEAEITQIQSHTSDMSVILSMDNNRNLDLDSIIDEVR
TQYEEIALKSKAEAEALYQTKFQELQLAAGRHGDDLKNTKNEISELTRLIQRIRSEIENVKKQASNLETAIADAEQRGDN
ALKDARAKLDELEGALHQAKEELARMLREYQELMSLKLALDMEIATYRKLLESEECRMSGEFPSPVSISIISSTSGGSVS
GYGGASGVGSGLGLGGGSSYSYGSGLGVGGGFSSSSGRAIGGGLSSVGGGSSTIKYTTTSSSSRKSYKH

The NOV10a amino acid sequence has 440 of 519 amino acid residues (84%) identical to, and 465 of 519 amino acid residues (89%) similar to, a Mus musculus 524 amino acid residue Type II cytokeratin (ptnr:SPTREMBL-ACC:Q9R0H5) (E=4.6e⁻²²¹).

NOV10a is expressed in at least the following tissues: skin, muscle, bone, cartilage, Colon carcinoma, lung. This information was derived by determining the tissue sources of the sequences that were included in the invention including but not limited to SeqCalling sources, Public EST sources, Genomic Clone sources, Literature sources, and/or RACE sources. In addition, NOV10a is predicted to be expressed in the following tissues because of the expression pattern of a closely related Mus musculus mRNA for type II cytokeratin (GENBANK-ID: gb:GENBANK-ID:AB033744|acc:AB033744.1): skin, muscle, bone, cartilage, Colon carcinoma, lung.

NOV10b

A disclosed NOV10b nucleic acid of 1601 nucleotides (also referred to GSAC055715_C) encoding a Type II Cytokeratin-like protein is shown in Table 10C. An open reading frame was identified beginning with an ATG initiation codon at nucleotides 66-68 and ending with a TGA codon at nucleotides 1599-1601. A putative untranslated region upstream from the intiation codon is underlined in Table 10C, and the start and stop codons are in bold letters.

TABLE 10C
NOV10b Nucleotide Sequence

(SEQ ID NO:45)

CAGGAGGGATTCATGGGGAGTTCACGGTGGAACAGACAAAAACTCGACCGGTACTTGCGAGCATGAGCCGCCAACTG
ACCCATTTCCCCCGCGGGGAGCGCCTGGGCTTCAGCGGTTGCTCCGCGGTCCTCTCTGGCGGGATCGGCAGCAGCTCCGC
CTCATTCCGGGCCCGGGTCAGGGCTCGGCCTCCTTTGGCAGCAAGAGCCTCTCCTGCCTTGGGGGGCAGCCGAAGCCTGG
CGCTCAGCGCTGCTGCACGGCGGGGCGGCGGCCGCCTGGGCGGCTTCGTGGGCACCGCCTTCGGCAGCGCCGGGCTGGGG
CCCAAGTGTCCCTCCGTGTGCCCACCCGGGGGCATCCCTCAGGTCACCGTCAACAAGAGCCTCCTGGCCCCGCTCAACGT
GGAGATGGACCCCGAGATCCAGAGGGTGCGCGCCCAGGAGCGGGAGCAGATCAAGGCGCTAAACAACAAGTTCGCCTCCT
TCATCGACAAGGTGCGGTTCCTGGAGCAGCAGAATCAGGTGCTAGAGACCAAGTGGAACCTCCTACAGCAGCTGGACTTG
AACAACTGCAGGAAGAACCTGGAGCCCATTTATGAGGGCTACATCAGCAACCTGCAGAAGCAGCTGGAGATGCTGTCTGG
GGACGGGGTGAGGCTGGATTCGGAGCTGAGGAACATGCAGGATTTGGTGGAGGACTACAAGAAGAGATATGAGGTGGAGA
TTAACAGACGCACAGCTGCTGAGAATGAGTTTGTGGTGCTCAAGAAGGACGTGGATGCTGCTTACATGAATAAGGTTGAG
CTCCAGGCCAAGGTGGACTCCTTGACAGATGAGATTAAATTCTTCAAGTGCCTTTATGAAGGGGAGATCACTCAGATCCA
GTCCCACATCAGCGACACGTCCATCGTCCTGTCAATGGACAACAACCGGGATCTGGACCTGGACAGCATCATTGCCGAGG
TCCGTGCCCAGTACGAGGAGATTGCCCTAAAGAGCAAGGCCGAGGCTGAGACCCTGTACCAGACCAAGATCCAGGAGCTG
CAGGTCACAGCAGGCCAGCATGGGGATGACCTCAAGCTCACCAAGGCTGAAATCTCTGAGCTCAACCGCCTGATCCAGAG
GATCCGCTCAGAGATAGGGAATGTGAAGAAGCAGTGTGCCGATCTGGAGACGGCCATCGCCGACGCTGAACAGCGGGGGG
ACTGCGCCCTGAAAGATGCCCGGGCCAAGCTGGATGAGCTGGAGGGCGCCCTGCACCAGGCCAAGGAGGAGCTGGCACGG
ATGCTGCGTGAGTACCAGGAGCTCGTGAGCCTGAAGCTGGCCCTGGATATGGAGATCGCCACCTACCGCAAGCTGCTGGA
GAGCGAGGAGTGCAGGATGTCTGGCGAATATCCAAATTCTGTGAGCATCTCCGTCATCAGCAGCACCAATGCTGGGGCAG
GAGGGGCTGGCTTCAGCATGGGCTTTGGCGCCTCAAGCAGTTATAGCTACAAAACTGCAGCTGCAGACGTCAAGACCAAA
GGCAGCTGTGGCAGTGAGCTCAAGGATCCCCTTGCCAAAACCTCGGGGAGCAGCTGTGCCACCAAAAAGGCCTCCAGATG
A

The disclosed NOV10b nucleic acid sequence, localized to chromosome 12, has 1004 of 1224 bases (82%) identical Mus musculus type II cytokeratin mRNA (gb:GENBANK-ID:AB033744|acc:AB033744.1) (E=4.8e⁻¹⁷⁶).

A disclosed NOV10b polypeptide (SEQ ID NO: 46) encoded by SEQ ID NO: 45 is 511 amino acid residues and is presented using the one-letter amino acid code in Table 10D. Signal P, Psort and/or Hydropathy results predict that NOV10b does not contain a signal peptide and is likely to be localized in the cytoplasm with a certainty of 0.4500.

TABLE 10D
Encoded NOV10b protein sequence.

(SEQ ID NO:46)

MSRQLTHFPRGERLGFSGCSAVLSGGIGSSSASFRARVKGSASFGSKSLSCLGGSRSLALSAAARRGGGRLGGFVGTAFG
SAGLGPKCPSVCPPCGTPQVTVNKSLLAPLNVEMDPETQRVRAQEREQIKALNNKFASFIDKVRFLEQQNQVLETKWNLL
QQLDLNNCRKNLEPIYEGYISNLQKQLEMLSGDGVRLDSELRNMQDLVEDYKKRYEVEINRRTAAENEFVVLKKDVDAAY
MNKVELQAKVDSLTDEIKFFKCLYEGEITQIQSHISDTSIVLSMDNNRDLDLDSIIAEVRAQYEEIALKSKAEAETLYQT
KIQELQVTAGQHGDDLKLTKAEISELNRLIQRIRSEIGNVKKQCADLETAIADAEQRGDCALKDARAKLDELEGALHQAK
EELARMLREYQELVSLKLALDMEIATYRKLLESEECRMSGEYPNSVSISVISSTNAGAGGAGFSMGFGASSSYSYKTAAA
DVKTKGSCGSELKDPLAKTSGSSCATKKASR

The NOV10b amino acid sequence has 380 of 524 amino acid residues (72%) identical to, and 430 of 524 amino acid residues (82%) similar to, a Mus musculus 524 amino acid residue Type II cytokeratin (ptnr:SPTREMBL-ACC:Q9R0H5) (E=1.1e⁻¹⁸⁵).

NOV10b is expressed in at least the following tissues: skin, muscle, bone, cartilage, Colon carcinoma, lung. This information was derived by determining the tissue sources of the sequences that were included in the invention including but not limited to SeqCalling sources, Public EST sources, Genomic Clone sources, Literature sources, and/or RACE sources. In addition, NOV10b is predicted to be expressed in the following tissues because of the expression pattern of a closely related Mus musculus mRNA for type II cytokeratin (GENBANK-ID: gb:GENBANK-ID:AB033744|acc:AB033744.1): skin, muscle, bone, cartilage, Colon carcinoma, lung.

NOV10c

A disclosed NOV10c nucleic acid of 1606 nucleotides (also referred to GSAC055715_B) encoding a Type II Cytokeratin-like protein is shown in Table 10E. An open reading frame was identified beginning with an ATG initiation codon at nucleotides 1-3 and ending with a TAG codon at nucleotides 1515-1517. A putative untranslated region downstream from the termination codon is underlined in Table 10E, and the start and stop codons are in bold letters.

TABLE 10E
NOV10c Nucleotide Sequence

(SEQ ID NO:47)

ATGAGCCCGCCAATTCACCTACAAGTCGGGAGCTGCTGCCAAGGGGGGCTTCAGCGGCTGCTCCGCTGTGCTCTCAGGGG
CAGCTCATCCTCCTACCGAGCAGGGGGCAAAGGGCTCAGTGGAGGCTTCAGCAGTCGGAGCCTTTACAGCCTGGGGGGTG
CCCGGAGCATCTCTTTCAATGTGGCCAGTGGCAGTGGGTGGGCAGGAGGCTATGGATTTGGCCGGGGCCGGGCCAGTGGC
TTTGCTGGCAGCATGTTTGGCAGTGTGGCCTTGGGGTCCGTGTGTCCGTCGTTGTGCCCGCCCGGGGGTATCCATCAGGT
CACCATCAACAAGAGCCTCCTGGCACCCCTGAACGTGGAGCTGGACCCTGAAATCCAGAAAGTGCGTGCCCAGGAGCGGG
AGCAGATCAAGGTGCTGAACAACAAGTTCGCCTCCTTCATTGACAAGGTGCGGTTCCTGGAGCAGCAGAACCAGGTGCTG
GAGACCAAGTGGGAGCTGCTACAGCAGCTGGACCTGAACAACTGCAAGAATAACCTGGAGCCCATCCTTGAGGGCTACAT
CAGCAACCTGCGGAAGCAGCTGGAQACGCTGTCTGGGGACAGGGTGAGGCTGGACTCGGAGCTGAGGAGCGTGCGCGAAG
TGGTGGAGGACTACAAGAAGAGGTATGAAGAAGAAATAAACAAGCGCACAACTGCTGAGAATGAATTTGTGGTGCTTAAG
AAGGACGTGGACGCAGCTTACACGAGCAAAGTQGAGCTGCAGGCCAAGGTGGATGCCCTGGATGGAGAAATCAAGTTCTT
CAAGTGTCTGTACGAGGGGGAGACTGCTCAGATCCAGTCCCACATCAGCGACACGTCCATCATCCTGTCCATGGACAACA
ACCGGAACCTGGACCTGGACAGCATCATTGCTGAGGTCCGTGCCCAGTATGAGGAGATCGCCCGGAAGAGCAAGGCCGAG
GCCGAGGCCCTGTACCAGACCAAGTTCCAGGAGCTGCAGCTAGCAGCCGGCCGGCATGGGGATGACCTGAAACACACCAA
AAATGAGATCTCAGAGCTGACCCGTCTCATCCAAGACTGCGCTCGGAGATTGAGAGTGTGAAGAAGCAGTGTGCCAAACC
TGGAGACGGCCATCGCTGACGCCGAGCAGCGGGGGGACTGTGCCCTCAAGGATGCCAGGGCCAAGCTGGATGAGCTGGAG
GGCGCCCTGCAGCAGGCCAAGGAGGAGCTGGCACGGATGCTGCGCGAGTACCAAGAGCTTTTGAGCGTGAAGCTGTCCCT
GGATATTGAGATCGCCACCTACCGCAAGCTGCTGGAGGGCGAGGAGTGCAGGATGTCCGGAGAATATACCAACTCCGTGA
GCATTTCGGTCATCAACAGCTCCATGGCCGGGATGGCAGGCACAGGGGCTGGCTTTGGATTCAGCAATGCTGGCACCTAC
GGCTACTGGCCCAGCTCTGTCAGCGGGGCTACAGCATGCTGCCTGGGGGCTGTGTCACTGGCAGTGGGAACTGTAGCCC
CCACACACACCCAGAGGGTCAGCCCCACTGGAAGTTTCCAGGGTGATCTTGGGAGTGATAACCCAGTAATTGGAGGCCA
GCAGGT

The disclosed NOV10c nucleic acid sequence, localized to chromosome 12, has 1271 of 1525 bases (83%) identical Mus musculus type II cytokeratin mRNA (gb:GENBANK-ID:AB033744|acc:AB033744.1) (E=5.0e⁻²²⁸).

A disclosed NOV10c polypeptide (SEQ ID NO: 48) encoded by SEQ ID NO: 47 is 521 amino acid residues and is presented using the one-letter amino acid code in Table 10F. Signal P, Psort and/or Hydropathy results predict that NOV10c does not contain a signal peptide and is likely to be localized in the cytoplasm with a certainty of 0.4500.

TABLE 10F
Encoded NOV10c protein sequence.

(SEQ ID NO:48)

MSRQFTYKSGAAAKGGFSGCSAVLSGGSSSSYRAGGKGLSGGFSSRSLYSLGGARSISFNVASGSGWAGGYGFGRGRASG
FAGSMFGSVALGSVCPSLCPPGGIHQVTINKSLLAPLNVELDPEIQKVRAQEREQIKVLNNKFASFIDKVRFLEQQNQVL
ETKWELLQQLDLNNCKNNLEPILEGYISNLRKQLETLSGDRVRLDSELRSVREVVEDYKKRYEEEINKRTTAENEFVVLK
KDVDAAYTSKVELQAKVDALDGETKFFKCLYEGETAQTQSHTSDTSTTLSMDNNPNLDLDSIIAEVRAQYEEIARKSKAE
AEALYQTKFQELQLAAGRHGDDLKHTKNEISELTRLIQRLRSEIESVKKQCANLETAIADAEQRGDCALKDARAKLDELE
GALQQAKEELARMLREYQELLSVKLSLDIEIATYRKLLEGEECRMSGEYTNSVSISVINSSMAGMAGTGAGFGFSNAGTY
GYWPSSVSGGYSMLPGGCVTGSGNCSPHTHPEGQPEWKFPG

The NOV10c amino acid sequence has 414 of 506 amino acid residues (81%) identical to, and 454 of 506 amino acid residues (89%) similar to, a Mus musculus 524 amino acid residue Type II cytokeratin (ptnr:SPTREMBL-ACC:Q9R0H5) (E=1.1e⁻²¹²).

NOV10c is expressed in at least the following tissues: skin, mammary gland, and lung. This information was derived by determining the tissue sources of the sequences that were included in the invention including but not limited to SeqCalling sources, Public EST sources, Genomic Clone sources, Literature sources, and/or RACE sources. In addition, NOV10c is predicted to be expressed in the following tissues because of the expression pattern of a closely related Mus musculus mRNA for type II cytokeratin (GENBANK-ID: gb:GENBANK-ID:AB033744|acc:AB033744.1): skin, mammary gland, and lung.

NOV10a, NOV10b and NOV10c are very closely homologous as is shown in the amino acid alignment in Table 10G.

[image]

[image]

Homologies to any of the above NOV10 proteins will be shared by the other NOV10 proteins insofar as they are homologous to each other as shown above. Any reference to NOV10 is assumed to refer to both of the NOV10 proteins in general, unless otherwise noted.

The disclosed NOV10a polypeptide has homology to the amino acid sequences shown in the BLASTP data listed in Table 10H.

TABLE 10H
BLAST results for NOV10a

| Gene Index/ | | Length | Identity | Positives | |
|---|---|---|---|---|---|
| Identifier | Protein/Organism | (aa) | (%) | (%) | Expect |
| gi\|15321302\|ref\| | keratin 6 irs | 523 | 384/425 | 386/425 | 0.0 |
| XP_053295.1\| | [Homo sapiens] | | (90%) | (90%) | |
| (XM_053295) | | | | | |
| gi\|15321300\|ref\| | hypothetical | 441 | 354/368 | 356/368 | 1e−179 |
| XP_053294.1\| | protein XP_053294 | | (96%) | (96%) | |
| (XM_053294) | [Homo sapiens] | | | | |
| gi\|9910294\|ref\| | keratin complex 2, | 524 | 359/424 | 377/424 | 1e−179 |
| NP_064340.1 | gene 6 g [Mus | | (84%) | (88%) | |
| (NM_019956) | musculus] | | | | |
| gi\|15321298\|ref\| | hypothetical | 336 | 249/263 | 251/263 | 1e−133 |
| XP_053296.1\| | protein XP_053296 | | (94%) | (94%) | |
| (XM_053296) | [Homo sapiens] | | | | |
| gi\|7161776\|emb\| | cytokeratin [Homo | 551 | 257/432 | 329/432 | 1e−132 |
| CAB76832.1\| (Y19212) | sapiens] | | (59%) | (75%) | |

The homology between these and other sequences is shown graphically in the ClustalW analysis shown in Table 10I.

[image]

[image]

Table 10J lists the domain description from DOMAIN analysis results against NOV10a. This indicates that the NOV10a sequence has properties similar to those of other proteins known to contain these domains.

TABLE 10J
Domain Analysis of NOV10a

gnl|Pfam|pfam00038, filament, Intermediate filament
protein. (SEQ ID NO: 206)
Length = 312 residues, 100.0% aligned
Score = 291 bits (745), Expect = 7e−80

| | | | |
|---|---|---|---|
| NOV10a | 144 | QEREQIKALNNKFASFIDKVRFLEQQNQVLETKWELLQQLDLNNCKNNLEPILEGYISNL | 203 |
| | | \|+\|\|++ \|\|++ \|\|+\|\|\|\|\|\|\|\|\|\|\|+ \|\| \| \| \|+\| + \| + \| \| \| | |
| 00038 | 1 | NEKEQMQNLNDRLASYIDKVRFLEQQNKELEVKIEELRQ-KQAPSVSRLYSLYETEIEEL | 59 |
| NOV10a | 204 | RKQLETLSGDRVRLDSELRNVRDVVEDYKKEYEEEINKRTAAENEFVLLKKVDVAAYANK | 263 |
| | | \|+\|++ \|+ +\| \|\| \|+ \|+\|+ \|\|++\|+\|\|+\|\|\| \| \|\|\|+ \| \|+\|\|+\| \| \| | |
| 00038 | 60 | PRQIDQLTNERARLQLEIDNLREAAEDFRKKYEDEINLRQEAENDLVGLRKDLKEATLAR | 119 |
| NOV10a | 264 | VELQAKVESMDQEIKFFRCLFEAEITQIQSHISDMSVILSMDNNRNLDLDSIIDEVRTQY | 323 |
| | | \|+\|+ \|\|\|\|+ +\|++\| + \| \|+ ++\|+ \| \| +\| + \|\| \| \|\|\| + \|+\| \|\| | |
| 00038 | 120 | VDLENKVESLQEELEFLKKNHEEEVKELQAQIQD-TVNVEMDAARKLDLTKALREIRAQY | 178 |
| NOV10a | 324 | EEIALKSKAEAEALYQTKFQELQLAAGRHGDDLKNTKNEISELTRLIQRIRSEIENVKKQ | 383 |
| | | \|\|\|\| \|++ \|\|\| \|++\| +\|\|\| \|\| \|+\|+ \|++ \| \|\|+\|\| \| \|\| + \|++++\| \| | |
| 00038 | 179 | EEIAKKNRQEAEEWYKSKLEELQTAAARNGEALRSAKEEITELRRQIQSLEIELQSLKAQ | 238 |
| NOV10a | 384 | ASNLETAIADAEQRGDNALKDARAKLDELEGALHQAKEELARMLREYQELMSLKLALDME | 443 |
| | | ++\|\| +\|+ \|+\| + \|+ +\| + +\|\| \| \| +\|\|+\|\| \|\|\|\|\|\|\|+ +\|\|\|\|\|+\| | 443 |
| 00038 | 239 | NASLERQLAELEERYELELRQYQALISQLEEELQQLREEMARQLREYQELLDVKLALDIE | 298 |
| NOV10a | 444 | IATYRKLLESEECR 457 | |
| | | \|\|\|\|\|\|\|\|\| \|\| \| | |
| 00038 | 299 | IATYRKLLEQEESR 312 | |

Cytokeratins are constituent proteins of intermediate filaments (IFs) that form heterotypic tetrameric IF subunits containing two polypeptide chains of each of the two cytokeratin subfamilies, i.e. the acidic (type I) and the basic (type II). To locate the molecular domains involved in the formation of these heterotypic complexes, a binding assay was developed. The results obtained indicate that: (1) the binding between cytokeratin polypeptides of the complementary type is stronger and more selective than interactions of cytokeratins with other IF and non-IF proteins; (2) both the head and the tail portions of the proteins are not required for heterotypic complex formation; (3) the complementarity information located in the alpha-helical portions of the rod domain, and in short sequences immediately flanking them, is sufficient to discriminate between the two types of cytokeratins and to secure the formation of heterotypic cytokeratin complexes; (4) both coils 1 and 2 of the rod can contribute to this association; and (5) the formation of the heterotypic cytokeratin complex is not critically dependent upon ionic interactions. These results are further compatible with the concept that the heterotypic binding takes place between cytokeratin homodimer coiled-coils (Hatzfeld et al., Cytokeratin domains involved in heterotypic complex formation determined by in-vitro binding assays. J Mol Biol 197(2):237-55, 1987).

Human liver parenchymal cells have a very simple cytokeratin composition and express only one cytokeratin pair: cytokeratin 8 (a type II cytokeratin, molecular weight 52 kD) and cytokeratin 18 (a type I cytokeratin, molecular weight 45 kD). Intrahepatic bile duct cells contain in addition to cytokeratins 8 and 18 also cytokeratins 7 (a type II cytokeratin, molecular weight 54 kD) and cytokeratin 19 (a type I cytokeratin, molecular weight 40 kD) (Van Eyken et al., Immunocytochemistry of cytokeratins in primary human liver tumors. APMIS Suppl 23:77-85 1991).

Three monoclonal antibodies, 1C7, 2D7 and 6B10, directed against cytokeratins of human esophagus were isolated and characterized by one- and two-dimensional gel electrophoresis and by immunohistochemical staining on sections of human epithelial tissues. In immunoblot experiments, antibodies of clones 1C7 (IgG2a) and 2D7 (IgG2b) react only with cytokeratin no. 13 of the acidic (type I) subfamily of cytokeratin polypeptides (Mr 54000; pI 5.1); antibodies of clone 6B10 (IgG1) detect only cytokeratin no. 4 (Mr 59000; pI 7.3) of the basic (type II) cytokeratin subfamily and allows the detection of this protein and possible degradation products at high sensitivity. These monoclonal antibodies are the first examples of antibodies specific for individual cytokeratin polypeptides characteristic of certain complex epithelia. They allow the identification of distinct minor populations of cells present in certain complex and glandular epithelia and in tumors derived therefrom which hitherto have not been distinguished. The possible reasons for the occurrence of cell type heterogeneity of cytokeratin expression in complex epithelia and in some carcinomas are discussed (van Muijen et al., Cell type heterogeneity of cytokeratin expression in complex epithelia and carcinomas as demonstrated by monoclonal antibodies specific for cytokeratins nos. 4 and 13. Exp Cell Res 162(1):97-113, 1986).

Studies have analyzed the possibility that cytoskeletal proteins may be the target of forskolin in living Caco-2 cells. It was shown that forskolin initiates dramatic changes in the spatial organization of the cytokeratin network that correlate with an increased phosphorylation of cytokeratin molecules, whereas microtubules, microfilaments and vimentin remain mainly unaffected. Indirect immunofluorescence studies showed that the cytokeratin network is redistributed from the cell periphery to the cytoplasm. Biochemical experiments indicate that forskolin doesn't interfere with the cytokeratin profile, since the three cytokeratins normally found in intestine (CK 8, CK 18, CK 19) are similarly expressed in both control and forskolin-Caco-2 cells. Analysis of 32P-labeled cytokeratin extracted from the two cell populations demonstrates that forskolin quantitatively increases the phosphorylation of type I cytokeratin (CK 18 and CK 19), whereas the phosphorylation of type II cytokeratin (CK 8) is altered both quantitatively and qualitatively with the emergence of a new phosphorylation site (Baricault et al., The network organization and the phosphorylation of cytokeratins are concomitantly modified by forskolin in the enterocyte-like differentiated Caco-2 cell line. J Cell Sci 107 (Pt 10):2909-18, 1994).

By using the subtractive hybridization method, two complementary DNA clones differently expressed in rat normal esophageal epithelium and squamous cell carcinoma induced by administration of precursors of N-nitrososarcosine ethyl ester were isolated. A rat homologue of the human 50-kDa type I cytokeratin 14 was cloned for the first time and shown to be expressed preferentially in squamous cell papillomas and carcinomas, whereas it was weakly expressed or absent in normal squamous epithelial cells and in hyperplastic lesions. A rat homologue of the mouse 57-kDa type II cytokeratin showed strong expression in both normal and tumor tissues. These results are well consistent with the reported alteration of keratin subspecies in human esophageal cancers, therefore, encouraging us to use this experimental system as a model for human esophageal carcinogenesis (Wang et al., Identification of cytokeratin subspecies altered in rat experimental esophageal tumors by subtractive cloning. Cancer Lett 108(1):119-27, 1996).

The protein similarity information, expression pattern, and map location for the NOV10 suggest that NOV10 may have important structural and/or physiological functions characteristic of the Type II Cytokeratin protein family. Therefore, the NOV10 nucleic acids and proteins of the invention are useful in potential therapeutic applications implicated in various diseases and disorders described below and/or other pathologies. For example, the NOV10 compositions of the present invention will have efficacy for treatment of patients suffering from inflammatory and infectious diseases such as AIDS; cancer, Neurologic diseases, Brain and/or autoimmune disorders like encephalomyelitis, neurodegenerative disorders, Alzheimer's Disease, Parkinson's Disorder, immune disorders, and hematopoietic disorders, endocrine diseases, muscle disorders, wound repair, bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease. The NOV10 nucleic acid encoding Type II Cytokeratin-like protein, and the Type II Cytokeratin-like protein of the invention, or fragments thereof, may further be useful in diagnostic applications, wherein the presence or amount of the nucleic acid or the protein are to be assessed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g. cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17, 19, 21, 23, 25, 27, 29, 31, 33, 35, 37, 39, 41, 43, 45 and 47, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19, 21, 23, 25, 27, 29, 31, 33, 35, 37, 39, 41, 43, 45 and47 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, 25, 27, 29, 31, 33, 35, 37, 39, 41, 43, 45 and 47, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, 25, 27, 29, 31, 33, 35, 37, 39, 41, 43, 45 and 47, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown NOS: 1, 3, 5, 7, 9, 11, 13, 15, 17, 19, 21, 23, 25, 27, 29, 31, 33, 35, 37, 39, 41, 43, 45 or 47 is one that is sufficiently complementary to the nucleotide sequence shown NOS: 1, 3, 5, 7, 9, 11, 13, 15, 17, 19, 21, 23, 25, 27, 29, 31, 33, 35, 37, 39,41, 43, 45 or 47 that it can hydrogen bond with little or no mismatches to the nucleotide sequence shown SEQ ID NOS: 1, 3, 5, 7, 9, 11, 13, 15, 17, 19, 21, 23, 25, 27, 29, 31, 33, 35, 37, 39, 41, 43, 45 and 47, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, 25, 27, 29, 31, 33, 35, 37, 39, 41, 43, 45 and 47, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, 25, 27, 29, 31, 33, 35, 37, 39, 41, 43, 45 and 47; or an anti-sense strand nucleotide sequence of SEQ ID NOS: 1, 3, 5, 7, 9, 11, 13, 15, 17, 19, 21, 23, 25, 27, 29, 31, 33, 35, 37, 39, 41, 43, 45 and 47; or of a naturally occurring mutant of SEQ ID NOS: 1, 3, 5, 7, 9, 11, 13, 15, 17, 19, 21, 23, 25, 27, 29, 31, 33, 35, 37, 39, 41, 43, 45 and 47.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, 17, 19, 21, 23, 25, 27, 29, 31, 33, 35, 37, 39, 41, 43, 45 and 47, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, 23, 25, 27, 29, 31, 33, 35, 37, 39, 41, 43, 45 and 47 due to degeneracy of the genetic code and thus encode the same NOVX proteins as that encoded by the nucleotide sequences shown in SEQ ID NOS: 1, 3, 5, 7, 9, 11, 13, 15, 17, 19, 21, 23, 25, 27, 29, 31, 33, 35, 37, 39, 41, 43, 45 and 47. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24, 26, 28, 30, 32, 34, 36, 38, 40, 42, 44, 46 and 48.

In addition to the human NOVX nucleotide sequences shown in SEQ ID NOS: 1, 3, 5, 7, 9, 11, 13, 15, 17, 19, 21, 23, 25, 27, 29, 31, 33, 35, 37, 39, 41, 43, 45 and 47, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, 17, 19, 21, 23, 25, 27, 29, 31, 33, 35, 37, 39, 41, 43, 45 and 47 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45 and 47. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, 23, 25, 27, 29, 31, 33, 35, 37, 39, 41, 43, 45 and 47, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45 and 47, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, 25, 27, 29, 31, 33, 35, 37, 39, 41, 43, 45 and 47, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, 23, 25, 27, 29, 31, 33, 35, 37, 39, 41, 43, 45 and 47, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS: 2, 4, 6, 8, 10, 12, 14, 16, 18, 20, 22, 24, 26, 28, 30, 32, 34, 36, 38, 40, 42, 44, 46 and 48. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 1, 3, 5, 7, 9, 11, 13, 15, 17, 19, 21, 23, 25, 27, 29, 31, 33, 35, 37, 39, 41, 43, 45 and 47 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS: 2, 4, 6, 8, 10, 12, 14, 16, 18, 20, 22, 24, 26, 28, 30, 32, 34, 36, 38, 40, 42, 44, 46 and 48. Preferably, the protein encoded by the nucleic acid molecule is at least about 60% homologous to SEQ ID NOS: 2, 4, 6, 8, 10, 12, 14, 16, 18, 20, 22, 24, 26, 28, 30, 32, 34, 36, 38, 40, 42, 44, 46 and 48; more preferably at least about 70% homologous SEQ ID NOS: 2, 4, 6, 8, 10, 12, 14, 16, 18, 20, 22, 24, 26, 28, 30, 32, 34, 36, 38, 40, 42, 44, 46 and 48; still more preferably at least about 80% homologous to SEQ ID NOS: 2, 4, 6, 8, 10, 12, 14, 16, 18, 20, 22, 24, 26, 28, 30, 32, 34, 36, 38, 40, 42, 44, 46 and 48; even more preferably at least about 90% homologous to SEQ ID NOS: 2, 4, 6, 8, 10, 12, 14, 16, 18, 20, 22, 24, 26, 28, 30, 32, 34, 36, 38, 40, 42, 44, 46 and 48; and most preferably at least about 95% homologous to SEQ ID NOS: 2, 4, 6, 8, 10, 12, 14, 16, 18, 20, 22, 24, 26, 28, 30, 32, 34, 36, 38, 40, 42, 44, 46 and 48.

An isolated nucleic acid molecule encoding an NOVX protein homologous to the protein of SEQ ID NOS: 2, 4, 6, 8, 10, 12, 14, 16, 18, 20, 22, 24, 26, 28, 30, 32, 34, 36, 38, 40, 42, 44, 46 and 48 can be created by introducing one or more nucleotide substitutions, additions or deletions into the nucleotide sequence of SEQ ID NOS: 1, 3, 5, 7, 9, 11, 13, 15, 17, 19, 21, 23, 25, 27, 29, 31, 33, 35, 37, 39, 41, 43, 45 and 47, such that one or more amino acid substitutions, additions or deletions are introduced into the encoded protein.

Mutations can be introduced into SEQ ID NOS: 1, 3, 5, 7, 9, 11, 13, 15, 17, 19, 21, 23, 25, 27, 29, 31, 33, 35, 37, 39, 41, 43, 45 and 47 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, 25, 27, 29, 31, 33, 35, 37, 39, 41, 43, 45 and 47, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STAG, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof, (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, 27, 29, 31, 33, 35, 37, 39, 41, 43, 45 and 47, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS: 2, 4, 6, 8, 10, 12, 14, 16, 18, 20, 22, 24, 26, 28, 30, 32, 34, 36, 38, 40, 42, 44, 46 and 48, or antisense nucleic acids complementary to an NOVX nucleic acid sequence of SEQ ID NOS: 1, 3, 5, 7, 9, 11, 13, 15, 17, 19, 21, 23, 25, 27, 29, 31, 33, 35, 37, 39, 41, 43, 45 and 47, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an (α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, 25, 27, 29, 31, 33, 35, 37, 39, 41, 43, 45 and 47). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996. supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS: 2, 4, 6, 8, 10, 12, 14, 16, 18, 20, 22, 24, 26, 28, 30, 32, 34, 36, 38, 40, 42, 44, 46 and 48. The invention also includes a mutant or variant protein any of whose residues may be changed from the corresponding residues shown in SEQ ID NOS: 2, 4, 6, 8, 10, 12, 14, 16, 18, 20, 22, 24, 26, 28, 30, 32, 34, 36, 38, 40, 42, 44, 46 and 48 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS: 2, 4, 6, 8, 10, 12, 14, 16, 18, 20, 22, 24, 26, 28, 30, 32, 34, 36, 38, 40, 42, 44, 46 and 48) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS: 2, 4, 6, 8, 10, 12, 14, 16, 18, 20, 22, 24, 26, 28, 30, 32, 34, 36, 38, 40, 42, 44, 46 and 48. In other embodiments, the NOVX protein is substantially homologous to SEQ ID NOS: 2, 4, 6, 8, 10, 12, 14, 16, 18, 20, 22, 24, 26, 28, 30, 32, 34, 36, 38, 40, 42, 44, 46 and 48, and retains the functional activity of the protein of SEQ ID NOS: 2, 4, 6, 8, 10, 12, 14, 16, 18, 20, 22, 24, 26, 28, 30, 32, 34, 36, 38, 40, 42, 44, 46 and 48, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS: 2, 4, 6, 8, 10, 12, 14, 16, 18, 20, 22, 24, 26, 28, 30, 32, 34, 36, 38, 40, 42, 44, 46 and 48, and retains the functional activity of the NOVX proteins of SEQ ID NOS: 2, 4, 6, 8, 10, 12, 14, 16, 18, 20, 22, 24, 26, 28, 30, 32, 34, 36, 38, 40, 42, 44, 46 and 48.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27, 29, 31, 33, 35, 37, 39, 41, 43, 45 and 47.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS: 2, 4, 6, 8, 10, 12, 14, 16, 18, 20, 22, 24, 26, 28, 30, 32, 34, 36, 38, 40, 42, 44, 46 and 48, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THRAY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g. NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nuc. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec 1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, 17, 19, 21, 23, 25, 27, 29, 31, 33, 35, 37, 39, 41, 43, 45 and 47 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene(e.g.,the cDNA of SEQ ID NOS: 1, 3, 5, 7, 9, 11, 13, 15, 17, 19, 21, 23, 25, 27, 29, 31, 33, 35, 37, 39, 41, 43, 45 and 47), but more preferably, is a non-human homologue of a human NOVX gene. For example, a mouse homologue of human NOVX gene of SEQ ID NOS: 1, 3, 5, 7, 9, 11, 13, 15, 17, 19, 21, 23, 25, 27, 29, 31, 33, 35, 37, 39, 41, 43, 45 and 47 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage PI. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g. by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e g., in a biological sample) or a genetic lesion in an NOVX gene, and to modulate NOVX activity, as described further, below. In addition, the NOVX proteins can be used to screen drags or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, 23, 25, 27, 29, 31, 33, 35, 37, 39, 41, 43, 45 and 47, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, 27, 29, 31, 33, 35, 37, 39, 41, 43, 45 and 47 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, 23, 25, 27, 29, 31, 33, 35, 37, 39, 41, 43, 45 and 47, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in clectrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g. Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof, (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators ( i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further usefull in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Nucleic Acids

TblastN using CuraGen Corporation's sequence file for polypeptides or homologs was run against the Genomic Daily Files made available by GenBank or from files downloaded from the individual sequencing centers. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The novel NOVX target sequences identified in the present invention were subjected to the exon linking process to confirm the sequence. PCR primers were designed by starting at the most upstream sequence available, for the forward primer, and at the most downstream sequence available for the reverse primer. PCR primer sequences were used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

### Example 2

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence.

### Example 3.

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C.. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqManq® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-1 3 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 kg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 gM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10 cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

AI_comprehensive panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-i anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the Al_comprehensive panel-v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

| | | |
|---|---|---|
| Donor 2 and 3 U | Mesenchymal Stem cells | Undifferentiated Adipose |
| Donor 2 and 3 AM | Adipose | AdiposeMidway |
| | | Differentiated |
| Donor 2 and 3 AD | Adipose | Adipose Differentiated |

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

NOV1a: Membrane Protein/neuropilin/metalloproteinase-like

Expression of the NOV1a gene (SC40376139) was assessed using the primer-probe set Ag2229 described in Table 11. Results from RTQ-PCR runs are shown in Tables 12 and 13.

TABLE 11
Probe Name Ag2229

| | | | | Start | SEQ ID |
|---|---|---|---|---|---|
| Primers | Sequences | TM | Length | Position | NO: |
| Forward | 5′-ACCAAATTTGGTGAAGGAGATT-3′ | 58.9 | 22 | 700 | 128 |
| Probe | TET-5′-CAACAATTCGTGTGATCAAATATAGTCCTG-3′-TAMRA | 65.5 | 30 | 722 | 129 |
| Reverse | 5′-CCATCTTCAAATCCACAATGAA-3′ | 59.8 | 22 | 773 | 130 |

TABLE 12
Panel 1.3D

| | Relative | | Relative |
|---|---|---|---|
| | Expression (%) | | Expression (%) |
| Tissue Name | tm4184t_ag2229 | Tissue Name | tm4184t_ag2229 |
| Liver adenocarcinoma | 0.0 | Kidney (fetal) | 1.0 |
| Pancreas | 0.0 | Renal ca. 786-0 | 6.6 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 24.0 | Liver | 0.0 |
| Brain (whole) | 13.8 | Liver (fetal) | 0.0 |
| Brain (amygdala) | 19.5 | Liver ca. (hepatoblast) HepG2 | 0.0 |
| Brain (cerebellum) | 4.6 | Lung | 0.0 |
| Brain (hippocampus) | 100.0 | Lung (fetal) | 0.0 |
| Brain (substantia nigra) | 6.6 | Lung ca. (small cell) LX-1 | 0.0 |
| Brain (thalamus) | 18.2 | Lung ca. (small cell) NCI-H69 | 9.4 |
| Cerebral Cortex | 82.4 | Lung ca. (s. cell var.) SHP-77 | 36.3 |
| Spinal cord | 50 | Lung ca. (large cell) NCI-H460 | 0.0 |
| CNS ca. (glio/astro) U87-MG | 0.0 | Lung ca. (non-sm. cell) A549 | 0.0 |
| CNS ca. (glio/astro) U-118-MG | 1.6 | Lung ca. (non-s. cell) NCI-H23 | 0.0 |
| CNS ca. (astro) SW1783 | 0.0 | Lung ca (non-s. cell) HOP-62 | 0.0 |
| CNS ca.* (neuro; met) SK-N- | 0.0 | Lung ca. (non-s. cl) NCI-H522 | 0.0 |
| AS | | | |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 0.0 |
| CNS ca. (glio) U251 | 0.0 | Breast ca.* (pl. effusion) MCF-7 | 1.6 |
| CNS ca. (glio) SF-295 | 0.0 | Breast ca.* (pl. ef) MDA-MB-231 | 0.0 |
| Heart (fetal) | 0.0 | Breast ca.* (pl. effusion) T47D | 6.4 |
| Heart | 0.0 | Breast ca. BT-549 | 0.0 |
| Fetal Skeletal | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 3.3 |
| Colorectal | 0.0 | Ovarian ca. IGROV-1 | 0.0 |
| Stomach | 0.0 | Ovarian ca.* (ascites) SK-OV-3 | 0.0 |
| Small intestine | 0.0 | Uterus | 0.0 |
| Colon ca. SW480 | 0.0 | Placenta | 0.0 |
| Colon ca.* (SW480 met)SW620 | 0.0 | Prostate | 0.0 |
| Colon ca. HT29 | 0.0 | Prostate ca.* (bone met) PC-3 | 0.0 |
| Colon ca. HCT-116 | 0.0 | Testis | 2.1 |
| Colon ca. CaCo-2 | 0.0 | Melanoma Hs688(A).T | 0.0 |
| 83219 CC Well to Mod Diff | 0.0 | Melanoma* (met) Hs688(B).T | 0.0 |
| (ODO3866) | | | |
| Colon ca. HCC-2998 | 0.0 | Melanoma UACC-62 | 4.2 |
| Gastric ca.* (liver met) NCI-N87 | 0.0 | Melanoma M14 | 1.7 |
| Bladder | 0.0 | Melanoma LOX IMVI | 0.0 |
| Trachea | 0.0 | Melanoma* (met) SK-MEL-5 | 15.4 |
| Kidney | 0.0 | Adipose | 0.0 |

TABLE 13
Panel CNS_neurodegeneration_v1.0

| | Relative | | Relative |
|---|---|---|---|
| | Expression (%) | | Expression (%) |
| | tm7007t_{—} | | tm7007t_{—} |
| Tissue Name | ag2229_a1_s2 | Tissue Name | ag2229_a1_s2 |
| AD 1 Hippo | 0.9 | Control (Path) 3 Temporal Ctx | 2.3 |
| AD 2 Hippo | 30.7 | Control (Path) 4 Temporal Ctx | 13.8 |
| AD 3 Hippo | 1.3 | AD 1 Occipital Ctx | 4.3 |
| AD 4 Hippo | 1.5 | AD 2 Occipital Ctx (Missing) | 1.2 |
| AD 5 hippo | 19.4 | AD 3 Occipital Ctx | 1.2 |
| AD 6 Hippo | 17.0 | AD 4 Occipital Ctx | 5.5 |
| Control 2 Hippo | 18.4 | AD 5 Occipital Ctx | 1.8 |
| Control 4 Hippo | 1.4 | AD 6 Occipital Ctx | 17.5 |
| Control (Path) 3 Hippo | 0.6 | Control 1 Occipital Ctx | 0.0 |
| AD 1 Temporal Ctx | 3.5 | Control 2 Occipital Ctx | 41.5 |
| AD 2 Temporal Ctx | 10.2 | Control 3 Occipital Ctx | 3.5 |
| AD 3 Temporal Ctx | 0.8 | Control 4 Occipital Ctx | 1.9 |
| AD 4 Temporal Ctx | 3.3 | Control (Path) 1 Occipital Ctx | 100.0 |
| AD 5 Inf Temporal Ctx | 10.1 | Control (Path) 2 Occipital Ctx | 3.1 |
| AD 5 SupTemporal Ctx | 2.8 | Control (Path) 3 Occipital Ctx | 0.9 |
| AD 6 Inf Temporal Ctx | 21.5 | Control (Path) 4 Occipital Ctx | 3.3 |
| AD 6 Sup Temporal Ctx | 30.3 | Control 1 Parietal Ctx | 1.5 |
| Control 1 Temporal Ctx | 5.5 | Control 2 Parietal Ctx | 3.5 |
| Control 2 Temporal Ctx | 22.2 | Control 3 Parietal Ctx | 7.6 |
| Control 3 Temporal Ctx | 4.5 | Control (Path) 1 Parietal Ctx | 47.8 |
| Control 4 Temporal Ctx | 2.7 | Control (Path) 2 Parietal Ctx | 11.4 |
| Control (Path) 1 Temporal Ctx | 29.1 | Control (Path) 3 Parietal Ctx | 1.8 |
| Control (Path) 2 Temporal Ctx | 15.5 | Control (Path) 4 Parietal Ctx | 30.0 |

Panel 1.3D Summary Expression of the NOV1 gene appears to be brain-specific.

Highest expression is detected in the hippocampus (CT=33) and the cerebral cortex, two regions that degenerate in Alzheimer's disease. Thus, this gene would be useful for distinguishing brain tissue from non-neural tissue, and may be beneficial as a drug target in neurodegenerative disease. Please see Panel_CNS_neurodegeneration for further discussion of potential utility in the central nervous system.

Panel 2D Summary Expression of the NOV1 gene is low/undetectable (Ct values >35) in all samples in this panel. (Data not shown.)

Panel 4D Summary Expression of the NOV1 gene is low/undetectable (Ct values >35) in all samples in this panel. (Data not shown.)

Panel CNS_{—}1 Summary Expression of the NOV1 gene is low/undetectable (Ct values >35) in all samples in this panel. (Data not shown.)

Panel CNS_neurodegeneration_v1.0 Summary The NOV1 gene is expressed at low but significant levels in the brain, as is seen in Panel 1.3D, with highest expression in the occipital cortex from the brain of a control patient (CT=32.2). Importantly, once the CT values are corrected for RNA quality/amount per well, there appears to be a down-regulation in the temporal cortex of the Alzheimer's diseased (AD) brain (in 5 of 6 diseased brains), a region that is known to undergo severe neurodegeneration. Conversely, in the occipital cortex, where neurodegeneration is not observed in AD, this gene does not appear to be differentially regulated. Neuropilins act as receptors for semaphorins, which mediate axon repulsion during neurodevelopment as well as neuroapoptosis. Thus, the downregulation of the expression of this gene may be critically involved in the neurodegeneration seen in Alzheimer's disease.

Therefore, selective antagonism of this receptor may slow neurodegeneration in this disease or enhance the compensatory synaptogenesis process. Furthermore, the NOV1 gene product may be useful in any clinical situation involving neuronal death, such as Parkinson's or Huntington's disease, spinocerebellar ataxia, head or spinal cord trauma, or stroke (Zhang et al., Up-regulation of neuropilin-1 in neovasculature after focal cerebral ischemia in the adult rat. J Cereb Blood Flow Metab. 21(5):541-9, 2001; Ziv et al., Semaphorins as mediators of neuronal apoptosis. J Neurochem. 73(3):961-71, 1999).

NOV2: Fibrillin-like protein

Expression of the NOV2a gene (GMAC022146_A) and variants NOV2b (153568997), NOV2c (CG88987-01), NOV2d (CG88987-02), NOV2e (CG88987-03) and NOV2f (CG88987-05) was assessed using the primer-probe sets Ag72, Ag390, Ag671, Ag766, Ag1211, Ag1928, Ag1938, Ag2233, Ag2241, Ag4154, and Ag4334 described in Tables 11-24. Please note that not all probe and primer sets match all the sequences. Table 25 provides a complete summary of the probe and primer sets that correspond to each sequence, where the “X” represents a match between the sequence and the probe and primer set. Results from RTQ-PCR runs are shown in Tables 26-35.

TABLE 14

Probe Name Ag72

| | | | | | |
|---|---|---|---|---|---|
| | | | | Start | SEQ ID |
| Primers | Sequences | TM | Length | Position | NO: |

| | | | | |
|---|---|---|---|---|
| Forward | 5′-CAGCGGAAAGACCCAGCA-3′ | 18 | 64 | 131 |
| Probe | FAM-5′-CGCCCGTTGCGACAGACTCCC-3′-TAMRA | 21 | 94 | 132 |
| Reverse | 5′-GATGTGAACGAGTGTGAGTCCTTC-3′ | 24 | 117 | 133 |

TABLE 15

Probe Name Ag390

| | | | | Start | SEQ ID |
|---|---|---|---|---|---|
| Primers | Sequences | TM | Length | Position | NO: |
| Forward | 5′-ACCAATGTCATCGGAGGCTT-3′ | | 20 | 175 | 134 |
| Probe | FMA-5′-TCAAAGCCGTCAGCACAGGCACA-3′-TAMRA | | 23 | 199 | 135 |
| Reverse | 5′-GATGTCCTCGCAGGTCATCAT-3′ | | 21 | 232 | 136 |

TABLE 16

Probe Name Ag671

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-ACTAACGGCGTCTGTGTCAA-3′ | 59.4 | 20 | 37 | 137 |
| Probe | FAM-5′-CCTTTGGCTACAGCCTGGACTTCACT-3′-TAMRA | 68.6 | 26 | 86 | 138 |
| Reverse | 5′-GTCTGTGTCCACACAGTTGATG-3′ | 59.1 | 22 | 114 | 139 |

TABLE 17 !Probe Name Ag766? !? ? ? ? Start? SEQ ID? ? !Primers? Sequences? TM? Length? Position? NO:

| | | | | | |
|---|---|---|---|---|---|
| Forward | 5′-TGCTACTACCTGCCTGGATATG-3′ | 59.3 | 22 | 839 | 140 |
| Probe | TET-5′-CAAGCCATGTACCTTCCTCTGCAAAA-3′-TAMRA | 68.2 | 26 | 881 | 141 |
| Reverse | 5′-GACAGCTGCACAGGAAACTG-3′ | 59.6 | 20 | 917 | 142 |

TALBE 18

Probe Name Ag1211

| | | | | Start | SEQ ID |
|---|---|---|---|---|---|
| Primers | Sequences | TM | Length | Position | NO: |
| Forward | 5′-CCCCTTGACATTGATGAGTGT-3′ | 59.8 | 21 | 367 | 143 |
| Probe | FAM-5′-AGATCCCCGCCATCTGTGCCAAT-3′-TAMRA | 71.7 | 23 | 392 | 144 |
| Reverse | 5′-ACTCCCGATCTGGTTTATGC-3′ | 59 | 20 | 422 | 145 |

TABLE 19

Probe Name Ag1928

| | | | | Start | SEQ ID |
|---|---|---|---|---|---|
| Primers | Sequences | TM | Length | Position | NO: |
| Forward | 5′-AATACCGAGGGCTCCTACCT-3′ | 59.1 | 20 | 566 | 146 |
| Probe | FAM-5′-ACCTGTCCAGCCCGCTACACCCT-3′-TAMRA | 71.1 | 23 | 590 | 147 |
| Reverse | 5′-CATTCATTGTCATCTCGACACA-3′ | 59.6 | 22 | 630 | 148 |

TABLE 20
Probe Name Ag1938

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-GCCCTGGCAACTCTAATATTG-3′ | 58.7 | 21 | 941 | 149 |
| Probe | TET-5′-CACTGCTACCCTCAACCAGACCATTG-3′-TAMRA | 68.8 | 26 | 963 | 150 |
| Reverse | 5′-ATTCAGACACAGGTTGGTGAAG-3′ | 59.1 | 22 | 1002 | 151 |

TABLE 21

Probe Name Ag2233

| | | | | Start | SEQ ID |
|---|---|---|---|---|---|
| Primers | Sequences | TM | Length | Position | NO: |
| Forward | 5′-CATGCATTTCTGACCTTGCT-3′ | 58.9 | 20 | 101 | 152 |
| Probe | FAM-5′-CCCACTCAAAGCTTTTCAAGGGCTCT-3′-TAMRA | 69.4 | 26 | 124 | 153 |
| Reverse | 5′-GTCATGCAGCTTTTGCTCAT-3′ | 59 | 20 | 166 | 154 |

TABLE 22

Probe Name Ag2241

| | | | | Start | SEQ ID |
|---|---|---|---|---|---|
| Primers | Sequences | TM | Length | Position | NO: |
| Forward | 5′-CCACAGTGACTTGCCACATT-3′ | 59.6 | 20 | 9500 | 155 |
| Probe | TET-5′-CCCCATTTGGAGAATGCTTTTATATCA-3′-TAMRA | 65.8 | 27 | 9524 | 156 |
| Reverse | 5′-AGGGCAGGCAGACTTAACC-3′ | 59.3 | 19 | 9580 | 157 |

TABLE 23

Probe Name Ag4154

| | | | | Start | SEQ ID |
|---|---|---|---|---|---|
| Primers | Sequences | TM | Length | Position | NO: |
| Forward | 5′-CTGTGAGGATATTGACGAATGC-3′ | 59.6 | 22 | 4867 | 158 |
| Probe | FAM-5′-TCCACACACTCCGGCATCTGTGG-3′-TAMRA | 71.8 | 23 | 4889 | 159 |
| Reverse | 5′-AGTTCCCCAGGGTGTTGTAG-3′ | 58.9 | 20 | 4924 | 160 |

TABLE 24

Probe Name Ag4334

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-CTGTGAGGATATTGACGAATGC-3′ | 59.6 | 22 | 4407 | 161 |
| Probe | FAM-5′-TCCACACACTCCGGCATCTGTGG-3′-TAMRA | 71.8 | 23 | 4429 | 162 |
| Reverse | 5′-TAGTTCCCCAGGGTGTTGTAG-3′ | 59 | 21 | 4464 | 163 |

TABLE 25

| | GMAC022146_A | 153568997 | CG88987-01 | CG88987-02 | CG88987-03 | CG88987-05 |
|---|---|---|---|---|---|---|
| Ag72 | X | X | X | X | X | X |
| Ag390 | | X | X | X | X | X |
| Ag671 | X | | X | X | | |
| Ag766 | X | X | X | X | X | X |
| Ag1211 | X | X | X | X | X | X |
| Ag1928 | | | | | | X |
| Ag1938 | X | X | X | X | X | X |
| Ag2233 | X | X | X | | | |
| Ag2241 | X | X | X | X | | X |
| Ag4154 | X | X | X | X | X | X |
| Ag4334 | X | X | X | X | X | X |

TABLE 26
Panel 1

| | Relative Expression(%) | | Relative Expression(%) | |
|---|---|---|---|---|
| Tissue Name | tm407f | tm486f | tm208f | tm362f |
| Endothelial cells | 0 | 0 | 0 | 0.1 |
| Endothelial cells (treated) | 0 | 0 | 0.1 | 0 |
| Pancreas | 0 | 0 | 0 | 0 |
| Pancreatic ca. CAPAN 2 | 0 | 0.8 | 0.3 | 0 |
| Adrenal gland | 0 | 0 | 0.1 | 0 |
| Thyroid | 7.4 | 5.1 | 4.5 | 0 |
| Salivary gland | 5.9 | 1.3 | 2.8 | 0.3 |
| Pituitary gland | 0 | 0.1 | 0 | 0 |
| Brain (fetal) | 100 | 60.7 | 100 | 96.6 |
| Brain (whole) | 3 | 3.6 | 0 | 0 |
| Brain (amygdala) | 0.8 | 1.1 | 0.5 | 0.1 |
| Brain (cerebellum) | 1.4 | 0.3 | 0.1 | 0 |
| Brain (hippocampus) | 2.8 | 1 | 0.7 | 0.2 |
| Brain (substantia nigra) | 7.3 | 0.9 | 0.4 | 0 |
| Brain (thalamus) | 2 | 1.1 | 0.4 | 0.1 |
| Brain (hypothalamus) | 5.6 | 1.7 | 2 | 0.2 |
| Spinal cord | 1.2 | 1.2 | 0.2 | 0 |
| CNS ca. (glio/astro) U87-MG | 0 | 0 | 0 | 0 |
| CNS ca. (glio/astro) U-118-MG | 0 | 0.1 | 0 | 0 |
| CNS ca. (astro) SW1783 | 0 | 0.1 | 0 | 0.1 |
| CNS ca.* (neuro; met) SK-N-AS | 0 | 0 | 0 | 0 |
| CNS ca. (astro) SF-539 | 0 | 0 | 0 | 0 |
| CNS ca. (astro) SNB-75 | 0 | 0 | 0 | 0 |
| CNS ca. (glio) SNB-19 | 2.7 | 2.3 | 1.4 | 0 |
| CNS ca. (glio) U251 | 0 | 0.8 | 0.5 | 0 |
| CNS ca. (glio) SF-295 | 0 | 0 | 0 | 0 |
| Heart | 0 | 0.5 | 0.1 | 0 |
| Skeletal muscle | 0 | 0.1 | 0 | 0.2 |
| Bone marrow | 0 | 0 | 0 | 0 |
| Thymus | 1.9 | 0 | 1.8 | 0 |
| Spleen | 0 | 0 | 0.3 | 0 |
| Lymph node | 0 | 0.1 | 0.1 | 0 |
| Colon (ascending) | 0.8 | 1.4 | 1.5 | 0.1 |
| Stomach | 0 | 0.2 | 0.1 | 0 |
| Small intestine | 0 | 0.1 | 0 | 0 |
| Colon ca. SW480 | 0 | 2.5 | 0 | 0 |
| Colon ca.* (SW480 met)SW620 | 0.2 | 3 | 0.5 | 0 |
| Colon ca. HT29 | 0 | 0 | 0.1 | 0 |
| Colon ca. HCT-116 | 2.7 | 6.5 | 0 | 0 |
| Colon ca. CaCo-2 | 21.9 | 17.4 | 7.4 | 6 |
| Colon ca. HCT-15 | 2 | 1.4 | 1.3 | 0.1 |
| Colon ca. HCC-2998 | 0 | 2.7 | 0.4 | 0 |
| Gastric ca.* (liver met) NCI-N87 | 91.4 | 51.8 | 38.4 | 100 |
| Bladder | 0 | 0.5 | 0 | 0 |
| Trachea | 0 | 1.2 | 0 | 0 |
| Kidney | 7.6 | 3.1 | 3.7 | 2.4 |
| Kidney (fetal) | 46.7 | 8.7 | 16.5 | 0 |
| Renal ca. 786-0 | 0 | 0 | 0 | 0.1 |
| Renal ca. A498 | 0 | 0 | 0 | 0 |
| Renal ca. RXF393 | 0 | 0 | 0 | 0 |
| Renal ca. ACHN | 0 | 0 | 0 | 0 |
| Renal ca. UO-31 | 0 | 0.1 | 0.1 | 0 |
| Renal ca. TK-10 | 0 | 0 | 0.4 | 0 |
| Liver | 0.1 | 0.2 | 0 | 0 |
| Liver (fetal) | 0 | 0.4 | 0.7 | 0 |
| Liver ca. (hepatoblast) HepG2 | 14.7 | 29.7 | 3.9 | 3.1 |
| Lung | 7.7 | 0 | 1.4 | 14.9 |
| Lung (fetal) | 81.8 | 100 | 49.7 | 38.2 |
| Lung ca. (small cell) LX-1 | 1.6 | 8.1 | 1 | 0 |
| Lung ca. (small cell) NCI-H69 | 0 | 2.2 | 0.8 | 0 |
| Lung ca. (s.cell var.) SHP-77 | 0.3 | 1.9 | 0 | 0 |
| Lung ca. (large cell) NCI-H460 | 0 | 0.1 | 0 | 0.1 |
| Lung ca. (non-sm. cell) A549 | 0.1 | 0.1 | 0.1 | 0 |
| Lung ca. (non-s.cell) NCI-H23 | 0 | 2 | 0 | 0 |
| Lung ca. (non-s.cell) HOP-62 | 0 | 0 | 0 | 0.1 |
| Lung ca. (non-s.cl) NCI-H522 | 1.3 | 0.7 | 0.7 | 0 |
| Lung ca. (squam.) SW 900 | 0 | 1.7 | 0.3 | 0 |
| Lung ca. (squam.) NCI-H596 | 0 | 0.6 | 0 | 0 |
| Mammary gland | 0.2 | 2.4 | 0.8 | 0 |
| Breast ca.* (pl. effusion) MCF-7 | 0 | 0.2 | 0.2 | 0 |
| Breast ca.* (pl.ef) MDA-MB-231 | 0 | 0.1 | 0 | 0 |
| Breast ca.* (pl. effusion) T47D | 3.6 | 5.1 | 2.9 | 0 |
| Breast ca. BT-549 | 0 | 0.1 | 0 | 0 |
| Breast ca. MDA-N | 0 | 46.7 | 0.6 | 0 |
| Ovary | 0.3 | 1.7 | 2 | 0 |
| Ovarian ca. OVCAR-3 | 7.5 | 18.8 | 4.9 | 0.6 |
| Ovarian ca. OVCAR-4 | 8.8 | 5.4 | 3.3 | 7.4 |
| Ovanan ca. OVCAR-5 | 0 | 0.7 | 1.3 | 0.5 |
| Ovarian ca. OVCAR-8 | 0 | 1.4 | 0.1 | 0 |
| Ovarian ca. IGROV-1 | 0 | 1.6 | 0.4 | 0 |
| Ovarian ca.* (ascites) SK-OV-3 | 0 | 0.1 | 0.3 | 0 |
| Uterus | 8.5 | 2 | 5.3 | 0.3 |
| Placenta | 0 | 0 | 0 | 0 |
| Prostate | 0.3 | 0.5 | 1.5 | 0 |
| Prostate ca.* (bone met)PC-3 | 0 | 0.1 | 0 | 0 |
| Testis | 6.2 | 1.5 | 4.2 | 0 |
| Melanoma Hs688(A).T | 0 | 0 | 0 | 0 |
| Melanoma* (met) Hs688(B).T | 0 | 0.2 | 0.2 | 0 |
| Melanoma UACC-62 | 0 | 0 | 0 | 0 |
| Melanoma M14 | 0 | 0.1 | 1.7 | 0 |
| Melanoma LOX IMVI | 0 | 0 | 0.2 | 0 |
| Melanoma* (met) SK-MEL-5 | 0 | 0.1 | 0 | 0 |
| Melanoma SK-MEL-28 | 0 | 0 | 0 | 0 |

TABLE 27
Panel 1.1

| | Relative | | Relative |
|---|---|---|---|
| | Expression(%) | | Expression(%) |
| | 1.1tm808f_{—} | | 1.1tm808f_{—} |
| Tissue Name | ag671 | Tissue Name | ag671 |
| Adrenal gland | 0.0 | Renal ca. UO-31 | 0.0 |
| Bladder | 0.0 | Renal ca. RXF 393 | 0.0 |
| Brain (amygdala) | 0.0 | Liver | 0.0 |
| Brain (cerebellum) | 0.0 | Liver (fetal) | 0.0 |
| Brain (hippocampus) | 0.0 | Liver ca. (hepatoblast) HepG2 | 4.3 |
| Brain (substantia nigra) | 0.5 | Lung | 0.0 |
| Brain (thalamus) | 0.0 | Lung (fetal) | 70.7 |
| Cerebral Cortex | 0.0 | Lung ca (non-s.cell) HOP-62 | 0.0 |
| Brain (fetal) | 100.0 | Lung ca. (large cell)NCI-H460 | 0.0 |
| Brain (whole) | 0.0 | Lung ca. (non-s.cell) NCI-H23 | 0.0 |
| CNS ca. (glio/astro) U-118-MG | 0.0 | Lung ca. (non-s.cl) NCI-H522 | 0.0 |
| CNS ca. (astro) SF-539 | 0.0 | Lung ca. (non-sm. cell) A549 | 0.0 |
| CNS ca. (astro) SNB-75 | 0.0 | Lung ca. (s.cell var.) SHP-77 | 0.0 |
| CNS ca. (astro) SW1783 | 0.0 | Lung ca. (small cell) LX-1 | 0.5 |
| CNS ca. (glio) U251 | 0.0 | Lung ca. (small cell) NCI-H69 | 0.0 |
| CNS ca. (glio) SF-295 | 0.0 | Lung ca. (squam.) SW 900 | 0.0 |
| CNS ca. (glio) SNB-19 | 0.0 | Lung ca. (squam.) NCI-H596 | 0.0 |
| CNS ca. (glio/astro) U87-MG | 0.0 | Lymph node | 0.0 |
| CNS ca.* (neuro; met) SK-N-AS | 0.0 | Spleen | 0.0 |
| Mammary gland | 0.0 | Thymus | 0.0 |
| Breast ca. BT-549 | 0.0 | Ovary | 0.0 |
| Breast ca. MDA-N | 0.0 | Ovarian ca. IGROV-1 | 0.0 |
| Breast ca.* (pl. effusion) T47D | 0.2 | Ovarian ca. OVCAR-3 | 20.3 |
| Breast ca.* (pl. effusion) MCF-7 | 0.0 | Ovarian ca. OVCAR-4 | 4.5 |
| Breast ca.* (pl.ef) MDA-MB-231 | 0.0 | Ovarian ca. OVCAR-5 | 0.0 |
| Small intestine | 0.0 | Ovarian ca. OVCAR-8 | 0.0 |
| Colorectal | 0.0 | Ovarian ca.* (ascites) SK-OV-3 | 0.0 |
| Colon ca. HT29 | 0.0 | Pancreas | 0.0 |
| Colon ca. CaCo-2 | 9.9 | Pancreatic ca. CAPAN 2 | 0.0 |
| Colon ca. HCT-15 | 0.0 | Pituitary gland | 0.6 |
| Colon ca. HCT-116 | 0.0 | Placenta | 0.0 |
| Colon ca. HCC-2998 | 0.0 | Prostate | 0.0 |
| Colon ca. SW480 | 0.0 | Prostate ca.* (bone met)PC-3 | 0.0 |
| Colon ca.* (SW480 met)SW620 | 0.0 | Salivary gland | 0.5 |
| Stomach | 0.0 | Trachea | 0.2 |
| Gastric ca.* (liver met) NCI-N87 | 66.0 | Spinal cord | 0.1 |
| Heart | 0.0 | Testis | 0.0 |
| Fetal Skeletal | 1.0 | Thyroid | 4.1 |
| Skeletal muscle | 0.0 | Uterus | 0.0 |
| Endothelial cells | 0.0 | Melanoma M14 | 0.0 |
| Heart (fetal) | 0.0 | Melanoma LOX IMVI | 0.0 |
| Kidney | 15.3 | Melanoma UACC-62 | 0.0 |
| Kidney (fetal) | 11.5 | Melanoma SK-MEL-28 | 0.0 |
| Renal ca. 786-0 | 0.0 | Melanoma* (met) SK-MEL-5 | 0.0 |
| Renal ca. A498 | 0.0 | Melanoma Hs688(A).T | 0.0 |
| Renal ca. ACHN | 0.0 | Melanoma* (met) Hs688(B).T | 0.0 |
| Renal ca. TK-10 | 0.0 | | |

TABLE 28
Panel 1.2

| | Relative | | Relative |
|---|---|---|---|
| | Expression(%) | | Expression(%) |
| | 1.2tm915t_{—} | | 1.2tm915t_{—} |
| Tissue Name | ag766 | Tissue Name | ag766 |
| Endothelial cells | 0.0 | Renal ca. 786-0 | 0.0 |
| Heart (fetal) | 0.0 | Renal ca. A498 | 0.0 |
| Pancreas | 0.3 | Renal ca. RXF 393 | 0.0 |
| Pancreatic ca. CAPAN 2 | 0.0 | Renal ca. ACHN | 0.0 |
| Adrenal Gland (new lot*) | 0.0 | Renal ca. UO-31 | 0.0 |
| Thyroid | 9.3 | Renal ca. TK-10 | 0.0 |
| Salivary gland | 6.2 | Liver | 0.5 |
| Pituitary gland | 6.7 | Liver (fetal) | 2.4 |
| Brain (fetal) | 100.0 | Liver ca. (hepatoblast) HepG2 | 6.5 |
| Brain (whole) | 3.7 | Lung | 3.7 |
| Brain (amygdala) | 2.2 | Lung (fetal) | 60.3 |
| Brain (cerebellum) | 0.0 | Lung ca. (small cell) LX-1 | 3.7 |
| Brain (hippocampus) | 5.4 | Lung ca. (small cell) NCI-H69 | 0.0 |
| Brain (thalamus) | 2.1 | Lung ca. (s.cell var.) SHP-77 | 0.2 |
| Cerebral Cortex | 2.5 | Lung ca. (large cell)NCI-H460 | 0.0 |
| Spinal cord | 2.0 | Lung ca. (non-sm. cell) A549 | 0.0 |
| CNS ca. (glio/astro) U87-MG | 0.0 | Lung ca. (non-s.cell) NCI-H23 | 0.1 |
| CNS ca. (glio/astro) U-118-MG | 0.0 | Lung ca. (non-s.cell) HOP-62 | 0.0 |
| CNS ca. (astro) SW1783 | 0.0 | Lung ca. (non-s.cl) NCI-H522 | 1.2 |
| CNS ca.* (neuro; met) SK-N-AS | 0.0 | Lung ca. (squam.) SW 900 | 0.3 |
| CNS ca. (astro) SF-539 | 0.0 | Lung ca. (squam.) NCI-H596 | 0.0 |
| CNS ca. (astro) SNB-75 | 0.0 | Mammary gland | 2.3 |
| CNS ca. (glio) SNB-19 | 0.6 | Breast ca.* (pl. effusion) MCF-7 | 0.0 |
| CNS ca. (glio) U251 | 0.0 | Breast ca.* (pl.ef) MDA-MB-231 | 0.0 |
| CNS ca. (glio) SF-295 | 0.0 | Breast ca.* (pl. effusion) T47D | 2.0 |
| Heart | 1.2 | Breast ca. BT-549 | 0.0 |
| Skeletal Muscle (new lot*) | 0.6 | Breast ca. MDA-N | 0.0 |
| Bone marrow | 0.0 | Ovary | 0.5 |
| Thymus | 0.6 | Ovarian ca. OVCAR-3 | 11.2 |
| Spleen | 0.0 | Ovarian ca. OVCAR-4 | 6.2 |
| Lymph node | 0.0 | Ovarian ca. OVCAR-5 | 0.1 |
| Colorectal | 0.0 | Ovarian ca. OVCAR-8 | 0.2 |
| Stomach | 0.1 | Ovarian ca. IGROV-1 | 1.0 |
| Small intestine | 0.3 | Ovarian ca.* (ascites) SK-OV-3 | 0.0 |
| Colon ca. SW480 | 0.6 | Uterus | 0.0 |
| Colon ca.* (SW480 met)SW620 | 1.3 | Placenta | 0.0 |
| Colon ca. HT29 | 0.0 | Prostate | 3.1 |
| Colon ca. HCT-116 | 1.6 | Prostate ca.* (bone met)PC-3 | 0.0 |
| Colon ca. CaCo-2 | 7.6 | Testis | 3.8 |
| 83219 CC Well to Mod Diff | 0.0 | Melanoma Hs688(A).T | 0.0 |
| (ODO3866) | | | |
| Colon ca. HCC-2998 | 0.7 | Melanoma* (met) Hs688(B).T | 0.0 |
| Gastric ca.* (liver met) NCI-N87 | 50.7 | Melanoma UACC-62 | 0.0 |
| Bladder | 0.0 | Melanoma M14 | 0.0 |
| Trachea | 2.5 | Melanoma LOX IMVI | 0.0 |
| Kidney | 9.0 | Melanoma* (met) SK-MEL-5 | 0.0 |
| Kidney (fetal) | 29.3 | | |

TABLE 29
Panel 1.3D

| | | | Relative |
|---|---|---|---|
| | Relative Expression(%) | | Expression(%) |
| | 1.3Dtm2819f_ag | 1.3Dtm2843f_ag | 1.3dtm4344f_{—} |
| Tissue Name | 1211 | 1211 | ag2233 |
| Liver adenocarcinoma | 30.6 | 39.0 | 14.2 |
| Pancreas | 0.0 | 0.4 | 0.0 |
| Pancreatic ca. CAPAN 2 | 0.0 | 0.0 | 0.0 |
| Adrenal gland | 0.7 | 0.0 | 0.0 |
| Thyroid | 2.8 | 5.7 | 4.4 |
| Salivary gland | 0.7 | 2.7 | 1.3 |
| Pituitary gland | 4.2 | 4.2 | 0.0 |
| Brain (fetal) | 47.6 | 49.7 | 100.0 |
| Brain (whole) | 1.5 | 0.7 | 2.2 |
| Brain (amygdala) | 0.6 | 2.3 | 0.8 |
| Brain (cerebellum) | 0.0 | 0.0 | 0.0 |
| Brain (hippocampus) | 4.1 | 4.0 | 3.2 |
| Brain (substantia nigra) | 1.2 | 1.2 | 0.9 |
| Brain (thalamus) | 2.0 | 3.1 | 1.7 |
| Cerebral Cortex | 8.1 | 5.6 | 4.1 |
| Spinal cord | 1.0 | 1.2 | 0.0 |
| CNS ca. (glio/astro) U87-MG | 0.0 | 0.0 | 0.0 |
| CNS ca. (glio/astro) U-118-MG | 0.0 | 0.3 | 0.8 |
| CNS ca. (astro) SW1783 | 0.0 | 0.0 | 0.0 |
| CNS ca.* (neuro; met) SK-N-AS | 0.0 | 0.0 | 0.0 |
| CNS ca. (astro) SF-539 | 0.0 | 0.0 | 0.0 |
| CNS ca. (astro) SNB-75 | 2.2 | 0.6 | 0.0 |
| CNS ca. (glio) SNB-19 | 0.1 | 0.4 | 1.2 |
| CNS ca. (glio) U251 | 0.7 | 0.0 | 0.0 |
| CNS ca. (glio) SF-295 | 0.0 | 0.0 | 0.0 |
| Heart (fetal) | 2.7 | 2.6 | 0.0 |
| Heart | 0.0 | 0.8 | 0.0 |
| Fetal Skeletal | 79.6 | 100.0 | 24.5 |
| Skeletal muscle | 0.5 | 2.5 | 0.0 |
| Bone marrow | 0.0 | 0.0 | 0.0 |
| Thymus | 2.2 | 0.0 | 1.5 |
| Spleen | 0.0 | 0.0 | 0.8 |
| Lymph node | 0.0 | 0.0 | 0.0 |
| Colorectal | 0.0 | 0.2 | 0.9 |
| Stomach | 0.0 | 0.0 | 0.0 |
| Small intestine | 0.0 | 0.5 | 0.0 |
| Colon ca. SW480 | 0.3 | 1.9 | 4.7 |
| Colon ca.* (SW480 met)SW620 | 0.2 | 0.0 | 0.0 |
| Colon ca. HT29 | 0.0 | 0.0 | 1.7 |
| Colon ca. HCT-116 | 1.6 | 1.3 | 2.1 |
| Colon ca. CaCo-2 | 15.1 | 12.8 | 11.5 |
| 83219 CC Well to Mod Diff (ODO3866) | 0.0 | 0.0 | 0.0 |
| Colon ca. HCC-2998 | 0.3 | 0.2 | 2.0 |
| Gastric ca.* (liver met) NCI-N87 | 94.6 | 93.3 | 53.6 |
| Bladder | 0.0 | 0.0 | 0.0 |
| Trachea | 2.9 | 2.0 | 12.0 |
| Kidney | 2.9 | 4.4 | 4.1 |
| Kidney (fetal) | 10.2 | 11.7 | 6.2 |
| Renal ca. 786-0 | 0.0 | 0.0 | 0.0 |
| Renal ca. A498 | 0.0 | 0.0 | 0.0 |
| Renal ca. RXF 393 | 0.0 | 0.0 | 0.0 |
| Renal ca. ACHN | 0.0 | 0.2 | 0.0 |
| Renal ca. UO-31 | 0.0 | 0.0 | 0.0 |
| Renal ca. TK-10 | 0.0 | 0.0 | 0.0 |
| Liver | 0.0 | 0.0 | 0.0 |
| Liver (fetal) | 1.4 | 1.8 | 0.3 |
| Liver ca. (hepatoblast) HepG2 | 12.2 | 4.8 | 9.5 |
| Lung | 6.7 | 1.8 | 5.1 |
| Lung (fetal) | 100.0 | 92.0 | 25.5 |
| Lung ca. (small cell) LX-1 | 1.5 | 2.6 | 0.9 |
| Lung ca. (small cell) NCI-H69 | 0.0 | 0.0 | 0.0 |
| Lung ca. (s.cell var.) SHP-77 | 0.0 | 0.3 | 0.0 |
| Lung ca. (large cell)NCI-H460 | 0.0 | 0.0 | 0.0 |
| Lung ca. (non-sm. cell) A549 | 0.6 | 0.0 | 0.0 |
| Lung ca. (non-s.cell) NCI-H23 | 1.0 | 0.0 | 0.0 |
| Lung ca (non-s.cell) HOP-62 | 0.0 | 0.5 | 0.0 |
| Lung ca. (non-s.cl) NCI-H522 | 0.3 | 0.2 | 0.0 |
| Lung ca. (squam.) SW 900 | 0.3 | 0.6 | 1.8 |
| Lung ca. (squam.) NCI-H596 | 0.0 | 0.0 | 0.0 |
| Mammary gland | 4.1 | 2.6 | 0.8 |
| Breast ca.* (pl. effusion) MCF-7 | 0.0 | 0.0 | 0.0 |
| Breast ca.* (pl.ef) MDA-MB-231 | 0.0 | 0.0 | 0.0 |
| Breast ca.* (pl. effusion) T47D | 1.5 | 0.7 | 1.5 |
| Breast ca. BT-549 | 0.0 | 0.0 | 1.6 |
| Breast ca. MDA-N | 0.0 | 0.0 | 0.0 |
| Ovary | 1.4 | 4.6 | 1.7 |
| Ovarian ca. OVCAR-3 | 15.3 | 12.9 | 7.9 |
| Ovarian ca. OVCAR-4 | 0.5 | 0.8 | 0.9 |
| Ovarian ca. OVCAR-5 | 0.0 | 0.0 | 0.7 |
| Ovarian ca. OVCAR-8 | 0.4 | 0.4 | 0.0 |
| Ovarian ca. IGROV-1 | 0.3 | 0.4 | 0.0 |
| Ovarian ca.* (ascites) SK-OV-3 | 0.0 | 0.0 | 0.0 |
| Uterus | 0.0 | 0.2 | 0.0 |
| Placenta | 0.0 | 0.0 | 0.0 |
| Prostate | 0.0 | 0.3 | 0.0 |
| Prostate ca.* (bone met)PC-3 | 0.0 | 0.0 | 0.0 |
| Testis | 1.2 | 0.8 | 1.7 |
| Melanoma Hs688(A).T | 0.0 | 0.0 | 0.0 |
| Melanoma* (met) Hs688(B).T | 0.0 | 0.0 | 0.0 |
| Melanoma UACC-62 | 0.0 | 0.0 | 0.0 |
| Melanoma M14 | 0.0 | 0.0 | 0.0 |
| Melanoma LOX IMVI | 0.0 | 0.0 | 0.0 |
| Melanoma* (met) SK-MEL-5 | 0.0 | 0.0 | 0.0 |
| Adipose | 0.4 | 0.4 | 0.0 |

TABLE 30
Panel General_screening_panel_v1.4

| | Relative | Relative | Relative |
|---|---|---|---|
| | Expression(%) | Expression(%) | Expression(%) |
| | 1.4x4tm7291f | 1.4tm7578f | 1.4x4tm7676f |
| Tissue Name | _ag1928_a2 | _ag4154 | _ag4334_b2 |
| D6005-01_Human adipose | 0.0 | 0.0 | 0.1 |
| 112193_Metastatic melanoma | 0.0 | 0.0 | 0.0 |
| 112192_Metastatic melanoma | 0.0 | 0.0 | 0.0 |
| 95280_Epidermis (metastatic melanoma) | 0.0 | 0.0 | 0.0 |
| 95279_Epidermis (metastatic melanoma) | 0.0 | 0.0 | 0.0 |
| Melanoma (met)_SK-MEL-5 | 0.0 | 0.0 | 0.0 |
| 112196_Tongue (oncology) | 0.0 | 0.0 | 0.0 |
| 113461_Testis Pool | 0.0 | 0.7 | 0.3 |
| Prostate ca.(bone met)_PC-3 | 0.0 | 0.0 | 0.0 |
| 113455_Prostate Pool | 0.0 | 0.3 | 0.5 |
| 103396_Placenta | 0.0 | 0.3 | 0.1 |
| 113463_Uterus Pool | 0.0 | 0.0 | 0.0 |
| Ovarian carcinoma_OVCAR-3 | 15.8 | 8.0 | 6.9 |
| Ovarian carcinoma(ascites)_SK-OV-3 | 0.0 | 0.0 | 0.0 |
| 95297_Adenocarcinoma (ovary) | 3.9 | 3.0 | 2.2 |
| Ovarian carcinoma_OVCAR-5 | 0.0 | 0.7 | 0.9 |
| Ovarian carcinoma_IGROV-1 | 0.0 | 1.3 | 0.5 |
| Ovarian carcinoma_OVCAR-8 | 0.0 | 0.2 | 0.1 |
| 103368_Ovary | 0.0 | 0.1 | 0.1 |
| MCF7_breast carcinoma(pleural effusion) | 0.0 | 0.1 | 0.2 |
| Breast ca. (pleural effusion)_MDA-MB-231 | 0.0 | 0.0 | 0.1 |
| 112189_ductal cell carcinoma(breast) | 0.0 | 0.0 | 0.0 |
| Breast ca. (pleural effusion)_T47D | 2.3 | 1.8 | 1.5 |
| Breast carcinoma_MDA-N | 0.0 | 0.0 | 0.0 |
| 113452_Breast Pool | 0.0 | 0.7 | 0.4 |
| 103398_Trachea | 0.0 | 3.9 | 2.1 |
| 112354_lung | 0.0 | 0.1 | 0.2 |
| 103374_Fetal Lung | 29.0 | 44.1 | 44.5 |
| 94921_Small cell carcinoma of the lung | 0.0 | 0.0 | 0.0 |
| Lung ca.(small cell)_LX-1 | 0.0 | 6.0 | 4.3 |
| 94919_Small cell carcinoma of the lung | 6.0 | 5.6 | 3.9 |
| Lung ca.(s.cell var.)_SHP-77 | 0.0 | 0.4 | 0.5 |
| 95268_Lung (Large cell carcinoma) | 0.0 | 0.0 | 0.0 |
| 94920_Small cell carcinoma of the lung | 0.0 | 0.0 | 0.0 |
| Lung ca.(non-s.cell)_NCI-H23 | 0.0 | 1.0 | 0.7 |
| Lung ca.(large cell)_NCI-H460 | 0.0 | 0.0 | 0.0 |
| Lung ca.(non-s.cell)_HOP-62 | 0.0 | 0.0 | 0.0 |
| Lung ca.(non-s.cl)_NCI-H522 | 0.0 | 0.5 | 0.9 |
| 103392_Liver | 0.0 | 0.0 | 0.0 |
| 103393_Fetal Liver | 0.0 | 1.6 | 1.6 |
| Liver ca.(hepatoblast)_HepG2 | 13.2 | 7.3 | 4.2 |
| 113465_Kidney Pool | 0.0 | 0.1 | 0.1 |
| 103373_Fetal Kidney | 9.4 | 33.2 | 25.4 |
| Renal ca._786-0 | 0.0 | 0.0 | 0.0 |
| 112188_renal cell carcinoma | 0.0 | 0.0 | 0.0 |
| Renal ca._ACHN | 0.0 | 0.0 | 0.0 |
| 112190_Renal cell carcinoma | 0.0 | 0.0 | 0.0 |
| Renal ca._TK-10 | 5.1 | 2.8 | 1.3 |
| Bladder | 0.0 | 0.3 | 0.2 |
| Gastric ca.(liver met)_NCI-N87 | 28.8 | 83.5 | 80.5 |
| 112197_Stomach | 3.6 | 4.7 | 3.0 |
| 94938_Colon Adenocarcinoma | 0.0 | 0.0 | 0.0 |
| Colon ca._SW480 | 1.2 | 8.8 | 3.7 |
| Colon ca.(SW480 met)_SW620 | 0.0 | 2.6 | 0.9 |
| Colon ca._HT29 | 0.0 | 0.0 | 0.0 |
| Colon ca._HCT-116 | 2.3 | 4.0 | 4.4 |
| Colon ca._CaCo-2 | 23.0 | 17.7 | 11.7 |
| 83219_CC Well to Mod Diff(ODO3866) | 0.0 | 0.0 | 0.0 |
| 94936_Colon Adenocarcinoma | 0.0 | 0.6 | 0.1 |
| 94930_Colon | 0.0 | 0.0 | 0.0 |
| 94935_Colon Adenocarcinoma | 0.0 | 0.0 | 0.0 |
| 113468_Colon Pool | 0.0 | 0.0 | 0.1 |
| 113457_Small Intestine Pool | 0.0 | 0.4 | 0.1 |
| 113460_Stomach Pool | 0.0 | 1.6 | 0.7 |
| 113467_Bone Marrow Pool | 0.0 | 0.0 | 0.0 |
| 103371_Fetal Heart | 0.0 | 0.9 | 0.7 |
| 113451_Heart Pool | 0.0 | 0.3 | 0.1 |
| 113466_Lymph Node Pool | 0.0 | 0.7 | 0.4 |
| 103372_Fetal Skeletal Muscle | 2.7 | 6.8 | 3.6 |
| 113456_Skeletal Muscle Pool | 0.0 | 0.9 | 0.1 |
| 113459_Spleen Pool | 0.0 | 0.0 | 0.1 |
| 113462_Thymus Pool | 0.0 | 2.7 | 1.5 |
| CNS ca. (glio/astro)_U87-MG | 0.0 | 0.0 | 0.0 |
| CNS ca. (glio/astro)_U-118-MG | 0.0 | 0.0 | 0.0 |
| CNS ca. (neuro;met)_SK-N-AS | 0.0 | 0.0 | 0.0 |
| 95264_Brain astrocytoma | 0.0 | 0.0 | 0.0 |
| CNS ca. (astro)_SNB-75 | 0.0 | 0.0 | 0.0 |
| CNS ca. (glio)_SNB-19 | 0.0 | 1.1 | 0.4 |
| CNS ca. (glio)_SF-295 | 0.0 | 0.1 | 0.0 |
| 113447_Brain (Amygdala) Pool | 0.0 | 0.3 | 0.5 |
| 103382_Brain (cerebellum) | 0.0 | 0.6 | 0.6 |
| 64019-1_brain (fetal) | 100.0 | 100.0 | 100.0 |
| 113448_Brain (Hippocampus) Pool | 0.0 | 1.1 | 0.2 |
| 113464_Cerebral Cortex Pool | 0.0 | 0.5 | 0.5 |
| 113449_Brain (Substantia nigra) Pool | 0.0 | 0.6 | 0.3 |
| 113450_Brain (Thalamus) Pool | 0.0 | 0.2 | 0.7 |
| 103384_Brain (whole) | 0.0 | 1.6 | 3.3 |
| 113458_Spinal Cord Pool | 0.0 | 0.9 | 0.5 |
| 103375_Adrenal Gland | 0.0 | 0.0 | 0.0 |
| 113454_Pituitary gland Pool | 0.0 | 0.2 | 1.1 |
| 103397_Salivary Gland | 0.0 | 2.6 | 0.8 |
| 103369_Thyroid (female) | 2.2 | 7.7 | 4.2 |
| Pancreatic ca._CAPAN2 | 0.0 | 0.4 | 0.3 |
| 113453_Pancreas Pool | 0.0 | 1.2 | 0.1 |

TABLE 31
Panel 2.2

| | Relative | | Relative |
|---|---|---|---|
| | Expression(%) | | Expression(%) |
| | 2.2x4tm6426t | | 2.2x4tm6426t |
| Tissue Name | _ag2241_a2 | Tissue Name | _ag2241_a2 |
| Normal Colon GENPAK 061003 | 0.0 | 83793 Kidney NAT (OD04348) | 0.0 |
| 97759 Colon cancer (OD06064) | 3.1 | 98938 Kidney malignant cancer | 1.9 |
| | | (OD06204B) | |
| 97760 Colon cancer NAT | 0.0 | 98939 Kidney normal adjacent | 0.0 |
| (OD06064) | | tissue (OD06204E) | |
| 97778 Colon cancer (OD06159) | 0.0 | 85973 Kidney Cancer (OD04450- | 0.0 |
| | | 01) | |
| 97779 Colon cancer NAT | 0.0 | 85974 Kidney NAT (OD04450-03) | 2.0 |
| (OD06159) | | | |
| 98861 Colon cancer (OD06297-04) | 0.0 | Kidney Cancer Clontech 8120613 | 0.0 |
| 98862 Colon cancer NAT | 0.0 | Kidney NAT Clontech 8120614 | 0.0 |
| (OD06297-015) | | | |
| 83237 CC Gr.2 ascend colon | 0.0 | Kidney Cancer Clontech 9010320 | 0.0 |
| (ODO3921) | | | |
| 83238 CC NAT (ODO3921) | 0.0 | Kidney NAT Clontech 9010321 | 1.4 |
| 97766 Colon cancer metastasis | 0.0 | Kidney Cancer Clontech 8120607 | 0.0 |
| (OD06104) | | | |
| 97767 Lung NAT (OD06104) | 3.6 | Kidney NAT Clontech 8120608 | 0.0 |
| 87472 Colon mets to lung | 0.0 | Normal Uterus GENPAK 061018 | 0.0 |
| (OD04451-01) | | | |
| 87473 Lung NAT (OD04451-02) | 0.0 | Uterus Cancer GENPAK 064011 | 0.0 |
| Normal Prostate Clontech A+ | 0.0 | Normal Thyroid Clontech A+ | 0.0 |
| 6546-1 (8090438) | | 6570-1 (7080817) | |
| 84140 Prostate Cancer (OD04410) | 0.0 | Thyroid Cancer GENPAK 064010 | 0.0 |
| 84141 Prostate NAT (OD04410) | 0.0 | Thyroid Cancer INVITROGEN | 0.0 |
| | | A302152 | |
| Normal Ovary Res. Gen. | 0.0 | Thyroid NAT INVITROGEN | 0.0 |
| | | A302153 | |
| 98863 Ovarian cancer (OD06283- | 0.0 | Normal Breast GENPAK 061019 | 0.0 |
| 03) | | | |
| 98865 Ovarian cancer | 0.0 | 84877 Breast Cancer (OD04566) | 0.0 |
| NAT/fallopian tube (OD06283-07) | | | |
| Ovarian Cancer GENPAK 064008 | 0.0 | Breast Cancer Res. Gen. 1024 | 0.0 |
| 97773 Ovarian cancer (OD06145) | 0.0 | 85975 Breast Cancer (OD04590- | 0.0 |
| | | 01) | |
| 97775 Ovarian cancer NAT | 0.0 | 85976 Breast Cancer Mets | 0.0 |
| (OD06145) | | (OD04590-03) | |
| 98853 Ovarian cancer (OD06455- | 0.0 | 87070 Breast Cancer Metastasis | 0.0 |
| 03) | | (OD04655-05) | |
| 98854 Ovarian NAT (OD06455- | 0.0 | GENPAK Breast Cancer 064006 | 0.0 |
| 07) Fallopian tube | | | |
| Normal Lung GENPAK 061010 | 0.0 | Breast Cancer Clontech 9100266 | 0.0 |
| 92337 Invasive poor diff lung | 0.0 | Breast NAT Clontech 9100265 | 0.0 |
| adeno (ODO4945-01) | | | |
| 92338 Lung NAT (ODO4945-03) | 0.0 | Breast Cancer INVITROGEN | 0.0 |
| | | A209073 | |
| 84136 Lung Malignant Cancer | 0.0 | Breast NAT INVITROGEN | 0.0 |
| (OD03126) | | A2090734 | |
| 84137 Lung NAT (OD03126) | 0.0 | 97763 Breast cancer (OD06083) | 0.0 |
| 90372 Lung Cancer (OD05014A) | 0.0 | 97764 Breast cancer node | 0.0 |
| | | metastasis (OD06083) | |
| 90373 Lung NAT (OD05014B) | 0.0 | Normal Liver GENPAK 061009 | 1.8 |
| 97761 Lung cancer (OD06081) | 0.0 | Liver Cancer Research Genetics | 0.0 |
| | | RNA 1026 | |
| 97762 Lung cancer NAT | 0.0 | Liver Cancer Research Genetics | 0.0 |
| (OD06081) | | RNA 1025 | |
| 85950 Lung Cancer (OD04237-01) | 0.0 | Paired Liver Cancer Tissue | 4.0 |
| | | Research Genetics RNA 6004-T | |
| 85970 Lung NAT (OD04237-02) | 0.0 | Paired Liver Tissue Research | 0.0 |
| | | Genetics RNA 6004-N | |
| 83255 Ocular Mel Met to Liver | 0.0 | Paired Liver Cancer Tissue | 0.0 |
| (ODO4310) | | Research Genetics RNA 6005-T | |
| 83256 Liver NAT (ODO4310) | 0.0 | Paired Liver Tissue Research | 0.0 |
| | | Genetics RNA 6005-N | |
| 84139 Melanoma Mets to Lung | 0.0 | Liver Cancer GENPAK 064003 | 0.0 |
| (OD04321) | | | |
| 84138 Lung NAT (OD04321) | 0.0 | Normal Bladder GENPAK 061001 | 0.0 |
| Normal Kidney GENPAK 061008 | 0.0 | Bladder Cancer Research Genetics | 0.0 |
| | | RNA 1023 | |
| 83786 Kidney Ca, Nuclear grade 2 | 0.0 | Bladder Cancer INVITROGEN | 0.0 |
| (OD04338) | | A302173 | |
| 83787 Kidney NAT (OD04338) | 0.0 | Normal Stomach GENPAK | 30.4 |
| | | 061017 | |
| 83788 Kidney Ca Nuclear grade | 100.0 | Gastric Cancer Clontech 9060397 | 0.0 |
| 1/2 (OD04339) | | | |
| 83789 Kidney NAT (OD04339) | 0.0 | NAT Stomach Clontech 9060396 | 0.0 |
| 83790 Kidney Ca, Clear cell type | 0.0 | Gastric Cancer Clontech 9060395 | 0.0 |
| (OD04340) | | | |
| 83791 Kidney NAT (OD04340) | 0.0 | NAT Stomach Clontech 9060394 | 0.0 |
| 83792 Kidney Ca, Nuclear grade 3 | 0.0 | Gastric Cancer GENPAK 064005 | 0.0 |
| (OD04348) | | | |

TABLE 32
Panel 2D

| | Relative Expression(%) | |
|---|---|---|
| | 2Dtm2820f_{—} | 2Dtm2841f_{—} |
| Tissue Name | ag1211 | ag1211 |
| Normal Colon GENPAK 061003 | 0.5 | 0.0 |
| 83219 CC Well to Mod Diff (ODO3866) | 0.0 | 0.1 |
| 83220 CC NAT (ODO3866) | 0.0 | 0.3 |
| 83221 CC Gr.2 rectosigmoid (ODO3868) | 0.0 | 0.0 |
| 83222 CC NAT (ODO3868) | 0.0 | 0.0 |
| 83235 CC Mod Diff (ODO3920) | 0.0 | 0.0 |
| 83236 CC NAT (ODO3920) | 0.1 | 0.0 |
| 83237 CC Gr.2 ascend colon (ODO3921) | 0.0 | 0.3 |
| 83238 CC NAT (ODO3921) | 0.0 | 0.3 |
| 83241 CC from Partial Hepatectomy (ODO4309) | 0.2 | 0.0 |
| 83242 Liver NAT (ODO4309) | 0.0 | 0.0 |
| 87472 Colon mets to lung (OD04451-01) | 0.0 | 0.0 |
| 87473 Lung NAT (OD04451-02) | 0.4 | 0.2 |
| Normal Prostate Clontech A+ 6546-1 | 3.3 | 4.5 |
| 84140 Prostate Cancer (OD04410) | 0.4 | 0.6 |
| 84141 Prostate NAT (OD04410) | 1.2 | 0.6 |
| 87073 Prostate Cancer (OD04720-01) | 2.1 | 3.5 |
| 87074 Prostate NAT (OD04720-02) | 3.4 | 6.0 |
| Normal Lung GENPAK 061010 | 10.8 | 10.9 |
| 83239 Lung Met to Muscle (ODO4286) | 0.0 | 0.3 |
| 83240 Muscle NAT (ODO4286) | 0.0 | 0.5 |
| 84136 Lung Malignant Cancer (OD03126) | 1.0 | 0.9 |
| 84137 Lung NAT (OD03126) | 6.1 | 5.9 |
| 84871 Lung Cancer (OD04404) | 1.4 | 0.6 |
| 84872 Lung NAT (OD04404) | 3.2 | 6.7 |
| 84875 Lung Cancer (OD04565) | 0.0 | 0.0 |
| 84876 Lung NAT (OD04565) | 4.9 | 4.0 |
| 85950 Lung Cancer (OD04237-01) | 12.8 | 20.9 |
| 85970 Lung NAT (OD04237-02) | 5.9 | 4.8 |
| 83255 Ocular Mel Met to Liver (ODO4310) | 0.0 | 0.0 |
| 83256 Liver NAT (ODO4310) | 0.0 | 0.0 |
| 84139 Melanoma Mets to Lung (OD04321) | 0.0 | 0.2 |
| 84138 Lung NAT (OD04321) | 2.9 | 3.0 |
| Normal Kidney GENPAK 061008 | 6.3 | 8.6 |
| 83786 Kidney Ca, Nuclear grade 2 (OD04338) | 0.0 | 0.0 |
| 83787 Kidney NAT (OD04338) | 3.3 | 5.3 |
| 83788 Kidney Ca Nuclear grade 1/2 (OD04339) | 0.0 | 0.0 |
| 83789 Kidney NAT (OD04339) | 4.0 | 5.2 |
| 83790 Kidney Ca, Clear cell type (OD04340) | 0.2 | 0.0 |
| 83791 Kidney NAT (OD04340) | 6.6 | 3.6 |
| 83792 Kidney Ca, Nuclear grade 3 (OD04348) | 0.0 | 0.0 |
| 83793 Kidney NAT (OD04348) | 3.4 | 2.1 |
| 87474 Kidney Cancer (OD04622-01) | 0.0 | 0.0 |
| 87475 Kidney NAT (OD04622-03) | 0.9 | 1.2 |
| 85973 Kidney Cancer (OD04450-01) | 0.0 | 0.2 |
| 85974 Kidney NAT (OD04450-03) | 7.9 | 5.4 |
| Kidney Cancer Clontech 8120607 | 0.0 | 0.0 |
| Kidney NAT Clontech 8120608 | 2.3 | 1.5 |
| Kidney Cancer Clontech 8120613 | 100.0 | 100.0 |
| Kidney NAT Clontech 8120614 | 4.2 | 8.1 |
| Kidney Cancer Clontech 9010320 | 0.0 | 0.0 |
| Kidney NAT Clontech 9010321 | 3.0 | 1.0 |
| Normal Uterus GENPAK 061018 | 0.0 | 0.0 |
| Uterus Cancer GENPAK 064011 | 0.4 | 0.0 |
| Normal Thyroid Clontech A+ 6570-1 | 7.1 | 7.0 |
| Thyroid Cancer GENPAK 064010 | 19.3 | 19.2 |
| Thyroid Cancer INVITROGEN A302152 | 5.9 | 4.5 |
| Thyroid NAT INVITROGEN A302153 | 5.8 | 3.6 |
| Normal Breast GENPAK 061019 | 2.1 | 1.6 |
| 84877 Breast Cancer (OD04566) | 0.5 | 0.8 |
| 85975 Breast Cancer (OD04590-01) | 0.2 | 0.0 |
| 85976 Breast Cancer Mets (OD04590-03) | 0.2 | 0.0 |
| 87070 Breast Cancer Metastasis (OD04655-05) | 1.0 | 1.1 |
| GENPAK Breast Cancer 064006 | 0.4 | 0.5 |
| Breast Cancer Res. Gen. 1024 | 2.0 | 3.1 |
| Breast Cancer Clontech 9100266 | 0.7 | 1.0 |
| Breast NAT Clontech 9100265 | 0.6 | 0.3 |
| Breast Cancer INVITROGEN A209073 | 1.1 | 0.9 |
| Breast NAT INVITROGEN A2090734 | 0.8 | 0.5 |
| Normal Liver GENPAK 061009 | 0.0 | 0.3 |
| Liver Cancer GENPAK 064003 | 7.3 | 8.1 |
| Liver Cancer Research Genetics RNA 1025 | 0.0 | 0.3 |
| Liver Cancer Research Genetics RNA 1026 | 0.4 | 0.6 |
| Paired Liver Cancer Tissue Research Genetics RNA 6004-T | 0.0 | 0.0 |
| Paired Liver Tissue Research Genetics RNA 6004-N | 0.1 | 0.0 |
| Paired Liver Cancer Tissue Research Genetics RNA 6005-T | 0.2 | 0.7 |
| Paired Liver Tissue Research Genetics RNA 6005-N | 0.2 | 0.0 |
| Normal Bladder GENPAK 061001 | 0.1 | 0.2 |
| Bladder Cancer Research Genetics RNA 1023 | 0.1 | 0.0 |
| Bladder Cancer INVITROGEN A302173 | 2.0 | 1.0 |
| 87071 Bladder Cancer (OD04718-01) | 0.0 | 0.0 |
| 87072 Bladder Normal Adjacent (OD04718-03) | 0.0 | 0.0 |
| Normal Ovary Res. Gen. | 0.9 | 2.1 |
| Ovarian Cancer GENPAK 064008 | 7.5 | 8.1 |
| 87492 Ovary Cancer (OD04768-07) | 0.6 | 0.3 |
| 87493 Ovary NAT (OD04768-08) | 0.0 | 0.0 |
| Normal Stomach GENPAK 061017 | 0.0 | 0.0 |
| Gastric Cancer Clontech 9060358 | 0.1 | 0.3 |
| NAT Stomach Clontech 9060359 | 0.0 | 0.2 |
| Gastric Cancer Clontech 9060395 | 0.0 | 0.2 |
| NAT Stomach Clontech 9060394 | 0.2 | 0.0 |
| Gastric Cancer Clontech 9060397 | 0.1 | 0.2 |
| NAT Stomach Clontech 9060396 | 0.2 | 0.0 |
| Gastric Cancer GENPAK 064005 | 0.1 | 0.0 |

TABLE 33
Panel 4D

| | Relative | Relative |
|---|---|---|
| | Expression(%) | Expression(%) |
| | 4dx4tm4491t | 4dx4tm4230f |
| Tissue Name | _ag1938 | _ag2233_b2 |
| 93768_Secondary Th1_anti-CD28/anti-CD3 | 0.0 | 0.0 |
| 93769_Secondary Th2_anti-CD28/anti-CD3 | 0.0 | 0.0 |
| 93770_Secondary Tr1_anti-CD28/anti-CD3 | 0.0 | 0.0 |
| 93573_Secondary Th1_resting day 4-6 in IL-2 | 0.0 | 0.0 |
| 93572_Secondary Th2_resting day 4-6 in IL-2 | 0.0 | 0.0 |
| 93571_Secondary Tr1_resting day 4-6 in IL-2 | 0.0 | 0.0 |
| 93568_primary Th1_anti-CD28/anti-CD3 | 0.0 | 0.0 |
| 93569_primary Th2_anti-CD28/anti-CD3 | 0.0 | 0.0 |
| 93570_primary Tr1_anti-CD28/anti-CD3 | 0.0 | 0.0 |
| 93565_primary Th1_resting dy 4-6 in IL-2 | 0.0 | 0.0 |
| 93566_primary Th2_resting dy 4-6 in IL-2 | 0.0 | 0.0 |
| 93567_primary Tr1_resting dy 4-6 in IL-2 | 0.0 | 0.0 |
| 93351_CD45RA CD4 lymphocyte_anti-CD28/anti-CD3 | 0.0 | 0.0 |
| 93352_CD45RO CD4 lymphocyte_anti-CD28/anti-CD3 | 0.0 | 0.0 |
| 93251_CD8 Lymphocytes_anti-CD28/anti-CD3 | 0.0 | 0.0 |
| 93353_chronic CD8 Lymphocytes 2ry_resting dy 4-6 in IL-2 | 0.0 | 0.0 |
| 93574_chronic CD8 Lymphocytes 2ry_activated CD3/CD28 | 0.0 | 0.0 |
| 93354_CD4_none | 0.0 | 0.0 |
| 93252_Secondary Th1/Th2/Tr1_anti-CD95 CH11 | 0.0 | 0.0 |
| 93103_LAK cells_resting | 0.0 | 0.0 |
| 93788_LAK cells_IL-2 | 0.0 | 0.0 |
| 93787_LAK cells_IL-2+IL-12 | 0.0 | 0.0 |
| 93789_LAK cells_IL-2+IFN gamma | 0.0 | 0.0 |
| 93790_LAK cells_IL-2+ IL-18 | 0.0 | 0.0 |
| 93104_LAK cells_PMA/ionomycin and IL-18 | 0.0 | 0.0 |
| 93578_NK Cells IL-2_resting | 0.0 | 0.0 |
| 93109_Mixed Lymphocyte Reaction_Two Way MLR | 0.0 | 0.0 |
| 93110_Mixed Lymphocyte Reaction_Two Way MLR | 0.0 | 0.0 |
| 93111_Mixed Lymphocyte Reaction_Two Way MLR | 0.0 | 0.0 |
| 93112_Mononuclear Cells (PBMCs)_resting | 0.0 | 0.0 |
| 93113_Mononuclear Cells (PBMCs)_PWM | 0.0 | 0.0 |
| 93114_Mononuclear Cells (PBMCs)_PHA-L | 0.0 | 0.0 |
| 93249_Ramos (B cell)_none | 0.0 | 0.0 |
| 93250_Ramos (B cell)_ionomycin | 0.0 | 0.0 |
| 93349_B lymphocytes_PWM | 0.0 | 0.0 |
| 93350_B lymphoytes_CD40L and IL-4 | 0.0 | 0.0 |
| 92665_EOL-1 (Eosinophil)_dbcAMP differentiated | 0.0 | 0.0 |
| 93248_EOL-1 (Eosinophil)_dbcAMP/PMAionomycin | 0.0 | 0.0 |
| 93356_Dendritic Cells_none | 0.0 | 0.0 |
| 93355_Dendritic Cells_LPS 100 ng/ml | 0.0 | 0.0 |
| 93775_Dendritic Cells_anti-CD40 | 0.0 | 0.0 |
| 93774_Monocytes_resting | 0.0 | 0.0 |
| 93776_Monocytes_LPS 50 ng/ml | 0.0 | 0.0 |
| 93581_Macrophages_resting | 0.0 | 0.0 |
| 93582_Macrophages_LPS 100 ng/ml | 0.0 | 0.0 |
| 93098_HUVEC (Endothelial)_none | 0.0 | 0.0 |
| 93099_HUVEC (Endothelial)_starved | 0.0 | 0.0 |
| 93100_HUVEC (Endothelial)_IL-1b | 0.0 | 0.0 |
| 93779_HUVEC (Endothelial)_IFN gamma | 0.0 | 0.0 |
| 93102_HUVEC (Endothelial)_TNF alpha + IFN gamma | 0.0 | 0.0 |
| 93101_HUVEC (Endothelial)_TNF alpha + IL4 | 0.0 | 0.0 |
| 93781_HUVEC (Endothelial)_IL-11 | 0.0 | 62.8 |
| 93583_Lung Microvascular Endothelial Cells_none | 0.0 | 0.0 |
| 93584_Lung Microvascular Endothelial Cells_TNFa (4 ng/ml) and | 0.0 | 0.0 |
| IL1b (1 ng/ml) | | |
| 92662_Microvascular Dermal endothelium_none | 0.0 | 0.0 |
| 92663_Microvascular Dermal endothelium_TNFa (4 ng/ml) and IL1b | 0.0 | 0.0 |
| (1 ng/ml) | | |
| 93773_Bronchial epithelium_TNFa (4 ng/ml) and IL1b (1 ng/ml)** | 0.0 | 0.0 |
| 93347_Small Airway Epithelium_none | 0.0 | 0.0 |
| 93348_Small Airway Epithelium_TNFa (4 ng/ml) and IL1b (1 ng/ml) | 0.0 | 0.0 |
| 92668_Coronery Artery SMC_resting | 0.0 | 0.0 |
| 92669_Coronery Artery SMC_TNFa (4 ng/ml) and IL1b (1 ng/ml) | 0.0 | 0.0 |
| 93107_astrocytes_resting | 0.0 | 0.0 |
| 93108_astrocytes_TNFa (4 ng/ml) and IL1b (1 ng/ml) | 8.3 | 0.0 |
| 92666_KU-812 (Basophil)_resting | 0.0 | 0.0 |
| 92667_KU-812 (Basophil)_PMA/ionoycin | 0.0 | 0.0 |
| 93579_CCD1106 (Keratinocytes)_none | 0.0 | 0.0 |
| 93580_CCD1106 (Keratinocytes)_TNFa and IFNg** | 0.0 | 0.0 |
| 93791_Liver Cirrhosis | 0.0 | 46.4 |
| 93792_Lupus Kidney | 0.0 | 0.0 |
| 93577_NCI-H292 | 0.0 | 0.0 |
| 93358_NCI-H292_IL-4 | 0.0 | 0.0 |
| 93360_NCI-H292_IL-9 | 0.0 | 0.0 |
| 93359_NCI-H292_IL-13 | 0.0 | 0.0 |
| 93357_NCI-H292_IFN gamma | 0.0 | 0.0 |
| 93777_HPAEC_- | 0.0 | 0.0 |
| 93778_HPAEC_IL-1 beta/TNA alpha | 0.0 | 0.0 |
| 93254_Normal Human Lung Fibroblast_none | 0.0 | 0.0 |
| 93253_Normal Human Lung Fibroblast_TNFa (4 ng/ml) and IL-1b (1 ng/ml) | 0.0 | 0.0 |
| 93257_Normal Human Lung Fibroblast_IL-4 | 0.0 | 0.0 |
| 93256_Normal Human Lung Fibroblast_IL-9 | 0.0 | 0.0 |
| 93255_Normal Human Lung Fibroblast_IL-13 | 0.0 | 0.0 |
| 93258_Normal Human Lung Fibroblast_IFN gamma | 0.0 | 0.0 |
| 93106_Dermal Fibroblasts CCD1070_resting | 0.0 | 0.0 |
| 93361_Dermal Fibroblasts CCD1070_TNF alpha 4 ng/ml | 0.0 | 0.0 |
| 93105_Dermal Fibroblasts CCD1070_IL-1 beta 1 ng/ml | 0.0 | 0.0 |
| 93772_dermal fibroblast_IFN gamma | 0.0 | 0.0 |
| 93771_dermal fibroblast_IL-4 | 0.0 | 0.0 |
| 93260_IBD Colitis 2 | 0.0 | 0.0 |
| 93261_IBD Crohns | 0.0 | 0.0 |
| 735010_Colon_normal | 0.0 | 47.3 |
| 735019_Lung_none | 50.3 | 55.5 |
| 64028-1_Thymus_none | 100.0 | 100.0 |
| 64030-1_Kidney_none | 57.7 | 0.0 |

TABLE 34
Panel 4.1D

| | Relative | Relative |
|---|---|---|
| | Expression(%) | Expression(%) |
| | 4.1dx4tm6273 | 4.1dx4tm6627 |
| Tissue Name | f_ag4154_b1 | f_ag4334_b1 |
| 93768_Secondary Th1_anti-CD28/anti-CD3 | 0.0 | 0.0 |
| 93769_Secondary Th2_anti-CD28/anti-CD3 | 0.0 | 0.0 |
| 93770_Secondary Tr1_anti-CD28/anti-CD3 | 0.0 | 0.0 |
| 93573_Secondary Th1_resting day 4-6 in IL-2 | 0.0 | 0.0 |
| 93572_Secondary Th2_resting day 4-6 in IL-2 | 0.0 | 0.0 |
| 93571_Secondary Tr1_resting day 4-6 in IL-2 | 0.0 | 0.0 |
| 93568_primary Th1_anti-CD28/anti-CD3 | 0.0 | 0.0 |
| 93569_primary Th2_anti-CD28/anti-CD3 | 0.0 | 0.0 |
| 93570_primary Tr1_anti-CD28/anti-CD3 | 0.0 | 0.0 |
| 93565_primary Th1_resting dy 4-6 in IL-2 | 0.0 | 12.1 |
| 93566_primary Th2_resting dy 4-6 in IL-2 | 0.0 | 0.0 |
| 93567_primary Tr1_resting dy 4-6 in IL-2 | 0.0 | 0.0 |
| 93351_CD45RA CD4 lymphocyte_anti-CD28/anti-CD3 | 0.0 | 0.0 |
| 93352_CD45RO CD4 lymphocyte_anti-CD28/anti-CD3 | 0.0 | 0.0 |
| 93251_CD8 Lymphocytes_anti-CD28/anti-CD3 | 0.0 | 0.0 |
| 93353_chronic CD8 Lymphocytes 2ry_resting dy 4-6 in IL-2 | 0.0 | 0.0 |
| 93574_chronic CD8 Lymphocytes 2ry_activated CD3/CD28 | 0.0 | 0.0 |
| 93354_CD4_none | 0.0 | 0.0 |
| 93252_Secondary Th1/Th2/Tr1_anti-CD95 CH11 | 0.0 | 0.0 |
| 93103_LAK cells_resting | 0.0 | 0.0 |
| 93788_LAK cells_IL-2 | 0.0 | 0.0 |
| 93787_LAK cells_IL-2 + IL-12 | 0.0 | 0.0 |
| 93789_LAK cells_IL-2 + IFN gamma | 0.0 | 0.0 |
| 93790_LAK cells_IL-2 + IL-18 | 0.0 | 0.0 |
| 93104_LAK cells_PMA/ionomycin and IL-18 | 0.0 | 0.0 |
| 93578_NK Cells IL-2_resting | 0.0 | 0.0 |
| 93109_Mixed Lymphocyte Reaction_Two Way MLR | 0.0 | 0.0 |
| 93110_Mixed Lymphocyte Reaction_Two Way MLR | 0.0 | 0.0 |
| 93111_Mixed Lymphocyte Reaction_Two Way MLR | 0.0 | 0.0 |
| 93112_Mononuclear Cells (PBMCs)_resting | 0.0 | 0.0 |
| 93113_Mononuclear Cells (PBMCs)_PWM | 0.0 | 0.0 |
| 93114_Mononuclear Cells (PBMCs)_PHA-L | 0.0 | 0.0 |
| 93249_Ramos (B cell)_none | 0.0 | 0.0 |
| 93250_Ramos (B cell)_ionomycin | 0.0 | 0.0 |
| 93349_B lymphocytes_PWM | 0.0 | 0.0 |
| 93350_B lymphoytes_CD40L and IL-4 | 0.0 | 0.0 |
| 92665_EOL-1 (Eosinophil)_dbcAMP differentiated | 0.0 | 0.0 |
| 93248_EOL-1 (Eosinophil)_dbcAMP/PMAionomycin | 0.0 | 0.0 |
| 93356_Dendritic Cells_none | 0.0 | 0.0 |
| 93355_Dendritic Cells_LPS 100 ng/ml | 0.0 | 0.0 |
| 93775_Dendritic Cells_anti-CD40 | 0.0 | 0.0 |
| 93774_Monocytes_resting | 0.0 | 0.0 |
| 93776_Monocytes_LPS 50 ng/ml | 0.0 | 0.0 |
| 93581_Macrophages_resting | 0.0 | 0.0 |
| 93582_Macrophages_LPS 100 ng/ml | 0.0 | 0.0 |
| 93098_HUVEC (Endothelial)_none | 0.0 | 0.0 |
| 93099_HUVEC (Endothelial)_starved | 0.0 | 0.0 |
| 93100_HUVEC (Endothelial)_IL-1b | 0.0 | 0.0 |
| 93779_HUVEC (Endothelial)_IFN gamma | 0.0 | 0.0 |
| 93102_HUVEC (Endothelial)_TNF alpha + IFN gamma | 0.0 | 0.0 |
| 93101_HUVEC (Endothelial)_TNF alpha + IL4 | 0.0 | 0.0 |
| 93781_HUVEC (Endothelial)_IL-11 | 0.0 | 0.0 |
| 93583_Lung Microvascular Endothelial Cells_none | 0.0 | 0.0 |
| 93584_Lung Microvascular Endothelial Cells_TNFa (4 ng/ml) and | 0.0 | 0.0 |
| IL1b (1 ng/ml) | | |
| 92662_Microvascular Dermal endothelium_none | 0.0 | 0.0 |
| 92663_Microvascular Dermal endothelium_TNFa (4 ng/ml) and IL1b | 0.0 | 0.0 |
| (1 ng/ml) | | |
| 93773_Bronchial epithelium_TNFa (4 ng/ml) and IL1b (1 ng/ml)** | 0.0 | 0.0 |
| 93347_Small Airway Epithelium_none | 0.0 | 0.0 |
| 93348_Small Airway Epithelium_TNFa (4 ng/ml) and IL1b (1 ng/ml) | 0.0 | 0.0 |
| 92668_Coronery Artery SMC_resting | 0.0 | 0.0 |
| 92669_Coronery Artery SMC_TNFa (4 ng/ml) and IL1b (1 ng/ml) | 0.0 | 0.0 |
| 93107_astrocytes_resting | 0.0 | 0.0 |
| 93108_astrocytes_TNFa (4 ng/ml) and IL1b (1 ng/ml) | 0.0 | 0.0 |
| 92666_KU-812 (Basophil)_resting | 0.0 | 0.0 |
| 92667_KU-812 (Basophil)_PMA/ionoycin | 0.0 | 0.0 |
| 93579_CCD1106 (Keratinocytes)_none | 0.0 | 0.0 |
| 93580_CCD1106 (Keratinocytes)_TNFa and IFNg** | 0.0 | 0.0 |
| 93791_Liver Cirrhosis | 0.0 | 0.0 |
| 93577_NCI-H292 | 0.0 | 0.0 |
| 93358_NCI-H292_IL-4 | 0.0 | 0.0 |
| 93360_NCI-H292_IL-9 | 0.0 | 0.0 |
| 93359_NCI-H292_IL-13 | 0.0 | 0.0 |
| 93357_NCI-H292_IFN gamma | 0.0 | 0.0 |
| 93777_HPAEC_- | 0.0 | 0.0 |
| 93778_HPAEC_IL-1 beta/TNA alpha | 0.0 | 0.0 |
| 93254_Normal Human Lung Fibroblast_none | 0.0 | 0.0 |
| 93253_Normal Human Lung Fibroblast_TNFa (4 ng/ml) and IL-1b (1 ng/ml) | 0.0 | 0.0 |
| 93257_Normal Human Lung Fibroblast_IL-4 | 0.0 | 0.0 |
| 93256_Normal Human Lung Fibroblast_IL-9 | 0.0 | 0.0 |
| 93255_Normal Human Lung Fibroblast_IL-13 | 0.0 | 0.0 |
| 93258_Normal Human Lung Fibroblast_IFN gamma | 1.8 | 0.0 |
| 93106_Dermal Fibroblasts CCD1070_resting | 0.0 | 0.0 |
| 93361_Dermal Fibroblasts CCD1070_TNF alpha 4 ng/ml | 0.0 | 0.0 |
| 93105_Dermal Fibroblasts CCD1070_IL-1 beta 1 ng/ml | 0.0 | 0.0 |
| 93772_dermal fibroblast_IFN gamma | 0.0 | 0.0 |
| 93771_dermal fibroblast_IL-4 | 0.0 | 0.0 |
| 93892_Dermal fibroblasts_none | 0.0 | 0.0 |
| 99202_Neutrophils_TNFa + LPS | 0.0 | 0.0 |
| 99203_Neutrophils_none | 0.0 | 0.0 |
| 735010_Colon_normal | 0.0 | 0.0 |
| 735019_Lung_none | 30.4 | 64.2 |
| 64028-1_Thymus_none | 26.5 | 48.7 |
| 64030-1_Kidney_none | 100.0 | 100.0 |

TABLE 35
Panel CNS_neurodegeneration_v1.0

| | Relative | Relative |
|---|---|---|
| | Expression(%) | Expression(%) |
| | tm7244f_{—} | tm7852f_{—} |
| Tissue Name | ag4154_a2_s1 | ag4334_b1_s2 |
| AD 1 Hippo | 4.0 | 12.0 |
| AD 2 Hippo | 0.0 | 5.3 |
| AD 3 Hippo | 5.5 | 5.6 |
| AD 4 Hippo | 37.6 | 0.0 |
| AD 5 Hippo | 24.3 | 4.9 |
| AD 6 Hippo | 2.8 | 0.0 |
| Control 2 Hippo | 19.7 | 15.6 |
| Control 4 Hippo | 6.0 | 0.0 |
| Control (Path) 3 Hippo | 4.2 | 8.0 |
| AD 1 Temporal Ctx | 0.0 | 7.7 |
| AD 2 Temporal Ctx | 36.6 | 15.9 |
| AD 3 Temporal Ctx | 3.6 | 0.0 |
| AD 4 Temporal Ctx | 50.5 | 23.3 |
| AD 5 Inf Temporal Ctx | 23.6 | 30.0 |
| AD 5 Sup Temporal Ctx | 16.9 | 19.3 |
| AD 6 Inf Temporal Ctx | 6.4 | 8.0 |
| AD 6 Sup Temporal Ctx | 26.8 | 8.2 |
| Control 1 Temporal Ctx | 10.5 | 18.0 |
| Control 2 Temporal Ctx | 17.8 | 30.7 |
| Control 3 Temporal Ctx | 0.0 | 26.7 |
| Control 3 Temporal Ctx | 22.5 | 5.5 |
| Control (Path) 1 Temporal Ctx | 92.5 | 42.0 |
| Control (Path) 2 Temporal Ctx | 21.1 | 29.9 |
| Control (Path) 3 Temporal Ctx | 0.0 | 0.0 |
| Control (Path) 4 Temporal Ctx | 100.0 | 10.9 |
| AD 1 Occipital Ctx | 0.0 | 6.8 |
| AD 2 Occipital Ctx (Missing) | 0.0 | 0.0 |
| AD 3 Occipital Ctx | 0.0 | 0.0 |
| AD 4 Occipital Ctx | 2.4 | 3.9 |
| AD 5 Occipital Ctx | 2.1 | 0.0 |
| AD 6 Occipital Ctx | 7.3 | 3.7 |
| Control 1 Occipital Ctx | 0.0 | 8.2 |
| Control 2 Occipital Ctx | 11.2 | 29.4 |
| Control 3 Occipital Ctx | 14.4 | 0.0 |
| Control 4 Occipital Ctx | 12.7 | 4.6 |
| Control (Path) 1 Occipital Ctx | 62.6 | 100.0 |
| Control (Path) 2 Occipital Ctx | 7.5 | 4.2 |
| Control (Path) 3 Occipital Ctx | 4.5 | 0.0 |
| Control (Path) 4 Occipital Ctx | 9.7 | 10.7 |
| Control 1 Parietal Ctx | 8.4 | 6.9 |
| Control 2 Parietal Ctx | 13.5 | 14.2 |
| Control 3 Parietal Ctx | 8.6 | 6.9 |
| Control (Path) 1 Parietal Ctx | 29.2 | 28.7 |
| Control (Path) 2 Parietal Ctx | 8.7 | 12.5 |
| Control (Path) 3 Parietal Ctx | 7.0 | 0.0 |
| Control (Path) 4 Parietal Ctx | 51.3 | 12.8 |

Panel 1 Summary A972/Ag390 Multiple runs with two different probe and primer sets show highest expression of the NOV2 gene in the fetal brain (CTs=25-27), fetal lung and a cell line derived from a gastric cancer. Thus, the expression of this gene could be used to distinguish fetal brain tissue from adult brain tissue and fetal lung from adult lung.

Significant expression is also seen in fetal kidney, when compared to expression in the adult kidney. Therefore, the expression of this gene could also be used to distinguish fetal kidney tissue from adult kidney tissue.

Panel 1.1 Summary Ag671 Highest expression of the NOV2 gene is seen in the fetal brain (CT=26.2), a result that is repeated in Panel 1. Significant expression is also seen in fetal lung and skeletal muscle (CTs=26.7-32.8) when compared to expression in the adult tissues.In addtion, low, but significant expression is seen in the gastric cancer cell line (NCI-N87) and in adult and fetal kidney. Thus, the expression of this gene could be used to distinguish fetal brain tissue from other tissues in the panel. In addition, the expression of this gene could be used to distinguish fetal lung and skeletal mucle tissue from adult lung and skeletal muscle tissue.

Panel 1.2 Summary Ag766 Highest expression of the NOV2 gene in this panel is in the fetal brain (CT=27). Low, but significant expression is also seen in other brain samples including the amygdala, hippocampus, thalamus, and cerebral cortex. The higher levels of expression present in the fetal brain when compared to expression in the adult brain suggest an ongoing role in CNS processes.

There is also substantial expression in samples derived from a gastric cancer cell line (NCI-N87), fetal lung tissue and adult and fetal kidney tissue. This expression profile is concordant with the expression seen in Panels 1, and 1.1. Of note is the difference in expression between fetal lung (CT=27.7) and its adult counterpart (CT=31.8). Thus, the expression of this gene could be used to distinguish fetal lung tissue from adult lung tissue.

Panel 1.3D Summary Ag1211/Ag2233 Three runs with two different probe and primer sets show highest expression in the fetal brain, lung and skeletal muscle (CTs=30). There is also substantial expression in samples derived from a gastric cancer cell line (NCI-N87). Thus, the expression of this gene could be used to distinguish fetal skeletal muscle or fetal lung from their respective adult tissue counterparts.

Ag2241 Expression of the NOV2 gene is low/undetectable (Ct values >35) in all samples in this panel. (Data not shown.)

Panel General_screening_panel_v1.0 Summary Ag1928/Ag4154/Ag4334 Multiple runs with different probe and primer sets show highest expression of the NOV2 gene in the fetal brain (CTs=26-33), indicating a probable developmental role for the NOV2 gene in the CNS. This finding is consistent with the expression seen in Panels 1, 1.1, 1.2 and 1.3D. There is also significant expression in the fetal kidney and lung and in the gastric cancer cell line (NCI-N87). Thus, the expression of this gene could be used to distinguish fetal brain tissue from adult brain. In addition, the expression of this gene could be used to distinguish fetal kidney tissue from adult kidney tissue and fetal lung tissue from adult lung tissue.

Panel 2.2. Summary Ag2241 The expression of the NOV2 gene appears to be exclusive to a sample derived from a kidney cancer. Thus, the expression of this gene could be used to distinguish this sample from others in the panel, including its normal adjacent tissue. Moreover, therapeutic modulation of this gene or its protein product, through the use of small molecule drugs, antibodies or protein therapeutics, may be useful in the treatment of kidney cancer.

Panel 2D Summary A1211 Two runs using the same probe and primer produce results that are in excellent agreement, with highest expression of the NOV2 gene in a kidney cancer (CTs=29.5). This expression profile is in concordance with the expression seen in Panel 2.2. Significant expression is also seen in a thyroid cancer sample and a series of normal adjacent tissues derived from kidney and lung cancer cases. Thus, the expression of this gene could be used to distinguish this kidney cancer sample from others in the panel, including its normal adjacent tissue. In addition, low levels of expression could be used to distinguish normal adjacent tissue from adjacent malignant tissue. Moreover, therapeutic modulation of this gene or its protein product, through the use of small molecule drugs, antibodies or protein therapeutics, might be of benefit in the treatment of kidney cancer.

Panel 4D Summary Ag1938/AR2233 Expression of the NOV2 gene is limited to a few samples in this panel, with highest expression seen in the thymus. This expression is consistent in two runs with two different probe and primer sets (CTs=33-34). Significant expression is also seen in the normal lung, a result that is replicated in Panel 4.1D and Panels 1, 1.2, and 1.3D. Please see Panel 4.1D for potential utility of this gene in the treatment of autoinflammatory related disease.

Ag1928/Ag2241 Expression of the NOV2 gene is low/undetectable (Ct values >35) in all samples in this panel. (Data not shown.)

Panel 4.1 D Summary Ag4154/Ag4334 Two experiments with two different probes and primers both show highest expression of the NOV2 gene in the kidney (CTs=33-34). There is also low but significant expression in normal lung and thymus. The expression in normal kidney is consistent with the expression seen in Panels 1, 1.1, 1.2, 1.3D and Panel 4. The expression of low levels of the NOV2 gene product, a secreted fibrillin-related homolog, in the normal lung, thymus, and kidney suggest that it could be used as a protein or antibody therapeutic to reduce or eliminate symptoms of patients with connective tissue diseases of the lung, thymus and kidney. These diseases include Marfan syndrome, ankylosing spondylitis, Sjogren's syndrome, and relapsing polychondritis.

Panel CNS_neurodegeneration_v1.0 Summary Ag4154/Ag4334Expression of the NOV2 gene is restricted to a few samples with highest expression in the cerebral cortex of a control patient. This gene does not appear to be expressed in the brains of patients with Alzheimer's disease. Please see Panel 1 for discussion of potential utility in the CNS.

Ag2241 Expression of the NOV2 gene is low/undetectable (Ct values >35) in all samples in this panel. (Data not shown.)(Tanoue, Pulmonary involvement in collagen vascular disease: a review of the pulmonary manifestations of the Marfan syndrome, ankylosing spondylitis, Sjogren's syndrome, and relapsing polychondritis. 7:62-77, 1992; Kanwar et al., Isolation of rat fibrillin-1 cDNA and its relevance in metanephric development. 275 (5 Pt 2):F710-23, 1998).

NOV3: KIAA1589-like

Expression of the NOV3 gene (GSAL442663.1_A) was assessed using the primer-probe set Ag1550 described in Table 36. Results from RTQ-PCR runs are shown in Table 37.

TABLE 36

Probe Name Ag1550

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-TGGAAATACTGGTGATGGAAAG-3′ | 59 | 22 | 900 | 164 |
| Probe | FAM-5′-TCAACCACACTTTCTTTTATGCTCGTG-3′-TAMRA | 66.2 | 27 | 932 | 165 |
| Reverse | 5′-TCGGGGAGGTTTTAAAGACTT-3′ | 59.1 | 21 | 959 | 166 |

TABLE 37
Panel 1.3D

| | Relative | | Relative |
|---|---|---|---|
| | Expression(%) | | Expression(%) |
| | 1.3Dtm2584f | | 1.3Dtm2584f |
| Tissue Name | _ag1550 | Tissue Name | _ag1550 |
| Liver adenocarcinoma | 20.2 | Kidney (fetal) | 10.9 |
| Pancreas | 4.7 | Renal ca. 786-0 | 12.9 |
| Pancreatic ca. CAPAN 2 | 4.7 | Renal ca. A498 | 24.0 |
| Adrenal gland | 15.1 | Renal ca. RXF 393 | 10.4 |
| Thyroid | 11.4 | Renal ca. ACHN | 21.9 |
| Salivary gland | 4.5 | Renal ca. UO-31 | 9.6 |
| Pituitary gland | 8.0 | Renal ca. TK-10 | 4.8 |
| Brain (fetal) | 9.5 | Liver | 3.2 |
| Brain (whole) | 15.0 | Liver (fetal) | 12.8 |
| Brain (amygdala) | 15.8 | Liver ca. (hepatoblast) HepG2 | 12.7 |
| Brain (cerebellum) | 6.7 | Lung | 10.3 |
| Brain (hippocampus) | 30.6 | Lung (fetal) | 12.8 |
| Brain (substantia nigra) | 6.8 | Lung ca. (small cell) LX-1 | 5.7 |
| Brain (thalamus) | 14.1 | Lung ca. (small cell) NCI-H69 | 9.2 |
| Cerebral Cortex | 45.1 | Lung ca. (s.cell var.) SHP-77 | 12.6 |
| Spinal cord | 8.4 | Lung ca. (large cell)NCI-H460 | 2.2 |
| CNS ca. (glio/astro) U87-MG | 30.4 | Lung ca. (non-sm. cell) A549 | 4.6 |
| CNS ca. (glio/astro) U-118-MG | 42.9 | Lung ca. (non-s.cell) NCI-H23 | 11.5 |
| CNS ca. (astro) SW1783 | 16.8 | Lung ca (non-s.cell) HOP-62 | 11.2 |
| CNS ca.* (neuro; met) SK-N-AS | 23.2 | Lung ca. (non-s.cl) NCI-H522 | 11.5 |
| CNS ca. (astro) SF-539 | 15.5 | Lung ca. (squam.) SW 900 | 17.1 |
| CNS ca. (astro) SNB-75 | 55.1 | Lung ca. (squam.) NCI-H596 | 4.0 |
| CNS ca. (glio) SNB-19 | 1.5 | Mammary gland | 27.0 |
| CNS ca. (glio) U251 | 12.8 | Breast ca.* (pl. effusion) MCF-7 | 34.4 |
| CNS ca. (glio) SF-295 | 9.2 | Breast ca.* (pl.ef) MDA-MB-231 | 24.8 |
| Heart (fetal) | 33.7 | Breast ca.* (pl. effusion) T47D | 31.4 |
| Heart | 3.3 | Breast ca. BT-549 | 33.0 |
| Fetal Skeletal | 100.0 | Breast ca. MDA-N | 21.2 |
| Skeletal muscle | 7.7 | Ovary | 36.1 |
| Bone marrow | 11.8 | Ovarian ca. OVCAR-3 | 19.8 |
| Thymus | 12.1 | Ovarian ca. OVCAR-4 | 5.3 |
| Spleen | 10.4 | Ovarian ca. OVCAR-5 | 12.9 |
| Lymph node | 8.1 | Ovarian ca. OVCAR-8 | 11.2 |
| Colorectal | 11.3 | Ovarian ca. IGROV-1 | 2.5 |
| Stomach | 17.3 | Ovarian ca.* (ascites) SK-OV-3 | 10.6 |
| Small intestine | 9.1 | Uterus | 11.0 |
| Colon ca. SW480 | 14.9 | Placenta | 14.8 |
| Colon ca.* (SW480 met)SW620 | 6.2 | Prostate | 6.4 |
| Colon ca. HT29 | 2.5 | Prostate ca.* (bone met)PC-3 | 41.8 |
| Colon ca. HCT-116 | 5.2 | Testis | 15.2 |
| Colon ca. CaCo-2 | 9.2 | Melanoma Hs688(A).T | 45.7 |
| 83219 CC Well to Mod Diff | 11.3 | Melanoma* (met) Hs688(B).T | 44.1 |
| (ODO3866) | | | |
| Colon ca. HCC-2998 | 5.6 | Melanoma UACC-62 | 12.5 |
| Gastric ca.* (liver met) NCI-N87 | 32.8 | Melanoma M14 | 7.6 |
| Bladder | 6.9 | Melanoma LOX IMVI | 3.7 |
| Trachea | 14.2 | Melanoma* (met) SK-MEL-5 | 26.4 |
| Kidney | 7.1 | Adipose | 5.8 |

Panel 1.3D Summary The NOV3 gene, a KIAA1589 homolog, is ubiquitously expressed in this panel, with highest expression seen in fetal skeletal muscle (CT=29). Expression of the gene appears to be much higher in fetal skeletal muscle than in adult skeletal muscle (CT=32.6). In addition, expression of the gene appears to be higher in fetal heart (CT=30.5) than in adult heart (CT=33.9). Thus, expression of this gene could be used to distinguish between adult and fetal sources of heart and skeletal muscle. In addition, the higher levels of expression of the NOV3 gene in fetal heart and skeletal muscle when compared to the levels of expression in adult tissue, suggests that the protein encoded by the NOV3 gene may be involved in the development of these tissues. Therefore, therapeutic modulation of this gene or its protein product may be effective in the treatment of diseases that affect the heart, such as atherosclerosis, hypertension, or aortic stenosis. Furthermore, the therapeutic modulation of this gene or gene product, through replacement therapy, could be used as a regenerative therapy for muscle disease.

Among tissues involved in central nervous system function, this gene is expressed at moderate levels in all brain regions examined. The NOV3 gene encodes a protein with a putative zinc-finger motif. Since these proteins are known to interact with nucleic acids, this suggests that the NOV3 gene product may play a potential role in transcription. Thus, therapeutic modulation of the NOV3 gene product may be used to regulate the transcription of disease-related proteins such as ataxin, huntingtin, or various apoptosis cascade proteins.

NOV4: WD40 Motif Protein

Expression of the NOV4 gene (GSAL442663.1_B) was assessed using the primer-probe sets Ag1551 and Ag3362 described in Tables 38 and 39. Results from RTQ-PCR runs are shown in Tables 40-44.

TABLE 38

Probe Name Ag1551

| | | | | Start | SEQ ID |
|---|---|---|---|---|---|
| Primers | Sequences | TM | Length | Position | NO: |
| Forward | 5′-GGCTCCAAGTATGGTATCATCA-3′ | 58.9 | 22 | 636 | 167 |
| Probe | TET-5′-TCTGAAGACCCCTACGCTCAAGGTGT-3′-TAMRA | 69.1 | 26 | 668 | 168 |
| Reverse | 5′-TGAAGTAGAGGTTTTCGTGCAT-3′ | 58.9 | 22 | 696 | 169 |

TABLE 39

Probe Name Ag3362

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-GTCGGGCAGGACCTTTACT-3′ | 59.2 | 19 | 1474 | 170 |
| Probe | FAM-5′-TCCTACAGCTAATTCTGCAGGGCACA-3′-TAMRA | 68.8 | 26 | 1498 | 171 |
| Reverse | 5′-TACGCTTTACTCCCGTAAGTCA-3′ | 59 | 22 | 1543 | 172 |

TABLE 40
Panel 1.3D

| | Relative | | Relative |
|---|---|---|---|
| | Expression(%) | | Expression(%) |
| | 1.3Dtm2585t_{—} | | 1.3Dtm2585t_{—} |
| Tissue Name | ag1551 | Tissue Name | ag1551 |
| Liver adenocarcinoma | 23.2 | Kidney (fetal) | 10.7 |
| Pancreas | 9.4 | Renal ca. 786-0 | 20.2 |
| Pancreatic ca. CAPAN 2 | 8.0 | Renal ca. A498 | 40.3 |
| Adrenal gland | 8.4 | Renal ca. RXF 393 | 26.1 |
| Thyroid | 17.3 | Renal ca. ACHN | 75.3 |
| Salivary gland | 11.1 | Renal ca. UO-31 | 36.6 |
| Pituitary gland | 7.2 | Renal ca. TK-10 | 31.6 |
| Brain (fetal) | 11.7 | Liver | 1.8 |
| Brain (whole) | 24.8 | Liver (fetal) | 8.7 |
| Brain (amygdala) | 17.8 | Liver ca. (hepatoblast) HepG2 | 68.8 |
| Brain (cerebellum) | 5.5 | Lung | 9.9 |
| Brain (hippocampus) | 37.6 | Lung (fetal) | 20.0 |
| Brain (substantia nigra) | 5.4 | Lung ca. (small cell) LX-1 | 22.1 |
| Brain (thalamus) | 17.0 | Lung ca. (small cell) NCI-H69 | 52.5 |
| Cerebral Cortex | 36.1 | Lung ca. (s.cell var.) SHP-77 | 41.8 |
| Spinal cord | 13.6 | Lung ca. (large cell)NCI-H460 | 10.9 |
| CNS ca. (glio/astro) U87-MG | 43.2 | Lung ca. (non-sm. cell) A549 | 22.1 |
| CNS ca. (glio/astro) U-118-MG | 55.5 | Lung ca. (non-s.cell) NCI-H23 | 11.1 |
| CNS ca. (astro) SW1783 | 45.4 | Lung ca. (non-s.cell) HOP-62 | 16.8 |
| CNS ca.* (neuro; met) SK-N-AS | 39.5 | Lung ca. (non-s.cl) NCI-H522 | 27.4 |
| CNS ca. (astro) SF-539 | 33.0 | Lung ca. (squam.) SW 900 | 5.3 |
| CNS ca. (astro) SNB-75 | 74.2 | Lung ca. (squam.) NCI-H596 | 17.1 |
| CNS ca. (glio) SNB-19 | 0.4 | Mammary gland | 47.3 |
| CNS ca. (glio) U251 | 5.9 | Breast ca.* (pl. effusion) MCF-7 | 100.0 |
| CNS ca. (glio) SF-295 | 22.8 | Breast ca.* (pl.ef) MDA-MB-231 | 67.4 |
| Heart (fetal) | 15.6 | Breast ca.* (pl. effusion) T47D | 25.9 |
| Heart | 1.2 | Breast ca. BT-549 | 57.8 |
| Fetal Skeletal | 60.7 | Breast ca. MDA-N | 58.2 |
| Skeletal muscle | 5.6 | Ovary | 21.2 |
| Bone marrow | 5.6 | Ovarian ca. OVCAR-3 | 24.5 |
| Thymus | 10.2 | Ovarian ca. OVCAR-4 | 10.5 |
| Spleen | 12.8 | Ovarian ca. OVCAR-5 | 19.1 |
| Lymph node | 21.2 | Ovarian ca. OVCAR-8 | 11.7 |
| Colorectal | 8.4 | Ovarian ca. IGROV-1 | 8.9 |
| Stomach | 33.7 | Ovarian ca.* (ascites) SK-OV-3 | 46.3 |
| Small intestine | 17.9 | Uterus | 17.2 |
| Colon ca. SW480 | 76.3 | Placenta | 28.1 |
| Colon ca.* (SW480 met)SW620 | 14.7 | Prostate | 9.5 |
| Colon ca. HT29 | 7.6 | Prostate ca.* (bone met)PC-3 | 81.2 |
| Colon ca. HCT-116 | 19.3 | Testis | 34.4 |
| Colon ca. CaCo-2 | 32.5 | Melanoma Hs688(A).T | 72.7 |
| 83219 CC Well to Mod Diff | 19.9 | Melanoma* (met) Hs688(B).T | 73.7 |
| (ODO3866) | | | |
| Colon ca. HCC-2998 | 30.8 | Melanoma UACC-62 | 11.6 |
| Gastric ca.* (liver met) NCI-N87 | 71.2 | Melanoma M14 | 14.1 |
| Bladder | 10.9 | Melanoma LOX IMVI | 12.2 |
| Trachea | 15.8 | Melanoma* (met) SK-MEL-5 | 18.7 |
| Kidney | 5.7 | Adipose | 4.9 |

TABLE 41
Panel General_screening_panel_v1.0

| | Relative | | Relative |
|---|---|---|---|
| | Expression(%) | | Expression(%) |
| | tm7276f_{—} | | tm7276f_{—} |
| Tissue Name | ag3362_b2 | Tissue Name | ag3362_b2 |
| D6005-01_Human adipose | 6.3 | Renal ca._TK-10 | 44.4 |
| 112193_Metastatic melanoma | 17.6 | Bladder | 9.4 |
| 112192_Metastatic melanoma | 18.3 | Gastric ca.(liver met)_NCI-N87 | 21.7 |
| 95280_Epidermis (metastatic | 17.2 | 112197_Stomach | 17.6 |
| melanoma) | | | |
| 95279_Epidermis (metastatic | 13.6 | 94938_Colon Adenocarcinoma | 5.8 |
| melanoma) | | | |
| Melanoma (met)_SK-MEL-5 | 19.6 | Colon ca._SW480 | 34.7 |
| 112196_Tongue (oncology) | 14.6 | Colon ca.(SW480 met)_SW620 | 14.2 |
| 113461_Testis Pool | 4.0 | Colon ca._HT29 | 7.3 |
| Prostate ca.(bone met)_PC-3 | 90.8 | Colon ca._HCT-116 | 14.3 |
| 113455_Prostate Pool | 4.1 | Colon ca._CaCo-2 | 19.8 |
| 103396_Placenta | 11.4 | 83219_CC Well to Mod Diff | 3.6 |
| | | (ODO3866) | |
| 113463_Uterus Pool | 2.1 | 94936_Colon Adenocarcinoma | 9.4 |
| Ovarian carcinoma_OVCAR-3 | 17.5 | 94930_Colon | 8.9 |
| Ovarian carcinoma(ascites)_SK- | 47.1 | 94935_Colon Adenocarcinoma | 13.3 |
| OV-3 | | | |
| 95297_Adenocarcinoma (ovary) | 14.7 | 113468_Colon Pool | 5.7 |
| Ovarian carcinoma_OVCAR-5 | 31.8 | 113457_Small Intestine Pool | 10.2 |
| Ovarian carcinoma_IGROV-1 | 12.9 | 113460_Stomach Pool | 6.2 |
| Ovarian carcinoma_OVCAR-8 | 6.7 | 113467_Bone Marrow Pool | 1.3 |
| 103368_Ovary | 12.6 | 103371_Fetal Heart | 1.1 |
| MCF7_breast carcinoma(pleural | 76.2 | 113451_Heart Pool | 3.4 |
| effusion) | | | |
| Breast ca. (pleural | 30.3 | 113466_Lymph Node Pool | 8.7 |
| effusion)_MDA-MB-231 | | | |
| 112189_ductal cell | 65.7 | 103372_Fetal Skeletal Muscle | 2.3 |
| carcinoma(breast) | | | |
| Breast ca. (pleural effusion)_T47D | 100.0 | 113456_Skeletal Muscle Pool | 9.4 |
| Breast carcinoma_MDA-N | 33.6 | 113459_Spleen Pool | 4.6 |
| 113452_Breast Pool | 4.6 | 113462_Thymus Pool | 7.4 |
| 103398_Trachea | 7.7 | CNS ca. (glio/astro)_U87-MG | 34.0 |
| 112354_lung | 4.9 | CNS ca. (glio/astro)_U-118-MG | 27.3 |
| 103374_Fetal Lung | 7.1 | CNS ca. (neuro; met)_SK-N-AS | 16.1 |
| 94921_Small cell carcinoma of the | 9.3 | 95264_Brain astrocytoma | 14.3 |
| lung | | | |
| Lung ca.(small cell)_LX-1 | 15.9 | CNS ca. (astro)_SNB-75 | 60.8 |
| 94919_Small cell carcinoma of the | 4.9 | CNS ca. (glio)_SNB-19 | 13.9 |
| lung | | | |
| Lung ca.(s.cell var.)_SHP-77 | 16.5 | CNS ca. (glio)_SF-295 | 28.6 |
| 95268_Lung (Large cell | 27.2 | 113447_Brain (Amygdala) Pool | 5.3 |
| carcinoma) | | | |
| 94920_Small cell carcinoma of the | 4.1 | 103382_Brain (cerebellum) | 5.0 |
| lung | | | |
| Lung ca.(non-s.cell)_NCI-H23 | 15.1 | 64019-1_brain(fetal) | 16.5 |
| Lung ca.(large cell)_NCI-H460 | 9.6 | 113448_Brain (Hippocampus) | 5.5 |
| | | Pool | |
| Lung ca.(non-s.cell)_HOP-62 | 7.6 | 113464_Cerebral Cortex Pool | 8.7 |
| Lung ca.(non-s.cl)_NCI-H522 | 18.2 | 113449_Brain (Substantia nigra) | 8.3 |
| | | Pool | |
| 103392_Liver | 0.0 | 113450_Brain (Thalamus) Pool | 6.3 |
| 103393_Fetal Liver | 7.3 | 103384_Brain (whole) | 7.0 |
| Liver ca.(hepatoblast)_HepG2 | 29.7 | 113458_Spinal Cord Pool | 5.6 |
| 113465_Kidney Pool | 17.7 | 103375_Adrenal Gland | 6.3 |
| 103373_Fetal Kidney | 4.6 | 113454_Pituitary gland Pool | 0.8 |
| Renal ca._786-0 | 17.2 | 103397_Salivary Gland | 5.6 |
| 112188_renal cell carcinoma | 5.1 | 103369_Thyroid (female) | 9.8 |
| Renal ca._ACHN | 17.4 | Pancreatic ca._CAPAN2 | 11.8 |
| 112190_Renal cell carcinoma | 11.1 | 113453_Pancreas Pool | 9.2 |

TABLE 42
Panel 2.2

| | Relative | | Relative |
|---|---|---|---|
| | Expression(%) | | Expression(%) |
| | 2.2x4tm6351t_{—} | | 2.2x4tm6351t_{—} |
| Tissue Name | ag1551_b1 | Tissue Name | ag1551_b1 |
| Normal Colon GENPAK 061003 | 26.8 | 83793 Kidney NAT (OD04348) | 100.0 |
| 97759 Colon cancer (OD06064) | 19.2 | 98938 Kidney malignant cancer | 24.5 |
| | | (OD06204B) | |
| 97760 Colon cancer NAT | 28.5 | 98939 Kidney normal adjacent | 10.9 |
| (OD06064) | | tissue (OD06204E) | |
| 97778 Colon cancer (OD06159) | 8.9 | 85973 Kidney Cancer (OD04450- | 51.3 |
| | | 01) | |
| 97779 Colon cancer NAT | 13.2 | 85974 Kidney NAT (OD04450-03) | 24.0 |
| (OD06159) | | | |
| 98861 Colon cancer (OD06297-04) | 8.9 | Kidney Cancer Clontech 8120613 | 7.0 |
| 98862 Colon cancer NAT | 24.3 | Kidney NAT Clontech 8120614 | 19.3 |
| (OD06297-015) | | | |
| 83237 CC Gr.2 ascend colon | 15.6 | Kidney Cancer Clontech 9010320 | 4.0 |
| (ODO3921) | | | |
| 83238 CC NAT (ODO3921) | 12.7 | Kidney NAT Clontech 9010321 | 7.3 |
| 97766 Colon cancer metastasis | 3.9 | Kidney Cancer Clontech 8120607 | 38.9 |
| (OD06104) | | | |
| 97767 Lung NAT (OD06104) | 6.8 | Kidney NAT Clontech 8120608 | 5.9 |
| 87472 Colon mets to lung | 37.3 | Normal Uterus GENPAK 061018 | 7.6 |
| (OD04451-01) | | | |
| 87473 Lung NAT (OD04451-02) | 14.9 | Uterus Cancer GENPAK 064011 | 13.6 |
| Normal Prostate Clontech A+ | 11.5 | Normal Thyroid Clontech A+ | 10.9 |
| 6546-1 (8090438) | | 6570-1 (7080817) | |
| 84140 Prostate Cancer (OD04410) | 7.9 | Thyroid Cancer GENPAK 064010 | 3.8 |
| 84141 Prostate NAT(OD04410) | 11.2 | Thyroid Cancer INVITROGEN | 35.1 |
| | | A302152 | |
| Normal Ovary Res. Gen. | 13.6 | Thyroid NAT INVITROGEN | 4.0 |
| | | A302153 | |
| 98863 Ovarian cancer (OD06283- | 9.3 | Normal Breast GENPAK 061019 | 27.5 |
| 03) | | | |
| 98865 Ovarian cancer | 16.2 | 84877 Breast Cancer (OD04566) | 13.7 |
| NAT/fallopian tube (OD06283-07) | | | |
| Ovarian Cancer GENPAK 064008 | 19.0 | Breast Cancer Res. Gen. 1024 | 24.2 |
| 97773 Ovarian cancer (OD06145) | 6.6 | 85975 Breast Cancer (OD04590- | 26.0 |
| | | 01) | |
| 97775 Ovarian cancer NAT | 20.4 | 85976 Breast Cancer Mets | 46.5 |
| (OD06145) | | (OD04590-03) | |
| 98853 Ovarian cancer (OD06455- | 13.6 | 87070 Breast Cancer Metastasis | 47.8 |
| 03) | | (OD04655-05) | |
| 98854 Ovarian NAT (OD06455- | 6.9 | GENPAK Breast Cancer 064006 | 32.9 |
| 07) Fallopian tube | | | |
| Normal Lung GENPAK 061010 | 13.6 | Breast Cancer Clontech 9100266 | 18.8 |
| 92337 Invasive poor diff. lung | 8.8 | Breast NAT Clontech 9100265 | 5.1 |
| adeno (ODO4945-01 | | | |
| 92338 Lung NAT (ODO4945-03) | 11.5 | Breast Cancer INVITROGEN | 3.0 |
| | | A209073 | |
| 84136 Lung Malignant Cancer | 6.2 | Breast NAT INVITROGEN | 33.1 |
| (OD03126) | | A2090734 | |
| 84137 Lung NAT (OD03126) | 7.2 | 97763 Breast cancer (OD06083) | 59.8 |
| 90372 Lung Cancer (OD05014A) | 8.1 | 97764 Breast cancer node | 37.3 |
| | | metastasis (OD06083) | |
| 90373 Lung NAT (OD05014B) | 11.2 | Normal Liver GENPAK 061009 | 19.4 |
| 97761 Lung cancer (OD06081) | 10.9 | Liver Cancer Research Genetics | 3.0 |
| | | RNA 1026 | |
| 97762 Lung cancer NAT | 8.5 | Liver Cancer Research Genetics | 18.1 |
| (OD06081) | | RNA 1025 | |
| 85950 Lung Cancer (OD04237-01) | 10.1 | Paired Liver Cancer Tissue | 12.0 |
| | | Research Genetics RNA 6004-T | |
| 85970 Lung NAT (OD04237-02) | 12.1 | Paired Liver Tissue Research | 3.2 |
| | | Genetics RNA 6004-N | |
| 83255 Ocular Mel Met to Liver | 41.2 | Paired Liver Cancer Tissue | 8.8 |
| (ODO4310) | | Research Genetics RNA 6005-T | |
| 83256 Liver NAT (ODO4310) | 3.6 | Paired Liver Tissue Research | 22.7 |
| | | Genetics RNA 6005-N | |
| 84139 Melanoma Mets to Lung | 20.2 | Liver Cancer GENPAK 064003 | 20.5 |
| (OD04321) | | | |
| 84138 Lung NAT (OD04321) | 3.5 | Normal Bladder GENPAK 061001 | 19.7 |
| Normal Kidney GENPAK 061008 | 13.4 | Bladder Cancer Research Genetics | 7.4 |
| | | RNA 1023 | |
| 83786 Kidney Ca, Nuclear grade 2 | 49.3 | Bladder Cancer INVITROGEN | 15.4 |
| (OD04338 | | A302173 | |
| 83787 Kidney NAT (OD04338) | 4.6 | Normal Stomach GENPAK | 39.3 |
| | | 061017 | |
| 83788 Kidney Ca Nuclear grade | 38.0 | Gastric Cancer Clontech 9060397 | 9.4 |
| 1/2 (OD04339) | | | |
| 83789 Kidney NAT (OD04339) | 19.1 | NAT Stomach Clontech 9060396 | 7.2 |
| 83790 Kidney Ca, Clear cell type | 7.8 | Gastric Cancer Clontech 9060395 | 14.1 |
| (OD04340) | | | |
| 83791 Kidney NAT (OD04340) | 12.5 | NAT Stomach Clontech 9060394 | 28.5 |
| 83792 Kidney Ca, Nuclear grade 3 | 5.2 | Gastric Cancer GENPAK 064005 | 15.9 |
| (OD04348) | | | |

TABLE 43
Panel 4D

| | Relative | | Relative |
|---|---|---|---|
| | Expression(%) | | Expression(%) |
| | 4dtm4721t_{—} | | 4dtm4721t_{—} |
| Tissue Name | ag1551 | Tissue Name | ag1551 |
| 93768_Secondary Th1_anti- | 12.4 | 93100_HUVEC (Endothelial)_IL- | 3.5 |
| CD28/anti-CD3 | | 1b | |
| 93769_Secondary Th2_anti- | 7.9 | 93779_HUVEC (Endothelial)_IFN | 10.7 |
| CD28/anti-CD3 | | gamma | |
| 93770_Secondary Tr1_anti- | 9.2 | 93102_HUVEC | 10.2 |
| CD28/anti-CD3 | | (Endothelial)_TNF alpha + IFN | |
| | | gamma | |
| 93573_Secondary Th1_resting day | 2.0 | 93101_HUVEC | 11.0 |
| 4-6 in IL-2 | | (Endothelial)_TNF alpha + IL4 | |
| 93572_Secondary Th2_resting day | 3.5 | 93781_HUVEC (Endothelial)_IL- | 3.2 |
| 4-6 in IL-2 | | 11 | |
| 93571_Secondary Tr1_resting day | 3.3 | 93583_Lung Microvascular | 12.0 |
| 4-6 in IL-2 | | Endothelial Cells_none | |
| 93568_primary Th1_anti- | 15.9 | 93584_Lung Microvascular | 15.8 |
| CD28/anti-CD3 | | Endothelial Cells_TNFa (4 ng/ml) | |
| | | and IL1b (1 ng/ml) | |
| 93569_primary Th2_anti- | 8.7 | 92662_Microvascular Dermal | 6.0 |
| CD28/anti-CD3 | | endothelium_none | |
| 93570_primary Tr1_anti- | 21.3 | 92663_Microsvasular Dermal | 9.2 |
| CD28/anti-CD3 | | endothelium_TNFa (4 ng/ml) and | |
| | | IL1b (1 ng/ml) | |
| 93565_primary Th1_resting dy 4-6 | 20.6 | 93773_Bronchial | 1.9 |
| in IL-2 | | epithelium_TNFa (4 ng/ml) and | |
| | | IL1b (1 ng/ml) ** | |
| 93566_primary Th2_resting dy 4-6 | 9.7 | 93347_Small Airway | 8.8 |
| in IL-2 | | Epithelium_none | |
| 93567_primary Tr1_resting dy 4-6 | 11.3 | 93348_Small Airway | 19.6 |
| in IL-2 | | Epithelium_TNFa (4 ng/ml) and | |
| | | IL1b (1 ng/ml) | |
| 93351_CD45RA CD4 | 8.7 | 92668_Coronery Artery | 12.5 |
| lymphocyte_anti-CD28/anti-CD3 | | SMC_resting | |
| 93352_CD45RO CD4 | 13.6 | 92669_Coronery Artery | 4.7 |
| lymphocyte_anti-CD28/anti-CD3 | | SMC_TNFa (4 ng/ml) and IL1b (1 ng/ml) | |
| 93251_CD8 Lymphocytes_anti- | 11.0 | 93107_astrocytes_resting | 9.5 |
| CD28/anti-CD3 | | | |
| 93353_chronic CD8 Lymphocytes | 16.2 | 93108_astrocytes_TNFa (4 ng/ml) | 8.0 |
| 2ry_resting dy 4-6 in IL-2 | | and IL1b (1 ng/ml) | |
| 93574_chronic CD8 Lymphocytes | 6.8 | 92666_KU-812 (Basophil)_resting | 36.3 |
| 2ry_activated CD3/CD28 | | | |
| 93354_CD4_none | 4.5 | 92667_KU-812 | 51.4 |
| | | (Basophil)_PMA/ionoycin | |
| 93252_Secondary | 6.2 | 93579_CCD1106 | 15.3 |
| Th1/Th2/Tr1_anti-CD95 CH11 | | (Keratinocytes)_none | |
| 93103_LAK cells_resting | 5.8 | 93580_CCD1106 | 2.0 |
| | | (Keratinocytes)_TNFa and IFNg** | |
| 93788_LAK cells_IL-2 | 11.1 | 93791_Liver Cirrhosis | 1.4 |
| 93787_LAK cells_IL-2 + IL-12 | 11.7 | 93792_Lupus Kidney | 1.6 |
| 93789_LAK cells_IL-2 + IFN | 19.9 | 93577_NCI-H292 | 14.5 |
| gamma | | | |
| 93790_LAK cells_IL-2 + IL-18 | 16.5 | 93358_NCI-H292_IL-4 | 14.9 |
| 93104_LAK | 7.0 | 93360_NCI-H292_IL-9 | 26.8 |
| cells_PMA/ionomycin and IL-18 | | | |
| 93578_NK Cells IL-2_resting | 5.7 | 93359_NCI-H292_IL-13 | 12.6 |
| 93109_Mixed Lymphocyte | 8.0 | 93357_NCI-H292_IFN gamma | 21.8 |
| Reaction_Two Way MLR | | | |
| 93110_Mixed Lymphocyte | 6.4 | 93777_HPAEC_- | 5.6 |
| Reaction_Two Way MLR | | | |
| 93111_Mixed Lymphocyte | 2.9 | 93778_HPAEC_IL-1 beta/TNA | 8.1 |
| Reaction_Two Way MLR | | alpha | |
| 93112_Mononuclear Cells | 3.3 | 93254_Normal Human Lung | 6.9 |
| (PBMCs)_resting | | Fibroblast_none | |
| 93113_Mononuclear Cells | 24.1 | 93253_Normal Human Lung | 3.5 |
| (PBMCs)_PWM | | Fibroblast_TNFa (4 ng/ml) and IL- | |
| | | 1b (1 ng/ml) | |
| 93114_Mononuclear Cells | 4.7 | 93257_Normal Human Lung | 28.9 |
| (PBMCs)_PHA-L | | Fibroblast_IL-4 | |
| 93249_Ramos (B cell)_none | 33.4 | 93256_Normal Human Lung | 27.7 |
| | | Fibroblast_IL-9 | |
| 93250_Ramos (B cell)_ionomycin | 100.0 | 93255_Normal Human Lung | 13.1 |
| | | Fibroblast_IL-13 | |
| 93349_B lymphocytes_PWM | 63.3 | 93258_Normal Human Lung | 22.7 |
| | | Fibroblast_IFN gamma | |
| 93350_B lymphoytes_CD40L and | 22.7 | 93106_Dermal Fibroblasts | 20.3 |
| IL-4 | | CCD1070_resting | |
| 92665_EOL-1 | 14.5 | 93361_Dermal Fibroblasts | 36.3 |
| (Eosinophil)_dbcAMP | | CCD1070_TNF alpha 4 ng/ml | |
| differentiated | | | |
| 93248_EOL-1 | 7.2 | 93105_Dermal Fibroblasts | 8.7 |
| (Eosinophil)_dbcAMP/PMAionomycin | | CCD1070_IL-1 beta 1 ng/ml | |
| 93356_Dendritic Cells_none | 6.0 | 93772_dermal fibroblast_IFN | 8.4 |
| | | gamma | |
| 93355_Dendritic Cells_LPS 100 ng/ml | 2.5 | 93771_dermal fibroblast_IL-4 | 17.1 |
| 93775_Dendritic Cells_anti-CD40 | 4.2 | 93260_IBD Colitis 2 | 0.8 |
| 93774_Monocytes_resting | 4.6 | 93261_IBD Crohns | 0.8 |
| 93776_Monocytes_LPS 50 ng/ml | 0.6 | 735010_Colon_normal | 14.8 |
| 93581_Macrophages_resting | 7.8 | 735019_Lung_none | 4.4 |
| 93582_Macrophages_LPS 100 ng/ml | 0.5 | 64028-1_Thymus_none | 14.4 |
| 93098_HUVEC | 8.7 | 64030-1_Kidney_none | 6.2 |
| (Endothelial)_none | | | |
| 93099_HUVEC | 10.7 | | |
| (Endothelial)_starved | | | |

TABLE 44
Panel CNS_neurodegeneration_V1.0

| | Relative | Relative |
|---|---|---|
| | Expression(%) | Expression(%) |
| | tm6962t_{—} | tm7090f_{—} |
| Tissue Name | ag1551_b1_s2 | ag3362_a1 |
| AD 1 Hippo | 19.0 | 9.9 |
| AD 2 Hippo | 33.6 | 33.3 |
| AD 3 Hippo | 8.2 | 4.3 |
| AD 4 Hippo | 10.7 | 16.5 |
| AD 5 hippo | 72.1 | 97.0 |
| AD 6 Hippo | 40.3 | 43.2 |
| Control 2 Hippo | 27.0 | 29.1 |
| Control 4 Hippo | 7.8 | 16.6 |
| Control (Path) 3 Hippo | 4.7 | 3.8 |
| AD 1 Temporal Ctx | 12.9 | 7.1 |
| AD 2 Temporal Ctx | 37.4 | 23.2 |
| AD 3 Temporal Ctx | 9.3 | 5.6 |
| AD 4 Temporal Ctx | 17.7 | 20.1 |
| AD 5 Inf Temporal Ctx | 79.0 | 100.0 |
| AD 5 SupTemporal Ctx | 35.4 | 44.0 |
| AD 6 Inf Temporal Ctx | 30.2 | 30.9 |
| AD 6 Sup Temporal Ctx | 35.8 | 69.8 |
| Control 1 Temporal Ctx | 12.1 | 9.1 |
| Control 2 Temporal Ctx | 42.8 | 59.1 |
| Control 3 Temporal Ctx | 18.0 | 11.7 |
| Control 4 Temporal Ctx | 9.7 | 8.2 |
| Control (Path) 1 Temporal Ctx | 78.6 | 56.2 |
| Control (Path) 2 Temporal Ctx | 45.4 | 34.2 |
| Control (Path) 3 Temporal Ctx | 8.4 | 0.0 |
| Control (Path) 4 Temporal Ctx | 37.9 | 24.3 |
| AD 1 Occipital Ctx | 15.2 | 2.0 |
| AD 2 Occipital Ctx (Missing) | 0.0 | 0.0 |
| AD 3 Occipital Ctx | 4.8 | 5.4 |
| AD 4 Occipital Ctx | 19.4 | 24.7 |
| AD 5 Occipital Ctx | 12.2 | 24.5 |
| AD 6 Occipital Ctx | 45.3 | 31.8 |
| Control 1 Occipital Ctx | 2.6 | 0.9 |
| Control 2 Occipital Ctx | 70.2 | 89.8 |
| Control 3 Occipital Ctx | 14.9 | 12.6 |
| Control 4 Occipital Ctx | 10.3 | 6.3 |
| Control (Path) 1 Occipital Ctx | 100.0 | 65.4 |
| Control (Path) 2 Occipital Ctx | 12.7 | 15.9 |
| Control (Path) 3 Occipital Ctx | 1.8 | 2.0 |
| Control (Path) 4 Occipital Ctx | 14.9 | 11.6 |
| Control 1 Parietal Ctx | 9.3 | 2.8 |
| Control 2 Parietal Ctx | 25.9 | 39.4 |
| Control 3 Parietal Ctx | 19.5 | 23.5 |
| Control (Path) 1 Parietal Ctx | 61.1 | 69.7 |
| Control (Path) 2 Parietal Ctx | 22.2 | 14.9 |
| Control (Path) 3 Parietal Ctx | 2.3 | 0.9 |
| Control (Path) 4 Parietal Ctx | 60.1 | 38.9 |

Panel 1.3D Summary Ag1551 The NOV4 gene is widely expressed in this panel, with highest expression in the breast cancer cell line MCF-7 (CT=29). Of note is the difference in expression between the fetal and adult heart and skeletal muscle, with higher expression seen in the tissues derived from the fetal source (CTs=30-32) than in tissues derived from the adult (CTs=34-36). Thus, expression of the NOV4 gene could be used to distinguish between fetal and adult heart and skeletal muscle. In general, expression of the NOV4 gene appears to be greater in the cancer cell lines and fetal cells than in the samples derived from normal tissues. Since normal cultured cell lines and fetal cells are highly proliferative, this observation may indicate that the expression of the NOV4 gene might be used to distinguish proliferating cells over resting or quiescent cells.

Among tissues involved in central nervous system function, this gene is expressed at moderate levels in all brain regions examined. The NOV4 gene encodes a protein with a putative WD40 motif, which is known to interact with the G protein beta subunit (Gbeta), suggesting a role in signal transduction. Several neurotransmitter receptors are GPCRs, including the dopamine receptor family, the serotonin receptor family, the GABAB receptor, muscarinic acetylcholine receptors, and others; thus this molecule may mediate signaling via a neurotransmitter receptor. Targeting various neurotransmitter receptors (dopamine, serotonin) has proven to be an effective therapy in psychiatric illnesses such as schizophrenia, bipolar disorder and depression. Therefore, therapeutic modulation of this gene or its protein product may be beneficial in one or more of these diseases, as may antagonism of the protein encoded by the gene.

Among tissues with metabolic function, the NOV4 gene is expressed in adipose, adrenal gland, adult and fetal heart, adult and fetal liver, pancreas, pituitary and thyroid. This putative nuclear protein may be important for the pathogenesis and/or treatment of disease in any or all of these tissues, including obesity and diabetes.

Panel General_screening_panel_v1.0 Summary Ag3362 The NOV4 gene is widely expressed in this panel, with highest expression in the breast cancer cell line T47D (CT=29). Significant expression is also seen in cell lines derived from prostate, breast and ovarian cancers. In general, expression of the NOV4 gene appears to be greater in the cancer cell lines than in normal tissue, an observation that is consistent with the results from Panel 1.3D. Thus, the expression of this gene could be used to distinguish these cell line types from others in the panel.

Panel 2.2 Summary Ag1551 Highest expression of the NOV4 gene is seen in normal kidney tissue adjacent to malignant kidney tissue. Thus, the expression of this gene could be used to distinguish this sample from others in the panel.

Panel 4D Summary Ag1551 The NOV4 gene is widely expressed in this panel, a pattern detected in the other panels as well, with highest expression in the ionomycin activated B cell line (Ramos) (CT=28.6). Significant expression is also detected in normal B lymphocytes, a B cell line (Ramos), and in normal B cells activated by pokeweed mitogen. Cytoplasmic and nuclear localization (PSORT) suggests that this G protein-beta-WD40 motif protein homolog may be used as a target for small molecule drug discovery. Activated B lymphocytes are important antigen presenting cells that participate in the stimulation of the immune response in numerous settings. Inhibitory small molecule drugs that reduce the function of activated B cells may reduce or eliminate symptoms in patients with autoimmune and inflammatory diseases such as lupus erythematosus, Crohn's disease, ulcerative colitis, asthma, psoriasis, and rheumatoid arthritis.

Please note that data from one run with the probe and primer set Ag3362 is not included because the amp plot corresponding to the run indicates that there were problems with the experiment.

Panel CNS_neurodegeneration_V1.0 Summary Ag1551/Ag336 Highest expression of the the NOV4 gene is seen in the occipital cortex of a control patient and the temporal cortex of an Alzheimer's patient. While the NOV4 gene does not appear to be preferentially expressed in Alzheimer's disease, this panel confirms expression of the NOV4 gene at moderate/high levels in the brain in an additional set of individuals. Please see Panel 1.3D for discussion of potential utility of this gene in the central nervous system (Zhu et al., Transcription activating property of autoantigen SG2NA and modulating effect of WD-40 repeats. Exp Cell Res. 269(2):312-21, 2001).

NOV5a and NOV5b: Novel Opioid Binding Cell Adhesion Molecule

Expression of the NOV5a gene (139785504) and the NOV5b variant (139785504_da1) was assessed using the primer-probe sets Ag3090 and Ag3092 described in Tables 45 and 46. Results from RTQ-PCR runs are shown in Tables 47-50.

TABLE 45

Probe Name Ag3090

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-ATGACAGACTGCTGAGCAGC-3′ | 59.3 | 20 | 706 | 173 |
| Probe | FAM-5′-AAGGCCTGAAGGTGCAGACGGAG-3′-TAMRA | 70 | 23 | 736 | 174 |
| Reverse | 5′-CGTTGGCAAAGAGAAGCAT-3′ | 59 | 19 | 789 | 175 |

TABLE 46

Probe Name Ag3092

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-ATGACAGACTGCTGAGCAGC-3′ | 59.3 | 20 | 707 | 176 |
| Probe | FAM-5′-AAGGCCTGAAGGTGCAGACCGAG-3′-TAMRA | 70 | 23 | 737 | 177 |
| Reverse | 5′-CGTTGGCAAAGAGAAGCAT-3′ | 59 | 19 | 772 | 178 |

TABLE 47
Panel 1.3D

| | Relative | Relative |
|---|---|---|
| | Expression(%) | Expression(%) |
| | 1.3dx4tm5823f_{—} | 1.3dx4tm5472f_{—} |
| Tissue Name | ag3090_a2 | ag3092_b1 |
| Liver adenocarcinoma | 0.3 | 0.0 |
| Pancreas | 0.0 | 0.0 |
| Pancreatic ca. CAPAN 2 | 0.0 | 0.0 |
| Adrenal gland | 1.5 | 0.0 |
| Thyroid | 0.0 | 0.0 |
| Salivary gland | 0.0 | 0.0 |
| Pituitary gland | 3.4 | 0.2 |
| Brain (fetal) | 100.0 | 53.3 |
| Brain (whole) | 29.4 | 51.5 |
| Brain (amygdala) | 31.8 | 50.9 |
| Brain (cerebellum) | 93.5 | 71.4 |
| Brain (hippocampus) | 41.1 | 91.1 |
| Brain (substantia nigra) | 27.0 | 24.5 |
| Brain (thalamus) | 20.3 | 100.0 |
| Cerebral Cortex | 89.4 | 37.4 |
| Spinal cord | 16.4 | 7.9 |
| CNS ca. (glio/astro) U87-MG | 0.0 | 0.0 |
| CNS ca. (glio/astro) U-118-MG | 0.0 | 0.0 |
| CNS ca. (astro) SW1783 | 0.0 | 0.0 |
| CNS ca.* (neuro; met) SK-N-AS | 2.2 | 3.2 |
| CNS ca. (astro) SF-539 | 0.0 | 0.0 |
| CNS ca. (astro) SNB-75 | 1.4 | 0.0 |
| CNS ca. (glio) SNB-19 | 0.0 | 0.2 |
| CNS ca. (glio) U251 | 0.8 | 0.0 |
| CNS ca. (glio) SF-295 | 0.0 | 0.1 |
| Heart (fetal) | 0.0 | 0.1 |
| Heart | 0.0 | 0.0 |
| Fetal Skeletal | 0.0 | 5.2 |
| Skeletal muscle | 0.2 | 0.0 |
| Bone marrow | 0.0 | 0.0 |
| Thymus | 0.0 | 1.6 |
| Spleen | 0.3 | 1.6 |
| Lymph node | 0.0 | 0.0 |
| Colorectal | 1.7 | 0.1 |
| Stomach | 0.0 | 0.0 |
| Small intestine | 0.5 | 0.0 |
| Colon ca. SW480 | 0.0 | 0.0 |
| Colon ca.* (SW480 met)SW620 | 0.4 | 0.0 |
| Colon ca. HT29 | 0.5 | 0.1 |
| Colon ca. HCT-116 | 0.0 | 0.2 |
| Colon ca. CaCo-2 | 0.5 | 0.0 |
| 83219 CC Well to Mod Diff | 5.8 | 0.9 |
| (ODO3866) | | |
| Colon ca. HCC-2998 | 0.7 | 1.0 |
| Gastric ca.* (liver met) NCI-N87 | 0.3 | 0.0 |
| Bladder | 0.0 | 0.0 |
| Trachea | 0.0 | 0.0 |
| Kidney | 0.0 | 0.0 |
| Kidney (fetal) | 13.3 | 4.4 |
| Renal ca. 786-0 | 0.2 | 0.0 |
| Renal ca. A498 | 0.0 | 0.1 |
| Renal ca. RXF 393 | 0.1 | 0.0 |
| Renal ca. ACHN | 0.0 | 0.0 |
| Renal ca. UO-31 | 0.0 | 0.0 |
| Renal ca. TK-10 | 0.5 | 0.0 |
| Liver | 0.0 | 0.8 |
| Liver (fetal) | 1.2 | 0.0 |
| Liver ca. (hepatoblast) HepG2 | 21.2 | 5.4 |
| Lung | 0.0 | 0.0 |
| Lung (fetal) | 1.8 | 2.1 |
| Lung ca. (small cell) LX-1 | 0.0 | 0.0 |
| Lung ca. (small cell) NCI-H69 | 0.2 | 0.0 |
| Lung ca. (s.cell var.) SHP-77 | 1.6 | 0.0 |
| Lung ca. (large cell) NCI-H460 | 0.0 | 2.0 |
| Lung ca. (non-sm. cell) A549 | 0.8 | 0.0 |
| Lung ca. (non-s.cell) NCI-H23 | 21.3 | 10.1 |
| Lung ca (non-s.cell) HOP-62 | 0.0 | 0.0 |
| Lung ca. (non-s.cl) NCI-H522 | 26.9 | 3.7 |
| Lung ca. (squam.) SW 900 | 0.0 | 0.1 |
| Lung ca. (squam.) NCI-H596 | 0.0 | 0.0 |
| Mammary gland | 0.0 | 0.0 |
| Breast ca.* (pl. effusion) MCF-7 | 0.0 | 0.0 |
| Breast ca.* (pl.ef) MDA-MB-231 | 0.5 | 0.0 |
| Breast ca.* (pl. effusion) T47D | 0.0 | 0.0 |
| Breast ca. BT-549 | 0.0 | 0.1 |
| Breast ca. MDA-N | 0.4 | 0.0 |
| Ovary | 8.9 | 2.0 |
| Ovarian ca. OVCAR-3 | 0.0 | 0.0 |
| Ovarian ca. OVCAR-4 | 1.1 | 0.0 |
| Ovarian ca. OVCAR-5 | 0.4 | 0.0 |
| Ovarian ca. OVCAR-8 | 0.5 | 0.4 |
| Ovarian ca. IGROV-1 | 0.0 | 0.1 |
| Ovarian ca.* (ascites) SK-OV-3 | 0.0 | 2.6 |
| Uterus | 0.0 | 0.0 |
| Placenta | 0.0 | 0.0 |
| Prostate | 0.0 | 0.3 |
| Prostate ca.* (bone met)PC-3 | 0.2 | 0.1 |
| Testis | 5.9 | 15.0 |
| Melanoma Hs688(A).T | 0.0 | 0.0 |
| Melanoma* (met) Hs688(B).T | 0.0 | 0.0 |
| Melanoma UACC-62 | 0.0 | 0.1 |
| Melanoma M14 | 0.0 | 0.1 |
| Melanoma LOX IMVI | 0.0 | 0.0 |
| Melanoma* (met) SK-MEL-5 | 0.0 | 0.0 |
| Adipose | 3.1 | 0.8 |

TABLE 48
Panel 4D

| | Relative | | Relative |
|---|---|---|---|
| | Expression(%) | | Expression(%) |
| | 4dx4tm5038f_{—} | | 4dx4tm5038f_{—} |
| Tissue Name | ag3092_b2 | Tissue Name | ag3092_b2 |
| 93768_Secondary Th1_anti- | 0.0 | 93100_HUVEC (Endothelial)_IL- | 0.0 |
| CD28/anti-CD3 | | 1b | |
| 93769_Secondary Th2_anti- | 100.0 | 93779_HUVEC (Endothelial)_IFN | 0.0 |
| CD28/anti-CD3 | | gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting day | 0.0 | 93101_HUVEC | 0.0 |
| 4-6 in IL-2 | | (Endothelial)_TNF alpha + IL4 | |
| 93572_Secondary Th2_resting day | 0.0 | 93781_HUVEC (Endothelial)_IL- | 0.0 |
| 4-6 in IL-2 | | 11 | |
| 93571_Secondary Tr1_resting day | 0.0 | 93583_Lung Microvascular | 0.0 |
| 4-6 in IL-2 | | Endothelial Cells_none | |
| 93568_primary Th1_anti- | 6.2 | 93584_Lung Microvascular | 4.9 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) and | |
| | | IL1b (1 ng/ml) | |
| 93565_primary Th1_resting dy 4-6 | 0.0 | 93773_Bronchial | 0.0 |
| in IL-2 | | epithelium_TNFa (4 ng/ml) and | |
| | | IL1b (1 ng/ml)** | |
| 93566_primary Th2_resting dy 4-6 | 0.0 | 93347_Small Airway | 0.0 |
| in IL-2 | | Epithelium_none | |
| 93567_primary Tr1_resting dy 4-6 | 0.0 | 93348_Small Airway | 0.0 |
| in IL-2 | | Epithelium_TNFa (4 ng/ml) and | |
| | | IL1b (1 ng/ml) | |
| 93351_CD45RA CD4 | 2.9 | 92668_Coronery Artery | 24.7 |
| lymphocyte_anti-CD28/anti-CD3 | | SMC_resting | |
| 93352_CD45RO CD4 | 5.3 | 92669_Coronery Artery | 0.0 |
| lymphocyte_anti-CD28/anti-CD3 | | SMC_TNFa (4 ng/ml) and IL1b (1 ng/ml) | |
| 93251_CD8 Lymphocytes_anti- | 0.0 | 93107_astrocytes_resting | 0.0 |
| CD28/anti-CD3 | | | |
| 93353_chronic CD8 Lymphocytes | 0.0 | 93108_astrocytes_TNFa (4 ng/ml) | 0.0 |
| 2ry_resting dy 4-6 in IL-2 | | and IL1b (1 ng/ml) | |
| 93574_chronic CD8 Lymphocytes | 0.0 | 92666_KU-812 (Basophil)_resting | 25.9 |
| 2ry_activated CD3/CD28 | | | |
| 93354_CD4_none | 0.0 | 92667_KU-812 | 23.4 |
| | | (Basophil)_PMA/ionoycin | |
| 93252_Secondary | 14.2 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and IFNg** | |
| 93788_LAK cells_IL-2 | 0.0 | 93791_Liver Cirrhosis | 25.4 |
| 93787_LAK cells_IL-2 + IL-12 | 0.0 | 93792_Lupus Kidney | 0.0 |
| 93789_LAK cells_IL-2 + IFN | 15.3 | 93577_NCI-H292 | 0.0 |
| gamma | | | |
| 93790_LAK cells_IL-2 + IL-18 | 0.0 | 93358_NCI-H292_IL-4 | 0.0 |
| 93104_LAK | 3.6 | 93360_NCI-H292_IL-9 | 0.0 |
| cells_PMA/ionomycin and IL-18 | | | |
| 93578_NK Cells IL-2_resting | 0.0 | 93359_NCI-H292_IL-13 | 0.0 |
| 93109_Mixed Lymphocyte | 0.0 | 93357_NCI-H292_IFN gamma | 0.0 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 14.6 | 93778_HPAEC_IL-1 beta/TNA | 2.2 |
| Reaction_Two Way MLR | | alpha | |
| 93112_Mononuclear Cells | 4.2 | 93254_Normal Human Lung | 0.0 |
| (PBMCs)_resting | | Fibroblast_none | |
| 93113_Mononuclear Cells | 14.2 | 93253_Normal Human Lung | 0.0 |
| (PBMCs)_PWM | | Fibroblast_TNFa (4 ng/ml) and IL- | |
| | | 1b (1 ng/ml) | |
| 93114_Mononuclear Cells | 37.1 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B cell)_ionomycin | 0.0 | 93255_Normal Human Lung | 0.0 |
| | | Fibroblast_IL-13 | |
| 93349_B lymphocytes_PWM | 2.6 | 93258_Normal Human Lung | 0.0 |
| | | Fibroblast_IFN gamma | |
| 93350_B lymphoytes_CD40L and | 0.0 | 93106_Dermal Fibroblasts | 0.0 |
| IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 37.9 | 93105_Dermal Fibroblasts | 0.0 |
| (Eosinophil)_dbcAMP/PMAionomycin | | CCD1070_IL-1 beta 1 ng/ml | |
| 93356_Dendritic Cells_none | 0.0 | 93772_dermal fibroblast_IFN | 0.0 |
| | | gamma | |
| 93355_Dendritic Cells_LPS 100 ng/ml | 17.3 | 93771_dermal fibroblast_IL-4 | 0.0 |
| 93775_Dendritic Cells_anti-CD40 | 0.0 | 93260_IBD Colitis 2 | 0.0 |
| 93774_Monocytes_resting | 0.0 | 93261_IBD Crohns | 0.0 |
| 93776_Monocytes_LPS 50 ng/ml | 0.0 | 735010_Colon_normal | 52.3 |
| 93581_Macrophages_resting | 0.0 | 735019_Lung none | 58.3 |
| 93582_Macrophages_LPS 100 ng/ml | 5.0 | 64028-1_Thymus_none | 0.0 |
| 93098_HUVEC | 0.0 | 64030-1_Kidney_none | 0.0 |
| (Endothelial)_none | | | |
| 93099_HUVEC | 0.0 | | |
| (Endothelial)_starved | | | |

TABLE 49
Panel CNS_1

| | Relative | | Relative |
|---|---|---|---|
| | Expression(%) | | Expression(%) |
| | cns1x4tm6194f_{—} | | cns1x4tm6194f_{—} |
| Tissue Name | ag3092_b2 | Tissue Name | ag3092_b2 |
| 102633_BA4 Control | 25.5 | 102605_BA17 PSP | 10.2 |
| 102641_BA4 Control2 | 26.5 | 102612_BA17 PSP2 | 11.5 |
| 102625_BA4 Alzheimer's2 | 6.8 | 102637_Sub Nigra Control | 52.7 |
| 102649_BA4 Parkinson's | 25.0 | 102645_Sub Nigra Control2 | 60.1 |
| 102656_BA4 Parkinson's2 | 84.7 | 102629_Sub Nigra Alzheimer's2 | 4.1 |
| 102664_BA4 Huntington's | 16.0 | 102660_Sub Nigra Parkinson's2 | 90.9 |
| 102671_BA4 Huntington's2 | 39.4 | 102667_Sub Nigra Huntington's | 66.9 |
| 102603_BA4 PSP | 20.0 | 102674_Sub Nigra Huntington's2 | 64.5 |
| 102610_BA4 PSP2 | 21.8 | 102614_Sub Nigra PSP2 | 7.2 |
| 102588_BA4 Depression | 13.8 | 102592_Sub Nigra Depression | 3.6 |
| 102596_BA4 Depression2 | 7.9 | 102599_Sub Nigra Depression2 | 11.0 |
| 102634_BA7 Control | 54.8 | 102636_Glob Palladus Control | 13.6 |
| 102642_BA7 Control2 | 100.0 | 102644_Glob Palladus Control2 | 9.3 |
| 102626_BA7 Alzheimer's2 | 7.2 | 102620_Glob Palladus Alzheimer's | 10.0 |
| 102650_BA7 Parkinson's | 16.9 | 102628_Glob Palladus | 0.0 |
| | | Alzheimer's2 | |
| 102657_BA7 Parkinson's2 | 35.3 | 102652_Glob Palladus Parkinson's | 28.2 |
| 102665_BA7 Huntington's | 20.5 | 102659_Glob Palladus | 16.1 |
| | | Parkinson's2 | |
| 102672_BA7 Huntington's2 | 27.6 | 102606_Glob Palladus PSP | 1.6 |
| 102604_BA7 PSP | 12.1 | 102613_Glob Palladus PSP2 | 4.1 |
| 102611_BA7 PSP2 | 13.9 | 102591_Glob Palladus Depression | 1.8 |
| 102589_BA7 Depression | 8.1 | 102638_Temp Pole Control | 14.1 |
| 102632_BA9 Control | 7.8 | 102646_Temp Pole Control2 | 26.2 |
| 102640_BA9 Control2 | 53.8 | 102622_Temp Pole Alzheimer's | 1.8 |
| 102617_BA9 Alzheimer's | 3.5 | 102630_Temp Pole Alzheimer's2 | 8.8 |
| 102624_BA9 Alzheimer's2 | 13.0 | 102653_Temp Pole Parkinson's | 13.6 |
| 102648_BA9 Parkinson's | 25.0 | 102661_Temp Pole Parkinson's2 | 33.6 |
| 102655_BA9 Parkinson's2 | 35.4 | 102668_Temp Pole Huntington's | 19.8 |
| 102663_BA9 Huntington's | 44.2 | 102607_Temp Pole PSP | 3.7 |
| 102670_BA9 Huntington's2 | 19.8 | 102615_Temp Pole PSP2 | 3.8 |
| 102602_BA9 PSP | 10.1 | 102600_Temp Pole Depression2 | 0.2 |
| 102609_BA9 PSP2 | 2.1 | 102639_Cing Gyr Control | 43.5 |
| 102587_BA9 Depression | 7.3 | 102647_Cing Gyr Control2 | 41.3 |
| 102595_BA9 Depression2 | 25.5 | 102623_Cing Gyr Alzheimer's | 2.0 |
| 102635_BA17 Control | 46.7 | 102631_Cing Gyr Alzheimer's2 | 12.6 |
| 102643_BA17 Control2 | 52.4 | 102654_Cing Gyr Parkinson's | 18.3 |
| 102627_BA17 Alzheimer's2 | 14.0 | 102662_Cing Gyr Parkinson's2 | 7.7 |
| 102651_BA17 Parkinson's | 26.5 | 102669_Cing Gyr Huntington's | 20.8 |
| 102658_BA17 Parkinson's2 | 38.5 | 102676_Cing Gyr Huntington's2 | 13.1 |
| 102666_BA17 Huntington's | 44.2 | 102608_Cing Gyr PSP | 0.0 |
| 102673_BA17 Huntington's2 | 13.7 | 102616_Cing Gyr PSP2 | 1.7 |
| 102590_BA17 Depression | 6.0 | 102594_Cing Gyr Depression | 7.6 |
| 102597_BA17 Depression2 | 28.0 | 102601_Cing Gyr Depression2 | 2.3 |

TABLE 50
Panel CNS_neurodegeneration_V1.0

| | Relative | Relative |
|---|---|---|
| | Expression(%) | Expression(%) |
| | tm7048f_{—} | tm7048f_{—} |
| Tissue Name | ag3090_a2_s2 | ag3092_b2_s1 |
| AD 1 Hippo | 15.6 | 21.3 |
| AD 2 Hippo | 28.0 | 33.7 |
| AD 3 Hippo | 6.0 | 9.7 |
| AD 4 Hippo | 9.0 | 13.5 |
| AD 5 hippo | 49.9 | 69.4 |
| AD 6 Hippo | 38.4 | 41.1 |
| Control 2 Hippo | 34.5 | 43.2 |
| Control 4 Hippo | 11.8 | 13.5 |
| Control (Path) 3 Hippo | 6.3 | 17.7 |
| AD 1 Temporal Ctx | 11.1 | 12.9 |
| AD 2 Temporal Ctx | 24.3 | 24.9 |
| AD 3 Temporal Ctx | 6.1 | 6.6 |
| AD 4 Temporal Ctx | 18.1 | 25.8 |
| AD 5 Inf Temporal Ctx | 34.6 | 51.3 |
| AD 5 SupTemporal Ctx | 19.4 | 25.9 |
| AD 6 Inf Temporal Ctx | 34.7 | 30.8 |
| AD 6 Sup Temporal Ctx | 33.4 | 28.3 |
| Control 1 Temporal Ctx | 14.1 | 12.1 |
| Control 2 Temporal Ctx | 79.3 | 52.5 |
| Control 3 Temporal Ctx | 14.4 | 20.2 |
| Control 4 Temporal Ctx | 15.4 | 13.6 |
| Control (Path) 1 Temporal Ctx | 39.4 | 58.3 |
| Control (Path) 2 Temporal Ctx | 34.7 | 46.7 |
| Control (Path) 3 Temporal Ctx | 4.4 | 4.9 |
| Control (Path) 4 Temporal Ctx | 34.1 | 49.6 |
| AD 1 Occipital Ctx | 18.3 | 20.0 |
| AD 2 Occipital Ctx (Missing) | 0.0 | 0.0 |
| AD 3 Occipital Ctx | 11.4 | 14.8 |
| AD 4 Occipital Ctx | 23.0 | 26.3 |
| AD 5 Occipital Ctx | 11.0 | 64.9 |
| AD 6 Occipital Ctx | 69.7 | 14.5 |
| Control 1 Occipital Ctx | 7.5 | 12.4 |
| Control 2 Occipital Ctx | 75.4 | 100.0 |
| Control 3 Occipital Ctx | 22.1 | 30.9 |
| Control 4 Occipital Ctx | 5.7 | 7.3 |
| Control (Path) 1 Occipital Ctx | 53.1 | 57.6 |
| Control (Path) 2 Occipital Ctx | 11.4 | 19.6 |
| Control (Path) 3 Occipital Ctx | 5.8 | 7.0 |
| Control (Path) 4 Occipital Ctx | 22.7 | 26.2 |
| Control 1 Parietal Ctx | 10.2 | 14.8 |
| Control 2 Parietal Ctx | 24.1 | 30.3 |
| Control 3 Parietal Ctx | 30.4 | 33.3 |
| Control (Path) 1 Parietal Ctx | 100.0 | 84.8 |
| Control (Path) 2 Parietal Ctx | 25.1 | 33.2 |
| Control (Path) 3 Parietal Ctx | 5.4 | 8.4 |
| Control (Path) 4 Parietal Ctx | 54.8 | 57.5 |

Panel 1.3D Summary Ag3090/Ag3092 Two experiments with two different probe and primer sets produce results that are in very good agreement, with highest expression of the NOV5A gene in regions of the brain, specifically the thalamus and fetal brain (CTS=29-30). Indeed, the NOV5A gene shows a highly brain-preferential expression, and is expressed at high levels in all brain regions examined. The protein encoded by the NOV5A gene is a homolog of opioid-binding cell adhesion molecule (OBCAM), which is believed to be involved in axonal outgrowth. In addition, this molecule may be involved in the synaptic machinery. The limited ability of the CNS to regenerate after injury is the major impediment to the treatment of stroke, spinal cord and head trauma, and neurodegenerative diseases. Therefore, selective modulation of this gene or its protein product may be useful in enhancing neuroregeneration in any or all of these clinical conditions.

Panel 2.2 Summary Ag3090/Ag3092 Expression of the NOV5A gene is low/undetectable (Ct values >35) in all samples in this panel. (Data not shown.)

Panel 4D Summary Ag3092 Expression of the NOV5A gene is limited to a sample derived from secondary Th2 cells (Ct=34.2). This expression profile suggests that the NOV5A gene product may be useful as a protein therapeutic or a target for the generation of therapeutic antibodies that reduce or eliminate the symptoms in patients with one or more diseases related to the functions of Th2 cells, including asthma and allergies. Ag3090 Expression of the NOV5A gene is low/undetectable (Ct values >35) in all samples in this panel. (Data not shown.)

Panel CNS_{—}1 Summary Ag3092 Highest expression of the NOV5A gene is seen in the parietal cortex (Brodman's Area 7) of a control patient (CT=31.2). The gene is also widely expressed across many regions of the brain in many disease states. Please see Panel 1.3D for discussion of potential utility in the central nervous system.

Panel CNS_neurodegeneration_V1.0 Ag3090/Ag3092 Two experiments with two different probe and primer sets produce results that are in very good agreement, with highest expression in the occipital cortex and the parietal cortex of a control patient (CT=30.3). While the expression of this gene does not appear to be specific to Alzheimer's disease, the results of this panel confirm expression of the NOV5A gene at moderate/high level in the brain in an independent set of individuals. Please see Panel 1.3D for a discussion of potential utility in the central nervous system. (Hachisuka et al., Localization of opioid-binding cell adhesion molecule (OBCAM) in adult rat brain. Brain Res. 842(2):482-6, 1999; Hachisuka et al., Developmental expression of opioid-binding cell adhesion molecule (OBCAM) in rat brain. Brain Res Dev Brain Res 122(2):183-91, 2000).

NOV5d: Opioid Binding Cell Adhesion Molecule

Expression of the NOV5d gene (CG51027-05) was assessed using the primer-probe set Ag565 described in Table 51. Results from RTQ-PCR runs are shown in Tables 52-54.

TABLE 51

Probe Name Ag565

| | | | | Start | SEQ ID |
|---|---|---|---|---|---|
| Primers | Sequences | TM | Length | Position | NO: |
| Forward | 5′-CACCAGCCGTACACCACTCA-3′ | | 20 | 166 | 179 |
| Probe | TET-5′-CCACGTCCCTGCCCGCGTT-3′-TAMRA | | 19 | 201 | 180 |
| Reverse | 5′-TCACAGGCGACGAGATGTTC-3′ | | 20 | 222 | 181 |

TABLE 52
Panel 1.1

| | Relative | | Relative |
|---|---|---|---|
| | Expression(%) | | Expression(%) |
| | 1.1tm760t_{—} | | 1.1tm760t_{—} |
| Tissue Name | ag565 | Tissue Name | ag565 |
| Adrenal gland | 2.7 | Renal ca. UO-31 | 0.1 |
| Bladder | 0.5 | Renal ca. RXF 393 | 0.2 |
| Brain (amygdala) | 10.7 | Liver | 3.2 |
| Brain (cerebellum) | 100.0 | Liver (fetal) | 0.8 |
| Brain (hippocampus) | 27.4 | Liver ca. (hepatoblast) HepG2 | 0.0 |
| Brain (substantia nigra) | 73.2 | Lung | 0.0 |
| Brain (thalamus) | 44.1 | Lung (fetal) | 0.9 |
| Cerebral Cortex | 51.8 | Lung ca (non-s.cell) HOP-62 | 6.6 |
| Brain (fetal) | 48.3 | Lung ca. (large cell) NCI-H460 | 0.0 |
| Brain (whole) | 41.2 | Lung ca. (non-s.cell) NCI-H23 | 10.7 |
| CNS ca. (glio/astro) U-118-MG | 0.0 | Lung ca. (non-s.cl) NCI-H522 | 50.0 |
| CNS ca. (astro) SF-539 | 0.0 | Lung ca. (non-sm. cell) A549 | 0.1 |
| CNS ca. (astro) SNB-75 | 0.0 | Lung ca. (s.cell var.) SHP-77 | 0.7 |
| CNS ca. (astro) SW1783 | 0.1 | Lung ca. (small cell) LX-1 | 0.0 |
| CNS ca. (glio) U251 | 0.0 | Lung ca. (small cell) NCI-H69 | 2.3 |
| CNS ca. (glio) SF-295 | 0.0 | Lung ca. (squam.) SW 900 | 0.4 |
| CNS ca. (glio) SNB-19 | 0.0 | Lung ca. (squam.) NCI-H596 | 1.3 |
| CNS ca. (glio/astro) U87-MG | 0.0 | Lymph node | 0.0 |
| CNS ca.* (neuro; met) SK-N-AS | 9.3 | Spleen | 0.0 |
| Mammary gland | 0.8 | Thymus | 0.0 |
| Breast ca. BT-549 | 0.0 | Ovary | 6.2 |
| Breast ca. MDA-N | 0.0 | Ovarian ca. IGROV-1 | 0.0 |
| Breast ca.* (pl. effusion) T47D | 0.2 | Ovarian ca. OVCAR-3 | 0.1 |
| Breast ca.* (pl. effusion) MCF-7 | 0.0 | Ovarian ca. OVCAR-4 | 0.2 |
| Breast ca.* (pl.ef) MDA-MB-231 | 0.0 | Ovarian ca. OVCAR-5 | 0.3 |
| Small intestine | 1.8 | Ovarian ca. OVCAR-8 | 5.8 |
| Colorectal | 0.0 | Ovarian ca.* (ascites) SK-OV-3 | 0.1 |
| Colon ca. HT29 | 0.0 | Pancreas | 1.2 |
| Colon ca. CaCo-2 | 0.0 | Pancreatic ca. CAPAN 2 | 0.0 |
| Colon ca. HCT-15 | 0.0 | Pituitary gland | 11.3 |
| Colon ca. HCT-116 | 0.0 | Placenta | 0.0 |
| Colon ca. HCC-2998 | 0.0 | Prostate | 2.9 |
| Colon ca. SW480 | 0.0 | Prostate ca.* (bone met)PC-3 | 0.0 |
| Colon ca.* (SW480 met)SW620 | 0.0 | Salivary gland | 0.0 |
| Stomach | 0.4 | Trachea | 0.0 |
| Gastric ca.* (liver met) NCI-N87 | 0.0 | Spinal cord | 10.7 |
| Heart | 0.1 | Testis | 5.7 |
| Fetal Skeletal | 0.0 | Thyroid | 0.1 |
| Skeletal muscle | 0.0 | Uterus | 0.1 |
| Endothelial cells | 0.0 | Melanoma M14 | 0.1 |
| Heart (fetal) | 0.8 | Melanoma LOX IMVI | 0.0 |
| Kidney | 0.0 | Melanoma UACC-62 | 0.1 |
| Kidney (fetal) | 4.7 | Melanoma SK-MEL-28 | 0.0 |
| Renal ca. 786-0 | 0.0 | Melanoma* (met) SK-MEL-5 | 0.3 |
| Renal ca. A498 | 0.0 | Melanoma Hs688(A).T | 0.0 |
| Renal ca. ACHN | 0.0 | Melanoma* (met) Hs688(B).T | 0.1 |
| Renal ca. TK-10 | 0.0 | | |

TABLE 53
Panel 4.1D

| | Relative | | Relative |
|---|---|---|---|
| | Expression(%) | | Expression(%) |
| | 4.1dx4tm6519 | | 4.1dx4tm6519 |
| Tissue Name | t_ag565_a1 | Tissue Name | t_ag565_a1 |
| 93768_Secondary Th1_anti- | 0.0 | 93100_HUVEC (Endothelial)_IL- | 0.0 |
| CD28/anti-CD3 | | 1b | |
| 93769_Secondary Th2_anti- | 0.0 | 93779_HUVEC (Endothelial)_IFN | 0.0 |
| CD28/anti-CD3 | | gamma | |
| 93770_Secondary Tr1_anti- | 1.9 | 93102_HUVEC | 0.0 |
| CD28/anti-CD3 | | (Endothelial)_TNF alpha + IFN | |
| | | gamma | |
| 93573_Secondary Th1_resting day | 0.0 | 93101_HUVEC | 0.0 |
| 4-6 in IL-2 | | (Endothelial)_TNF alpha + IL4 | |
| 93572_Secondary Th2_resting day | 0.0 | 93781_HUVEC (Endothelial)_IL- | 0.0 |
| 4-6 in IL-2 | | 11 | |
| 93571_Secondary Tr1_resting day | 0.0 | 93583_Lung Microvascular | 0.0 |
| 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 3.1 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) and | |
| | | IL1b (1 ng/ml) | |
| 93565_primary Th1_resting dy 4-6 | 2.4 | 93773_Bronchial | 0.0 |
| in IL-2 | | epithelium_TNFa (4 ng/ml) and | |
| | | IL1b (1 ng/ml)** | |
| 93566_primary Th2_resting dy 4-6 | 0.0 | 93347_Small Airway | 0.0 |
| in IL-2 | | Epithelium_none | |
| 93567_primary Tr1_resting dy 4-6 | 0.0 | 93348_Small Airway | 0.3 |
| in IL-2 | | Epithelium_TNFa (4 ng/ml) and | |
| | | IL1b (1 ng/ml) | |
| 93351_CD45RA CD4 | 0.0 | 92668_Coronery Artery | 0.0 |
| lymphocyte_anti-CD28/anti-CD3 | | SMC_resting | |
| 93352_CD45RO CD4 | 0.0 | 92669_Coronery Artery | 0.0 |
| lymphocyte_anti-CD28/anti-CD3 | | SMC_TNFa (4 ng/ml) and IL1b (1 ng/ml) | |
| 93251_CD8 Lymphocytes_anti- | 0.0 | 93107_astrocytes_resting | 7.9 |
| CD28/anti-CD3 | | | |
| 93353_chronic CD8 Lymphocytes | 0.0 | 93108_astrocytes_TNFa (4 ng/ml) | 2.8 |
| 2ry_resting dy 4-6 in IL-2 | | and IL1b (1 ng/ml) | |
| 93574_chronic CD8 Lymphocytes | 0.0 | 92666_KU-812 (Basophil)_resting | 0.0 |
| 2ry_activated CD3/CD28 | | | |
| 93354_CD4_none | 0.0 | 92667_KU-812 | 4.5 |
| | | (Basophil)_PMA/ionoycin | |
| 93252_Secondary | 0.0 | 93579_CCD1106 | 0.0 |
| Th1/Th2/Tr1_anti-CD95 CH11 | | (Keratinocytes)_none | |
| 93103_LAK cells_resting | 2.4 | 93580_CCD1106 | 5.3 |
| | | (Keratinocytes)_TNFa and IFNg** | |
| 93788_LAK cells_IL-2 | 0.0 | 93791_Liver Cirrhosis | 2.3 |
| 93787_LAK cells_IL-2 + IL-12 | 0.0 | 93577_NCI-H292 | 0.0 |
| 93789_LAK cells_IL-2 + IFN | 0.0 | 93358_NCI-H292_IL-4 | 0.0 |
| gamma | | | |
| 93790_LAK cells_IL-2 + IL-18 | 0.0 | 93360_NCI-H292_IL-9 | 0.0 |
| 93104_LAK | 6.0 | 93359_NCI-H292 IL-13 | 0.0 |
| cells_PMA/ionomycin and IL-18 | | | |
| 93578_NK Cells IL-2_resting | 1.9 | 93357_NCI-H292_IFN gamma | 0.0 |
| 93109_Mixed Lymphocyte | 1.4 | 93777_HPAEC_- | 0.0 |
| Reaction_Two Way MLR | | | |
| 93110_Mixed Lymphocyte | 1.2 | 93778_HPAEC_IL-1 beta/TNA | 0.0 |
| Reaction_Two Way MLR | | alpha | |
| 93111_Mixed Lymphocyte | 0.0 | 93254_Normal Human Lung | 0.0 |
| Reaction_Two Way MLR | | Fibroblast_none | |
| 93112_Mononuclear Cells | 2.2 | 93253_Normal Human Lung | 2.1 |
| (PBMCs)_resting | | Fibroblast_TNFa (4 ng/ml) and IL- | |
| | | 1b (1 ng/ml) | |
| 93113_Mononuclear Cells | 0.0 | 93257_Normal Human Lung | 0.0 |
| (PBMCs)_PWM | | Fibroblast_IL-4 | |
| 93114_Mononuclear Cells | 0.8 | 93256_Normal Human Lung | 0.0 |
| (PBMCs)_PHA-L | | Fibroblast_IL-9 | |
| 93249_Ramos (B cell)_none | 0.0 | 93255_Normal Human Lung | 0.0 |
| | | Fibroblast_IL-13 | |
| 93250_Ramos (B cell)_ionomycin | 0.0 | 93258_Normal Human Lung | 0.0 |
| | | Fibroblast_IFN gamma | |
| 93349_B lymphocytes_PWM | 0.0 | 93106_Dermal Fibroblasts | 0.0 |
| | | CCD1070_resting | |
| 93350_B lymphoytes_CD40L and | 1.0 | 93361_Dermal Fibroblasts | 0.0 |
| IL-4 | | CCD1070_TNF alpha 4 ng/ml | |
| 92665_EOL-1 | 0.0 | 93105_Dermal Fibroblasts | 0.0 |
| (Eosinophil)_dbcAMP | | CCD1070 IL-1 beta 1 ng/ml | |
| differentiated | | | |
| 93248_EOL-1 | 18.2 | 93772_dermal fibroblast_IFN | 0.0 |
| (Eosinophil)_dbcAMP/PMAionomycin | | gamma | |
| 93356_Dendritic Cells_none | 0.0 | 93771_dermal fibroblast_IL-4 | 0.0 |
| 93355_Dendritic Cells_LPS 100 ng/ml | 0.0 | 93892_Dermal fibroblasts_none | 0.0 |
| 93775_Dendritic Cells_anti-CD40 | 0.0 | 99202_Neutrophils_TNFa+LPS | 0.0 |
| 93774_Monocytes_resting | 0.0 | 99203_Neutrophils_none | 0.0 |
| 93776_Monocytes_LPS 50 ng/ml | 0.0 | 735010_Colon_normal | 1.6 |
| 93581_Macrophages_resting | 0.0 | 735019_Lung_none | 1.4 |
| 93582_Macrophages_LPS 100 ng/ml | 0.0 | 64028-1_Thymus_none | 31.4 |
| 93098_HUVEC | 0.0 | 64030-1_Kidney_none | 100.0 |
| (Endothelial)_none | | | |
| 93099_HUVEC | 0.0 | | |
| (Endothelial)_starved | | | |

TABLE 54
Panel CNS_neurodegeneration_v1.0

| | Relative | | Relative |
|---|---|---|---|
| | Expression(%) | | Expression(%) |
| | tm6933t_{—} | | tm6933t_{—} |
| Tissue Name | ag565_b1_s2 | Tissue Name | ag565_b1_s2 |
| 106655_4951 Hippo | 19.7 | 106677_4624 BA21 | 3.2 |
| 106657_4986 Hippo | 36.4 | 106681_4640 BA21 | 35.3 |
| 106652_4933 Hippo | 8.0 | 106654_4951 BA17 | 12.6 |
| 106649_4901 Hippo | 7.7 | cns_water | 0.0 |
| 110138_3087 hippo | 91.7 | 106651_4933 BA17 | 7.3 |
| 110121_3027 Hippo | 41.1 | 106648_4901 BA17 | 20.6 |
| 106670_4971 Hippo | 48.3 | 110123_3027 Occ Ctx | 10.9 |
| 106666_4867 Hippo | 6.9 | 110140_3087 occ ctx | 56.3 |
| 106680_4624 Hippo | 3.3 | 106659_4595 BA17 | 1.9 |
| 106653_4951 BA21 | 9.6 | 106668_4971 BA17 | 100.0 |
| 106656_4986 BA21 | 29.4 | 106662_4737 BA17 | 11.0 |
| 106650_4933 BA21 | 4.0 | 106665_4867 BA17 | 7.5 |
| 106647_4901 BA21 | 14.4 | 106675_3975 BA17 | 82.9 |
| 110136_3087 inf temp ctx | 49.8 | 106672_3954 BA17 | 10.5 |
| 110137_3087 sup temp ctx | 27.3 | 106678_4624 BA17 | 0.9 |
| 110118_3027 Inf Temp Ctx | 25.1 | 106682_4640 BA17 | 20.9 |
| 110119_3027 Sup Temp Ctx | 40.8 | 106660_4595 BA7 | 5.6 |
| 106658_4595 BA21 | 5.1 | 113670_106669 pool | 20.3 |
| 106667_4971 BA21 | 82.8 | 106663_4737 BA7 | 24.8 |
| 106661_4737 BA21 | 19.6 | 106676_3975 BA7 | 97.3 |
| 106664_4867 BA21 | 7.9 | 106673_3954 BA7 | 26.4 |
| 106674_3975 BA21 | 77.6 | 106679_4624 BA7 | 2.9 |
| 106671_3954 BA21 | 38.0 | 106683_4640 BA7 | 55.7 |

Panel 1.1 Summary The NOV5D gene is expressed most highly in the cerebellum (CT=22). Indeed, this gene shows a highly brain-preferential expression, and is expressed at high levels in all brain regions examined. The protein encoded by the NOV5D gene is a homolog of opioid-binding cell adhesion molecule (OBCAM), which is believed to be involved in axonal outgrowth. In addition, this molecule may be involved in the synaptic machinery. The limited ability of the CNS to regenerate after injury is the major impediment to treatment of stroke, spinal cord and head trauma, and neurodegenerative diseases. Therefore, selective modulation of this gene or its protein product may be useful in enhancing neuroregeneration in any or all of these clinical conditions.

There is also significant expression in tissue derived from fetal heart and kidney (CTs=26-29) when compared to the level of expression in the adult source of these tissues (CTs=32-37). Thus, expression of this gene could be used to differentiate between the adult and fetal sources of heart and kidney tissue.

The NOV5D gene encodes a putative intracellular protein with moderate expression in many metabolic tissues including adrenal, adult and fetal liver, pancreas, pituitary and thyroid.

Therefore, this protein may be important for the pathogenesis and/or treatment of disease in any or all of these tissues, including obesity and diabetes.

Panel General_screening panel_v1.4 Summary Please note that data from this experiment is not included because the amp plot corresponding to the run indicates that there were problems with the run.

Panel 4.1D Summary Expression is limited to a few samples in this panel, with highest expression in the kidney (CT=30.4) and thymus. Moderate expression is seen dibutyryl-cAMP-differentiated and phorbol ester plus ionomycin-activated eosinophil cell line EOL-1, with low but significant levels of expression detected in unstimulated astrocytes. This expression profile suggests that the NOV5D gene product, an opioid-binding cell adhesion molecule homolog, may be useful as a therapeutic protein to reduce or eliminate symptoms resulting from diseases of the thymus and kidney. Furthermore, the protein encoded by the NOV5D gene may also reduce or eliminate allergies in which activated eosinophils play a role and may be effective in the treatment of multiple sclerosis where astrocytes present antigens to T lymphocytes.

Panel CNS_neurodegeneration_v1.0 Summary The NOV5D gene is widely expressed in this panel, with highest expression detected in the occipital cortex of a control brain (CT=29. 1). The expression seen across all regions of the brain is in agreement with the expression seen in Panel 1.1 While the expression of this gene does not appear be specific to Alzheimer's disease, this panel confirms expression at the moderate/high level in the brain in an additional set of individuals. Please see Panel 1.1 for discussion of potential utility in the central nervous system (Hachisuka et al., Localization of opioid-binding cell adhesion molecule (OBCAM) in adult rat brain. Brain Res. 842(2):482-6, 1999; Hachisuka et al., Developmental expression of opioid-binding cell adhesion molecule (OBCAM) in rat brain. Brain Res Dev Brain Res 122(2):183-91, 2000).

NOV6: Triacyiglycerol lipase-like

Expression of the NOV6a gene (SC122982104_A) and the NOV6b variant (CG58608-02) was assessed using the primer-probe sets Ag2179 and Ag3927 described in Tables 55 and 56. Please note that only the probe and primer set Ag3927 match the CG58608-02 sequence. Results from RTQ-PCR runs are shown in Table 57.

TABLE 55

Probe Name Ag2179

| | | | | Start | SEQ ID |
|---|---|---|---|---|---|
| Primers | Sequences | TM | Length | Position | NO: |
| Forward | 5′-AAATGGAAGTCAATAGGCAACA-3′ | 58.6 | 22 | 739 | 182 |
| Probe | TET-5′-AAGACTTCTTGCCTAAAACCTCATTTAAAA-3′-TAMRA | 63.7 | 30 | 761 | 183 |
| Reverse | 5′-ACACAGCTTTGAACCAATGAAT-3′ | 58.6 | 22 | 792 | 184 |

TABLE 56

Probe Name Ag3927

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-GTCCTGATCTGAACTTGGTTCA-3′ | 59.2 | 22 | 980 | 185 |
| Probe | TET-5′-CAGACAACGTCTCCATTATACAACATGACA-3′-TAMRA | 66.9 | 30 | 1009 | 186 |
| Reverse | 5′-TTACCATTCCAAATTGCAGTTG-3′ | 59.9 | 22 | 1052 | 187 |

TABLE 57
Panel General_screening_panel_v1.4

| | Relative |
|---|---|
| | Expression(%) |
| | tm7491t_{—} |
| Tissue Name | ag3927 |
| D6005-01_Human adipose | 1.5 |
| 112193_Metastatic melanoma | 1.6 |
| 112192_Metastatic melanoma | 0.0 |
| 95280_Epidermis (metastatic melanoma) | 0.0 |
| 95279_Epidermis (metastatic melanoma) | 1.5 |
| Melanoma (met)_SK-MEL-5 | 0.0 |
| 112196_Tongue (oncology) | 0.0 |
| 113461_Testis Pool | 100.0 |
| Prostate ca.(bone met)_PC-3 | 0.0 |
| 113455_Prostate Pool | 1.0 |
| 103396_Placenta | 0.0 |
| 113463_Uterus Pool | 0.0 |
| Ovarian carcinoma_OVCAR-3 | 2.1 |
| Ovarian carcinoma(ascites)_SK-OV-3 | 0.0 |
| 95297_Adenocarcinoma (ovary) | 0.0 |
| Ovarian carcinoma_OVCAR-5 | 11.8 |
| Ovarian carcinoma_IGROV-1 | 6.2 |
| Ovarian carcinoma_OVCAR-8 | 8.8 |
| 103368_Ovary | 1.2 |
| MCF7_breast carcinoma(pleural effusion) | 5.0 |
| Breast ca. (pleural effusion)_MDA-MB-231 | 0.0 |
| 112189_ductal cell carcinoma(breast) | 3.1 |
| Breast ca. (pleural effusion)_T47D | 0.0 |
| Breast carcinoma_MDA-N | 0.0 |
| 113452_Breast Pool | 2.1 |
| 103398_Trachea | 7.8 |
| 112354_lung | 0.0 |
| 103374_Fetal Lung | 2.7 |
| 94921_Small cell carcinoma of the lung | 0.0 |
| Lung ca.(small cell)_LX-1 | 5.7 |
| 94919_Small cell carcinoma of the lung | 0.0 |
| Lung ca.(s.cell var.)_SHP-77 | 0.0 |
| 95268_Lung (Large cell carcinoma) | 0.0 |
| 94920_Small cell carcinoma of the lung | 0.0 |
| Lung ca.(non-s.cell)_NCI-H23 | 2.5 |
| Lung ca.(large cell)_NCI-H460 | 0.0 |
| Lung ca.(non-s.cell)_HOP-62 | 0.0 |
| Lung ca.(non-s.cl)_NCI-H522 | 2.7 |
| 103392_Liver | 0.0 |
| 103393_Fetal Liver | 0.0 |
| Liver ca. (hepatoblast)_HepG2 | 0.0 |
| 113465_Kidney Pool | 32.8 |
| 103373_Fetal Kidney | 2.9 |
| Renal ca._786-0 | 0.0 |
| 112188_renal cell carcinoma | 0.0 |
| Renal ca._ACHN | 0.0 |
| 112190_Renal cell carcinoma | 0.0 |
| Renal ca._TK-10 | 2.4 |
| Bladder | 3.0 |
| Gastric ca.(liver met)_NCI-N87 | 13.4 |
| 112197_Stomach | 0.0 |
| 94938_Colon Adenocarcinoma | 0.0 |
| Colon ca._SW480 | 0.0 |
| Colon ca. (SW480 met)_SW620 | 10.0 |
| Colon ca._HT29 | 1.5 |
| Colon ca._HCT-116 | 0.0 |
| Colon ca._CaCo-2 | 1.4 |
| 83219_CC Well to Mod Diff (ODO3866) | 3.1 |
| 94936_Colon Adenocarcinoma | 0.0 |
| 94930_Colon | 0.0 |
| 94935_Colon Adenocarcinoma | 0.0 |
| 113468_Colon Pool | 7.7 |
| 113457_Small Intestine Pool | 14.9 |
| 113460_Stomach Pool | 1.8 |
| 113467_Bone Marrow Pool | 1.1 |
| 103371_Fetal Heart | 0.0 |
| 113451_Heart Pool | 0.8 |
| 113466_Lymph Node Pool | 2.7 |
| 103372_Fetal Skeletal Muscle | 12.5 |
| 113456_Skeletal Muscle Pool | 1.7 |
| 113459_Spleen Pool | 0.0 |
| 113462_Thymus Pool | 2.5 |
| CNS ca. (glio/astro)_U87-MG | 0.0 |
| CNS ca. (glio/astro)_U-118-MG | 0.0 |
| CNS ca. (neuro; met)_SK-N-AS | 1.1 |
| 95264_Brain astrocytoma | 0.0 |
| CNS ca. (astro)_SNB-75 | 0.0 |
| CNS ca. (glio)_SNB-19 | 0.0 |
| CNS ca. (glio)_SF-295 | 2.8 |
| 113447_Brain (Amygdala) Pool | 7.7 |
| 103382_Brain (cerebellum) | 3.7 |
| 64019-1_brain(fetal) | 27.5 |
| 113448_Brain (Hippocampus) Pool | 0.0 |
| 113464_Cerebral Cortex Pool | 9.6 |
| 113449_Brain (Substantia nigra) Pool | 2.4 |
| 113450_Brain (Thalamus) Pool | 9.0 |
| 103384_Brain (whole) | 1.2 |
| 113458_Spinal Cord Pool | 3.0 |
| 103375_Adrenal Gland | 0.0 |
| 113454_Pituitary gland Pool | 0.0 |
| 103397_Salivary Gland | 0.9 |
| 103369_Thyroid (female) | 24.7 |
| Pancreatic ca._CAPAN2 | 1.4 |
| 113453_Pancreas Pool | 1.8 |

Panel General_screening_panel_v1.4 Summary A 3927 Expression of the NOV6a gene in this panel is limited to samples originating from the testis (CT=33) and the kidney. Thus, expression of this gene could be used to distinguish testis and kidney tissue from the other tissues in this panel.

Ag2179 Expression of the NOV6a gene is low/undetectable (Ct values >35) in all samples in Panels 1.3D, 2D, 4D, and CNS_neurodegeneration_v1.0. (Data not shown.)

NOV7a: IGE Receptor Beta Subunit

Expression of the NOV7a gene (SC126624027_A) was assessed using the primer-probe set Ag2178 described in Table 58. Results from RTQ-PCR runs are shown in Table 59.

TABLE 58

Probe Name Ag2178

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-TTGAGAAAGGCAGAGAAATGAA-3′ | 59.1 | 22 | 85 | 188 |
| Probe | TET-5′-CAATAATGCTGAAAGTCATCAATGTAATCA-3′-TAMRA | 64.2 | 30 | 112 | 189 |
| Reverse | 5′-ACTGTCCTGTTCTTGGGAATTT-3′ | 59 | 22 | 142 | 190 |

TABLE 59
Panel 1.3D

| | Relative | | Relative |
|---|---|---|---|
| | Expression(%) | | Expression(%) |
| | 1.3dx4tm5487 | | 1.3dx4tm5487 |
| Tissue Name | t_ag2178_b1 | Tissue Name | t_ag2178_b1 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. (hepatoblast) HepG2 | 0.0 |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s.cell var.) SHP-77 | 0.0 |
| Spinal cord | 0.0 | Lung ca. (large cell) NCI-H460 | 0.0 |
| CNS ca. (glio/astro) U87-MG | 0.0 | Lung ca. (non-sm. cell) A549 | 0.0 |
| CNS ca. (glio/astro) U-118-MG | 0.0 | Lung ca. (non-s.cell) NCI-H23 | 0.0 |
| CNS ca. (astro) SW1783 | 0.0 | Lung ca. (non-s.cell) HOP-62 | 0.0 |
| CNS ca.* (neuro; met) SK-N-AS | 0.0 | Lung ca. (non-s.cl) NCI-H522 | 0.0 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 0.0 |
| CNS ca. (glio) U251 | 0.0 | Breast ca.* (pl. effusion) MCF-7 | 0.0 |
| CNS ca. (glio) SF-295 | 0.0 | Breast ca.* (pl.ef) MDA-MB-231 | 0.0 |
| Heart (fetal) | 0.0 | Breast ca.* (pl. effusion) T47D | 0.0 |
| Heart | 0.0 | Breast ca. BT-549 | 0.0 |
| Fetal Skeletal | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca.* (ascites) SK-OV-3 | 0.0 |
| Small intestine | 0.0 | Uterus | 0.0 |
| Colon ca. SW480 | 0.0 | Placenta | 0.0 |
| Colon ca.* (SW480 met)SW620 | 0.0 | Prostate | 0.0 |
| Colon ca. HT29 | 0.0 | Prostate ca.* (bone met)PC-3 | 0.0 |
| Colon ca. HCT-116 | 0.0 | Testis | 100.0 |
| Colon ca. CaCo-2 | 0.0 | Melanoma Hs688(A).T | 0.0 |
| 83219 CC Well to Mod Diff | 0.0 | Melanoma* (met) Hs688(B).T | 0.0 |
| (ODO3866) | | | |
| Colon ca. HCC-2998 | 0.0 | Melanoma UACC-62 | 0.0 |
| Gastric ca.* (liver met) NCI-N87 | 0.0 | Melanoma M14 | 0.0 |
| Bladder | 0.0 | Melanoma LOX IMVI | 0.0 |
| Trachea | 0.0 | Melanoma* (met) SK-MEL-5 | 0.0 |
| Kidney | 0.0 | Adipose | 0.0 |

Panel 1.3D Summary Expression of the NOV7a gene is restricted to the testis (CT=32.9). Thus, the expression of this gene could be used to distinguish testis tissue from the other samples on the panel.

Expression of the NOV7a gene is low/undetectable (Ct values >35) in all samples in Panels 2D and 4D. (Data not shown.)

NOV8: Munc18-like

Expression of the NOV8 gene (SC138745558_A) was assessed using the primer-probe set Ag2177 described in Table 60. Results from RTQ-PCR runs are shown in Tables 61-64.

TABLE 60

Probe Name Ag2177

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-TCCAGTGTTCAAAGCTTCTTTC-3′ | 58.6 | 22 | 937 | 191 |
| Probe | FAM-5′-CCCAAGCAAATTTGTCCTTATCACCG-3′-TAMRA | 68.9 | 26 | 983 | 192 |
| Reverse | 5′-CGAGCTTTCCAAATGTATCAAG-3′ | 58.9 | 22 | 1014 | 193 |

TABLE 61
Panel 1.3D

| | Relative | | Relative |
|---|---|---|---|
| | Expression(%) | | Expression(%) |
| | 1.3dtm4176f_{—} | | 1.3dtm4176f_{—} |
| Tissue Name | ag2177 | Tissue Name | ag2177 |
| Liver adenocarcinoma | 14.6 | Kidney (fetal) | 8.4 |
| Pancreas | 3.3 | Renal ca. 786-0 | 1.3 |
| Pancreatic ca. CAPAN 2 | 7.4 | Renal ca. A498 | 13.1 |
| Adrenal gland | 9.8 | Renal ca. RXF 393 | 0.9 |
| Thyroid | 13.4 | Renal ca. ACHN | 11.1 |
| Salivary gland | 3.3 | Renal ca. UO-31 | 24.7 |
| Pituitary gland | 16.8 | Renal ca. TK-10 | 6.7 |
| Brain (fetal) | 8.1 | Liver | 1.9 |
| Brain (whole) | 13.9 | Liver (fetal) | 6.5 |
| Brain (amygdala) | 16.0 | Liver ca. (hepatoblast) HepG2 | 0.4 |
| Brain (cerebellum) | 7.6 | Lung | 12.0 |
| Brain (hippocampus) | 72.7 | Lung (fetal) | 6.8 |
| Brain (substantia nigra) | 6.0 | Lung ca. (small cell) LX-1 | 1.6 |
| Brain (thalamus) | 16.2 | Lung ca. (small cell) NCI-H69 | 14.8 |
| Cerebral Cortex | 45.1 | Lung ca. (s.cell var.) SHP-77 | 14.3 |
| Spinal cord | 8.4 | Lung ca. (large cell)NCI-H460 | 8.0 |
| CNS ca. (glio/astro) U87-MG | 28.7 | Lung ca. (non-sm. cell) A549 | 12.1 |
| CNS ca. (glio/astro) U-118-MG | 34.4 | Lung ca. (non-s.cell) NCI-H23 | 18.9 |
| CNS ca. (astro) SW1783 | 14.8 | Lung ca (non-s.cell) HOP-62 | 13.1 |
| CNS ca.* (neuro; met) SK-N-AS | 56.3 | Lung ca. (non-s.cl) NCI-H522 | 10.4 |
| CNS ca. (astro) SF-539 | 14.2 | Lung ca. (squam.) SW 900 | 4.7 |
| CNS ca. (astro) SNB-75 | 21.5 | Lung ca. (squam.) NCI-H596 | 6.3 |
| CNS ca. (glio) SNB-19 | 7.9 | Mammary gland | 14.4 |
| CNS ca. (glio) U251 | 8.1 | Breast ca.* (pl. effusion) MCF-7 | 4.4 |
| CNS ca. (glio) SF-295 | 22.4 | Breast ca.* (pl.ef) MDA-MB-231 | 39.8 |
| Heart (fetal) | 12.0 | Breast ca.* (pl. effusion) T47D | 6.2 |
| Heart | 2.8 | Breast ca. BT-549 | 100.0 |
| Fetal Skeletal | 42.0 | Breast ca. MDA-N | 10.4 |
| Skeletal muscle | 4.6 | Ovary | 31.0 |
| Bone marrow | 5.8 | Ovarian ca. OVCAR-3 | 6.7 |
| Thymus | 5.6 | Ovarian ca. OVCAR-4 | 4.5 |
| Spleen | 9.0 | Ovarian ca. OVCAR-5 | 8.0 |
| Lymph node | 4.5 | Ovarian ca. OVCAR-8 | 2.1 |
| Colorectal | 13.9 | Ovarian ca. IGROV-1 | 0.9 |
| Stomach | 7.4 | Ovarian ca.* (ascites) SK-OV-3 | 13.3 |
| Small intestine | 8.4 | Uterus | 10.8 |
| Colon ca. SW480 | 8.1 | Placenta | 10.4 |
| Colon ca.* (SW480 met)SW620 | 2.5 | Prostate | 4.0 |
| Colon ca. HT29 | 0.5 | Prostate ca.* (bone met)PC-3 | 4.1 |
| Colon ca. HCT-116 | 5.6 | Testis | 14.2 |
| Colon ca. CaCo-2 | 0.1 | Melanoma Hs688(A).T | 10.6 |
| 83219 CC Well to Mod Diff | 6.7 | Melanoma* (met) Hs688(B).T | 4.2 |
| (ODO3866) | | | |
| Colon ca. HCC-2998 | 39.8 | Melanoma UACC-62 | 3.7 |
| Gastric ca.* (liver met) NCI-N87 | 1.4 | Melanoma M14 | 3.9 |
| Bladder | 3.5 | Melanoma LOX IMVI | 4.5 |
| Trachea | 12.9 | Melanoma* (met) SK-MEL-5 | 9.5 |
| Kidney | 5.1 | Adipose | 7.2 |

TABLE 62
Panel 2D

| | Relative | | Relative |
|---|---|---|---|
| | Expression(%) | | Expression(%) |
| | 2dtm4177f_{—} | | 2dtm4177f_{—} |
| Tissue Name | ag2177 | Tissue Name | ag2177 |
| Normal Colon GENPAK 061003 | 80.1 | Kidney NAT Clontech 8120608 | 24.7 |
| 83219 CC Well to Mod Diff | 9.1 | Kidney Cancer Clontech 8120613 | 31.9 |
| (ODO3866) | | | |
| 83220 CC NAT (ODO3866) | 16.3 | Kidney NAT Clontech 8120614 | 31.2 |
| 83221 CC Gr.2 rectosigmoid | 12.5 | Kidney Cancer Clontech 9010320 | 31.6 |
| (ODO3868) | | | |
| 83222 CC NAT (ODO3868) | 6.3 | Kidney NAT Clontech 9010321 | 43.2 |
| 83235 CC Mod Diff (ODO3920) | 7.7 | Normal Uterus GENPAK 061018 | 12.3 |
| 83236 CC NAT (ODO3920) | 21.6 | Uterus Cancer GENPAK 064011 | 35.8 |
| 83237 CC Gr.2 ascend colon | 27.7 | Normal Thyroid Clontech A+ | 26.2 |
| (ODO3921) | | 6570-1 | |
| 83238 CC NAT (ODO3921) | 23.5 | Thyroid Cancer GENPAK 064010 | 43.5 |
| 83241 CC from Partial | 25.7 | Thyroid Cancer INVITROGEN | 23.2 |
| Hepatectomy (ODO4309) | | A302152 | |
| 83242 Liver NAT (ODO4309) | 24.1 | Thyroid NAT INVITROGEN | 37.9 |
| | | A302153 | |
| 87472 Colon mets to lung | 10.3 | Normal Breast GENPAK 061019 | 35.1 |
| (OD04451-01) | | | |
| 87473 Lung NAT (OD04451-02) | 18.7 | 84877 Breast Cancer (OD04566) | 37.4 |
| Normal Prostate Clontech A+ | 9.4 | 85975 Breast Cancer (OD04590- | 53.6 |
| 6546-1 | | 01) | |
| 84140 Prostate Cancer (OD04410) | 41.8 | 85976 Breast Cancer Mets | 42.9 |
| | | (OD04590-03) | |
| 84141 Prostate NAT (OD04410) | 36.3 | 87070 Breast Cancer Metastasis | 45.7 |
| | | (OD04655-05) | |
| 87073 Prostate Cancer (OD04720- | 33.2 | GENPAK Breast Cancer 064006 | 19.9 |
| 01) | | | |
| 87074 Prostate NAT (OD04720- | 64.2 | Breast Cancer Res. Gen. 1024 | 42.3 |
| 02) | | | |
| Normal Lung GENPAK 061010 | 71.2 | Breast Cancer Clontech 9100266 | 46.3 |
| 83239 Lung Met to Muscle | 36.1 | Breast NAT Clontech 9100265 | 32.8 |
| (ODO4286) | | | |
| 83240 Muscle NAT (ODO4286) | 22.8 | Breast Cancer INVITROGEN | 26.6 |
| | | A209073 | |
| 84136 Lung Malignant Cancer | 48.0 | Breast NAT INVITROGEN | 23.5 |
| (OD03126) | | A2090734 | |
| 84137 Lung NAT (OD03126) | 53.6 | Normal Liver GENPAK 061009 | 9.4 |
| 84871 Lung Cancer (OD04404) | 31.6 | Liver Cancer GENPAK 064003 | 6.2 |
| 84872 Lung NAT (OD04404) | 26.2 | Liver Cancer Research Genetics | 10.7 |
| | | RNA 1025 | |
| 84875 Lung Cancer (OD04565) | 11.6 | Liver Cancer Research Genetics | 11.0 |
| | | RNA 1026 | |
| 84876 Lung NAT (OD04565) | 11.5 | Paired Liver Cancer Tissue | 12.2 |
| | | Research Genetics RNA 6004-T | |
| 85950 Lung Cancer (OD04237-01) | 66.0 | Paired Liver Tissue Research | 12.2 |
| | | Genetics RNA 6004-N | |
| 85970 Lung NAT (OD04237-02) | 23.2 | Paired Liver Cancer Tissue | 9.5 |
| | | Research Genetics RNA 6005-T | |
| 83255 Ocular Mel Met to Liver | 23.7 | Paired Liver Tissue Research | 5.3 |
| (ODO4310) | | Genetics RNA 6005-N | |
| 83256 Liver NAT (ODO4310) | 17.9 | Normal Bladder GENPAK 061001 | 30.4 |
| 84139 Melanoma Mets to Lung | 42.3 | Bladder Cancer Research Genetics | 11.0 |
| (OD04321) | | RNA 1023 | |
| 84138 Lung NAT (OD04321) | 42.0 | Bladder Cancer INVITROGEN | 16.3 |
| | | A302173 | |
| Normal Kidney GENPAK 061008 | 85.3 | 87071 Bladder Cancer (OD04718- | 56.6 |
| | | 01) | |
| 83786 Kidney Ca, Nuclear grade 2 | 100.0 | 87072 Bladder Normal Adjacent | 44.1 |
| (OD04338) | | (OD04718-03) | |
| 83787 Kidney NAT (OD04338) | 49.7 | Normal Ovary Res. Gen. | 19.3 |
| 83788 Kidney Ca Nuclear grade | 38.2 | Ovarian Cancer GENPAK 064008 | 31.6 |
| 1/2 (OD04339) | | | |
| 83789 Kidney NAT (OD04339) | 59.5 | 87492 Ovary Cancer (0D04768- | 34.6 |
| | | 07) | |
| 83790 Kidney Ca, Clear cell type | 68.8 | 87493 Ovary NAT (OD04768-08) | 8.2 |
| (OD04340) | | | |
| 83791 Kidney NAT (OD04340) | 50.7 | Normal Stomach GENPAK | 25.3 |
| | | 061017 | |
| 83792 Kidney Ca, Nuclear grade 3 | 13.5 | Gastric Cancer Clontech 9060358 | 12.2 |
| (OD04348) | | | |
| 83793 Kidney NAT (OD04348) | 25.9 | NAT Stomach Clontech 9060359 | 22.4 |
| 87474 Kidney Cancer (OD04622- | 23.5 | Gastric Cancer Clontech 9060395 | 30.8 |
| 01) | | | |
| 87475 Kidney NAT (OD04622-03) | 9.1 | NAT Stomach Clontech 9060394 | 24.8 |
| 85973 Kidney Cancer (OD04450- | 45.1 | Gastric Cancer Clontech 9060397 | 18.4 |
| 01) | | | |
| 85974 Kidney NAT (OD04450-03) | 37.4 | NAT Stomach Clontech 9060396 | 12.4 |
| Kidney Cancer Clontech 8120607 | 30.6 | Gastric Cancer GENPAK 064005 | 31.2 |

TABLE 63
Panel 4D

| | Relative | | Relative |
|---|---|---|---|
| | Expression(%) | | Expression(%) |
| | 4dtm4178f_{—} | | 4dtm4178f_{—} |
| Tissue Name | ag2177 | Tissue Name | ag2177 |
| 93768_Secondary Th1_anti- | 11.6 | 93100_HUVEC (Endothelial)_IL- | 25.0 |
| CD28/anti-CD3 | | 1b | |
| 93769_Secondary Th2_anti- | 42.9 | 93779_HUVEC (Endothelial)_IFN | 52.1 |
| CD28/anti-CD3 | | gamma | |
| 93770_Secondary Tr1_anti- | 39.8 | 93102_HUVEC | 35.1 |
| CD28/anti-CD3 | | (Endothelial)_TNF alpha + IFN | |
| | | gamma | |
| 93573_Secondary Th1_resting day | 16.7 | 93101_HUVEC | 41.8 |
| 4-6 in IL-2 | | (Endothelial)_TNF alpha + IL4 | |
| 93572_Secondary Th2_resting day | 24.8 | 93781_HUVEC (Endothelial)_IL- | 19.1 |
| 4-6 in IL-2 | | 11 | |
| 93571_Secondary Tr1_resting day | 19.2 | 93583_Lung Microvascular | 40.6 |
| 4-6 in IL-2 | | Endothelial Cells_none | |
| 93568_primary Th1_anti- | 44.4 | 93584_Lung Microvascular | 37.6 |
| CD28/anti-CD3 | | Endothelial Cells_TNFa (4 ng/ml) | |
| | | and IL1b (1 ng/ml) | |
| 93569_primary Th2_anti- | 43.2 | 92662_Microvascular Dermal | 58.2 |
| CD28/anti-CD3 | | endothelium_none | |
| 93570_primary Tr1_anti- | 60.7 | 92663_Microsvasular Dermal | 35.1 |
| CD28/anti-CD3 | | endothelium_TNFa (4 ng/ml) and | |
| | | IL1b (1 ng/ml) | |
| 93565_primary Th1_resting dy 4-6 | 84.7 | 93773_Bronchial | 31.6 |
| in IL-2 | | epithelium_TNFa (4 ng/ml) and | |
| | | IL1b (1 ng/ml)** | |
| 93566_primary Th2_resting dy 4-6 | 55.5 | 93347_Small Airway | 17.7 |
| in IL-2 | | Epithelium_none | |
| 93567_primary Tr1_resting dy 4-6 | 42.0 | 93348_Small Airway | 72.2 |
| in IL-2 | | Epithelium_TNFa (4 ng/ml) and | |
| | | IL1b (1 ng/ml) | |
| 93351_CD45RA CD4 | 31.0 | 92668_Coronery Artery | 38.4 |
| lymphocyte_anti-CD28/anti-CD3 | | SMC_resting | |
| 93352_CD45RO CD4 | 37.6 | 92669_Coronery Artery | 0.0 |
| lymphocyte_anti-CD28/anti-CD3 | | SMC_TNFa (4 ng/ml) and IL1b (1 ng/ml) | |
| 93251_CD8 Lymphocytes_anti- | 21.0 | 93107_astrocytes_resting | 27.9 |
| CD28/anti-CD3 | | | |
| 93353_chronic CD8 Lymphocytes | 24.0 | 93108_astrocytes_TNFa (4 ng/ml) | 17.2 |
| 2ry_resting dy 4-6 in IL-2 | | and IL1b (1 ng/ml) | |
| 93574_chronic CD8 Lymphocytes | 22.1 | 92666_KU-812 (Basophil)_resting | 22.8 |
| 2ry_activated CD3/CD28 | | | |
| 93354_CD4_none | 14.5 | 92667_KU-812 | 49.0 |
| | | (Basophil)_PMA/ionoycin | |
| 93252_Secondary | 30.1 | 93579_CCD1106 | 13.8 |
| Th1/Th2/Tr1_anti-CD95 CH11 | | (Keratinocytes)_none | |
| 93103_LAK cells_resting | 38.7 | 93580_CCD1106 | 5.6 |
| | | (Keratinocytes)_TNFa and IFNg** | |
| 93788_LAK cells_IL-2 | 29.5 | 93791_Liver Cirrhosis | 7.9 |
| 93787_LAK cells_IL-2 + IL-12 | 21.6 | 93792_Lupus Kidney | 4.6 |
| 93789_LAK cells_IL-2 + IFN | 38.7 | 93577_NCI-H292 | 48.6 |
| gamma | | | |
| 93790_LAK cells_IL-2 + IL-18 | 49.3 | 93358_NCI-H292_IL-4 | 63.3 |
| 93104_LAK | 17.4 | 93360_NCI-H292_IL-9 | 66.4 |
| cells_PMA/ionomycin and IL-18 | | | |
| 93578_NK Cells IL-2_resting | 25.3 | 93359_NCI-H292_IL-13 | 34.6 |
| 93109_Mixed Lymphocyte | 31.6 | 93357_NCI-H292_IFN gamma | 31.6 |
| Reaction_Two Way MLR | | | |
| 93110_Mixed Lymphocyte | 15.7 | 93777_HPAEC_- | 32.5 |
| Reaction_Two Way MLR | | | |
| 93111_Mixed Lymphocyte | 17.1 | 93778_HPAEC_IL-1 beta/TNA | 41.8 |
| Reaction_Two Way MLR | | alpha | |
| 93112_Mononuclear Cells | 10.0 | 93254_Normal Human Lung | 37.6 |
| (PBMCs)_resting | | Fibroblast_none | |
| 93113_Mononuclear Cells | 66.9 | 93253_Normal Human Lung | 24.0 |
| (PBMCs)_PWM | | Fibroblast_TNFa (4 ng/ml) and IL- | |
| | | 1b (1 ng/ml) | |
| 93114_Mononuclear Cells | 35.8 | 93257_Normal Human Lung | 71.7 |
| (PBMCs)_PHA-L | | Fibroblast_IL-4 | |
| 93249_Ramos (B cell)_none | 27.2 | 93256_Normal Human Lung | 55.5 |
| | | Fibroblast_IL-9 | |
| 93250_Ramos (B cell)_ionomycin | 62.0 | 93255_Normal Human Lung | 41.8 |
| | | Fibroblast_IL-13 | |
| 93349_B lymphocytes_PWM | 76.8 | 93258_Normal Human Lung | 61.1 |
| | | Fibroblast_IFN gamma | |
| 93350_B lymphoytes_CD40L and | 58.2 | 93106_Dermal Fibroblasts | 57.8 |
| IL-4 | | CCD1070_resting | |
| 92665_EOL-1 | 29.3 | 93361_Dermal Fibroblasts | 95.3 |
| (Eosinophil)_dbcAMP | | CCD1070_TNF alpha 4 ng/ml | |
| differentiated | | | |
| 93248_EOL-1 | 20.9 | 93105_Dermal Fibroblasts | 31.0 |
| (Eosinophil)_dbcAMP/PMAionomycin | | CCD1070_IL-1 beta 1 ng/ml | |
| 93356_Dendritic Cells_none | 24.8 | 93772_dermal fibroblast_IFN | 32.8 |
| | | gamma | |
| 93355_Dendritic Cells_LPS 100 ng/ml | 20.6 | 93771_dermal fibroblast_IL-4 | 70.2 |
| 93775_Dendritic Cells_anti-CD40 | 28.9 | 93260_IBD Colitis 2 | 2.2 |
| 93774_Monocytes_resting | 30.4 | 93261_IBD Crohns | 2.8 |
| 93776_Monocytes_LPS 50 ng/ml | 21.9 | 735010_Colon_normal | 30.1 |
| 93581_Macrophages_resting | 51.8 | 735019_Lung_none | 29.7 |
| 93582_Macrophages_LPS 100 ng/ml | 18.3 | 64028-1_Thymus_none | 49.3 |
| 93098_HUVEC | 52.9 | 64030-1_Kidney_none | 35.6 |
| (Endothelial)_none | | | |
| 93099_HUVEC | 100.0 | | |
| (Endothelial)_starved | | | |

TABLE 64
Panel CNS_neurodegeneration_v1.0

| | Relative | | Relative |
|---|---|---|---|
| | Expression(%) | | Expression(%) |
| | tm6900f_{—} | | tm6900f_{—} |
| Tissue Name | ag2177_a1s1 | Tissue Name | ag2177_a1s1 |
| AD 1 Hippo | 0.0 | Control (Path) 3 Temporal Ctx | 4.5 |
| AD 2 Hippo | 21.5 | Control (Path) 4 Temporal Ctx | 34.0 |
| AD 3 Hippo | 5.6 | AD 1 Occipital Ctx | 13.8 |
| AD 4 Hippo | 6.8 | AD 2 Occipital Ctx (Missing) | 0.0 |
| AD 5 Hippo | 66.5 | AD 3 Occipital Ctx | 6.4 |
| AD 6 Hippo | 34.0 | AD 4 Occipital Ctx | 29.1 |
| Control 2 Hippo | 38.5 | AD 5 Occipital Ctx | 45.7 |
| Control 4 Hippo | 11.4 | AD 6 Occipital Ctx | 0.1 |
| Control (Path) 3 Hippo | 0.0 | Control 1 Occipital Ctx | 4.5 |
| AD 1 Temporal Ctx | 11.4 | Control 2 Occipital Ctx | 69.7 |
| AD 2 Temporal Ctx | 38.6 | Control 3 Occipital Ctx | 16.1 |
| AD 3 Temporal Ctx | 4.8 | Control 4 Occipital Ctx | 8.2 |
| AD 4 Temporal Ctx | 0.0 | Control (Path) 1 Occipital Ctx | 70.8 |
| AD 5 Inf Temporal Ctx | 100.0 | Control (Path) 2 Occipital Ctx | 0.0 |
| AD 5 Sup Temporal Ctx | 30.4 | Control (Path) 3 Occipital Ctx | 4.2 |
| AD 6 Inf Temporal Ctx | 58.9 | Control (Path) 4 Occipital Ctx | 12.6 |
| AD 6 Sup Temporal Ctx | 36.5 | Control 1 Parietal Ctx | 7.8 |
| Control 1 Temporal Ctx | 9.7 | Control 2 Parietal Ctx | 28.9 |
| Control 2 Temporal Ctx | 57.5 | Control 3 Parietal Ctx | 0.0 |
| Control 3 Temporal Ctx | 14.4 | Control (Path) 1 Parietal Ctx | 82.8 |
| Control 3 Temporal Ctx | 7.6 | Control (Path) 2 Parietal Ctx | 24.3 |
| Control (Path) 1 Temporal Ctx | 58.9 | Control (Path) 3 Parietal Ctx | 4.0 |
| Control (Path) 2 Temporal Ctx | 30.2 | Control (Path) 4 Parietal Ctx | 33.8 |

Panel 1.3D Summary Expression of the NOV8 gene is ubiquitous among the samples in this panel, with highest expression in a sample derived from a breast cancer cell line (BT-549) (CT=27.4). In addition, there is substantial expression in a neuroblastoma cell line (SK-N-AS). Of note is the difference in expression between the sample of fetal skeletal muscle and its adult counterpart. Thus, the expression of this gene could be used to distinguish skeletal muscle from fetal and adult sources.

In addition, there is substantial expression of this gene in the hippocampus and cerebral cortex. Munc 18 appears to be critically involved in the process of synaptic vesicle docking prior to neurotransmitter release. Since the NOV8 gene product is a Munc 18 homolog, inhibition of the function of this protein may have therapeutic benefit in any disease in which selective decreases of neurotransmission has been shown to ameliorate symptomology (e.g., epilepsy or other seizure disorders, schizophrenia, bipolar disorder or anxiety).

The NOV8 gene encodes a putative intracellular protein with moderate expression in many metabolic tissues including adipose, adrenal gland, adult and fetal heart, adult and fetal liver, adult and fetal skeletal muscle, pancreas, pituitary and thyroid. Thus, this protein may be important for the pathogenesis and/or treatment of disease in any or all of these tissues, including obesity and diabetes.

Panel 2D Summary Expression of the NOV8 gene is ubiquitous among the samples in this panel, with highest expression in a kidney cancer sample (CT=27.1). Thus, the expression of this gene could be used to distinguish between this kidney cancer and the other samples in the panel.

Panel 4D Summary The NOV8 gene is widely expressed among the samples in this panel, with highest expression in endothelial (HUVEC) cells (CT=26.7). Significantly, expression of the NOV8 gene is higher in in activated B cells than in resting B cells. Small molecule drugs that inhibit the function of the NOV8 gene product may reduce the function of activated B cells and may reduce or eliminate the symptoms in patients with autoimmune and inflammatory diseases such as lupus erythematosus, Crohn's disease, ulcerative colitis, asthma, psoriasis, and rheumatoid arthritis.

Panel CNS_neurodegeneration_v1.0 Summary The NOV8 gene is widely expressed among the samples in this panel, with highest expression in the temporal cortex of an Alzheimer's patient (CT=26.6). While the expression of this gene does not appear be specific to Alzheimer's disease, this panel confirms expression at the moderate/high level in the brain in an additional set of individuals (Voets et al., Munc18-1 promotes large dense-core vesicle docking. Neuron. 31(4):581-91, 2001).

NOV9: Immunoglobulin-like

Expression of the NOV9a gene (SC138673511_A) and the NOV9b variant (CG106625-02) was assessed using the primer-probe set Ag2176 described in Table 65. Results from RTQ-PCR runs are shown in Tables 66-70.

TABLE 65

Probe Name Ag2176

| | | | | Start | SEQ ID |
|---|---|---|---|---|---|
| Primers | Sequences | TM | Length | Position | NO: |
| Forward | 5′-GGATAACGGCACCTACACTTG-3′ | 59.5 | 21 | 1006 | 194 |
| Probe | TET-5′-AGGCGTCCAATAAGCACGGCCAT-3′-TAMRA | 71.4 | 23 | 1029 | 195 |
| Reverse | 5′-CAGGGTCGTAGACCACAAGTAC-3′ | 58.7 | 22 | 1067 | 196 |

TABLE 66
Panel 1.3D

| | Relative | | Relative |
|---|---|---|---|
| | Expression(%) | | Expression(%) |
| | 1.3dtm4274t_{—} | | 1.3dtm4274t_{—} |
| Tissue Name | ag2176 | Tissue Name | ag2176 |
| Liver adenocarcinoma | 6.4 | Kidney (fetal) | 3.6 |
| Pancreas | 1.5 | Renal ca. 786-0 | 0.0 |
| Pancreatic ca. CAPAN 2 | 0.4 | Renal ca. A498 | 3.3 |
| Adrenal gland | 4.5 | Renal ca. RXF 393 | 0.1 |
| Thyroid | 1.0 | Renal ca. ACHN | 1.0 |
| Salivary gland | 1.3 | Renal ca. UO-31 | 2.8 |
| Pituitary gland | 8.7 | Renal ca. TK-10 | 1.7 |
| Brain (fetal) | 13.6 | Liver | 0.6 |
| Brain (whole) | 28.5 | Liver (fetal) | 0.7 |
| Brain (amygdala) | 35.4 | Liver ca. (hepatoblast) HepG2 | 2.5 |
| Brain (cerebellum) | 9.8 | Lung | 1.3 |
| Brain (hippocampus) | 100.0 | Lung (fetal) | 1.6 |
| Brain (substantia nigra) | 21.8 | Lung ca. (small cell) LX-1 | 0.4 |
| Brain (thalamus) | 48.0 | Lung ca. (small cell) NCI-H69 | 1.8 |
| Cerebral Cortex | 10.2 | Lung ca. (s.cell var.) SHP-77 | 2.3 |
| Spinal cord | 24.1 | Lung ca. (large cell)NCI-H460 | 0.7 |
| CNS ca. (glio/astro) U87-MG | 0.3 | Lung ca. (non-sm cell) A549 | 1.2 |
| CNS ca. (glio/astro) U-118-MG | 0.3 | Lung ca. (non-s.cell) NCI-H23 | 1.0 |
| CNS ca. (astro) SW1783 | 0.4 | Lung ca (non-s.cell) HOP-62 | 1.5 |
| CNS ca.* (neuro; met) SK-N-AS | 2.8 | Lung ca. (non-s.cl) NCI-H522 | 1.9 |
| CNS ca. (astro) SF-539 | 0.2 | Lung ca. (squam.) SW 900 | 1.5 |
| CNS ca. (astro) SNB-75 | 7.4 | Lung ca. (squam.) NCI-H596 | 0.1 |
| CNS ca. (glio) SNB-19 | 4.9 | Mammary gland | 4.3 |
| CNS ca. (glio) U251 | 2.4 | Breast ca.* (pl. effusion) MCF-7 | 0.8 |
| CNS ca. (glio) SF-295 | 3.0 | Breast ca.* (pl.ef) MDA-MB-231 | 0.3 |
| Heart (fetal) | 2.1 | Breast ca.* (pl. effusion) T47D | 0.5 |
| Heart | 0.8 | Breast ca. BT-549 | 3.4 |
| Fetal Skeletal | 4.6 | Breast ca. MDA-N | 0.3 |
| Skeletal muscle | 0.3 | Ovary | 1.4 |
| Bone marrow | 0.3 | Ovarian ca. OVCAR-3 | 1.0 |
| Thymus | 0.3 | Ovarian ca. OVCAR-4 | 1.0 |
| Spleen | 0.4 | Ovarian ca. OVCAR-5 | 2.3 |
| Lymph node | 0.2 | Ovarian ca. OVCAR-8 | 0.4 |
| Colorectal | 0.3 | Ovarian ca. IGROV-1 | 0.6 |
| Stomach | 2.6 | Ovarian ca.* (ascites) SK-OV-3 | 1.6 |
| Small intestine | 2.5 | Uterus | 2.0 |
| Colon ca. SW480 | 11.0 | Placenta | 0.1 |
| Colon ca.* (SW480 met)SW620 | 0.6 | Prostate | 4.7 |
| Colon ca. HT29 | 0.4 | Prostate ca.* (bone met)PC-3 | 0.8 |
| Colon ca. HCT-116 | 1.5 | Testis | 2.1 |
| Colon ca. CaCo-2 | 0.9 | Melanoma Hs688(A).T | 0.1 |
| 83219 CC Well to Mod Diff | 0.3 | Melanoma* (met) Hs688(B).T | 0.0 |
| (ODO3866) | | | |
| Colon ca. HCC-2998 | 1.0 | Melanoma UACC-62 | 1.1 |
| Gastric ca.* (liver met) NCI-N87 | 1.5 | Melanoma M14 | 0.0 |
| Bladder | 0.4 | Melanoma LOX IMVI | 0.0 |
| Trachea | 6.8 | Melanoma* (met) SK-MEL-5 | 0.7 |
| Kidney | 2.6 | Adipose | 0.1 |

TABLE 67
Panel 2D

| | Relative | | Relative |
|---|---|---|---|
| | Expression(%) | | Expression(%) |
| | 2dtm4275t_{—} | | 2dtm4275t_{—} |
| Tissue Name | ag2176 | Tissue Name | ag2176 |
| Normal Colon GENPAK 061003 | 31.0 | Kidney NAT Clontech 8120608 | 45.7 |
| 83219 CC Well to Mod Diff | 6.7 | Kidney Cancer Clontech 8120613 | 46.3 |
| (ODO3866) | | | |
| 83220 CC NAT (ODO3866) | 10.0 | Kidney NAT Clontech 8120614 | 66.0 |
| 83221 CC Gr.2 rectosigmoid | 1.4 | Kidney Cancer Clontech 9010320 | 11.2 |
| (ODO3868) | | | |
| 83222 CC NAT (ODO3868) | 6.7 | Kidney NAT Clontech 9010321 | 100.0 |
| 83235 CC Mod Diff (ODO3920) | 3.4 | Normal Uterus GENPAK 061018 | 5.0 |
| 83236 CC NAT (ODO3920) | 3.2 | Uterus Cancer GENPAK 064011 | 16.5 |
| 83237 CC Gr.2 ascend colon | 6.5 | Normal Thyroid Clontech A+ | 12.5 |
| (ODO3921) | | 6570-1 | |
| 83238 CC NAT (ODO3921) | 8.8 | Thyroid Cancer GENPAK 064010 | 6.2 |
| 83241 CC from Partial | 5.2 | Thyroid Cancer INVITROGEN | 1.0 |
| Hepatectomy (ODO4309) | | A302152 | |
| 83242 Liver NAT (ODO4309) | 8.2 | Thyroid NAT INVITROGEN | 14.7 |
| | | A302153 | |
| 87472 Colon mets to lung | 6.8 | Normal Breast GENPAK 061019 | 27.7 |
| (OD04451-01) | | | |
| 87473 Lung NAT (OD04451-02) | 4.9 | 84877 Breast Cancer (OD04566) | 17.4 |
| Normal Prostate Clontech A+ | 61.6 | 85975 Breast Cancer (OD04590- | 35.6 |
| 6546-1 | | 01) | |
| 84140 Prostate Cancer (OD04410) | 37.6 | 85976 Breast Cancer Mets | 38.2 |
| | | (OD04590-03) | |
| 84141 Prostate NAT (OD04410) | 29.7 | 87070 Breast Cancer Metastasis | 23.7 |
| | | (OD04655-05) | |
| 87073 Prostate Cancer (OD04720- | 28.7 | GENPAK Breast Cancer 064006 | 10.2 |
| 01) | | | |
| 87074 Prostate NAT (OD04720- | 67.8 | Breast Cancer Res. Gen. 1024 | 34.2 |
| 02) | | | |
| Normal Lung GENPAK 061010 | 14.0 | Breast Cancer Clontech 9100266 | 17.8 |
| 83239 Lung Met to Muscle | 3.1 | Breast NAT Clontech 9100265 | 19.9 |
| (ODO4286) | | | |
| 83240 Muscle NAT (ODO4286) | 5.2 | Breast Cancer INVITROGEN | 41.8 |
| | | A209073 | |
| 84136 Lung Malignant Cancer | 14.6 | Breast NAT INVITROGEN | 25.2 |
| (OD03126) | | A2090734 | |
| 84137 Lung NAT (OD03126) | 6.3 | Normal Liver GENPAK 061009 | 2.4 |
| 84871 Lung Cancer (OD04404) | 21.9 | Liver Cancer GENPAK 064003 | 1.2 |
| 84872 Lung NAT (OD04404) | 8.2 | Liver Cancer Research Genetics | 3.4 |
| | | RNA 1025 | |
| 84875 Lung Cancer (OD04565) | 13.5 | Liver Cancer Research Genetics | 2.8 |
| | | RNA 1026 | |
| 84876 Lung NAT (OD04565) | 3.7 | Paired Liver Cancer Tissue | 7.3 |
| | | Research Genetics RNA 6004-T | |
| 85950 Lung Cancer (OD04237-01) | 35.1 | Paired Liver Tissue Research | 1.0 |
| | | Genetics RNA 6004-N | |
| 85970 Lung NAT (OD04237-02) | 9.2 | Paired Liver Cancer Tissue | 1.9 |
| | | Research Genetics RNA 6005-T | |
| 83255 Ocular Mel Met to Liver | 6.7 | Paired Liver Tissue Research | 2.1 |
| (ODO4310) | | Genetics RNA 6005-N | |
| 83256 Liver NAT (ODO4310) | 6.2 | Normal Bladder GENPAK 061001 | 15.8 |
| 84139 Melanoma Mets to Lung | 60.7 | Bladder Cancer Research Genetics | 4.0 |
| (OD04321) | | RNA 1023 | |
| 84138 Lung NAT (OD04321) | 7.5 | Bladder Cancer INVITROGEN | 13.2 |
| | | A302173 | |
| Normal Kidney GENPAK 061008 | 35.1 | 87071 Bladder Cancer (OD04718- | 29.5 |
| | | 01) | |
| 83786 Kidney Ca, Nuclear grade 2 | 85.9 | 87072 Bladder Normal Adjacent | 4.7 |
| (OD04338) | | (OD04718-03) | |
| 83787 Kidney NAT (OD04338) | 28.7 | Normal Ovary Res. Gen. | 16.6 |
| 83788 Kidney Ca Nuclear grade | 6.8 | Ovarian Cancer GENPAK 064008 | 26.4 |
| 1/2 (OD04339) | | | |
| 83789 Kidney NAT (OD04339) | 64.6 | 87492 Ovary Cancer (OD04768- | 9.5 |
| | | 07) | |
| 83790 Kidney Ca, Clear cell type | 2.8 | 87493 Ovary NAT (OD04768-08) | 3.0 |
| (OD04340) | | | |
| 83791 Kidney NAT (OD04340) | 27.2 | Normal Stomach GENPAK | 15.9 |
| | | 061017 | |
| 83792 Kidney Ca, Nuclear grade 3 | 2.7 | Gastric Cancer Clontech 9060358 | 2.8 |
| (OD04348) | | | |
| 83793 Kidney NAT (OD04348) | 14.9 | NAT Stomach Clontech 9060359 | 9.6 |
| 87474 Kidney Cancer (OD04622- | 8.7 | Gastric Cancer Clontech 9060395 | 21.5 |
| 01) | | | |
| 87475 Kidney NAT (OD04622-03) | 9.9 | NAT Stomach Clontech 9060394 | 10.5 |
| 85973 Kidney Cancer (OD04450- | 36.9 | Gastric Cancer Clontech 9060397 | 25.7 |
| 01) | | | |
| 85974 Kidney NAT (OD04450-03) | 16.6 | NAT Stomach Clontech 9060396 | 5.1 |
| Kidney Cancer Clontech 8120607 | 20.2 | Gastric Cancer GENPAK 064005 | 6.9 |

TABLE 68
Panel 4D

| | Relative Expression(%) | |
|---|---|---|
| | 4dx4tm4234t_{—} | 4dtm4276t_{—} |
| Tissue Name | ag2176_a2 | ag2176 |
| 93768_Secondary Th1_anti-CD28/anti-CD3 | 0.8 | 0.9 |
| 93769_Secondary Th2_anti-CD28/anti-CD3 | 0.6 | 0.7 |
| 93770_Secondary Tr1_anti-CD28/anti-CD3 | 0.8 | 1.7 |
| 93573_Secondary Th1_resting day 4-6 in IL-2 | 0.2 | 1.3 |
| 93572_Secondary Th2_resting day 4-6 in IL-2 | 0.2 | 0.3 |
| 93571_Secondary Tr1_resting day 4-6 in IL-2 | 0.3 | 1.4 |
| 93568_primary Th1_anti-CD28/anti-CD3 | 0.4 | 0.8 |
| 93569_primary Th2_anti-CD28/anti-CD3 | 0.0 | 0.9 |
| 93570_primary Tr1_anti-CD28/anti-CD3 | 0.4 | 0.2 |
| 93565_primary Th1_resting dy 4-6 in IL-2 | 0.9 | 2.2 |
| 93566_primary Th2_resting dy 4-6 in IL-2 | 0.4 | 0.9 |
| 93567_primary Tr1_resting dy 4-6 in IL-2 | 0.1 | 0.7 |
| 93351_CD45RA CD4 lymphocyte_anti-CD28/anti-CD3 | 0.8 | 1.6 |
| 93352_CD45RO CD4 lymphocyte_anti-CD28/anti-CD3 | 0.6 | 0.5 |
| 93251_CD8 Lymphocytes_anti-CD28/anti-CD3 | 0.7 | 0.8 |
| 93353_chronic CD8 Lymphocytes 2ry_resting dy 4-6 in IL-2 | 0.4 | 0.3 |
| 93574_chronic CD8 Lymphocytes 2ry_activated CD3/CD28 | 0.0 | 1.1 |
| 93354_CD4_none | 0.1 | 0.2 |
| 93252_Secondary Th1/Th2/Tr1_anti-CD95 CH11 | 0.6 | 0.2 |
| 93103_LAK cells_resting | 0.7 | 1.0 |
| 93788_LAK cells_IL-2 | 0.3 | 0.5 |
| 93787_LAK cells_IL-2 + IL-12 | 0.4 | 0.4 |
| 93789_LAK cells_IL-2 + IFN gamma | 0.4 | 0.2 |
| 93790_LAK cells_IL-2 + IL-18 | 0.6 | 0.9 |
| 93104_LAK cells_PMA/ionomycin and IL-18 | 0.3 | 1.5 |
| 93578_NK Cells IL-2_resting | 0.2 | 1.2 |
| 93109_Mixed Lymphocyte Reaction_Two Way MLR | 0.6 | 1.1 |
| 93110_Mixed Lymphocyte Reaction_Two Way MLR | 0.4 | 0.2 |
| 93111_Mixed Lymphocyte Reaction_Two Way MLR | 0.5 | 0.2 |
| 93112_Mononuclear Cells (PBMCs)_resting | 0.1 | 0.1 |
| 93113_Mononuclear Cells (PBMCs)_PWM | 0.7 | 1.2 |
| 93114_Mononuclear Cells (PBMCs)_PHA-L | 0.2 | 0.5 |
| 93249_Ramos (B cell)_none | 0.0 | 0.0 |
| 93250_Ramos (B cell)_ionomycin | 0.0 | 0.3 |
| 93349_B lymphocytes_PWM | 0.8 | 0.6 |
| 93350_B lymphoytes_CD40L and IL-4 | 1.1 | 0.3 |
| 92665_EOL-1 (Eosinophil)_dbcAMP differentiated | 0.8 | 0.8 |
| 93248_EOL-1 (Eosinophil)_dbcAMP/PMAionomycin | 8.9 | 11.6 |
| 93356_Dendritic Cells_none | 0.7 | 0.0 |
| 93355_Dendritic Cells_LPS 100 ng/ml | 0.8 | 0.2 |
| 93775_Dendritic Cells_anti-CD40 | 0.9 | 0.4 |
| 93774_Monocytes_resting | 0.7 | 0.5 |
| 93776_Monocytes_LPS 50 ng/ml | 0.5 | 0.4 |
| 93581_Macrophages_resting | 0.0 | 0.8 |
| 93582_Macrophages_LPS 100 ng/ml | 0.4 | 0.2 |
| 93098_HUVEC (Endothelial)_none | 1.3 | 2.3 |
| 93099_HUVEC (Endothelial)_starved | 2.8 | 6.0 |
| 93100_HUVEC (Endothelial)_IL-1b | 1.0 | 1.8 |
| 93779_HUVEC (Endothelial)_IFN gamma | 3.6 | 6.9 |
| 93102_HUVEC (Endothelial)_TNF alpha + IFN gamma | 0.6 | 0.4 |
| 93101_HUVEC (Endothelial)_TNF alpha + IL4 | 1.6 | 2.6 |
| 93781_HUVEC (Endothelial)_IL-11 | 6.4 | 3.2 |
| 93583_Lung Microvascular Endothelial Cells_none | 2.5 | 5.0 |
| 93584_Lung Microvascular Endothelial Cells_TNFa (4 ng/ml) and | 1.3 | 1.6 |
| IL1b (1 ng/ml) | | |
| 92662_Microvascular Dermal endothelium_none | 5.3 | 4.9 |
| 92663_Microvascular Dermal endothelium_TNFa (4 ng/ml) and IL1b | 1.2 | 3.3 |
| (1 ng/ml) | | |
| 93773_Bronchial epithelium_TNFa (4 ng/ml) and IL1b (1 ng/ml)** | 1.7 | 7.3 |
| 93347_Small Airway Epithelium_none | 2.3 | 3.2 |
| 93348_Small Airway Epithelium_TNFa (4 ng/ml) and IL1b (1 ng/ml) | 6.3 | 7.9 |
| 92668_Coronery Artery SMC_resting | 0.5 | 1.7 |
| 92669_Coronery Artery SMC_TNFa (4 ng/ml) and IL1b (1 ng/ml) | 0.9 | 3.5 |
| 93107_astrocytes_resting | 100.0 | 100.0 |
| 93108_astrocytes_TNFa (4 ng/ml) and IL1b (1 ng/ml) | 70.6 | 65.5 |
| 92666_KU-812 (Basophil)_resting | 0.9 | 0.6 |
| 92667_KU-812 (Basophil)_PMA/ionoycin | 2.7 | 1.5 |
| 93579_CCD1106 (Keratinocytes)_none | 3.5 | 2.4 |
| 93580_CCD1106 (Keratinocytes)_TNFa and IFNg** | 0.8 | 3.6 |
| 93791_Liver Cirrhosis | 1.1 | 1.5 |
| 93792_Lupus Kidney | 2.7 | 2.1 |
| 93577_NCI-H292 | 8.7 | 9.6 |
| 93358_NCI-H292_IL-4 | 10.3 | 14.3 |
| 93360_NCI-H292_IL-9 | 10.4 | 15.5 |
| 93359_NCI-H292_IL-13 | 7.9 | 14.0 |
| 93357_NCI-H292_IFN gamma | 6.8 | 10.9 |
| 93777_HPAEC_- | 2.4 | 3.8 |
| 93778_HPAEC_IL-1 beta/TNA alpha | 2.1 | 3.1 |
| 93254_Normal Human Lung Fibroblast_none | 1.7 | 1.4 |
| 93253_Normal Human Lung Fibroblast_TNFa (4 ng/ml) and IL-1b (1 ng/ml) | 2.1 | 0.4 |
| 93257_Normal Human Lung Fibroblast_IL-4 | 1.2 | 2.8 |
| 93256_Normal Human Lung Fibroblast_IL-9 | 1.9 | 1.3 |
| 93255_Normal Human Lung Fibroblast_IL-13 | 0.9 | 3.9 |
| 93258_Normal Human Lung Fibroblast_IFN gamma | 2.4 | 2.0 |
| 93106_Dermal Fibroblasts CCD1070_resting | 0.7 | 1.3 |
| 93361_Dermal Fibroblasts CCD1070_TNF alpha 4 ng/ml | 1.1 | 0.9 |
| 93105_Dermal Fibroblasts CCD1070_IL-1 beta 1 ng/ml | 0.2 | 0.9 |
| 93772_dermal fibroblast_IFN gamma | 0.4 | 0.7 |
| 93771_dermal fibroblast_IL-4 | 0.9 | 0.0 |
| 93260_IBD Colitis 2 | 0.6 | 1.2 |
| 93261_IBD Crohns | 0.4 | 0.7 |
| 735010_Colon_normal | 3.3 | 6.2 |
| 735019_Lung_none | 4.6 | 4.4 |
| 64028-1_Thymus_none | 25.4 | 3.6 |
| 64030-1_Kidney_none | 2.7 | 5.8 |

TABLE 69
Panel CNS_1

| | Relative | | Relative |
|---|---|---|---|
| | Expression(%) | | Expression(%) |
| | CNS1x4tm6184t_{—} | | CNS1x4tm6184t_{—} |
| Tissue Name | ag2176_b1 | Tissue Name | ag2176_b1 |
| 102633_BA4 Control | 37.2 | 102662_Cing Gyr Parkinson's2 | 28.6 |
| 102634_BA7 Control | 40.3 | 102664_BA4 Huntington's | 29.3 |
| 102632_BA9 Control | 28.7 | 102665_BA7 Huntington's | 32.3 |
| 102635_BA17 Control | 27.7 | 102663_BA9 Huntington's | 44.6 |
| 102636_Glob Palladus Control | 32.9 | 102666_BA17 Huntington's | 28.3 |
| 102637_Sub Nigra Control | 59.6 | 102667_Sub Nigra Huntington's | 100.0 |
| 102638_Temp Pole Control | 10.7 | 102668_Temp Pole Huntington's | 30.3 |
| 102639_Cing Gyr Control | 40.2 | 102669_Cing Gyr Huntington's | 77.9 |
| 102641_BA4 Control2 | 48.9 | 102671_BA4 Huntington's2 | 4.8 |
| 102642_BA7 Control2 | 55.4 | 102672_BA7 Huntington's2 | 27.0 |
| 102640_BA9 Control2 | 56.1 | 102670_BA9 Huntington's2 | 12.9 |
| 102643_BA17 Control2 | 55.4 | 102673_BA17 Huntington's2 | 11.0 |
| 102644_Glob Palladus Control2 | 15.7 | 102674_Sub Nigra Huntington's2 | 44.3 |
| 102645_Sub Nigra Control2 | 72.5 | 102676_Cing Gyr Huntington's2 | 31.8 |
| 102646_Temp Pole Control2 | 39.5 | 102603_BA4 PSP | 13.2 |
| 102647_Cing Gyr Control2 | 27.5 | 102604_BA7 PSP | 27.3 |
| 102617_BA9 Alzheimer's | 9.7 | 102602_BA9 PSP | 24.5 |
| 102620_Glob Palladus Alzheimer's | 42.7 | 102605_BA17 PSP | 24.6 |
| 102622_Temp Pole Alzheimer's | 7.1 | 102606_Glob Palladus PSP | 6.2 |
| 102623_Cing Gyr Alzheimer's | 27.8 | 102607_Temp Pole PSP | 3.5 |
| 102625_BA4 Alzheimer's2 | 1.4 | 102608_Cing Gyr PSP | 23.1 |
| 102626_BA7 Alzheimer's2 | 6.5 | 102610_BA4 PSP2 | 40.9 |
| 102624_BA9 Alzheimer's2 | 11.1 | 102611_BA7 PSP2 | 26.1 |
| 102627_BA17 Alzheimer's2 | 5.4 | 102609_BA9 PSP2 | 10.1 |
| 102628_Glob Palladus | 12.8 | 102612_BA17 PSP2 | 11.8 |
| Alzheimer's2 | | | |
| 102629_Sub Nigra Alzheimer's2 | 23.1 | 102613_Glob Palladus PSP2 | 11.9 |
| 102630_Temp Pole Alzheimer's2 | 4.4 | 102614_Sub Nigra PSP2 | 17.0 |
| 102631_Cing Gyr Alzheimer's2 | 11.6 | 102615_Temp Pole PSP2 | 4.0 |
| 102649_BA4 Parkinson's | 31.4 | 102616_Cing Gyr PSP2 | 10.3 |
| 102650_BA7 Parkinson's | 13.4 | 102588_BA4 Depression | 10.9 |
| 102648_BA9 Parkinson's | 21.8 | 102589_BA7 Depression | 8.3 |
| 102651_BA17 Parkinson's | 37.8 | 102587_BA9 Depression | 6.2 |
| 102652_Glob Palladus Parkinson's | 60.3 | 102590_BA17 Depression | 15.7 |
| 102653_Temp Pole Parkinson's | 23.3 | 102591_Glob Palladus Depression | 9.4 |
| 102654_Cing Gyr Parkinson's | 35.4 | 102592_Sub Nigra Depression | 18.2 |
| 102656_BA4 Parkinson's2 | 44.2 | 102594_Cing Gyr Depression | 13.4 |
| 102657_BA7 Parkinson's2 | 26.2 | 102596_BA4 Depression2 | 5.8 |
| 102655_BA9 Parkinson's2 | 29.4 | 102595_BA9 Depression2 | 4.5 |
| 102658_BA17 Parkinson's2 | 34.7 | 102597_BA17 Depression2 | 21.1 |
| 102659_Glob Palladus | 26.5 | 102599_Sub Nigra Depression2 | 15.0 |
| Parkinson's2 | | | |
| 102660_Sub Nigra Parkinson's2 | 95.6 | 102600_Temp Pole Depression2 | 9.0 |
| 102661_Temp Pole Parkinson's2 | 19.7 | 102601_Cing Gyr Depression2 | 22.5 |

TABLE 70
Panel CNS_neurodegeneration_V1.0

| | Relative | | Relative |
|---|---|---|---|
| | Expression(%) | | Expression(%) |
| | tm7006t_{—} | | tm7006t_{—} |
| Tissue Name | ag2176_b1_s1 | Tissue Name | ag2176_b1_s1 |
| AD 1 Hippo | 15.4 | Control (Path) 3 Temporal Ctx | 7.2 |
| AD 2 Hippo | 42.7 | Control (Path) 4 Temporal Ctx | 22.1 |
| AD 3 Hippo | 7.0 | AD 1 Occipital Ctx | 16.7 |
| AD 4 Hippo | 10.4 | AD 2 Occipital Ctx (Missing) | 0.0 |
| AD 5 Hippo | 63.1 | AD 3 Occipital Ctx | 6.4 |
| AD 6 Hippo | 68.0 | AD 4 Occipital Ctx | 27.4 |
| Control 2 Hippo | 47.6 | AD 5 Occipital Ctx | 57.8 |
| Control 4 Hippo | 19.3 | AD 6 Occipital Ctx | 18.3 |
| Control (Path) 3 Hippo | 6.6 | Control 1 Occipital Ctx | 6.3 |
| AD 1 Temporal Ctx | 16.8 | Control 2 Occipital Ctx | 80.9 |
| AD 2 Temporal Ctx | 41.3 | Control 3 Occipital Ctx | 15.8 |
| AD 3 Temporal Ctx | 5.5 | Control 4 Occipital Ctx | 16.1 |
| AD 4 Temporal Ctx | 23.2 | Control (Path) 1 Occipital Ctx | 100.0 |
| AD 5 Inf Temporal Ctx | 96.7 | Control (Path) 2 Occipital Ctx | 8.3 |
| AD 5 Sup Temporal Ctx | 55.1 | Control (Path) 3 Occipital Ctx | 6.6 |
| AD 6 Inf Temporal Ctx | 57.5 | Control (Path) 4 Occipital Ctx | 9.7 |
| AD 6 Sup Temporal Ctx | 60.0 | Control 1 Parietal Ctx | 13.2 |
| Control 1 Temporal Ctx | 9.6 | Control 2 Parietal Ctx | 32.2 |
| Control 2 Temporal Ctx | 51.3 | Control 3 Parietal Ctx | 23.8 |
| Control 3 Temporal Ctx | 17.4 | Control (Path) 1 Parietal Ctx | 68.7 |
| Control 3 Temporal Ctx | 17.3 | Control (Path) 2 Parietal Ctx | 28.2 |
| Control (Path) 1 Temporal Ctx | 51.9 | Control (Path) 3 Parietal Ctx | 6.4 |
| Control (Path) 2 Temporal Ctx | 28.7 | Control (Path) 4 Parietal Ctx | 29.5 |

Panel 1.3D Summary Expression of the NOV9A gene is ubiquitous among the samples in this panel, with highest expression seen in the hippocampus (CT=25. 1). The highly brain-preferential pattern of expression of this gene indicates a role in normal adult brain function. Please see Panel CNS_{—}1 for further discussion of potential utility in the central nervous system.

Among tissues with metabolic function, the NOV9A gene has moderate expression in a variety of metabolic tissues including adrenal gland, adult and fetal heart, adult and fetal liver, adult and fetal skeletal muscle, pancreas and thyroid. Thus, this gene product may be a protein therapeutic for the treatment of diseases that affect these tissues, including obesity and diabetes.

Panel 2D Summary The NOV9A gene is widely expressed among the samples in this panel, with highest expression detected in normal kidney tissue adjacent to a kidney cancer (CT=28.7). In addition, other tissue samples derived from normal margin to kidney cancers appear to express this gene. Thus, the expression of this gene could be used to distinguish normal kidney tissue adjacent to malignant kidney. Moreover, therapeutic modulation of the expression or function of this gene or gene product, through the use of small molecule drugs, antibodies or protein therapeutics, might be of benefit in the treatment of kidney cancer.

Panel 4D Summary The results from two experiments with the same probe and primer set are in very good agreement, with highest expression in astrocytes (CTs=27-28). Furthermore, expression of the NOV9A gene, which encodes a putative immunoglobulin domain containing protein, is higher in activated astrocytes when compared to resting astrocytes. Activated astrocytes play a role in the pathogenesis of multiple sclerosis (See reference 1). Thus, the NOV9A gene product could be used as a protein therapeutic for the reduction or elimination of the symptoms in patients suffering from multiple sclerosis.

Panel CNS_{—}1 Summary This gene appears to be widely expressed across all regions of the brain and in all disease states represented in this panel. Apparent reduced expression in the primary motor strip (BA4 region) of an Alzheimer's brain suggests an association of the function of the NOV9A gene product with this neurodegenerative pathology. In addition, a less pronounced, but still evident, decreased expression of the NOV9A gene in the primary motor strip (BA4) of Huntington's disease brains suggests a possible general role in neurodegeneration in this brain region. The combination of brain-preferential expression and dysregulation in neurodegenerative diseases seen in this panel indicates potential utility of the NOV9A gene product as a specific mediator of CNS disorders. Therefore, agents that modulate the action of the protein encoded by the NOV9A gene may have utility in the treatment of CNS disorders.

Panel CNS_neurodegeneration_V1.0 Summary The NOV9A gene is expressed across all regions of the brain, with highest expression detected in the occipital cortex of a control patient (CT=25.5). The gene does not appear to be differentially expressed in Alzheimer's disease. Please see Panel CNS_{—}1 for potential utility in the central nervous system (Satoh and Kuroda Differing effects of IFN beta vs IFN gamma in MS: gene expression in cultured astrocytes. Neurology. 57:681-5, 2001).

NOV10a: Type II Cytokeratin

Expression of the NOV10a gene (GSAC055715.12_D) was assessed using the primer-probe set Ag1858 described in Table 71. Results from RTQ-PCR runs are shown in Tables 72 and 73.

TABLE 71

Probe Name Ag1858

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-ACATGTCTGTCATCCTGTCCAT-3′ | 59.3 | 22 | 952 | 197 |
| Probe | FAM-5′-CCTAGACCTGGACAGCATCATTGACG-3′-TAMRA | 68.9 | 26 | 989 | 198 |
| Reverse | 5′-TCTTCAAGGCAATCTCCTCATA-3′ | 58.9 | 22 | 1029 | 199 |

TABLE 72
Panel 1.3D

| | Relative | | Relative |
|---|---|---|---|
| | Expression(%) | | Expression(%) |
| | 1.3dtm4363f_{—} | | 1.3dtm4363f_{—} |
| Tissue Name | ag1858 | Tissue Name | ag1858 |
| Liver adenocarcinoma | 4.9 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.8 | Liver ca. (hepatoblast) HepG2 | 0.0 |
| Brain (cerebellum) | 0.8 | Lung | 0.0 |
| Brain (hippocampus) | 0.0 | Lung (fetal) | 0.0 |
| Brain (substantia nigra) | 0.0 | Lung ca. (small cell) LX-1 | 1.2 |
| Brain (thalamus) | 0.0 | Lung ca. (small cell) NCI-H69 | 0.0 |
| Cerebral Cortex | 0.0 | Lung ca. (s.cell var.) SHP-77 | 0.0 |
| Spinal cord | 1.5 | Lung ca. (large cell) NCI-H460 | 0.0 |
| CNS ca. (glio/astro) U87-MG | 0.0 | Lung ca. (non-sm. cell) A549 | 0.0 |
| CNS ca. (glio/astro) U-118-MG | 1.2 | Lung ca. (non-s.cell) NCI-H23 | 0.0 |
| CNS ca. (astro) SW1783 | 0.0 | Lung ca (non-s.cell) HOP-62 | 0.6 |
| CNS ca.* (neuro; met) SK-N-AS | 0.0 | Lung ca. (non-s.cl) NCI-H522 | 0.0 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 0.0 |
| CNS ca. (glio) U251 | 0.0 | Breast ca.* (pl. effusion) MCF-7 | 0.0 |
| CNS ca. (glio) SF-295 | 0.0 | Breast ca.* (pl.ef) MDA-MB-231 | 0.0 |
| Heart (fetal) | 0.0 | Breast ca.* (pl. effusion) T47D | 0.0 |
| Heart | 0.0 | Breast ca. BT-549 | 0.0 |
| Fetal Skeletal | 1.5 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.8 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 1.0 | Ovarian ca. IGROV-1 | 0.0 |
| Stomach | 0.0 | Ovarian ca.* (ascites) SK-OV-3 | 0.0 |
| Small intestine | 0.0 | Uterus | 0.0 |
| Colon ca. SW480 | 100.0 | Placenta | 0.0 |
| Colon ca.* (SW480 met)SW620 | 0.0 | Prostate | 0.0 |
| Colon ca. HT29 | 0.0 | Prostate ca.* (bone met)PC-3 | 0.0 |
| Colon ca. HCT-116 | 0.0 | Testis | 1.7 |
| Colon ca. CaCo-2 | 5.1 | Melanoma Hs688(A).T | 0.0 |
| 83219 CC Well to Mod Diff | 0.0 | Melanoma* (met) Hs688(B).T | 0.0 |
| (ODO3866) | | | |
| Colon ca. HCC-2998 | 0.0 | Melanoma UACC-62 | 0.0 |
| Gastric ca.* (liver met) NCI-N87 | 0.0 | Melanoma M14 | 1.2 |
| Bladder | 0.0 | Melanoma LOX IMVI | 0.0 |
| Trachea | 0.0 | Melanoma* (met) SK-MEL-5 | 0.0 |
| Kidney | 0.0 | Adipose | 0.5 |

TABLE 73
Panel 4D

| | Relative | | Relative |
|---|---|---|---|
| | Expression(%) | | Expression(%) |
| | 4dtm4365f_{—} | | 4dtm4365f_{—} |
| Tissue Name | ag1858 | Tissue Name | ag1858 |
| 93768_Secondary Th1_anti- | 0.0 | 93100_HUVEC (Endothelial)_IL- | 0.0 |
| CD28/anti-CD3 | | 1b | |
| 93769_Secondary Th2_anti- | 0.0 | 93779_HUVEC (Endothelial)_IFN | 0.0 |
| CD28/anti-CD3 | | gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting day | 0.0 | 93101_HUVEC | 0.0 |
| 4-6 in IL-2 | | (Endothelial)_TNF alpha + IL4 | |
| 93572_Secondary Th2_resting day | 0.0 | 93781_HUVEC (Endothelial)_IL- | 0.0 |
| 4-6 in IL-2 | | 11 | |
| 93571_Secondary Tr1_resting day | 0.0 | 93583_Lung Microvascular | 0.0 |
| 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 11.0 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) and | |
| | | IL1b (1 ng/ml) | |
| 93565_primary Th1_resting dy 4-6 | 3.5 | 93773_Bronchial | 0.0 |
| in IL-2 | | epithelium_TNFa (4 ng/ml) and | |
| | | IL1b (1 ng/ml) ** | |
| 93566_primary Th2_resting dy 4-6 | 1.6 | 93347_Small Airway | 0.0 |
| in IL-2 | | Epithelium_none | |
| 93567_primary Tr1_resting dy 4-6 | 9.0 | 93348_Small Airway | 20.4 |
| in IL-2 | | Epithelium_TNFa (4 ng/ml) and | |
| | | IL1b (1 ng/ml) | |
| 93351_CD45RA CD4 | 0.0 | 92668_Coronery Artery | 0.0 |
| lymphocyte_anti-CD28/anti-CD3 | | SMC_resting | |
| 93352_CD45RO CD4 | 0.0 | 92669_Coronery Artery | 0.0 |
| lymphocyte_anti-CD28/anti-CD3 | | SMC_TNFa (4 ng/ml) and IL1b (1 ng/ml) | |
| 93251_CD8 Lymphocytes_anti- | 0.0 | 93107_astrocytes_resting | 0.0 |
| CD28/anti-CD3 | | | |
| 93353_chronic CD8 Lymphocytes | 0.0 | 93108_astrocytes_TNFa (4 ng/ml) | 10.3 |
| 2ry_resting dy 4-6 in IL-2 | | and IL1b (1 ng/ml) | |
| 93574_chronic CD8 Lymphocytes | 0.0 | 92666_KU-812 (Basophil)_resting | 0.0 |
| 2ry_activated CD3/CD28 | | | |
| 93354_CD4_none | 2.0 | 92667_KU-812 | 0.0 |
| | | (Basophil)_PMA/ionoycin | |
| 93252_Secondary | 0.0 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and IFNg** | |
| 93788_LAK cells_IL-2 | 1.4 | 93791_Liver Cirrhosis | 33.0 |
| 93787_LAK cells_IL-2 + IL-12 | 20.3 | 93792_Lupus Kidney | 2.4 |
| 93789_LAK cells_IL-2 + IFN | 0.0 | 93577_NCI-H292 | 71.7 |
| gamma | | | |
| 93790_LAK cells_IL-2 + IL-18 | 0.0 | 93358_NCI-H292_IL-4 | 0.0 |
| 93104_LAK | 0.0 | 93360_NCI-H292_IL-9 | 0.0 |
| cells_PMA/ionomycin and IL-18 | | | |
| 93578_NK Cells IL-2_resting | 0.0 | 93359_NCI-H292_IL-13 | 0.0 |
| 93109_Mixed Lymphocyte | 0.0 | 93357_NCI-H292_IFN gamma | 0.0 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 0.0 |
| (PBMCs)_resting | | Fibroblast_none | |
| 93113_Mononuclear Cells | 0.0 | 93253_Normal Human Lung | 0.0 |
| (PBMCs)_PWM | | Fibroblast_TNFa (4 ng/ml) and IL- | |
| | | 1b (1 ng/ml) | |
| 93114_Mononuclear Cells | 0.0 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B cell)_ionomycin | 0.0 | 93255_Normal Human Lung | 100.0 |
| | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L and | 0.0 | 93106_Dermal Fibroblasts | 0.0 |
| IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/PMAionomycin | | CCD1070_IL-1 beta 1 ng/ml | |
| 93356_Dendritic Cells_none | 0.0 | 93772_dermal fibroblast_IFN | 0.0 |
| | | gamma | |
| 93355_Dendritic Cells_LPS 100 ng/ml | 0.0 | 93771_dermal fibroblast_IL-4 | 0.0 |
| 93775_Dendritic Cells_anti-CD40 | 0.0 | 93260_IBD Colitis 2 | 0.0 |
| 93774_Monocytes_resting | 10.6 | 93261_IBD Crohns | 0.0 |
| 93776_Monocytes_LPS 50 ng/ml | 0.0 | 735010_Colon_normal | 7.5 |
| 93581_Macrophages_resting | 0.0 | 735019_Lung_none | 29.1 |
| 93582_Macrophages_LPS 100 ng/ml | 0.0 | 64028-1_Thymus_none | 0.0 |
| 93098_HUVEC | 0.0 | 64030-1_Kidney_none | 74.2 |
| (Endothelial)_none | | | |
| 93099_HUVEC | 0.0 | | |
| (Endothelial)_starved | | | |

Panel 1.3D Summary Expression of the NOV10A gene is limited to a sample derived from a colon cancer cell line (CT=31.4) in this panel. Of note is the difference between SW480 cells and the genetically related metastatic variant, SW620, which showed much lower expression of this gene. Thus, the expression of this gene could be used to distinguish SW480 cells from the rest of the samples on the panel, and specifically the SW620 metastatic variant.

Panel 2.2 Summary Expression of the NOV1 OA gene is low/undetectable (Ct values >35) in all samples in Panels 2.2. (Data not shown.)

Panel 4D Summary Expression of the NOV1 OA gene in panel 4D is limited to IL-13 stimulated lung fibroblasts (CT=33.5) and the mucoepidermoid cell line NCI-H292. The NOV10A gene, a basic cytokeratin homolog, may be useful as a small molecule target for the discovery of therapeutics to reduce or eliminate the symptoms of lung diseases such as asthma, emphysema, and chronic obstructive pulmonary disease. Furthermore, the NOV10A gene product may be useful as a diagnostic marker for the cells in these diseases.

NOV10b: Type II Cytokeratin

Expression of the NOV10b gene (GSAC055715_C) was assessed using the primer-probe set Ag1857 described in Table 74. Results from RTQ-PCR runs are shown in Tables 75-77.

TABLE 74

Probe Name Ag1857

| | | | | | |
|---|---|---|---|---|---|
| | | | | Start | SEQ ID |
| Primers | Sequences | TM | Length | Position | NO: |

| | | | | | |
|---|---|---|---|---|---|
| Forward | 5′-ATCAGGTGCTAGAGACCAAGTG-3′ | 58.5 | 22 | 514 | 200 |
| Probe | TET-5′-CCTCCTACAGCAGCTGGACTTGAACA-3′-TAMRA | 68.8 | 26 | 539 | 201 |
| Reverse | 5′-ATAAATGGGCTCCAGCTTCTT-3′ | 59 | 21 | 573 | 202 |

TABLE 75
Panel 1.3D

| | Relative | | Relative |
|---|---|---|---|
| | Expression(%) | | Expression(%) |
| | 1.3dtm4357t_{—} | | 1.3dtm4357t_{—} |
| Tissue Name | ag1857 | Tissue Name | ag1857 |
| Liver adenocarcinoma | 0.0 | Kidney (fetal) | 0.3 |
| Pancreas | 0.0 | Renal ca. 786-0 | 0.0 |
| Pancreatic ca. CAPAN 2 | 0.3 | Renal ca. A498 | 0.0 |
| Adrenal gland | 0.6 | Renal ca. RXF 393 | 0.0 |
| Thyroid | 0.0 | Renal ca. ACHN | 0.0 |
| Salivary gland | 2.6 | Renal ca. UO-31 | 0.0 |
| Pituitary gland | 0.0 | Renal ca. TK-10 | 0.0 |
| Brain (fetal) | 0.0 | Liver | 0.7 |
| Brain (whole) | 0.3 | Liver (fetal) | 0.3 |
| Brain (amygdala) | 0.0 | Liver ca. (hepatoblast) HepG2 | 0.3 |
| Brain (cerebellum) | 0.4 | Lung | 6.7 |
| Brain (hippocampus) | 0.0 | Lung (fetal) | 0.8 |
| Brain (substantia nigra) | 0.0 | Lung ca. (small cell) LX-1 | 0.0 |
| Brain (thalamus) | 0.0 | Lung ca. (small cell) NCI-H69 | 0.0 |
| Cerebral Cortex | 0.6 | Lung ca. (s.cell var.) SHP-77 | 0.0 |
| Spinal cord | 0.0 | Lung ca. (large cell) NCI-H460 | 0.0 |
| CNS ca. (glio/astro) U87-MG | 0.0 | Lung ca. (non-sm. cell) A549 | 0.0 |
| CNS ca. (glio/astro) U-118-MG | 0.0 | Lung ca. (non-s.cell) NCI-H23 | 0.0 |
| CNS ca. (astro) SW1783 | 0.0 | Lung ca (non-s.cell) HOP-62 | 0.0 |
| CNS ca.* (neuro; met) SK-N-AS | 0.3 | Lung ca. (non-s.cl) NCI-H522 | 0.0 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 0.0 |
| CNS ca. (glio) U251 | 0.0 | Breast ca.* (pl. effusion) MCF-7 | 0.0 |
| CNS ca. (glio) SF-295 | 0.0 | Breast ca.* (pl.ef) MDA-MB-231 | 0.0 |
| Heart (fetal) | 0.0 | Breast ca.* (pl. effusion) T47D | 0.0 |
| Heart | 0.0 | Breast ca. BT-549 | 0.9 |
| Fetal Skeletal | 0.9 | Breast ca. MDA-N | 0.0 |
| Skeletal muscle | 0.0 | Ovary | 0.0 |
| Bone marrow | 2.5 | Ovarian ca. OVCAR-3 | 0.0 |
| Thymus | 1.8 | Ovarian ca. OVCAR-4 | 0.0 |
| Spleen | 6.8 | Ovarian ca. OVCAR-5 | 0.0 |
| Lymph node | 19.9 | Ovarian ca. OVCAR-8 | 0.0 |
| Colorectal | 0.0 | Ovarian ca. IGROV-1 | 0.4 |
| Stomach | 2.6 | Ovarian ca.* (ascites) SK-OV-3 | 0.0 |
| Small intestine | 22.5 | Uterus | 0.0 |
| Colon ca. SW480 | 0.9 | Placenta | 0.5 |
| Colon ca.* (SW480 met) SW620 | 0.0 | Prostate | 0.0 |
| Colon ca. HT29 | 0.0 | Prostate ca.* (bone met) PC-3 | 0.0 |
| Colon ca. HCT-116 | 0.0 | Testis | 100.0 |
| Colon ca. CaCo-2 | 0.0 | Melanoma Hs688(A).T | 0.0 |
| 83219 CC Well to Mod Diff | 0.0 | Melanoma* (met) Hs688(B).T | 0.0 |
| (ODO3866) | | | |
| Colon ca. HCC-2998 | 0.0 | Melanoma UACC-62 | 0.0 |
| Gastric ca.* (liver met) NCI-N87 | 0.0 | Melanoma M14 | 0.0 |
| Bladder | 0.0 | Melanoma LOX IMVI | 0.0 |
| Trachea | 3.8 | Melanoma* (met) SK-MEL-5 | 0.0 |
| Kidney | 0.0 | Adipose | 0.3 |

TABLE 76
Panel 2D

| | Relative | | Relative |
|---|---|---|---|
| | Expression(%) | | Expression(%) |
| | 2dtm4358t_{—} | | 2dtm4358t_{—} |
| Tissue Name | ag1857 | Tissue Name | ag1857 |
| Normal Colon GENPAK 061003 | 0.0 | Kidney NAT Clontech 8120608 | 0.0 |
| 83219 CC Well to Mod Diff | 6.2 | Kidney Cancer Clontech 8120613 | 5.0 |
| (ODO3866) | | | |
| 83220 CC NAT (ODO3866) | 11.4 | Kidney NAT Clontech 8120614 | 0.0 |
| 83221 CC Gr.2 rectosigmoid | 0.0 | Kidney Cancer Clontech 9010320 | 0.0 |
| (ODO3868) | | | |
| 83222 CC NAT (ODO3868) | 0.0 | Kidney NAT Clontech 9010321 | 0.0 |
| 83235 CC Mod Diff (ODO3920) | 5.6 | Normal Uterus GENPAK 061018 | 0.0 |
| 83236 CC NAT (ODO3920) | 0.0 | Uterus Cancer GENPAK 064011 | 0.0 |
| 83237 CC Gr.2 ascend colon | 0.0 | Normal Thyroid Clontech A+ | 4.3 |
| (ODO3921) | | 6570-1 | |
| 83238 CC NAT (ODO3921) | 50.0 | Thyroid Cancer GENPAK 064010 | 10.7 |
| 83241 CC from Partial | 11.6 | Thyroid Cancer INVITROGEN | 4.8 |
| Hepatectomy (ODO4309) | | A302152 | |
| 83242 Liver NAT (ODO4309) | 0.0 | Thyroid NAT INVITROGEN | 3.9 |
| | | A302153 | |
| 87472 Colon mets to lung | 0.0 | Normal Breast GENPAK 061019 | 6.0 |
| (OD04451-01) | | | |
| 87473 Lung NAT (OD04451-02) | 0.0 | 84877 Breast Cancer (OD04566) | 0.0 |
| Normal Prostate Clontech A+ | 0.0 | 85975 Breast Cancer (OD04590- | 0.0 |
| 6546-1 | | 01) | |
| 84140 Prostate Cancer (OD04410) | 0.0 | 85976 Breast Cancer Mets | 0.0 |
| | | (OD04590-03) | |
| 84141 Prostate NAT (OD04410) | 0.0 | 87070 Breast Cancer Metastasis | 85.3 |
| | | (OD04655-05) | |
| 87073 Prostate Cancer (OD04720- | 0.0 | GENPAK Breast Cancer 064006 | 5.5 |
| 01) | | | |
| 87074 Prostate NAT (OD04720- | 0.0 | Breast Cancer Res. Gen. 1024 | 46.3 |
| 02) | | | |
| Normal Lung GENPAK 061010 | 97.3 | Breast Cancer Clontech 9100266 | 92.7 |
| 83239 Lung Met to Muscle | 4.5 | Breast NAT Clontech 9100265 | 100.0 |
| (ODO4286) | | | |
| 83240 Muscle NAT (ODO4286) | 0.0 | Breast Cancer INVITROGEN | 19.2 |
| | | A209073 | |
| 84136 Lung Malignant Cancer | 7.0 | Breast NAT INVITROGEN | 0.0 |
| (OD03126) | | A2090734 | |
| 84137 Lung NAT (OD03126) | 17.6 | Normal Liver GENPAK 061009 | 11.7 |
| 84871 Lung Cancer (OD04404) | 6.5 | Liver Cancer GENPAK 064003 | 0.0 |
| 84872 Lung NAT (OD04404) | 0.0 | Liver Cancer Research Genetics | 0.0 |
| | | RNA 1025 | |
| 84875 Lung Cancer (OD04565) | 0.0 | Liver Cancer Research Genetics | 0.0 |
| | | RNA 1026 | |
| 84876 Lung NAT (OD04565) | 11.2 | Paired Liver Cancer Tissue | 0.0 |
| | | Research Genetics RNA 6004-T | |
| 85950 Lung Cancer (OD04237-01) | 0.0 | Paired Liver Tissue Research | 9.6 |
| | | Genetics RNA 6004-N | |
| 85970 Lung NAT (OD04237-02) | 0.0 | Paired Liver Cancer Tissue | 0.0 |
| | | Research Genetics RNA 6005-T | |
| 83255 Ocular Mel Met to Liver | 0.0 | Paired Liver Tissue Research | 0.0 |
| (ODO4310) | | Genetics RNA 6005-N | |
| 83256 Liver NAT (ODO4310) | 0.0 | Normal Bladder GENPAK 061001 | 1.4 |
| 84139 Melanoma Mets to Lung | 0.0 | Bladder Cancer Research Genetics | 36.3 |
| (OD04321) | | RNA 1023 | |
| 84138 Lung NAT (OD04321) | 0.0 | Bladder Cancer INVITROGEN | 68.3 |
| | | A302173 | |
| Normal Kidney GENPAK 061008 | 42.6 | 87071 Bladder Cancer (OD04718- | 0.0 |
| | | 01) | |
| 83786 Kidney Ca, Nuclear grade 2 | 0.0 | 87072 Bladder Normal Adjacent | 0.0 |
| (OD04338) | | (OD04718-03) | |
| 83787 Kidney NAT (OD04338) | 7.1 | Normal Ovary Res. Gen. | 0.0 |
| 83788 Kidney Ca Nuclear grade | 15.0 | Ovarian Cancer GENPAK 064008 | 5.9 |
| 1/2 (OD04339) | | | |
| 83789 Kidney NAT (OD04339) | 0.0 | 87492 Ovary Cancer (OD04768- | 0.0 |
| | | 07) | |
| 83790 Kidney Ca, Clear cell type | 0.0 | 87493 Ovary NAT (OD04768-08) | 0.0 |
| (OD04340) | | | |
| 83791 Kidney NAT (OD04340) | 0.0 | Normal Stomach GENPAK | 0.0 |
| | | 061017 | |
| 83792 Kidney Ca, Nuclear grade 3 | 0.0 | Gastric Cancer Clontech 9060358 | 0.0 |
| (OD04348) | | | |
| 83793 Kidney NAT (OD04348) | 8.1 | NAT Stomach Clontech 9060359 | 0.0 |
| 87474 Kidney Cancer (OD04622- | 0.0 | Gastric Cancer Clontech 9060395 | 4.2 |
| 01) | | | |
| 87475 Kidney NAT (OD04622-03) | 0.0 | NAT Stomach Clontech 9060394 | 13.3 |
| 85973 Kidney Cancer (OD04450- | 0.0 | Gastric Cancer Clontech 9060397 | 0.0 |
| 01) | | | |
| 85974 Kidney NAT (OD04450-03) | 0.0 | NAT Stomach Clontech 9060396 | 0.0 |
| Kidney Cancer Clontech 8120607 | 0.0 | Gastric Cancer GENPAK 064005 | 24.3 |

TABLE 77
Panel 4D

| | Relative | | Relative |
|---|---|---|---|
| | Expression(%) | | Expression(%) |
| | 4dtm4359t_{—} | | 4dtm4359t_{—} |
| Tissue Name | ag1857 | Tissue Name | ag1857 |
| 93768_Secondary Th1_anti- | 0.0 | 93100_HUVEC (Endothelial)_IL- | 0.0 |
| CD28/anti-CD3 | | 1b | |
| 93769_Secondary Th2_anti- | 0.0 | 93779_HUVEC (Endothelial)_IFN | 0.0 |
| CD28/anti-CD3 | | gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting day | 1.6 | 93101_HUVEC | 0.0 |
| 4-6 in IL-2 | | (Endothelial)_TNF alpha + IL4 | |
| 93572_Secondary Th2_resting day | 0.9 | 93781_HUVEC (Endothelial)_IL- | 0.0 |
| 4-6 in IL-2 | | 11 | |
| 93571_Secondary Tr1_resting day | 4.9 | 93583_Lung Microvascular | 1.9 |
| 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 1.6 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) and | |
| | | IL1b (1 ng/ml) | |
| 93565_primary Th1_resting dy 4-6 | 100.0 | 93773_Bronchial | 0.0 |
| in IL-2 | | epithelium_TNFa (4 ng/ml) and | |
| | | IL1b (1 ng/ml)** | |
| 93566_primary Th2 resting dy 4-6 | 42.3 | 93347_Small Airway | 0.0 |
| in IL-2 | | Epithelium_none | |
| 93567_primary Tr1_resting dy 4-6 | 15.0 | 93348_Small Airway | 0.0 |
| in IL-2 | | Epithelium_TNFa (4 ng/ml) and | |
| | | IL1b (1 ng/ml) | |
| 93351_CD45RA CD4 | 1.8 | 92668_Coronery Artery | 1.9 |
| lymphocyte_anti-CD28/anti-CD3 | | SMC_resting | |
| 93352_CD45RO CD4 | 9.2 | 92669_Coronery Artery | 0.0 |
| lymphocyte_anti-CD28/anti-CD3 | | SMC_TNFa (4 ng/ml) and IL1b (1 ng/ml) | |
| 93251_CD8 Lymphocytes_anti- | 2.2 | 93107_astrocytes_resting | 0.0 |
| CD28/anti-CD3 | | | |
| 93353_chronic CD8 Lymphocytes | 6.3 | 93108_astrocytes_TNFa (4 ng/ml) | 0.0 |
| 2ry_resting dy 4-6 in IL-2 | | and IL1b (1 ng/ml) | |
| 93574_chronic CD8 Lymphocytes | 0.0 | 92666_KU-812 (Basophil)_resting | 3.5 |
| 2ry_activated CD3/CD28 | | | |
| 93354_CD4_none | 81.2 | 92667_KU-812 | 1.9 |
| | | (Basophil)_PMA/ionoycin | |
| 93252_Secondary | 10.0 | 93579_CCD1106 | 0.0 |
| Th1/Th2/Tr1_anti-CD95 CH11 | | (Keratinocytes)_none | |
| 93103_LAK cells_resting | 9.6 | 93580_CCD1106 | 0.0 |
| | | (Keratinocytes)_TNFa and IFNg** | |
| 93788_LAK cells_IL-2 | 18.4 | 93791_Liver Cirrhosis | 7.0 |
| 93787_LAK cells_IL-2 + IL-12 | 1.8 | 93792_Lupus Kidney | 0.8 |
| 93789_LAK cells_IL-2 + IFN | 2.3 | 93577_NCI-H292 | 0.0 |
| gamma | | | |
| 93790_LAK cells_IL-2 + IL-18 | 3.9 | 93358_NCI-H292_IL-4 | 0.0 |
| 93104_LAK | 0.7 | 93360_NCI-H292_IL-9 | 0.0 |
| cells_PMA/ionomycin and IL-18 | | | |
| 93578_NK Cells IL-2_resting | 13.6 | 93359_NCI-H292_IL-13 | 0.0 |
| 93109_Mixed Lymphocyte | 19.5 | 93357_NCI-H292_IFN gamma | 0.0 |
| Reaction_Two Way MLR | | | |
| 93110_Mixed Lymphocyte | 1.0 | 93777_HPAEC_- | 0.0 |
| Reaction_Two Way MLR | | | |
| 93111_Mixed Lymphocyte | 10.2 | 93778_HPAEC_IL-1 beta/TNA | 0.0 |
| Reaction_Two Way MLR | | alpha | |
| 93112_Mononuclear Cells | 32.1 | 93254_Normal Human Lung | 0.0 |
| (PBMCs)_resting | | Fibroblast_none | |
| 93113_Mononuclear Cells | 2.2 | 93253_Normal Human Lung | 0.0 |
| (PBMCs)_PWM | | Fibroblast_TNFa (4 ng/ml) and IL- | |
| | | 1b (1 ng/ml) | |
| 93114_Mononuclear Cells | 3.1 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B cell)_ionomycin | 0.0 | 93255_Normal Human Lung | 0.0 |
| | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L and | 1.0 | 93106_Dermal Fibroblasts | 0.0 |
| IL-4 | | CCD1070_resting | |
| 92665_EOL-1 | 0.0 | 93361_Dermal Fibroblasts | 1.8 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/PMAionomycin | | CCD1070_IL-1 beta 1 ng/ml | |
| 93356_Dendritic Cells_none | 1.0 | 93772_dermal fibroblast_IFN | 0.0 |
| | | gamma | |
| 93355_Dendritic Cells_LPS 100 ng/ml | 0.0 | 93771_dermal fibroblast_IL-4 | 0.7 |
| 93775_Dendritic Cells_anti-CD40 | 0.0 | 93260_IBD Colitis 2 | 0.0 |
| 93774_Monocytes_resting | 2.7 | 93261_IBD Crohns | 2.2 |
| 93776_Monocytes_LPS 50 ng/ml | 0.0 | 735010_Colon_normal | 8.0 |
| 93581_Macrophages_resting | 4.8 | 735019_Lung_none | 2.8 |
| 93582_Macrophages_LPS 100 ng/ml | 0.0 | 64028-1_Thymus_none | 0.0 |
| 93098_HUVEC | 0.0 | 64030-1_Kidney_none | 17.2 |
| (Endothelial)_none | | | |
| 93099_HUVEC | 0.0 | | |
| (Endothelial)_starved | | | |

Panel 1.3D Summary Expression of the NOV10b gene is highest in the testis (CT=29.4), with low but significant expression also detected in the lung, lymph node and small intestine. Thus, the expression of the NOV10b gene could be used to distinguish testis from the other samples in the panel.

Panel 2D Summary Expression of the NOV10b gene is highest in a sample from normal breast tissue adjacent to a breast cancer (CT=32.9) with low but significant expression also detected in the breast cancer, bladder cancer, normal lung tissue, and a sample derived from normal tissue adjacent to a colon cancer. Thus, the expression of the NOV10b gene could be used to distinguish these tissue samples from the rest of the samples in the panel.

Panel 4D Summary Expression of the NOV10b gene is highest in resting Th1 lymphocytes (CT=30.7), with significant expression also seen in resting Th2 cells. Lower levels of expression levels are detected in activated Th1 and Th2 cells. The NOV10b gene encodes a basic cytokeratin homolog and may be useful as a small molecule target for the discovery of therapeutics that can reduce or eliminate the symptoms of autoimmune and inflammatory diseases such as lupus erythematosus, Crohn's disease, ulcerative colitis, asthma, psoriasis, and rheumatoid arthritis. Furthermore, the NOV10b gene product may be useful as a diagnostic marker for Th1 and Th2 lymphocytes.

The NOV10b gene and the NOV10c gene (please see next entry) that encode these cytokeratin homologs are both found in the cytokeratin locus on chromosome 12. However, while all cytokeratins to date have been identified as epithelial cell markers, these novel gene products are expressed in a restricted subset of T lymphocytes, as seen from the expression profile in Panel 4D. CLUSTALW alignment of these two novel lymphocyte cytokeratins is shown in Table 10G and demonstrates similarity along the entire length of the proteins.

NOV10c: Type II Cytokeratin

Expression of the NOV10c gene (GSAC055715_B) was assessed using the primer-probe set Ag1856 described in Table 78. Results from RTQ-PCR runs are shown in Tables 79-81.

TABLE 78

Probe Name Ag1856

| | | | | Start | SEQ ID |
|---|---|---|---|---|---|
| Primers | Sequences | TM | Length | Position | NO: |
| Forward | 5′-AGCTGTCCCTGGATATTGAGAT-3′ | 59.1 | 22 | 1271 | 203 |
| Probe | FAM-5′-CACCTACCGCAAGCTGCTGGAGG-3′-TAMRA | 71.2 | 23 | 1296 | 204 |
| Reverse | 5′-TGGTATATTCTCCGGACATCCT-3′ | 59.7 | 22 | 1330 | 205 |

TABLE 79
Panel 1.3D

| | Relative | | Relative |
|---|---|---|---|
| | Expression(%) | | Expression (%) |
| | 1.3dtm4360f_{—} | | 1.3dtm4360f_{—} |
| Tissue Name | ag1856 | Tissue Name | ag1856 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 3.3 |
| Adrenal gland | 2.4 | Renal ca. RXF 393 | 0.0 |
| Thyroid | 0.0 | Renal ca. ACHN | 0.0 |
| Salivary gland | 7.1 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 2.8 | Liver (fetal) | 3.2 |
| Brain (amygdala) | 0.0 | Liver ca. (hepatoblast) HepG2 | 0.0 |
| Brain (cerebellum) | 0.0 | Lung | 52.9 |
| Brain (hippocampus) | 1.7 | Lung (fetal) | 13.0 |
| Brain (substantia nigra) | 0.0 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s.cell var.) SHP-77 | 0.0 |
| Spinal cord | 1.2 | Lung ca. (large cell)NCI-H460 | 0.0 |
| CNS ca. (glio/astro) U87-MG | 0.0 | Lung ca. (non-sm. cell) A549 | 0.0 |
| CNS ca. (glio/astro) U-118-MG | 0.0 | Lung ca. (non-s.cell) NCI-H23 | 0.0 |
| CNS ca. (astro) SW1783 | 0.0 | Lung ca (non-s.cell) HOP-62 | 0.0 |
| CNS ca.* (neuro; met) SK-N-AS | 0.0 | Lung ca. (non-s.cl) NCI-H522 | 0.0 |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB-19 | 0.0 | Mammary gland | 1.5 |
| CNS ca. (glio) U251 | 0.0 | Breast ca.* (pl. effusion) MCF-7 | 0.0 |
| CNS ca. (glio) SF-295 | 0.0 | Breast ca.* (pl.ef) MDA-MB-231 | 0.0 |
| Heart (fetal) | 0.0 | Breast ca.* (pl. effusion) T47D | 0.0 |
| Heart | 0.0 | Breast ca. BT-549 | 0.0 |
| Fetal Skeletal | 2.1 | Breast ca. MDA-N | 0.0 |
| Skeletal muscle | 0.0 | Ovary | 0.0 |
| Bone marrow | 14.4 | Ovarian ca. OVCAR-3 | 0.0 |
| Thymus | 42.3 | Ovarian ca. OVCAR-4 | 0.0 |
| Spleen | 36.3 | Ovarian ca. OVCAR-5 | 0.0 |
| Lymph node | 59.0 | Ovarian ca. OVCAR-8 | 0.0 |
| Colorectal | 2.7 | Ovarian ca. IGROV-1 | 0.0 |
| Stomach | 5.7 | Ovarian ca.* (ascites) SK-OV-3 | 0.0 |
| Small intestine | 100.0 | Uterus | 0.0 |
| Colon ca. SW480 | 0.0 | Placenta | 3.0 |
| Colon ca.* (SW480 met) SW620 | 0.0 | Prostate | 0.0 |
| Colon ca. HT29 | 0.0 | Prostate ca.* (bone met)PC-3 | 0.0 |
| Colon ca. HCT-116 | 0.0 | Testis | 2.0 |
| Colon ca. CaCo-2 | 0.0 | Melanoma Hs688(A).T | 0.0 |
| 83219 CC Well to Mod Diff | 0.0 | Melanoma* (met) Hs688(B).T | 0.0 |
| (ODO3866) | | | |
| Colon ca. HCC-2998 | 0.0 | Melanoma UACC-62 | 0.0 |
| Gastric ca.* (liver met) NCI-N87 | 0.0 | Melanoma M14 | 0.0 |
| Bladder | 0.0 | Melanoma LOX IMVI | 0.0 |
| Trachea | 21.3 | Melanoma* (met) SK-MEL-5 | 0.0 |
| Kidney | 0.0 | Adipose | 0.0 |

TABLE 80
Panel 2D

| | Relative | | Relative |
|---|---|---|---|
| | Expression(%) | | Expression(%) |
| | 2dtm4361f_{—} | | 2dtm4361f_{—} |
| Tissue Name | ag1856 | Tissue Name | ag1856 |
| Normal Colon GENPAK 061003 | 0.0 | Kidney NAT Clontech 8120608 | 0.0 |
| 83219 CC Well to Mod Diff | 0.0 | Kidney Cancer Clontech 8120613 | 0.0 |
| (ODO3866) | | | |
| 83220 CC NAT (ODO3866) | 19.5 | Kidney NAT Clontech 8120614 | 0.0 |
| 83221 CC Gr.2 rectosigmoid | 0.0 | Kidney Cancer Clontech 9010320 | 0.0 |
| (ODO3868) | | | |
| 83222 CC NAT (ODO3868) | 0.0 | Kidney NAT Clontech 9010321 | 0.0 |
| 83235 CC Mod Diff (ODO3920) | 0.0 | Normal Uterus GENPAK 061018 | 0.0 |
| 83236 CC NAT (ODO3920) | 2.7 | Uterus Cancer GENPAK 064011 | 0.0 |
| 83237 CC Gr.2 ascend colon | 0.0 | Normal Thyroid Clontech A+ | 6.7 |
| (ODO3921) | | 6570-1 | |
| 83238 CC NAT (ODO3921) | 13.2 | Thyroid Cancer GENPAK 064010 | 0.0 |
| 83241 CC from Partial | 12.5 | Thyroid Cancer INVITROGEN | 0.0 |
| Hepatectomy (ODO4309) | | A302152 | |
| 83242 Liver NAT (ODO4309) | 0.0 | Thyroid NAT INVITROGEN | 0.0 |
| | | A302153 | |
| 87472 Colon mets to lung | 12.6 | Normal Breast GENPAK 061019 | 6.8 |
| (OD04451-01) | | | |
| 87473 Lung NAT (OD04451-02) | 0.0 | 84877 Breast Cancer (OD04566) | 0.0 |
| Normal Prostate Clontech A+ | 0.0 | 85975 Breast Cancer (OD04590- | 0.0 |
| 6546-1 | | 01) | |
| 84140 Prostate Cancer (OD04410) | 0.0 | 85976 Breast Cancer Mets | 18.7 |
| | | (OD04590-03) | |
| 84141 Prostate NAT (OD04410) | 7.5 | 87070 Breast Cancer Metastasis | 85.9 |
| | | (OD04655-05) | |
| 87073 Prostate Cancer (OD04720- | 0.0 | GENPAK Breast Cancer 064006 | 6.2 |
| 01) | | | |
| 87074 Prostate NAT (OD04720- | 0.0 | Breast Cancer Res. Gen. 1024 | 0.0 |
| 02) | | | |
| Normal Lung GENPAK 061010 | 55.9 | Breast Cancer Clontech 9100266 | 62.0 |
| 83239 Lung Met to Muscle | 0.0 | Breast NAT Clontech 9100265 | 100.0 |
| (ODO4286) | | | |
| 83240 Muscle NAT (ODO4286) | 0.0 | Breast Cancer INVITROGEN | 0.0 |
| | | A209073 | |
| 84136 Lung Malignant Cancer | 0.0 | Breast NAT INVITROGEN | 0.0 |
| (OD03126) | | A2090734 | |
| 84137 Lung NAT (OD03126) | 10.7 | Normal Liver GENPAK 061009 | 0.0 |
| 84871 Lung Cancer (OD04404) | 6.7 | Liver Cancer GENPAK 064003 | 0.0 |
| 84872 Lung NAT (OD04404) | 11.0 | Liver Cancer Research Genetics | 0.0 |
| | | RNA 1025 | |
| 84875 Lung Cancer (OD04565) | 0.0 | Liver Cancer Research Genetics | 0.0 |
| | | RNA 1026 | |
| 84876 Lung NAT (OD04565) | 20.4 | Paired Liver Cancer Tissue | 0.0 |
| | | Research Genetics RNA 6004-T | |
| 85950 Lung Cancer (OD04237-01) | 0.0 | Paired Liver Tissue Research | 0.0 |
| | | Genetics RNA 6004-N | |
| 85970 Lung NAT (OD04237-02) | 5.0 | Paired Liver Cancer Tissue | 0.0 |
| | | Research Genetics RNA 6005-T | |
| 83255 Ocular Mel Met to Liver | 0.0 | Paired Liver Tissue Research | 0.0 |
| (ODO4310) | | Genetics RNA 6005-N | |
| 83256 Liver NAT (ODO4310) | 0.0 | Normal Bladder GENPAK 061001 | 0.0 |
| 84139 Melanoma Mets to Lung | 0.0 | Bladder Cancer Research Genetics | 31.2 |
| (OD04321) | | RNA 1023 | |
| 84138 Lung NAT (OD04321) | 8.5 | Bladder Cancer INVITROGEN | 41.5 |
| | | A302173 | |
| Normal Kidney GENPAK 061008 | 6.7 | 87071 Bladder Cancer (OD04718- | 0.0 |
| | | 01) | |
| 83786 Kidney Ca, Nuclear grade 2 | 0.0 | 87072 Bladder Normal Adjacent | 18.6 |
| (OD04338) | | (OD04718-03) | |
| 83787 Kidney NAT (OD04338) | 0.0 | Normal Ovary Res. Gen. | 0.0 |
| 83788 Kidney Ca Nuclear grade | 0.0 | Ovarian Cancer GENPAK 064008 | 4.2 |
| 1/2 (OD04339) | | | |
| 83789 Kidney NAT (OD04339) | 0.0 | 87492 Ovary Cancer (OD04768- | 0.0 |
| | | 07) | |
| 83790 Kidney Ca, Clear cell type | 0.0 | 87493 Ovary NAT (OD04768-08) | 0.0 |
| (OD04340) | | | |
| 83791 Kidney NAT (OD04340) | 0.0 | Normal Stomach GENPAK | 6.7 |
| | | 061017 | |
| 83792 Kidney Ca, Nuclear grade 3 | 6.5 | Gastric Cancer Clontech 9060358 | 0.0 |
| (OD04348) | | | |
| 83793 Kidney NAT (OD04348) | 17.2 | NAT Stomach Clontech 9060359 | 0.0 |
| 87474 Kidney Cancer (OD04622- | 0.0 | Gastric Cancer Clontech 9060395 | 0.0 |
| 01) | | | |
| 87475 Kidney NAT (OD04622-03) | 0.0 | NAT Stomach Clontech 9060394 | 6.0 |
| 85973 Kidney Cancer (OD04450- | 0.0 | Gastric Cancer Clontech 9060397 | 0.0 |
| 01) | | | |
| 85974 Kidney NAT (OD04450-03) | 8.0 | NAT Stomach Clontech 9060396 | 0.0 |
| Kidney Cancer Clontech 8120607 | 0.0 | Gastric Cancer GENPAK 064005 | 5.3 |

TABLE 81
Panel 4D

| | Relative | | Relative |
|---|---|---|---|
| | Expression (%) | | Expression (%) |
| | 4dtm4362f_{—} | | 4dtm4362f_{—} |
| Tissue Name | ag1856 | Tissue Name | ag1856 |
| 93768_Secondary Th1_anti- | 0.0 | 93100_HUVEC (Endothelial)_IL- | 0.0 |
| CD28/anti-CD3 | | 1b | |
| 93769_Secondary Th2_anti- | 0.0 | 93779_HUVEC (Endothelial)_IFN | 0.0 |
| CD28/anti-CD3 | | gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting day | 1.4 | 93101_HUVEC | 0.0 |
| 4-6 in IL-2 | | (Endothelial)_TNF alpha + IL4 | |
| 93572_Secondary Th2_resting day | 3.6 | 93781_HUVEC (Endothelial)_IL- | 0.0 |
| 4-6 in IL-2 | | 11 | |
| 93571_Secondary Tr1_resting day | 1.3 | 93583_Lung Microvascular | 0.0 |
| 4-6 in IL-2 | | Endothelial Cells_none | |
| 93568_primary Th1_anti- | 0.0 | 93584_Lung Microvascular | 1.2 |
| CD28/anti-CD3 | | Endothelial Cells_TNFa (4 ng/ml) | |
| | | and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 0.0 | 92663_Microsvasular Dermal | 0.0 |
| CD28/anti-CD3 | | endothelium_TNFa (4 ng/ml) and | |
| | | IL1b (1 ng/ml) | |
| 93565_primary Th1_resting dy 4-6 | 99.3 | 93773_Bronchial | 0.0 |
| in IL-2 | | epithelium_TNFa (4 ng/ml) and | |
| | | IL1b (1 ng/ml) ** | |
| 93566_primary Th2_resting dy 4-6 | 57.4 | 93347_Small Airway | 0.0 |
| in IL-2 | | Epithelium_none | |
| 93567_primary Tr1_resting dy 4-6 | 25.2 | 93348_Small Airway | 0.0 |
| in IL-2 | | Epithelium_TNFa (4 ng/ml) and | |
| | | IL1b (1 ng/ml) | |
| 93351_CD45RA CD4 | 4.6 | 92668_Coronery Artery | 0.0 |
| lymphocyte_anti-CD28/anti-CD3 | | SMC_resting | |
| 93352_CD45RO CD4 | 7.0 | 92669_Coronery Artery | 0.0 |
| lymphocyte_anti-CD28/anti-CD3 | | SMC_TNFa (4 ng/ml) and IL1b (1 | |
| | | ng/ml) | |
| 93251_CD8 Lymphocytes_anti- | 2.8 | 93107_astrocytes_resting | 0.0 |
| CD28/anti-CD3 | | | |
| 93353_chronic CD8 Lymphocytes | 3.1 | 93108_astrocytes_TNFa (4 ng/ml) | 0.0 |
| 2ry_resting dy 4-6 in IL-2 | | and IL1b (1 ng/ml) | |
| 93574_chronic CD8 Lymphocytes | 0.0 | 92666_KU-812 (Basophil)_resting | 6.4 |
| 2ry_activated CD3/CD28 | | | |
| 93354_CD4_none | 100.0 | 92667_KU-812 | 3.0 |
| | | (Basophil)_PMA/ionoycin | |
| 93252_Secondary | 20.4 | 93579_CCD1106 | 0.0 |
| Th1/Th2/Tr1_anti-CD95 CH11 | | (Keratinocytes)_none | |
| 93103_LAK cells_resting | 11.4 | 93580_CCD1106 | 0.0 |
| | | (Keratinocytes)_TNFa and IFNg ** | |
| 93788_LAK cells_IL-2 | 49.0 | 93791_Liver Cirrhosis | 1.3 |
| 93787_LAK cells_IL-2 + IL-12 | 5.6 | 93792_Lupus Kidney | 0.0 |
| 93789_LAK cells_IL-2 + IFN | 8.1 | 93577_NCI-H292 | 0.0 |
| gamma | | | |
| 93790_LAK cells_IL-2 + IL-18 | 3.8 | 93358_NCI-H292_IL-4 | 0.0 |
| 93104_LAK | 1.0 | 93360_NCI-H292_IL-9 | 0.0 |
| cells_PMA/ionomycin and IL-18 | | | |
| 93578_NK Cells IL-2_resting | 17.1 | 93359_NCI-H292_IL-13 | 0.0 |
| 93109_Mixed Lymphocyte | 20.7 | 93357_NCI-H292_IFN gamma | 0.0 |
| Reaction_Two Way MLR | | | |
| 93110_Mixed Lymphocyte | 4.6 | 93777_HPAEC_- | 0.0 |
| Reaction_Two Way MLR | | | |
| 93111_Mixed Lymphocyte | 9.7 | 93778_HPAEC_IL-1 beta/TNA | 0.0 |
| Reaction_Two Way MLR | | alpha | |
| 93112_Mononuclear Cells | 36.9 | 93254_Normal Human Lung | 0.0 |
| (PBMCs)_resting | | Fibroblast_none | |
| 93113_Mononuclear Cells | 0.7 | 93253_Normal Human Lung | 0.0 |
| (PBMCs)_PWM | | Fibroblast_TNFa (4 ng/ml) and IL- | |
| | | 1b (1 ng/ml) | |
| 93114_Mononuclear Cells | 0.8 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B cell)_ionomycin | 0.0 | 93255_Normal Human Lung | 0.0 |
| | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L and | 0.0 | 93106_Dermal Fibroblasts | 0.0 |
| IL-4 | | CCD1070_resting | |
| 92665_EOL-1 | 0.0 | 93361_Dermal Fibroblasts | 1.7 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/PMAionomycin | | CCD1070_IL-1 beta 1 ng/ml | |
| 93356_Dendritic Cells_none | 0.7 | 93772_dermal fibroblast_IFN | 0.0 |
| | | gamma | |
| 93355_Dendritic Cells_LPS 100 | 0.0 | 93771_dermal fibroblast_IL-4 | 0.0 |
| ng/ml | | | |
| 93775_Dendritic Cells_anti-CD40 | 0.0 | 93260_IBD Colitis 2 | 0.0 |
| 93774_Monocytes_resting | 1.2 | 93261_IBD Crohns | 0.3 |
| 93776_Monocytes_LPS 50 ng/ml | 0.6 | 735010_Colon_normal | 2.8 |
| 93581_Macrophages_resting | 3.7 | 735019_Lung_none | 1.7 |
| 93582_Macrophages_LPS 100 | 0.0 | 64028-1_Thymus_none | 0.0 |
| ng/ml | | | |
| 93098_HUVEC | 0.0 | 64030-1_Kidney_none | 19.5 |
| (Endothelial)_none | | | |
| 93099_HUVEC | 0.0 | | |
| (Endothelial)_starved | | | |

Panel 1.3D Summary Highest expression of the NOV10c gene is detected in the small intestine (CT=31.4). The expression of this gene is highest in a sample derived from small intestine. In addition, this gene has an expression pattern restricted to lymph node, bone marrow, spleen, thymus, trachea, adult lung and fetal lung tissue. Thus the expression of this gene could be used to distinguish these tissues from other tissues in the panel.

Panel 2D Summary Highest expression of the NOV10c gene is seen in normal breast tissue adjacent to a breast cancer (CT=31.8). Low, but significant expression is also seen in normal lung tissue, as is seen in panel 1.3D and in some breast cancer samples. Thus, the expression of this gene could be used to distinguish these tissue samples from the rest of the samples in the panel. Moreover, therapeutic modulation of the expression of this gene or the function of its protein product, through the use of small molecule drugs, antibodies or protein therapeutics, might be of benefit for the treatment of breast cancer.

Panel 4D Summary Highest expression of the gene is seen in untreated CD4 cells (CT=28.8). Significant expression is also seen in resting Th1 and Th2 lymphocytes, with lower levels of expression in activated Th1 and Th2 cells. The NOV10c gene encodes a cytokeratin homolog and may be useful as a small molecule target for the discovery of therapeutics that can reduce or eliminate the symptoms of autoimmune and inflammatory diseases such as lupus erythematosus, Crohn's disease, ulcerative colitis, asthma, psoriasis, and rheumatoid arthritis. Furthermore, the NOV10c gene may be useful as a diagnostic marker for Th1 and Th2 lymphocytes.

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46 and/or 48;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46 and/or 48, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46 and/or 48; and
(d) a variant of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46 and/or 48 wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46 and/or 48.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45 and/or 47.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46 and/or 48;
(b) a variant of a mature form of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46 and/or 48, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46 and/or 48;
(d) a variant of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46 and/or 48, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, 26, 28, 30, 32, 34, 36, 38, 40, 42, 44, 46 and/or 48, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45 and/or 47.

9. The nucleic acid molecule of claim 5, wherein said nucleic acid molecule comprises a nucleotide sequence selected from the group consisting of
(a) a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45 and/or 47;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45 and/or 47, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS: 1, 3, 5, 7, 9, 11, 13, 15, 17, 19, 21, 23, 25, 27, 29, 31, 33, 35, 37, 39, 41, 43, 45 and/or 47, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

24. The method of claim 23, wherein said subject is a human.

25. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

26. The method of claim 25, wherein said subject is a human.

27. A method of treating or preventing a NOVX-associated disorder, said method comprising administering to a subject in which such treatment or prevention is desired the antibody of claim 15 in an amount sufficient to treat or prevent said NOVX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a NOVX-associated disorder, wherein said therapeutic is selected from the group consisting of a NOVX polypeptide, a NOVX nucleic acid, and a NOVX antibody.

36. A method for screening for a modulator of activity or of latency or predisposition to a NOVX-associated disorder, said method comprising:
(a) administering a test compound to a test animal at increased risk for a NOVX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a NOVX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24, 26, 28, 30, 32, 34, 36, 38, 40, 42, 44, 46 and/or 48, or a biologically active fragment thereof.

41. A method of treating a pathological state in a mammal, the method comprising administering to the mammal the antibody, of claim 15 in an amount sufficient to alleviate the pathological state.
